(19) United States
(12) Patent Application Publication
(10) Pub. No.: US 2021/0284702 A1
(43) Pub. Date: Sep. 16, 2021

(54) FUSION PROTEINS COMPRISING PROGRANULIN

(71) Applicant: Denali Therapeutics Inc., South San Francisco, CA (US)

(72) Inventors: Gilbert Di Paolo, South San Francisco, CA (US); Todd P. Logan, South San Francisco, CA (US); Kathryn M. Monroe, South San Francisco, CA (US); Ankita Srivastava, South San Francisco, CA (US); Bettina Van Lengerich, South San Francisco, CA (US)

(73) Assignee: Denali Therapeutics Inc., South San Francisco, CA (US)

(21) Appl. No.: 17/253,391

(22) PCT Filed: Jun. 18, 2019
(86) PCT No.: PCT/US2019/037695

§ 371 Date: Dec. 17, 2020

Related U.S. Application Data
(60) Provisional application No. 62/746,338, filed on Oct. 16, 2018.
(60) Provisional application No. 62/686,579, filed on Jun. 18, 2018.

(51) Int. Cl. C07K 14/475

(52) U.S. Cl. CPC C07K 14/475; A61K 38/00

(57) ABSTRACT

Provided herein are fusion proteins that comprise progranulin and an Fc polypeptide. Methods of using such proteins to treat progranulin-associated disorders (e.g., a neurodegenerative disease, such as frontotemporal dementia (FTD)) are also provided herein.

### CROSS-REFERENCE TO RELATED APPLICATIONS

This application claims priority to U.S. Provisional Application No. 62/686,579, filed Jun. 18, 2018, and U.S. Provisional Application No. 62/746,338, filed Oct. 16, 2018, the disclosures of which are hereby incorporated by reference in their entirety for all purposes.

### BACKGROUND

Frontotemporal dementia (FTD) is a progressive neurodegenerative disorder which accounts for 5-10% of all patients with dementia and 10-20% of patients with an onset of dementia before 65 years (Rademakers et al., Nat Rev Neurol. 8(8):423-34, 2012). While several genes have been linked to FTD, one of the most frequently mutated genes in FTD is GRN, which maps to human chromosome 17q21 and encodes the cysteine-rich protein progranulin (PGRN) (also known as proepithelin and acrogranin). Highly-penetrant mutations in GRN were first reported in 2006 as a cause of autosomal dominant forms of familial FTD (Baker et al., Nature. 442(7105):916-9, 2006; Cruts et al., Nature. 2006 Aug. 24; 442(7105):920-4; Gass et al., Hum Mol Genet. 15(20):2988-3001, 2006). Recent estimates suggest that GRN mutations account for 5-20% of FTD patients with positive family history and 1-5% of sporadic cases (Rademakers et al., supra).

### DESCRIPTION

Provided herein are fusion proteins comprising a progranulin polypeptide or a variant thereof and methods of use thereof for treating progranulin-associated disorders (e.g., a neurodegenerative disease, such as frontotemporal dementia (FTD)).

An aspect of the disclosure includes a protein comprising: (a) a modified Fc polypeptide dimer that specifically binds TfR; and (b) a progranulin polypeptide. In some embodiments, the protein is used in therapy. In some embodiments, the protein is used in the treatment of a neurodegenerative disease.

Another aspect of the disclosure includes a protein comprising: (a) an Fc polypeptide; and (b) a progranulin polypeptide. In some embodiments of this aspect, the Fc polypeptide: (i) does not include a variable domain, (ii) includes a hinge or partial hinge region, and/or (iii) includes modifications such that the Fc polypeptide specifically binds transferrin receptor.

In another aspect, provided herein is a protein comprising: (a) a single progranulin polypeptide or a variant thereof; (b) a first Fc polypeptide that is linked to the progranulin polypeptide or the variant thereof of (a); and (c) a second Fc polypeptide that forms an Fc dimer with the first Fc polypeptide. In some embodiments, the first Fc polypeptide and/or the second Fc polypeptide does not include an immunoglobulin heavy and/or light chain variable region sequence or an antigen-binding portion thereof. The protein can comprise exactly one progranulin polypeptide or a variant thereof.

In some embodiments of this aspect, the progranulin polypeptide comprises an amino acid sequence having greater than 90% identity, or at least 95% identity to the amino acid sequence of SEQ ID NO:212. In certain embodiments, the progranulin polypeptide comprises the amino acid sequence of SEQ ID NO:212.

In some embodiments, the first Fc polypeptide is linked to the progranulin polypeptide or the variant thereof by a peptide bond or by a polypeptide linker. In some embodiments, the polypeptide linker is 1 to 50, 1 to 25, 1 to 20, or 1 to 10 amino acids in length. For example, the polypeptide linker may be 3 to 50, 3 to 25, 5 to 50, 5 to 25, 5 to 20, or 10 to 25 amino acids in length. In some embodiments, the polypeptide linker is a flexible polypeptide linker. The flexible polypeptide linker may be a glycine-rich linker, e.g., G₄S (SEQ ID NO:277) or (G₄S)₂ (SEQ ID NO:276).

In some embodiments, the N-terminus or the C-terminus of the first Fc polypeptide is linked to the progranulin polypeptide.

In another aspect, provided herein is a protein comprising: (a) a first Fc polypeptide that is linked to a first progranulin polypeptide or a variant thereof; and (b) a second Fc polypeptide that is linked to a second progranulin polypeptide or a variant thereof, wherein the first Fc polypeptide and the second Fc polypeptide form an Fc dimer and wherein the first Fc polypeptide and/or the second Fc polypeptide specifically binds to a transferrin receptor.

In some embodiments of this aspect, the first Fc polypeptide and/or the second Fc polypeptide does not include an immunoglobulin heavy and/or light chain variable region sequence or an antigen-binding portion thereof.

In some embodiments, each of the first and second progranulin polypeptides comprises an amino acid sequence having greater 90%, or at least 95% identity to the amino acid sequence of SEQ ID NO:212. In certain embodiments, each of the first and second progranulin polypeptides comprises the amino acid sequence of SEQ ID NO:212.

In some embodiments, the first Fc polypeptide is linked to the first progranulin polypeptide or the variant thereof by a peptide bond or by a polypeptide linker and wherein the second Fc polypeptide is linked to the second progranulin polypeptide or the variant thereof by a peptide bond or by a polypeptide linker. In some embodiments, the polypeptide linker is 1 to 50, 1 to 25, 1 to 20, or 1 to 10 amino acids in length. For example, the polypeptide linker may be 3 to 50, 3 to 25, 5 to 50, 5 to 25, 5 to 20, or 10 to 25 amino acids in length. In some embodiments, the polypeptide linker is a flexible polypeptide linker. The flexible polypeptide linker may be a glycine-rich linker, e.g., G₄S (SEQ ID NO:277) or (G₄S)₂ (SEQ ID NO:276). In some embodiments, the first Fc polypeptide and/or the second Fc polypeptide does not include an immunoglobulin heavy and/or light chain variable region sequence or an antigen-binding portion thereof.

In some embodiments, the N-terminus or the C-terminus of the first Fc polypeptide is linked to the first progranulin polypeptide, and the N-terminus or the C-terminus of the second Fc polypeptide is linked to the second progranulin polypeptide.

In some embodiments, the N-terminus of the first Fc polypeptide is linked to the C-terminus of the first progranulin polypeptide, and the N-terminus of the second Fc polypeptide is linked to the C-terminus of the second progranulin polypeptide.

In some embodiments, the N-terminus of the first Fc polypeptide is linked to the C-terminus of the first progranulin polypeptide, and the C-terminus of the second Fc polypeptide is linked to the N-terminus of the second progranulin polypeptide.

In some embodiments, the C-terminus of the first Fc polypeptide is linked to the N-terminus of the first progranulin polypeptide, and the C-terminus of the second Fc polypeptide is linked to the N-terminus of the second progranulin polypeptide.

In some embodiments of the previous two aspects, the first Fc polypeptide is a modified Fc polypeptide and/or the second Fc polypeptide is a modified Fc polypeptide. In some embodiments, the first Fc polypeptide and the second Fc polypeptide each contain modifications that promote heterodimerization. In some embodiments, the Fc dimer is an Fc heterodimer.

In some embodiments of the previous two aspects, one of the Fc polypeptides has a T366W substitution and the other Fc polypeptide has T366S, L368A, and Y407V substitutions, according to EU numbering. In some embodiments, the first Fc polypeptide contains the T366S, L368A, and Y407V substitutions and the second Fc polypeptide contains the T366W substitution.

In some embodiments of the first aspect, the first Fc polypeptide is linked to the progranulin polypeptide and comprises the amino acid sequence of any one of SEQ ID NOS:213, 214, 225, and 226, and the second Fc polypeptide comprises the amino acid sequence of SEQ ID NO:261.

In some embodiments of the second aspect, the first Fc polypeptide is linked to the first progranulin polypeptide and comprises the amino acid sequence of any one of SEQ ID NOS:213, 214, 225, and 226, and the second Fc polypeptide is linked to the second progranulin polypeptide and comprises the amino acid sequence of any one of SEQ ID NOS:237, 238, 249, and 250.

In some embodiments of the previous two aspects, the first Fc polypeptide contains the T366W substitution and the second Fc polypeptide contains the T366S, L368A, and Y407V substitutions.

In some embodiments of the first aspect, the first Fc polypeptide is linked to the progranulin polypeptide and comprises the amino acid sequence of any one of SEQ ID NOS:237, 238, 249, and 250, and the second Fc polypeptide comprises the sequence of SEQ ID NO:267.

In some embodiments of the second aspect, the first Fc polypeptide is linked to the first progranulin polypeptide and comprises the amino acid sequence of any one of SEQ ID NOS:237, 238, 249, and 250, and the second Fc polypeptide is linked to the second progranulin polypeptide and comprises the amino acid sequence of any one of SEQ ID NOS:213, 214, 225, and 226.

In some embodiments, the first Fc polypeptide and/or the second Fc polypeptide comprises a native FcRn binding site.

In some embodiments, the first Fc polypeptide and the second Fc polypeptide do not have effector function.

In some embodiments, the first Fc polypeptide and/or the second Fc polypeptide includes a modification that reduces effector function. In certain embodiments, the modification that reduces effector function is the substitutions of Ala at position 234 and Ala at position 235, according to EU numbering.

In some embodiments, the first Fc polypeptide and/or the second Fc polypeptide comprises amino acid changes relative to the native Fc sequence that extend serum half-life. In certain embodiments, the amino acid changes comprise substitutions of Leu at position 428 and Ser at position 434, according to EU numbering. In certain embodiments, the amino acid changes comprise a substitution of Ser or Ala at position 434, according to EU numbering.

In some embodiments, the first Fc polypeptide and/or the second Fc polypeptide specifically binds to a transferrin receptor. In certain embodiments, the first Fc polypeptide and/or the second Fc polypeptide binds to the apical domain of the transferrin receptor.

In some embodiments, the first Fc polypeptide and/or the second Fc polypeptide comprises at least two substitutions at positions selected from the group consisting of 384, 386, 387, 388, 389, 390, 413, 416, and 421, according to EU numbering. In some embodiments, the first Fc polypeptide and/or the second Fc polypeptide includes substitutions at least three, four, five, six, seven, eight, or nine of the positions. In some embodiments, the first Fc polypeptide and/or the second Fc polypeptide further comprises one, two, three, or four substitutions at positions comprising 380, 391, 392, and 415, according to EU numbering. In certain embodiments, the first Fc polypeptide and/or the second Fc polypeptide further comprises one, two, or three substitutions at positions comprising 414, 424, and 426, according to EU numbering. In particular embodiments, the first Fc polypeptide and/or the second Fc polypeptide comprises Trp at position 388. In some embodiments, the first Fc polypeptide and/or the second Fc polypeptide comprises an aromatic amino acid at position 421 (e.g., Trp or Phe at position 421).

In some embodiments, the first Fc polypeptide and/or the second Fc polypeptide comprises at least one position selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the first Fc polypeptide and/or the second Fc polypeptide comprises 2, 3, 4, 5, 6, 7, 8, 9, 10, or 11 positions selected from the following: position 380 is Tip, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe. In certain embodiments, the first Fc polypeptide and/or the second Fc polypeptide comprises 11 positions as follows: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the first Fc polypeptide and/or the second Fc polypeptide has a CH3 domain with at least 85% identity, at least 90% identity, or at least 95% identity to amino acids 111-217 of any one of SEQ ID NOS:34-38, 58, 60-90, 136, and 137-210.

In certain embodiments, the residues at least 5, 6, 7, 8, 9, 10, 11, 12, 13, 14, 15, or 16 of the positions corresponding to EU index positions 380, 384, 386, 387, 388, 389, 390, 391, 392, 413, 414, 415, 416, 421, 424 and 426 of any one of SEQ ID NOS:34-38, 58, 60-90, 136, and 137-210 are not deleted or substituted. In some embodiments, the first Fc polypeptide and/or the second Fc polypeptide comprises the amino acid sequence of any one of SEQ ID NOS:136-210. In certain embodiments, the first Fc polypeptide and/or the second Fc polypeptide comprises the amino acid sequence of any one of SEQ ID NOS:136, 138, 150, 162, 174, 186, and 198. In certain embodiments, the first Fc polypeptide and/or the second Fc polypeptide comprises the amino acid sequence of SEQ ID NO:150. In certain embodiments, the first Fc polypeptide and/or the second Fc polypeptide comprises the amino acid sequence of SEQ ID NO:136.

In some embodiments, the binding of the protein to the transferrin receptor does not substantially inhibit binding of transferrin to the transferrin receptor.

In some embodiments, the first Fc polypeptide and/or the second Fc polypeptide has an amino acid sequence identity of at least 75%, or at least 80%, 85%, 90%, 92%, or 95%, as compared to the corresponding wild-type Fc polypeptide. The corresponding wild-type Fc polypeptide may be a human IgG1, IgG2, IgG3, or IgG4 Fc polypeptide.

In some embodiments, uptake of the progranulin polypeptide into the brain is at least ten-fold greater as compared to the uptake of the progranulin polypeptide in the absence of the first Fc polypeptide and/or the second Fc polypeptide or as compared to the uptake of the progranulin polypeptide without the modifications to the first Fc polypeptide and/or the second Fc polypeptide that result in transferrin receptor binding.

In some embodiments, the first Fc polypeptide is not modified to bind to a blood-brain barrier receptor and the second Fc polypeptide is modified to specifically bind to a transferrin receptor. In some embodiments, the first Fc polypeptide is modified to specifically bind to a transferrin receptor and the second Fc polypeptide is not modified to bind to a blood-brain barrier receptor.

In some embodiments, the progranulin polypeptide or the variant thereof binds to sortilin or prosaposin. In particular embodiments, the progranulin polypeptide or the variant thereof binds to sortilin.

In another aspect, provided herein is a protein comprising: (a) a first Fc polypeptide that is linked to a progranulin polypeptide through a polypeptide linker; and (b) a second Fc polypeptide that forms an Fc dimer with the first Fc polypeptide, wherein the first Fc polypeptide comprises hole mutations T366S, L368A, and Y407V and the second Fc polypeptide comprises a knob mutation T366W, according to EU numbering scheme.

In some embodiments, (a) comprises the sequence of any one of SEQ ID NOS:213, 214, 225, and 226 and (b) comprises the sequence of SEQ ID NO:261.

In some embodiments, the second Fc polypeptide further comprises TfR-binding mutations.

In some embodiments, (a) comprises the sequence of any one of SEQ ID NOS:213, 214, 225, and 226 and (b) comprises the sequence of SEQ ID NO:273.

In another aspect, provided herein is a protein comprising: (a) a first Fc polypeptide that is linked to a progranulin polypeptide through a polypeptide linker; and (b) a second Fc polypeptide that forms an Fc dimer with the first Fc polypeptide, wherein the first Fc polypeptide comprises a knob mutation T366W and the second Fc polypeptide comprises hole mutations T366S, L368A, and Y407V, according to EU numbering scheme.

In some embodiments, (a) comprises the sequence of any one of SEQ ID NOS:237, 238, 249, and 250 and (b) comprises the sequence of SEQ ID NO:267.

In some embodiments, the first Fc polypeptide further comprises TfR-binding mutations.

In some embodiments, (a) comprises the sequence of SEQ ID NO:274 or 275 and (b) comprises the sequence of SEQ ID NO:267.

In another aspect, provided herein is a protein comprising: (a) a first Fc polypeptide that is linked to a first progranulin polypeptide through a polypeptide linker; and (b) a second Fc polypeptide that is linked to a second progranulin polypeptide through a polypeptide linker, wherein the first and second Fc polypeptides form an Fc dimer, the first Fc polypeptide comprises hole mutations T366S, L368A, and Y407V, the second Fc polypeptide comprises a knob mutation T366W, and wherein the N-terminus of the first progranulin polypeptide is linked to the C-terminus of the first Fc polypeptide through the polypeptide linker and the N-terminus of the second progranulin polypeptide is linked to the C-terminus of the second Fc polypeptide through the polypeptide linker.

In some embodiments, (a) comprises the sequence of SEQ ID NO:213 or 214 and (b) comprises the sequence of SEQ ID NO:237 or 238.

In some embodiments, the second Fc polypeptide further comprises TfR-binding mutations.

In some embodiments, (a) comprises the sequence of SEQ ID NO:213 or 214 and (b) comprises the sequence of SEQ ID NO:274.

In another aspect, provided herein is a protein comprising: (a) a first Fc polypeptide that is linked to a first progranulin polypeptide through a polypeptide linker; and (b) a second Fc polypeptide that is linked to a second progranulin polypeptide through a polypeptide linker, wherein the first and second Fc polypeptides form an Fc dimer, the first Fc polypeptide comprises hole mutations T366S, L368A, and Y407V, the second Fc polypeptide comprises a knob mutation T366W, and wherein the C-terminus of the first progranulin polypeptide is linked to the N-terminus of the first Fc polypeptide through the polypeptide linker and the C-terminus of the second progranulin polypeptide is linked to the N-terminus of the second Fc polypeptide through the polypeptide linker.

In some embodiments, (a) comprises the sequence of SEQ ID NO:225 or 226 and (b) comprises the sequence of SEQ ID NO:249 or 250.

In some embodiments, the second Fc polypeptide further comprises TfR-binding mutations.

In some embodiments, (a) comprises the sequence of SEQ ID NO:225 or 226 and (b) comprises the sequence of SEQ ID NO:275.

In another aspect, provided herein is a protein comprising: (a) a first Fc polypeptide that is linked to a first progranulin polypeptide through a polypeptide linker; and (b) a second Fc polypeptide that is linked to a second progranulin polypeptide through a polypeptide linker, wherein the first and second Fc polypeptides form an Fc dimer, the first Fc polypeptide comprises hole mutations T366S, L368A, and Y407V, the second Fc polypeptide comprises a knob mutation T366W, and wherein the N-terminus of the first progranulin polypeptide is linked to the C-terminus of the first Fc polypeptide through the polypeptide linker and the C-terminus of the second progranulin polypeptide is linked to the N-terminus of the second Fc polypeptide through the polypeptide linker.

In some embodiments, (a) comprises the sequence of SEQ ID NO:213 or 214 and (b) comprises the sequence of SEQ ID NO:249 or 250.

In some embodiments, the second Fc polypeptide further comprises TfR-binding mutations.

In some embodiments, (a) comprises the sequence of SEQ ID NO:213 or 214 and (b) comprises the sequence of SEQ ID NO:275.

In another aspect, provided herein is a protein comprising: (a) a first Fc polypeptide that is linked to a progranulin polypeptide through a polypeptide linker; and (b) a second Fc polypeptide that forms an Fc dimer with the first Fc polypeptide, wherein the first Fc polypeptide comprises hole mutations T366S, L368A, and Y407V and the second Fc polypeptide comprises a knob mutation T366W, according to EU numbering scheme, and TfR-binding mutations.

In some embodiments of this aspect, (a) comprises the sequence of any one of SEQ ID NOS:213, 214, 225, and 226 and (b) comprises the sequence of SEQ ID NO:281.

In some embodiments of this aspect, (a) comprises the sequence of any one of SEQ ID NOS:213, 214, 225, and 226 and (b) comprises the sequence of SEQ ID NO:286.

In some embodiments of this aspect, the second Fc polypeptide further comprises L234A and L235A mutations with or without P329G mutation, and/or M428L and N434S mutations, according to EU numbering scheme. In particular embodiments, (a) comprises the sequence of any one of SEQ ID NOS:213, 214, 225, and 226 and (b) comprises the sequence of any one of SEQ ID NOS:210 and 282-285. In particular embodiments, (a) comprises the sequence of any one of SEQ ID NOS:213, 214, 225, and 226 and (b) comprises the sequence of any one of SEQ ID NOS:209 and 287-290. In particular embodiments, (a) comprises the sequence of any one of SEQ ID NOS:213, 214, 225, and 226 and (b) comprises the sequence of SEQ ID NO: 291.

In some embodiments of this aspect, the first Fc polypeptide further comprises L234A and L235A mutations with or without P329G mutation, and/or M428L and N434S mutations, according to EU numbering scheme. In particular embodiments, (a) comprises the sequence of any one of SEQ ID NOS:215-224 and 227-236 and (b) comprises the sequence of SEQ ID NO:281. In particular embodiments, (a) comprises the sequence of any one of SEQ ID NOS:215-224 and 227-236 and (b) comprises the sequence of SEQ ID NO:286.

In some embodiments of this aspect, each of the first and second Fc polypeptides further comprises L234A and L235A mutations with or without P329G mutation, and/or M428L and N434S mutations, according to EU numbering scheme. In particular embodiments, (a) comprises the sequence of any one of SEQ ID NOS:215-224 and 227-236 and (b) comprises the sequence of SEQ ID NO:210 and 282-285. In particular embodiments, (a) comprises the sequence of any one of SEQ ID NOS:215-224 and 227-236 and (b) comprises the sequence of SEQ ID NO:209 and 287-290. In particular embodiments, (a) comprises the sequence of any one of SEQ ID NOS:215-224 and 227-236 and (b) comprises the sequence of SEQ ID NO:291.

In particular embodiments, (a) comprises the sequence of SEQ ID NO:215 and (b) comprises the sequence of SEQ ID NO:210. In particular embodiments, (a) comprises the sequence of SEQ ID NO:227 and (b) comprises the sequence of SEQ ID NO:210. In particular embodiments, (a) comprises the sequence of SEQ ID NO:215 and (b) comprises the sequence of SEQ ID NO:291. In particular embodiments, (a) comprises the sequence of SEQ ID NO:227 and (b) comprises the sequence of SEQ ID NO:291.

In another aspect, provided herein is a polypeptide comprising an Fc polypeptide that is linked to a progranulin polypeptide or a variant thereof, wherein the Fc polypeptide contains one or more modifications that promote its heterodimerization to another Fc polypeptide.

In some embodiments, the progranulin polypeptide comprises an amino acid sequence having greater than 90%, or at least 95% identity to the amino acid sequence of SEQ ID NO:212. In certain embodiments, the progranulin polypeptide comprises the amino acid sequence of SEQ ID NO:212.

In some embodiments, the Fc polypeptide is linked to the progranulin polypeptide or the variant thereof by a peptide bond or by a polypeptide linker. In some embodiments, the polypeptide linker is 1 to 50, 1 to 25, 1 to 20, or 1 to 10 amino acids in length. For example, the polypeptide linker may be 3 to 50, 3 to 25, 5 to 50, 5 to 25, 5 to 20, or 10 to 25 amino acids in length. In certain embodiments, the polypeptide linker is a flexible polypeptide linker. The flexible polypeptide linker may be a glycine-rich linker (e.g., G₄S (SEQ ID NO:277) or (G₄S)₂ (SEQ ID NO:276)).

In some embodiments, the N-terminus or the C-terminus of the Fc polypeptide is linked to the progranulin polypeptide.

In some embodiments, the Fc polypeptide contains T366S, L368A, and Y407V substitutions, according to EU numbering.

In some embodiments, the polypeptide comprises the amino acid sequence of any one of SEQ ID NOS:213, 214, 225, and 226.

In certain embodiments, the Fc polypeptide contains a T366W substitution.

In some embodiments, the polypeptide comprises the amino acid sequence of any one of SEQ ID NOS:237, 238, 249, and 250.

In some embodiments, the Fc polypeptide comprises a modification that reduces effector function. In certain embodiments, the modification that reduces effector function is the substitutions of Ala at position 234 and Ala at position 235, according to EU numbering.

In some embodiments, the polypeptide comprises the amino acid sequence of any one of SEQ ID NOS:215, 216, 227, 228, 239, 240, 251, and 252.

In some embodiments, the Fc polypeptide comprises amino acid changes relative to the native Fc sequence that extend serum half-life. In some embodiments, the amino acid changes comprise substitutions of Leu at position 428 and Ser at position 434, according to EU numbering.

In some embodiments, the polypeptide comprises the amino acid sequence of any one of SEQ ID NOS:219-222, 231-234, 243-246, and 255-258.

In some embodiments, the Fc polypeptide further comprises TfR-binding mutations.

In some embodiments, the progranulin polypeptide or the variant thereof binds to sortilin or prosaposin. In particular embodiments, the progranulin polypeptide or the variant thereof binds to sortilin.

In another aspect, provided herein is a method of treating a progranulin-associated disorder, the method comprising administering a protein or polypeptide disclosed herein to a patient in need thereof, wherein the progranulin-associated disorder is selected from the group consisting of a neurodegenerative disease, atherosclerosis, a disorder associated with TDP-43, and age-related macular degeneration (AMD).

In another aspect, provided herein is a method of increasing the amount of a progranulin polypeptide or a variant thereof in a patient having a progranulin-associated disorder, the method comprising administering a protein or polypeptide disclosed herein to the patient, wherein the progranulin-associated disorder is selected from the group consisting of a neurodegenerative disease, atherosclerosis, a disorder associated with TDP-43, and age-related macular degeneration (AMD).

In another aspect, provided herein is a method of decreasing cathepsin D activity in a patient having a progranulin-associated disorder, the method comprising administering a protein or polypeptide disclosed herein to the patient, wherein the progranulin-associated disorder is selected from the group consisting of a neurodegenerative disease, atherosclerosis, a disorder associated with TDP-43, and age-related macular degeneration (AMD).

In another aspect, provided herein is a method of increasing lysosomal degradation in a patient having a progranulin-associated disorder, the method comprising administering a protein or polypeptide disclosed herein to the patient, wherein the progranulin-associated disorder is selected from the group consisting of a neurodegenerative disease, atherosclerosis, a disorder associated with TDP-43, and age-related macular degeneration (AMD).

In some embodiments of the methods described herein, the progranulin-associated disorder is a neurodegenerative disease. In some embodiments of the methods described herein, the neurodegenerative disease is selected from the group consisting of frontotemporal dementia (FTD), neuronal ceroid lipofuscinosis (NCL), Niemann-Pick disease type A (NPA), Niemann-Pick disease type B (NPB), Niemann-Pick disease type C (NPC), C9ORF72-associated amyotrophic lateral sclerosis (ALS)/FTD, sporadic ALS, Alzheimer's disease (AD), Gaucher's disease (e.g., Gaucher's disease types 2 and 3), and Parkinson's disease. In some embodiments, the neurodegenerative disease is frontotemporal dementia (FTD).

In some embodiments, the patient has a mutation in a gene encoding the progranulin polypeptide.

In another aspect, provided herein is a pharmaceutical composition comprising any of the proteins described herein or any of the peptides described herein and a pharmaceutically acceptable carrier.

In another aspect, provided is a method for evaluating a compound or monitoring a subject's response to a compound, pharmaceutical composition, or dosing regimen thereof (e.g., response to a Fc dimer:PGRN fusion protein described herein) for treating a progranulin-associated disorder, the method comprising: (a) measuring an abundance of one or more bis(monoacylglycero)phosphate (BMP) species in a test sample from a subject having a progranulin-associated disorder, wherein the test sample or subject has been treated with the compound or pharmaceutical composition thereof (e.g., treated with a Fc dimer:PGRN fusion protein described herein); (b) comparing the difference in abundance between the one or more BMP species measured in (a) and one or more reference values; and (c) determining from the comparison whether the compound, pharmaceutical composition, or dosing regimen thereof (e.g., a Fc dimer:PGRN fusion protein described herein) improves one or more BMP species levels for treating a progranulin-associated disorder.

In some embodiments, the methods provided herein further comprise treating another test sample or subject with another compound and selecting a candidate compound that improves the one or more BMP species levels.

In some embodiments, the methods provided herein further comprise (d) maintaining or adjusting the amount or frequency of administration of the compound (e.g., a Fc dimer:PGRN fusion protein described herein) to the test sample or subject; and (e) administering the compound to the test sample or to the subject.

In another aspect, provided is a method for identifying a subject having, or at risk of having, a progranulin-associated disorder, the method comprising: (a) measuring the abundance of one or more BMP species in a test sample from the subject; (b) comparing the difference in abundance between the one or more BMP species measured in (a) and one or more reference values; and (c) determining from the comparison whether the subject has a progranulin-associated disorder.

In some embodiments, the methods provided herein further comprise administering to the subject a compound (e.g., a Fc dimer:PGRN fusion protein described herein) for improving the one or more BMP species levels for treating a progranulin-associated disorder. In some embodiments, at least one of the one or more signs or symptoms of a progranulin-associated disorder are ameliorated following treatment.

In some embodiments, treatment comprises administering progranulin, a derivative thereof, or a pharmaceutical composition thereof (e.g., administering a Fc dimer:PGRN fusion protein described herein) to the subject. In some embodiments, the progranulin derivative contains a chemical moiety or peptide fragment that allows the progranulin to cross the blood brain barrier (e.g., a progranulin derivative such as a Fc dimer:PGRN fusion protein described herein). In some embodiments, treatment comprises administering a library of compounds to a plurality of subjects or test samples.

In some embodiments, the reference value is measured in a reference sample obtained from a reference subject or a population of reference subjects. In some embodiments, the reference value is the abundance of the one or more BMP species measured in a reference sample. In some embodiments, the reference sample is the same type of cell, tissue, or fluid as the test sample. In some embodiments, at least two reference values from different types of cell, tissue, or fluid is measured.

In some embodiments, the reference sample is a healthy control. In some embodiments, the reference subject or population of reference subjects do not have a progranulin-associated disorder or a decreased level of progranulin. In particular embodiments, the reference subject or population of reference subjects do not have any signs or symptoms of such a disorder.

In some embodiments, BMP species levels are increased in bone marrow-derived macrophages that are derived in vitro from bone marrow cells of a subject having, or at risk of having, a progranulin-associated disorder as compared to a healthy control or a control not related to a progranulin-associated disorder.

In some embodiments, BMP species levels are decreased in liver, brain, cerebrospinal fluid, plasma, or urine of a subject having, or at risk of having, a progranulin-associated disorder as compared to a healthy control or a control not related to a progranulin-associated disorder.

In some embodiments, the abundance of a BMP species in the test sample of a subject having, or at risk of having, a progranulin-associated disorder has at least about a 1.2-fold, 1.5-fold, or 2-fold difference compared to a reference value of a control such as a healthy control or a control not related to a progranulin-associated disorder. In other embodiments, the abundance of a BMP species in the test sample of a subject having, or at risk of having, a progranulin-associated disorder has about a 1.2-fold to about 4-fold difference compared to a reference value of a control such as a healthy control or a control not related to a progranulin-associated disorder. In some embodiments, the difference compared to a reference value is about 2-fold to about 3-fold. In some embodiments, the subject has a disorder associated with a decreased level of progranulin and/or one or more signs or symptoms of a disorder associated with a decreased level of progranulin.

In some embodiments, the reference value is the BMP species value prior to treatment. In some embodiments, the subject is treated for a decreased level of progranulin or a progranulin-associated disorder, and the test sample comprises one or more pre-treatment test samples that are obtained from the subject before treatment has started and one or more post-treatment test samples that are obtained from the subject after treatment has started. In some embodiments, the method further comprises determining that the subject is responding to the treatment when the abundance of at least one of the one or more BMP species post-treatment shows an improvement over the one or more BMP species pre-treatment relative to a healthy control.

In some embodiments, the methods comprise (a) measuring an abundance of one or more bis(monoacylglycero)phosphate (BMP) species in a test sample obtained from a subject; (b) treating the test sample or subject with a compound, pharmaceutical composition, or dosing regimen thereof (e.g., treating the test sample or subject with a Fc dimer:PGRN fusion protein described herein); (c) measuring an abundance of one or more BMP species in a test sample obtained from the treated subject, and (d) comparing the abundance of the one or more BMP species measured in steps (a) and (c); and (e) determining whether the compound or a dosing regimen improves BMP levels for treating a progranulin-associated disorder.

In some embodiments, two or more post-treatment test samples are obtained at different time points after treatment has started, and the method further comprises determining that the subject is responding to treatment when the abundance of at least one of the one or more BMP species measured in a post-treatment sample is a) lower in bone marrow-derived macrophage (BMDM) or b) higher in liver, brain, cerebrospinal fluid, plasma, or urine than the abundance of the corresponding one or more BMP species measured in the pre-treatment sample. In some embodiments, the subject is determined to be responding to the treatment when the abundance of at least one of the one or more BMP species measured in a post-treatment sample is a) at least about 1.2-fold lower in BMDM or b) at least about 1.2-fold higher in liver, brain, cerebrospinal fluid, plasma, or urine than the abundance of the corresponding one or more BMP species measured in the pre-treatment sample.

In some embodiments, the improved BMP species level is an improvement over the BMP species level prior to treatment relative to the reference value of a control such as a healthy control or a control not related to a progranulin-associated disorder. In some embodiments, the improved BMP species level is closer in value to the control than the pre-treatment BMP species level is to the control. In some embodiments, the improved BMP species level has a difference compared to the control of less than 15%, 10%, or 5%. In some embodiments, the improved BMP species level has a difference compared to a healthy control of less than 10% or 5%. In some embodiments, the improved BMP species level has a difference compared to a healthy control of less than 5%.

In some embodiments, the method further comprises determining that the subject is responding to the treatment when the abundance of at least one of the one or more BMP species measured in at least one of the one or more post-treatment test samples is about the same as the corresponding reference value of a healthy control.

In some embodiments, the test or reference sample or one or more reference values comprises or relates to a cell, a tissue, whole blood, plasma, serum, cerebrospinal fluid, interstitial fluid, sputum, urine, feces, bronchioalveolar lavage fluid, lymph, semen, breast milk, amniotic fluid, or a combination thereof. In some embodiments, the cell is a peripheral blood mononuclear cell (PBMC), a bone marrow-derived macrophage (BMDM), a retinal pigmented epithelial (RPE) cell, a blood cell, an erythrocyte, a leukocyte, a neural cell, a microglial cell, a brain cell, a cerebral cortex cell, a spinal cord cell, a bone marrow cell, a liver cell, a kidney cell, a splenic cell, a lung cell, an eye cell, a chorionic villus cell, a muscle cell, a skin cell, a fibroblast, a heart cell, a lymph node cell, or a combination thereof. In some embodiments, the cell is a cultured cell. In some embodiments, the cultured cell is a BMDM or an RPE cell.

In some embodiments, the tissue comprises brain tissue, cerebral cortex tissue, spinal cord tissue, liver tissue, kidney tissue, muscle tissue, heart tissue, eye tissue, retinal tissue, a lymph node, bone marrow, skin tissue, blood vessel tissue, lung tissue, spleen tissue, valvular tissue, or a combination thereof. In some embodiments, the test and/or reference sample is purified from a cell and/or a tissue and comprises an endosome, a lysosome, an extracellular vesicle, an exosome, a microvesicle, or a combination thereof.

In some embodiments, the one or more BMP species comprise two or more BMP species. In some embodiments, the one or more BMP species comprise BMP(16:0_18:1), BMP(16:0_18:2), BMP(18:0_18:0), BMP(18:0_18:1), BMP(18:1_18:1), BMP(16:0_20:3), BMP(18:1_20:2), BMP(18:0_20:4), BMP(16:0_22:5), BMP(20:4_20:4), BMP(22:6_22:6), BMP(20:4_20:5), BMP(18:2_18:2), BMP(16:0_20:4), BMP(18:0_18:2), BMP(18:0e_22:6), BMP(18:1e_20:4), BMP(18:3_22:5), BMP(20:4_22:6), BMP(18:0e_20:4), BMP(18:2_20:4), BMP(18:1_22:6), BMP(18:1_20:4), BMP(18:0_22:6), or a combination thereof.

In some embodiments, the one or more BMP species comprise BMP(18:1_18:1), BMP(18:0_20:4), BMP(20:4_20:4), BMP(22:6_22:6), BMP(20:4_22:6), BMP(18:1_22:6), BMP(18:1_20:4), BMP(18:0_22:6), BMP(18:3_22:5), or a combination thereof.

In some embodiments, the test sample comprises a cultured cell and the one or more BMP species comprise BMP(18:1_18:1). In some embodiments, the test sample comprises plasma, tissue, urine, cerebrospinal fluid (CSF), and/or brain or liver tissue, and the one or more BMP species comprise BMP(22:6_22:6). In some embodiments, the test sample comprises liver tissue and the one or more BMP species comprise BMP(22:6_22:6), BMP(18:3_22:5), or a combination thereof. In some embodiments, the test sample comprises CSF or urine and the one or more BMP species comprise BMP(22:6_22:6). In some embodiments, the test sample comprises microglia and the one or more BMP species comprise BMP(18:3_22:5).

In some embodiments, the abundance of the one or more BMP species is measured using a method selected from the group consisting of liquid chromatography-mass spectrometry (LC-MS), liquid chromatography-tandem mass spectrometry (LC-MS/MS), gas chromatography-mass spectrometry (GC-MS), gas chromatography-tandem mass spectrometry (GC-MS/MS), enzyme-linked immunosorbent assay (ELISA), and a combination thereof. In some embodiments, an internal BMP standard is used to measure the abundance of the one or more BMP species in step (a) and/or determine the corresponding reference value. In some embodiments, the internal BMP standard comprises a BMP species that is not naturally present in the subject and/or the reference subject or population of reference subjects. In some embodiments, the internal BMP standard comprises BMP(14:0_14:0).

In some embodiments, the progranulin-associated disorder is a disorder related to progranulin expression, processing, glycosylation, cellular uptake, trafficking, and/or function. In some embodiments, the subject and/or the reference subject or population of reference subjects have a decreased level of progranulin and/or a disorder associated with a decreased level of progranulin, and the test sample has been contacted with a candidate compound (e.g., a Fc dimer:PGRN fusion protein described herein). In some embodiments, the subject and/or the reference subject or population of reference subjects have one or more signs or symptoms of the disorder associated with a decreased level of progranulin. In some embodiments, the subject and/or the reference subject or population of reference subjects have a mutation in a granulin (GRN) gene. In some embodiments, the mutation in the GRN gene decreases progranulin expression and/or activity. In some embodiments, the progranulin-associated disorder is atherosclerosis, Gaucher's disease, or age-related macular degeneration (AMD). In some embodiments, the progranulin-associated disorder is Gaucher's disease type 1. In some embodiments, the progranulin-associated disorder is a disorder associated with TDP-43. In other embodiments the TDP-43 associated disorder is AD or ALS.

In some embodiments, the subject and/or the reference subject is a human, a non-human primate, a rodent, a dog, or a pig.

In another aspect, the present disclosure provides a kit for monitoring progranulin levels in a subject. In some embodiments, the kit comprises a bis(monoacylglycero)phosphate (BMP) standard for measuring the abundance of one or more BMP species in a test sample obtained from the subject and/or a reference sample obtained from a reference subject or a population of reference subjects. In some embodiments, the BMP standard comprises a BMP species that is not naturally present in the subject and/or reference subject. In some embodiments, the BMP standard comprises BMP(14:0_14:0).

In some embodiments, the kit further comprises reagents for obtaining the sample from the subject and/or reference subject, processing the sample, measuring the abundance of the one or more BMP species, or a combination thereof. In some embodiments, the kit further comprises instructions for use.

In another aspect, the present disclosure provides a non-human transgenic animal comprising (a) a nucleic acid that encodes a chimeric TfR polypeptide comprising: (i) an apical domain having at least 90% identity to SEQ ID NO:296 and (ii) the transferrin binding site of the native TfR polypeptide of the animal, and (b) a knockout of the GRN gene, and wherein the chimeric TfR polypeptide is expressed in the brain of the animal.

In some embodiments, the apical domain comprises the amino acid sequence of SEQ ID NO:296. In some embodiments, the apical domain comprises the amino acid sequence of SEQ ID NO:297, SEQ ID NO:298, or SEQ ID NO:299.

In some embodiments, the chimeric TfR polypeptide comprises an amino acid sequence having at least 95% identity to SEQ ID NO:300.

In some embodiments, the animal expresses a level of the chimeric TfR polypeptide in brain, liver, kidney, or lung tissue within 20% (e.g., 18%, 16%, 14%, 12%, 10%, 8%, 6%, or 4%) of the level of expression of TfR in the same tissue of a corresponding wild-type animal of the same species.

In some embodiments, the animal comprises a red blood cell count, level of hemoglobin, or level of hematocrit within 20% (e.g., 18%, 16%, 14%, 12%, 10%, 8%, 6%, or 4%) of the red blood cell count, level of hemoglobin, or level of hematocrit in a corresponding wild-type animal of the same species.

In some embodiments, the nucleic acid sequence encoding the apical domain comprises a nucleic acid sequence having at least 95% (e.g., 97%, 98%, or 99%) identity to SEQ ID NO:301.

In some embodiments, the animal is homozygous or heterozygous for the nucleic acid encoding the chimeric TfR polypeptide.

In some embodiments, the knockout of the GRN gene comprises a deletion of exons 1-4 of the GRN gene.

In some embodiments, the animal is a mouse or a rat.

In another aspect, the present disclosure provides a protein comprising: (a) a modified Fc polypeptide dimer that specifically binds TfR; and (b) a progranulin polypeptide. In some embodiment, the protein further comprises: (c) a polypeptide linker, wherein the polypeptide linker links the modified Fc polypeptide dimer to the progranulin polypeptide.

In some embodiments, the modified Fc polypeptide dimer specifically binds to the apical domain of TfR.

In some embodiments, the modified Fc polypeptide dimer comprises at least two substitutions at positions selected from the group consisting of 384, 386, 387, 388, 389, 390, 413, 416, and 421, according to EU numbering, of one Fc polypeptide. In some embodiments, the modified Fc polypeptide dimer comprises substitutions at at least three, four, five, six, seven, eight, or nine of the positions of one Fc polypeptide. In some embodiments, the modified Fc polypeptide dimer further comprises one, two, three, or four substitutions at positions comprising 380, 391, 392, and 415, according to EU numbering, of one Fc polypeptide. In some embodiments, the modified Fc polypeptide dimer further comprises one, two, or three substitutions at positions comprising 414, 424, and 426, according to EU numbering, of one Fc polypeptide.

In certain embodiments, the modified Fc polypeptide dimer comprises Trp at position 388 of one Fc polypeptide. In certain embodiments, the modified Fc polypeptide dimer comprises at least one position selected from the following: position 380 is Tip, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe of one Fc polypeptide.

In some embodiments, the modified Fc polypeptide dimer comprises 2, 3, 4, 5, 6, 7, 8, 9, 10, or 11 positions selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe of one Fc polypeptide.

In some embodiments, the modified Fc polypeptide dimer comprises a first Fc polypeptide CH3 domain with at least 85% identity, at least 90% identity, or at least 95% identity to amino acids 111-217 of any one of SEQ ID NOS:34-38, 58, 60-90, 136, and 137-210. In certain embodiments, the modified Fc polypeptide dimer comprises the amino acid sequence of any one of SEQ ID NOS:136-210. In certain embodiments, the modified Fc polypeptide dimer comprises the amino acid sequence of any one of SEQ ID NOS:136, 138, 150, 162, 174, 186, and 198.

In some embodiments, the modified Fc polypeptide dimer comprises an Fc polypeptide having an amino acid sequence identity of at least 75%, or at least 80%, 85%, 90%, 92%, or 95%, as compared to the corresponding wild-type Fc polypeptide.

In some embodiments, the modified Fc polypeptide dimer does not include an immunoglobulin heavy and/or light chain variable region sequence or an antigen-binding portion thereof.

In some embodiments, the C-terminus of an Fc polypeptide in the modified Fc polypeptide dimer is linked to the N-terminus of the progranulin polypeptide. In some embodiments, the the polypeptide linker links the C-terminus of the Fc polypeptide in the modified Fc polypeptide dimer to the N-terminus of the progranulin polypeptide.

In some embodiments, the C-terminus of the progranulin polypeptide is linked to the N-terminus of an Fc polypeptide in the modified Fc polypeptide dimer. In some embodiments, the polypeptide linker links the C-terminus of the progranulin polypeptide to the N-terminus of the Fc polypeptide in the modified Fc polypeptide dimer.

In some embodiments, any of the proteins described herein can be used in therapy.

In some embodiments, any of the proteins described herein can be used in treating a neurodegenerative disease.

In some embodiments, any of the proteins described herein can be used in treating a neurodegenerative disease selected from the group consisting of Alzheimer's disease, primary age-related tauopathy, lewy body dementia, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:213 and (b) comprises the sequence of SEQ ID NO:273.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:214 and (b) comprises the sequence of SEQ ID NO:273.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:225 and (b) comprises the sequence of SEQ ID NO:273.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that is linked to a progranulin polypeptide through a polypeptide linker, wherein the second Fc polypeptide forms an Fc dimer with the first Fc polypeptide, and wherein (a) comprises the sequence of SEQ ID NO:213 and (b) comprises the sequence of SEQ ID NO:274.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that is linked to a progranulin polypeptide through a polypeptide linker, wherein the second Fc polypeptide forms an Fc dimer with the first Fc polypeptide, and wherein (a) comprises the sequence of SEQ ID NO:213 and (b) comprises the sequence of SEQ ID NO:275.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that is linked to a progranulin polypeptide through a polypeptide linker, wherein the second Fc polypeptide forms an Fc dimer with the first Fc polypeptide, and wherein (a) comprises the sequence of SEQ ID NO:225 and (b) comprises the sequence of SEQ ID NO:275.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:213 and (b) comprises the sequence of SEQ ID NO:261.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:225 and (b) comprises the sequence of SEQ ID NO:261.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:215 and (b) comprises the sequence of SEQ ID NO:110.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:227 and (b) comprises the sequence of SEQ ID NO:110.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:215 and (b) comprises the sequence of SEQ ID NO:273.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:227 and (b) comprises the sequence of SEQ ID NO:273.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:215 and (b) comprises the sequence of SEQ ID NO:282.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:227 and (b) comprises the sequence of SEQ ID NO:282.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:215 and (b) comprises the sequence of SEQ ID NO:284.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:227 and (b) comprises the sequence of SEQ ID NO:284.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:215 and (b) comprises the sequence of SEQ ID NO:285.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:227 and (b) comprises the sequence of SEQ ID NO:285.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:215 and (b) comprises the sequence of SEQ ID NO:210.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:227 and (b) comprises the sequence of SEQ ID NO:210.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:215 and (b) comprises the sequence of SEQ ID NO:291.

In another aspect, the present disclosure provides a protein for use in treating a neurodegenerative disease, comprising (a) a first Fc polypeptide that is linked to a progranulin polypeptide through a polypeptide linker; and (b) a second Fc polypeptide that forms an Fc dimer with the first Fc polypeptide, wherein (a) comprises the sequence of SEQ ID NO:227 and (b) comprises the sequence of SEQ ID NO:291.

### BRIEF DESCRIPTION OF THE DRAWINGS

FIG. 1A shows a schematic drawing of three Fc dimer:PGRN fusion proteins Fusion 1, Fusion 2, and Fusion 3. In Fusion 1 and Fusion 2, the N-terminus of PGRN is fused to the C-terminus of the Fc polypeptide that does not contain TfR-binding mutations (indicated by star) by way of a (G₄S)₂ linker (SEQ ID NO:276) and a G₄S linker (SEQ ID NO:277), respectively. In Fusion 3, the C-terminus of PGRN is fused to the N-terminus of the Fc polypeptide that does not contain TfR-binding mutations (indicated by star) by way of a (G₄S)₂ linker.

FIG. 1B shows SDS-PAGE gels demonstrating that fusion proteins Fusion 1, Fusion 2, and Fusion 3 each containing one PGRN molecule were purified to greater than 85% purity.

FIG. 1C is a schematic drawing showing the Fc dimer:PGRN fusion proteins Fusion 4, Fusion 5, and Fusion 6. In Fusion 4, each of the two PGRN molecules is fused to the C-terminus of an Fc polypeptide by way of the linker (G₄S)₂ (SEQ ID NO:276). One PGRN molecule is fused to C-terminus of the Fc polypeptide containing TfR-binding mutations (indicated by star), while the other PGRN molecule is fused to C-terminus of the Fc polypeptide without the TfR-binding mutations. In Fusion 5, one PGRN molecule is fused to the N-terminus of the Fc polypeptide containing TfR-binding mutations (indicated by star) by way of the linker (G₄S)₂, while the other PGRN molecule is fused to the C-terminus of the other Fc polypeptide without the TfR-binding mutations. In Fusion 6, each of the two PGRN molecules is fused to the N-terminus of an Fc polypeptide by way of the linker (G₄S)₂. One PGRN molecule is fused to N-terminus of the Fc polypeptide containing TfR-binding mutations (indicated by star), while the other PGRN molecule is fused to N-terminus of the Fc polypeptide without the TfR-binding mutations.

FIG. 1D shows that Fc dimer:PGRN fusion proteins Fusion 4 and Fusion 5 each containing two PGRN molecules were purified to greater than 85% purity.

FIG. 1E is a schematic drawing showing the Fc dimer:PGRN fusion proteins Fusion 7 and Fusion 8. Both fusion proteins Fusion 7 and Fusion 8 contain Fc polypeptides that do not contain TfR-binding mutations. In Fusion 7, the N-terminus of PGRN is fused to the C-terminus of an Fc polypeptide by way of the linker (G₄S)₂ (SEQ ID NO:276). In Fusion 8, the C-terminus of PGRN is fused to the N-terminus of an Fc polypeptide by way of the linker (G₄S)₂.

FIGS. 2A and 2B show efficient cellular uptake of recombinant PGRN and Fc dimer:PGRN fusion proteins (Fusion 1, Fusion 2, and Fusion 3) as indicated by both PGRN (FIG. 2A) and Fc stainings (FIG. 2B).

FIG. 3 shows Fc dimer:PGRN fusion proteins (Fusion 1, Fusion 2, and Fusion 3) were able to rescue of the proteolytic deficits in GRN KO BMDMs.

FIG. 4 shows the dose-response of Fc dimer:PGRN fusion proteins (Fusion 1, Fusion 3, Fusion 4, and Fusion 5) in an endo-lysosomal proteolysis assay using a Bodipy-BSA conjugate (DQ-BSA).

FIG. 5 shows Fc dimer:PGRN fusion proteins (Fusion 1, Fusion 2, and Fusion 3) were able to rescue elevated cathepsin D activity observed in the GRN KO BMDMs.

FIGS. 6A-6D show Fc dimer:PGRN fusion protein Fusion 1 was able to rescue elevated mRNA levels of the lysosomal genes Cts1, Tmem106b, and Psap in the GRN KO BMDMs.

FIGS. 7A and 7B show that Fc dimer:PGRN fusion proteins Fusion 1 and Fusion 3 displayed similar pharmacokinetic profiles in plasma and liver samples of WT mice dosed with these fusion proteins.

FIGS. 8A and 8B show scatter plots with mean and SEM indicated for concentrations of Fc dimer:PGRN fusion proteins Fusion 1 and Fusion 3 in nM that were measured in brain lysates and liver lysates, respectively, of WT and hTfR mice at 4 hours post-dosing. Data were generated using a biotinylated goat polyclonal human progranulin detection antibody (R&D Systems #BAF2420).

FIG. 9A shows a scatter plot with mean and SEM indicated for brain:plasma ratios of Fc dimer:PGRN fusion proteins Fusion 1 and Fusion 3 normalized to Fusion 3 in WT.

FIG. 9B shows a scatter plot with mean and SEM indicated for brain:liver ratios of Fc dimer:PGRN fusion proteins Fusion 1 and Fusion 3 normalized to Fusion 3 in WT.

FIGS. 10A and 10B show a scatter plot with mean and SEM indicated for concentrations of Fc dimer:PGRN fusion proteins Fusion 1 and Fusion 3 in nM that were measured in brain lysates and liver lysates, respectively, of WT and hTfR mice at 4 hours post-dosing. Data were generated using a detection antibody targeting a site in Fc.

FIGS. 11A and 111B show a scatter plot with mean and SEM indicated for concentrations of Fc dimer:PGRN fusion proteins Fusion 1 and Fusion 3 in ng/mg total protein that were measured in brain lysates and liver lysates, respectively, of WT and hTfR mice at 4 hours post-dosing. Data were generated using a detection antibody targeting a site in Fc.

FIGS. 12A and 12B show semi-log plots of the time course of mean plasma concentrations in nM of Fc dimer:PGRN fusion proteins Fusion 1 and Fusion 3, respectively, in hTfR and WT mice.

FIGS. 12C and 12D show scatter plots with mean and SEM indicated for plasma concentrations in nM of Fc dimer:PGRN fusion proteins Fusion 1 and Fusion 3 at 0.25 hr and 4 hr post-dosing, respectively.

FIGS. 13A and 13B show increased levels of bis(monoacylglycero)phosphate (BMP) species in bone marrow-derived macrophages (BMDMs) of GRN knockout mice compared to wild-type. FIG. 13A shows that treating GRN knockout mouse BMDMs with Fc dimer:PGRN fusion proteins Fusion 11 and Fusion 12 shown in Table 1 of Example 1 reduced elevated BMP(18:1_18:1) levels. FIG. 13B shows that treating GRN knockout mouse BMDMs with Fc dimer:PGRN fusion proteins Fusion 11 and Fusion 12 reduced elevated BMP (20:4_20:4) levels.

FIGS. 13C and 13D show that treating GRN knockout mouse BMDMs with recombinant progranulin (Adipogen) or progranulin expressed by lentivirus reduced elevated BMP levels (total BMP as well as 18:1_18:1).

FIG. 14A shows decreased BMP 44:12 in the periphery liver, plasma, and urine of GRN knockout mice compared to wild-type.

FIG. 14B shows decreased BMP 44:12 in cerebrospinal fluid (CSF) and brain of the central nervous system (CNS) compared to wild-type.

FIG. 15 shows that GRN knockout mice ranging from 2 to 19 months exhibited age-independent reduction in plasma BMP 44:12 compared to wild-type.

FIGS. 16A and 16B show that treatment of GRN knockout mice with Fc dimer:PGRN fusion proteins Fusion 11 and Fusion 12 increased liver BMP 44:12 and 20:4_20:4 levels.

FIG. 17 shows that treatment of GRN knockout mice with Fc dimer:PGRN fusion proteins Fusion 11 and Fusion 12 increased plasma BMP 44:12 levels.

FIG. 18 shows that treatment of GRN knockout mice with Fc dimer:PGRN fusion proteins Fusion 11 and Fusion 12 increased urine BMP 44:12 levels (normalized to creatine).

FIG. 19 shows that treatment of GRN knockout mice with Fc dimer:PGRN fusion proteins Fusion 11 and Fusion 12 increased CSF BMP 44:12 levels.

FIGS. 20A and 20B show that treatment of GRN knockout mice with Fc dimer:PGRN fusion proteins Fusion 11 and Fusion 12 increased brain BMP 44:12 and 20:4_20:4 levels, respectively.

FIGS. 21A-21C show that Fusion 11 and Fusion 12 were able to cross the BBB in the brain of GRN KO/hTfR.KI mice.

### DETAILED DESCRIPTION

### I. Introduction

We have developed fusion proteins that include a progranulin polypeptide or a variant thereof linked to an Fc polypeptide. These proteins can be used to treat progranulin-associated disorders (e.g., a neurodegenerative disease, such as frontotemporal dementia (FTD)). In some cases, the protein includes a dimeric Fc polypeptide, where one of the Fc polypeptide monomers is linked to the progranulin polypeptide. The Fc polypeptides can increase progranulin levels and, in some cases, can be modified to confer additional functional properties onto the protein.

Progranulin (PGRN) (also known as proepithelin and acrogranin) is a cysteine-rich protein encoded by the gene GRN, which maps to human chromosome 17q21. Progranulin is a lysosomal protein as well as a secreted protein consisting of seven and a half tandem repeats of conserved granulin peptides, each of which is about 60 amino acid long and can be released through cleavage by various extracellular proteases (e.g., elastase) and lysosomal proteases (e.g., cathepsin L)(Kao et al., Nat Rev Neurosci. 18(6):325-333, 2017). Generally, progranulin is believed to play both cell-autonomous and non-cell autonomous roles in the control of innate immunity as well as the function of lysosomes, where it regulates the activity and levels of various cathepsins and other hydrolases (Kao et al., supra). Progranulin also has a neurotrophic function and promotes neurite outgrowth and neuronal survival (Kao et al., supra).

Also described herein are fusion proteins that facilitate delivery of a progranulin polypeptide across the blood-brain barrier (BBB). These proteins comprise an Fc polypeptide and a modified Fc polypeptide that form a dimer, and a progranulin polypeptide linked to the Fc region and/or the modified Fc region. The modified Fc region can specifically bind to a BBB receptor such as a transferrin receptor (TfR).

### II. Definitions

As used herein, the singular forms “a,” “an,” and “the” include plural referents unless the content clearly dictates otherwise. Thus, for example, reference to “a polypeptide” may include two or more such molecules, and the like.

As used herein, the terms “about” and “approximately,” when used to modify an amount specified in a numeric value or range, indicate that the numeric value as well as reasonable deviations from the value known to the skilled person in the art, for example 20%, f 10%, or ±5%, are within the intended meaning of the recited value.

As used herein, the term “BMP” refers to bis(monoacylglycero)phosphate. BMP is a glycerophospholipid that is negatively charged (e.g. at the pH normally present within late endosomes and lysosomes) having the structure depicted in Formula I:

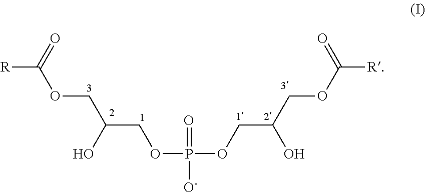

BMP molecules comprise two fatty acid side chains. R and R′ in Formula I represent independently selected saturated or unsaturated aliphatic chains, each of which typically contains 14, 16, 18, 20, or 22 carbon atoms. When a fatty acid side chain is unsaturated, it can contain 1, 2, 3, 4, 5, 6, or more carbon-carbon double bonds. Furthermore, a BMP molecule can contain one or two alkyl ether substituents, wherein the carbonyl oxygen of one or both fatty acid side chains is replaced with two hydrogen atoms. Nomenclature that is used herein to describe a particular BMP species refers to a species having two fatty acid side-chains, wherein the structures of the fatty acid side chains are indicated within parentheses in the BMP format (e.g., BMP(18:1_18:1)). The numerals follow the standard fatty acid notation format of number of “fatty acid carbon atoms:number of double bonds.” An “e-” prefix is used to indicate the presence of an alkyl ether substituent wherein the carbonyl oxygen of the fatty acid side chain is replaced with two hydrogen atoms. For example, the “e” in “BMP(16:0e_18:0)” denotes that the side chain having 16 carbon atoms is an alkyl ether substituent.

A “progranulin polypeptide” or “PGRN polypeptide” refers to a cysteine-rich, lysosomal protein encoded by the gene GRN. A progranulin polypeptide may comprise a human progranulin sequence, e.g., the sequence of SEQ ID NO:211 or 212. A progranulin polypeptide may be a pre-mature progranulin having the sequence of SEQ ID NO:211, in which the first 17 amino acids indicate the signal peptide. A progranulin polypeptide may be a mature progranulin in which the 17-amino acid signal peptide is cleaved. A mature progranulin may comprise the sequence of SEQ ID NO:212. A progranulin polypeptide may include a sequence from a non-human species, e.g., mouse (accession no. NP_032201.2), rat (NP_058809.2 or NP_001139314.1), and chimpanzee (XP_016787144.1 or XP_016787145.1) in either pre-mature or mature form.

A “progranulin polypeptide variant” or “PGRN polypeptide variant” refers to a functional variant of a wild-type progranulin that has at least 90% sequence identity (e.g., 92%, 94%, 96%, 98%, or 99% sequence identity) to a mature wild-type progranulin polypeptide (e.g., SEQ ID NO:212). A progranulin polypeptide variant may have similar functions as those of a wild-type progranulin, e.g., where the progranulin polypeptide variant also binds sortilin or prosaposin, regulates the activity and levels of various lysosomal proteins (e.g., cathepsins), promotes neurite outgrowth and neuronal survival, and/or any other function described herein. The progranulin polypeptide variant comprises granulins G, F, B, A, C, D, and E of a full-length progranulin (e.g., SEQ ID NO:212).

The term “progranulin-associated disorder” refers to any pathological condition relating to progranulin including expression, processing, glycosylation, cellular uptake, trafficking, and/or function. The term “disorder associated with a decreased level of progranulin” refers to any pathological condition that directly or indirectly results from a level of progranulin that is insufficient to enable (i.e., is too low to enable) normal physiological function within a cell, a tissue, and/or a subject, as well as a precursors to such a condition. In some embodiments, the progranulin-associated disorder is a neurodegenerative disease (e.g., frontotemporal dementia (FTD)) or a lysosomal storage disorder.

The term “progranulin level” refers to the amount, concentration, and/or activity level of progranulin that is present, either in a subject or in a sample (e.g., a sample obtained from a subject). A progranulin level can refer to an absolute amount, concentration, and/or activity level of progranulin that is present, or can refer to a relative amount, concentration, and/or activity level. The term also refers to the amount or concentration of a progranulin polypeptide and/or progranulin mRNA (e.g., expressed from a GRN gene) that is present.

The term “bone marrow-derived macrophage” or “BMDM” refers to a macrophage cell that is generated or derived in vitro from a mammalian bone marrow (e.g., a bone marrow obtained from a subject). As a non-limiting example, BMDMs can be generated by culturing undifferentiated bone marrow cells in the presence of a cytokine such as macrophage colony-stimulating factor (M-CSF).

A “transferrin receptor” or “TfR” as used in the context of this disclosure refers to transferrin receptor protein 1. The human transferrin receptor 1 polypeptide sequence is set forth in SEQ ID NO:92. Transferin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion, or insertion, as compared to a wild-type immunoglobulin heavy chain Fc polypeptide sequence, but retains the overall Ig fold or structure of the native Fc polypeptide.

As used herein, the term “Fc polypeptide dimer” refers to a dimer of two Fc polypeptides. In some embodiments, the two Fc polypeptides dimerize by the interaction between the two CH3 domains. If hinge regions or parts of the hinge regions are present in the two Fc polypeptides, one or more disulfide bonds can also form between the hinge regions of the two dimerizing Fc polypeptides.

A “modified Fc polypeptide dimer” refers to a dimer of two Fc polypeptides in which at least one Fc polypeptide is a modified Fc polypeptide that has at least one mutation, e.g., a substitution, deletion, or insertion, as compared to a wild-type immunoglobulin heavy chain Fc polypeptide sequence. For example, a modified Fc polypeptide dimer specifically binds TfR and has at least one modified Fc polypeptide having at least one mutation, e.g., a substitution, deletion, or insertion, as compared to a wild-type immunoglobulin heavy chain Fc polypeptide sequence.

The term “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a β2-microglobulin of about 15 kDa in size.

As used herein, an “FcRn binding site” refers to the region of an Fc polypeptide that binds to FcRn. In human IgG, the FcRn binding site, as numbered using the EU index, includes T250, L251, M252, I253, S254, R255, T256, T307, E380, M428, H433, N434, H435, and Y436. These positions correspond to positions 20 to 26, 77, 150, 198, and 203 to 206 of SEQ ID NO:1.

As used herein, a “native FcRn binding site” refers to a region of an Fc polypeptide that binds to FcRn and that has the same amino acid sequence as the region of a naturally occurring Fc polypeptide that binds to FcRn.

The terms “CH3 domain” and “CH2 domain” as used herein refer to immunoglobulin constant region domain polypeptides. For purposes of this application, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:91).

The terms “wild-type,” “native,” and “naturally occurring” with respect to a CH3 or CH2 domain are used herein to refer to a domain that has a sequence that occurs in nature.

In the context of this disclosure, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and 0-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e. two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:1, when the residue aligns with the amino acid in SEQ ID NO:1 when optimally aligned to SEQ ID NO:1. The polypeptide that is aligned to the reference sequence need not be the same length as the reference sequence.

A “binding affinity” as used herein refers to the strength of the non-covalent interaction between two molecules, e.g., a single binding site on a polypeptide and a target, e.g., transferrin receptor, to which it binds. Thus, for example, the term may refer to 1:1 interactions between a polypeptide and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{D}'s are calculated that may reflect avid binding.

The phrase “specifically binds” or “selectively binds” to a target, e.g., transferrin receptor, when referring to a polypeptide comprising a transferrin receptor-binding modified Fc polypeptide as described herein, refers to a binding reaction whereby the polypeptide binds to the target with greater affinity, greater avidity, and/or greater duration than it binds to a structurally different target, e.g., a target not in the transferrin receptor family. In typical embodiments, the polypeptide has at least 5-fold, 10-fold, 25-fold, 50-fold, 100-fold, 1000-fold, 10,000-fold, or greater affinity for a transferrin receptor compared to an unrelated target when assayed under the same affinity assay conditions. The term “specific binding,” “specifically binds to,” or “is specific for” a particular target (e.g., TfR), as used herein, can be exhibited, for example, by a molecule having an equilibrium dissociation constant KD for the target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some embodiments, a modified Fc polypeptide specifically binds to an epitope on a transferrin receptor that is conserved among species (e.g., structurally conserved among species), e.g., conserved between non-human primate and human species (e.g., structurally conserved between non-human primate and human species). In some embodiments, a polypeptide may bind exclusively to a human transferrin receptor.

The terms “treatment,” “treating,” and the like are used herein to generally mean obtaining a desired pharmacologic and/or physiologic effect. “Treating” or “treatment” may refer to any indicia of success in the treatment or amelioration of a disease, including progranulin-associated disorders, such as neurodegenerative diseases (e.g., frontotemporal dementia (FTD), neuronal ceroid lipofuscinosis (NCL), Niemann-Pick disease type A (NPA), Niemann-Pick disease type B (NPB), Niemann-Pick disease type C (NPC), C9ORF72-associated amyotrophic lateral sclerosis (ALS)/FTD, sporadic ALS, Alzheimer's disease (AD), Gaucher's disease (e.g., Gaucher's disease types 2 and 3), and Parkinson's disease), atherosclerosis, a disorder associated with TDP-43, and age-related macular degeneration (AMD), including any objective or subjective parameter such as abatement, remission, improvement in patient survival, increase in survival time or rate, diminishing of symptoms or making the disorder more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The term “pharmaceutically acceptable excipient” refers to a non-active pharmaceutical ingredient that is biologically or pharmacologically compatible for use in humans or animals, such as but not limited to a buffer, carrier, or preservative.

As used herein, a “therapeutic amount” or “therapeutically effective amount” of an agent is an amount of the agent that treats symptoms of a disease in a subject.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, the polypeptides described herein are administered intravenously.

### III. Progranulin Replacement Therapy

In some aspects, described herein is a fusion protein that comprises: (i) an Fc polypeptide, which may contain modifications (e.g., one or more modifications that promote heterodimerization) or may be a wild-type Fc polypeptide; and a progranulin polypeptide; and (ii) an Fc polypeptide, which may contain modifications (e.g., one or more modifications that promote heterodimerization) or may be a wild-type Fc polypeptide; and optionally a progranulin polypeptide. In some embodiments, one or both Fc polypeptides may contain modifications that result in binding to a blood-brain barrier (BBB) receptor, e.g., a transferrin receptor (TfR). The progranulin polypeptide may be deficient in neurodegenerative diseases. The progranulin polypeptide may be deficient in frontotemporal dementia (FTD), as well as in other diseases, such as Gaucher's disease and Alzheimer's disease (AD). A progranulin polypeptide incorporated into the fusion protein may bind to sortilin or prosaposin. In particular embodiments, the progranulin polypeptide incorporated into the fusion protein to sortilin.

In some embodiments, a fusion protein comprising a progranulin polypeptide and optionally a modified Fc polypeptide that binds to a BBB receptor, e.g., a TfR-binding Fc polypeptide, comprises a variant of a progranulin polypeptide.

In some embodiments, a progranulin polypeptide or a variant thereof, that is present in a fusion protein described herein, retains at least 25% of its activity compared to its activity when not joined to an Fc polypeptide or a TfR-binding Fc polypeptide. In some embodiments, a progranulin polypeptide or a variant thereof, that is present in a fusion protein described herein, retains at least 10%, or at least 15%, 20%, 25%, 30%, 35%, 40%, 45%, 50%, 55%, 60%, 65%, 70%, 75%, 80%, 85%, 90%, or 95%, of its activity compared to its activity when not joined to an Fc polypeptide or a TfR-binding Fc polypeptide. In some embodiments, a progranulin polypeptide or a variant thereof, that is present in a fusion protein described herein, retains at least 80%, 85%, 90%, or 95% of its activity compared to its activity when not joined to an Fc polypeptide or a TfR-binding Fc polypeptide. A fusion protein described herein can be an Fc dimer:PGRN fusion protein comprising: (a) a sequence that has at least 85% identity, at least 90% identity, at least 95% identity, or 100% identity to the sequence of SEQ ID NO:215, and (b) a sequence that has at least 85% identity, at least 90% identity, at least 95% identity, or 100% identity to the sequence of SEQ ID NO:210. A fusion protein described herein can be an Fc dimer:PGRN fusion protein comprising: (a) a sequence that has at least 85% identity, at least 90% identity, at least 95% identity, or 100% identity to the sequence of SEQ ID NO:227, and (b) a sequence that has at least 85% identity, at least 90% identity, at least 95% identity, or 100% identity to the sequence of SEQ ID NO:210. A fusion protein described herein can be an Fc dimer:PGRN fusion protein comprising: (a) a sequence that has at least 85% identity, at least 90% identity, at least 95% identity, or 100% identity to the sequence of SEQ ID NO:215, and (b) a sequence that has at least 85% identity, at least 90% identity, at least 95% identity, or 100% identity to the sequence of SEQ ID NO:291. A fusion protein described herein can be an Fc dimer:PGRN fusion protein comprises: an Fc dimer:PGRN fusion protein comprising: (a) a sequence that has at least 85% identity, at least 90% identity, at least 95% identity, or 100% identity to the sequence of SEQ ID NO:227, and (b) a sequence that has at least 85% identity, at least 90% identity, at least 95% identity, or 100% identity to the sequence of SEQ ID NO:291.

In some embodiments, fusion to an Fc polypeptide does not decrease the expression and/or activity of the progranulin polypeptide or variant thereof. In some embodiments, fusion to a TfR-binding Fc polypeptide does not decrease the expression and/or activity of the progranulin polypeptide.

### IV. Indications

In some embodiments, the fusion proteins described herein comprising a progranulin polypeptide are useful in the treatment of one or more neurodegenerative diseases selected from the group consisting of Alzheimer's disease, primary age-related tauopathy, lewy body dementia, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia.

A progranulin-associated disorder may be a neurodegenerative disease. A number of neurodegenerative diseases may be caused by or linked to lysosomal storage disorders characterized by the accumulation of undigested or partially digested macromolecules, which ultimately results in cellular and organismal dysfunction as well as clinical abnormalities. Lysosomal storage disorders are defined by the type of accumulated substrate, and may be classified as cholesterol storage disorders, sphingolipidoses, oligosaccharidoses, mucolipidoses, mucopolysaccharidoses, lipoprotein storage disorders, neuronal ceroid lipofuscinoses, and others. In some cases, lysosomal storage disorders also include deficiencies or defects in proteins that result in accumulation of macromolecules, such as proteins necessary for normal post-translational modification of lysosomal enzymes, or proteins important for proper lysosomal trafficking. Examples of neurodegenerative diseases that may be caused by or linked to lysosomal storage disorders include, e.g., frontotemporal dementia (FTD), neuronal ceroid lipofuscinosis (NCL), Niemann-Pick disease type A (NPA), Niemann-Pick disease type B (NPB), Niemann-Pick disease type C (NPC), C9ORF72-associated amyotrophic lateral sclerosis (ALS)/FTD, sporadic ALS, Alzheimer's disease (AD), Gaucher's disease (e.g., Gaucher's disease types 2 and 3), and Parkinson's disease. Examples of other progranulin-associated disorders include, e.g., atherosclerosis, a disorder associated with TDP-43, and age-related macular degeneration (AMD). Such progranulin-associated disorders (e.g., a neurodegenerative disease, such as frontotemporal dementia (FTD)) may benefit from the fusion proteins or polypeptides comprising a progranulin polypeptide or a variant thereof as described herein.

Frontotemporal dementia (FTD) is a progressive neurodegenerative disorder. FTD includes a spectrum of clinically, pathologically, and genetically heterogeneous diseases presenting selective involvement of the frontal and temporal lobes (Gazzina et al., Eur J Pharmacol. 817:76-85, 2017). Clinical manifestations of FTD include alterations in behavior and personality, frontal executive deficits, and language dysfunction. Based on the diversity of clinical phenotypes, different presentations have been identified, such as behavioral variants of FTD (bvFTD) and primary progressive aphasia (PPA), which can either be the nonfluent/agrammatic variant PPA (avPPA) or the semantic variant PPA (svPPA). These clinical presentations can also overlap with atypical parkinsonism, such as corticobasal syndrome (CBS), progressive supranuclear palsy (PSP), and amyotrophic lateral sclerosis (ALS) (Gazzina et al., Eur J Pharmacol. 817:76-85, 2017). FTD is associated with various neuropathological hallmarks, including tau pathology in neurons and astrocytes or cytoplasmic ubiquitin inclusions in neurons. The Trans-activating DNA-binding Protein with a molecular weight of 43 kDa (TDP-43) is the most prominent, ubiquitinated protein pathology accumulating in the majority of cases of FTD as well as in ALS (Petkau and Leavitt, Trends Neurosci. 37(7):388-98, 2014). FTD is a significant cause of early-onset dementia with up to 80% of cases presenting between ages 45 and 64. The disease also presents a significant familial component, with about 30-50% of cases reporting family history of the disease (Petkau and Leavitt, supra).

While several genes have been linked to FTD, one of the most frequently mutated genes in FTD is GRN, which maps to human chromosome 17q21 and encodes the cysteine-rich protein progranulin (also known as proepithelin and acrogranin). Recent estimates suggest that GRN mutations account for 5-20% of FTD patients with positive family history and 1-5% of sporadic cases (Rademakers et al., supra). The precise molecular and cellular mechanisms underlying neurodegeneration and disease processes in GRN-associated FTD are unknown, although phenotypic characterization of GRN-knockout mice combined with histological analyses of patients' brain suggests that both inflammation and lysosomal defects are central to the disease (Kao et al., Nat Rev Neurosci. 18(6):325-333, 2017). Indeed, massive gliosis is present in cortical regions of patients (Lui et al., Cell. 165(4):921-35, 2016) and lipofuscin, a lysosomal pigment denoting lysosomal disorder, has been reported in the eye and cortex of mutated GRNcarriers including both presymptomatic individuals and patients (Ward et al., Sci Transl Med. 9(385), 2017).

More than seventy GRN disease mutations have been reported and mapped throughout the gene, where they result in confirmed or predicted loss of function (LOF) alleles (Ji et al. J Med Genet. 54:145-154, 2017). Most heterozygous mutations linked to FTD cause about 50% reduction in mRNA level primarily as a result of non-sense mRNA decay and a comparable reduction in progranulin protein level (Petkau and Leavitt, supra: Kao et al., supra). Lower levels of progranulin are also found in the blood (serum) and cerebrospinal fluid (CSF) of carriers, including presymptomatic individuals (Finch et al., Nat Rev Neurosci. 18(6):325-333, 2017; Goossens et al., Alzheimers Res Ther. 10(1):31, 2018; Meeter et al., Dement Geriatr Cogn Dis Extra. 6(2):330-340, 2016). Therefore, haplo-insufficiency is believed to be the main disease mechanism in GRN-associated FTD, suggesting that therapeutic approaches that elevate progranulin levels in carriers may delay the age of onset as well as the progression of FTD (Petkau and Leavitt, supra; Kao et al., supra). This notion is supported by human genetic studies indicating that a variant of the gene TMFM106B both enhances the levels of progranulin by 25% and delay the age of onset of GRN-associated FTD by 13 years (Nicholson and Rademakers, Acta Neuropathol. 132(5):639-651, 2016).

Homozygous GRN mutations have also been reported, although carriers present a vastly different clinical phenotype known as neuronal ceroid lipofuscinosis (NCL) (Batten disease; incidence 1-2.5 in 100,000 live births; Cotman et al., Curr Neurol Neurosci Rep. 13(8):366, 2013), which is a lysosomal storage disorder (Smith et al., Am J Hum Genet. 90(6):1102-7, 2012; Almeida et al., Neurobiol Aging. 41:200.e1-200.e5, 2016). GRN is in fact one of the 14 ceroid-lipofuscinosis neuronal (CLN) genes reported to be linked to NCL and GRN is also known as CLN11 (Kollmann et al., Biochim Biophys Acta. 1832(11):1866-81, 2013). The fusion proteins or polypeptides comprising a progranulin polypeptide or a variant thereof as described herein may exhibit anti-inflammatory properties and enhances lysosomal function, either of which may be beneficial in NCL.

Patients with Gaucher's disease who carry homozygous mutations in the GBA gene have lower levels of progranulin in their serum (Jian et al., EBioMedicine 11:127-137, 2016). Parkinson's disease patients with heterozygous mutations in GBA may also have lower levels of progranulin. The fusion proteins or polypeptides comprising a progranulin polypeptide or a variant thereof as described herein may provide therapeutic benefits in treating Gaucher's disease or Parkinson's disease.

Variants in GRN have been linked to AD (Rademakers et al., supra: Brouwers et al., Neurology. 71(9):656-64, 2008; Lee et al., Neurodegener Dis. 8(4):216-20, 2011; Viswanathan et al., Am J Med Genet B Neuropsychiatr Genet. 150B(5):747-50, 2009) and the TDP-43 pathology is common in the brain of AD patients (Youmans and Wolozin, Exp Neurol. 237(1):90-5, 2012). Progranulin gene delivery has also been shown to decrease amyloid burden in mouse models of AD (van Kampen and Kay, PLoS One. 12(8):e0182896, 2017). Thus, the fusion proteins or polypeptides comprising a progranulin polypeptide or a variant thereof as described herein may also confer therapeutic benefits in treating AD.

Niemann-Pick disease types A and B (NPA and NPB) result from mutations in the gene encoding acid sphingomyelinase (SMPD1). Niemann-Pick disease type C (NPC) results from mutations in the genes involved in cholesterol transport, i.e., NPC1 and NPC2 (Kolter and Sandhoff, Annu Rev Cell Dev Biol. 21:81-103, 2005; Kobayashi et al., Nat Cell Biol. 1(2):113-8, 1999). The fusion proteins or polypeptides comprising a progranulin polypeptide or a variant thereof as described herein may provide therapeutic benefits in treating NPA, NPB, and/or NPC.

The vast majority of ALS cases present the TDP-43 pathology, which is also shared with patients harboring GRN mutations (Petkau and Leavitt, Trends Neurosci. 37(7):388-98, 2014; Rademakers et al., Nat Rev Neurol. 8(8):423-34, 2012). Among all ALS cases, GGGGCC repeat expansions within the C9ORF72 gene are the most common cause of ALS and a significant cause of FTD. The average mutation frequencies reported in North American and European populations are 37% for familial ALS, 6% for sporadic ALS, 21% for familial FTD, and 6% for sporadic FTD patients (Rademakers et al., supra). Additionally, the TMEM106B variant that is protective in GRN-associated FTD is also protective in FTD patients harboring repeat expansions in the C9ORF72 gene (van Blitterswijk et al., Acta Neuropathol. 127(3):397-406, 2014). The fusion proteins or polypeptides comprising a progranulin polypeptide or a variant thereof as described herein may reduce TDP-43 pathology in C9ORF72-associated ALS/FTD by promoting lysosomal function and/or decreasing inflammation.

AMD is a degenerative disease and a major cause of blindness in the developed world. It causes damage to the macula, a small spot near the center of the retina and the part of the eye needed for sharp, central vision. The degenerative changes in the eye and loss of vision may be caused by impaired function of lysosomes and harmful protein accumulations behind the retina (Viiri et al., PloS One. 8(7):e69563, 2013). As the disease progresses, retinal sensory cells in the central vision area are damaged, leading to loss of central vision. The fusion proteins or polypeptides comprising a progranulin polypeptide or a variant thereof as described herein may provide therapeutic benefits in treating AMD.

### V. FC Polypeptide Modifications for Blood-Brain Barrier (BBB) Receptor Binding

In some aspects, provided herein are fusion proteins that are capable of being transported across the blood-brain barrier (BBB). Such a protein comprises a modified Fc polypeptide that binds to a BBB receptor. BBB receptors are expressed on BBB endothelia, as well as other cell and tissue types. In some embodiments, the BBB receptor is transferrin receptor (TfR).

Amino acid residues designated in various Fc modifications, including those introduced in a modified Fc polypeptide that binds to a BBB receptor, e.g., TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein.

A modified (e.g., enhancing heterodimerization and/or BBB receptor-binding) Fc polypeptide present in a fusion protein described herein can have at least 70% identity, at least 75% identity, at least 80% identity, at least 85% identity, at least 90% identity, or at least 95% identity to a native Fc region sequence or a fragment thereof, e.g., a fragment of at least 50 amino acids or at least 100 amino acids, or greater in length. In some embodiments, the native Fc amino acid sequence is the Fc region sequence of SEQ ID NO:1. In some embodiments, the modified Fc polypeptide has at least 70% identity, at least 75% identity, at least 80% identity, at least 85% identity, at least 90% identity, or at least 95% identity to amino acids 1-110 of SEQ ID NO:1, or to amino acids 111-217 of SEQ ID NO:1, or a fragment thereof, e.g., a fragment of at least 50 amino acids or at least 100 amino acids, or greater in length.

In some embodiments, a modified (e.g., enhancing heterodimerization and/or BBB receptor-binding) Fc polypeptide comprises at least 50 amino acids, or at least 60, 65, 70, 75, 80, 85, 90, or 95 or more, or at least 100 amino acids, or more, that correspond to a native Fc region amino acid sequence. In some embodiments, the modified Fc polypeptide comprises at least 25 contiguous amino acids, or at least 30, 35, 40, or 45 contiguous amino acids, or 50 contiguous amino acids, or at least 60, 65, 70, 75, 80 85, 90, or 95 or more contiguous amino acids, or 100 or more contiguous amino acids, that correspond to a native Fc region amino acid sequence, such as SEQ ID NO:1.

In some embodiments, the domain that is modified for BBB receptor-binding activity is a human Ig CH3 domain, such as an IgG1 CH3 domain. The CH3 domain can be of any IgG subtype, i.e., from IgG1, IgG2, IgG3, or IgG4. In the context of IgG1 antibodies, a CH3 domain refers to the segment of amino acids from about position 341 to about position 447 as numbered according to the EU numbering scheme.

In some embodiments, the domain that is modified for BBB receptor-binding activity is a human Ig CH2 domain, such as an IgG CH2 domain. The CH2 domain can be of any IgG subtype, i.e., from IgG1, IgG2, IgG3, or IgG4. In the context of IgG1 antibodies, a CH2 domain refers to the segment of amino acids from about position 231 to about position 340 as numbered according to the EU numbering scheme.

In some embodiments, a modified (e.g., BBB receptor-binding) Fc polypeptide present in a fusion protein described herein comprises at least one, two, or three substitutions; and in some embodiments, at least four five, six, seven, eight, nine, or ten substitutions at amino acid positions comprising 266, 267, 268, 269, 270, 271, 295, 297, 298, and 299, according to the EU numbering scheme.

In some embodiments, a modified (e.g., BBB receptor-binding) Fc polypeptide present in a fusion protein described herein comprises at least one, two, or three substitutions; and in some embodiments, at least four, five, six, seven, eight, or nine substitutions at amino acid positions comprising 274, 276, 283, 285, 286, 287, 288, 289, and 290, according to the EU numbering scheme.

In some embodiments, a modified (e.g., BBB receptor-binding) Fc polypeptide present in a fusion protein described herein comprises at least one, two, or three substitutions; and in some embodiments, at least four, five, six, seven, eight, nine, or ten substitutions at amino acid positions comprising 268, 269, 270, 271, 272, 292, 293, 294, 296, and 300, according to the EU numbering scheme.

In some embodiments, a modified (e.g., BBB receptor-binding) Fc polypeptide present in a fusion protein described herein comprises at least one, two, or three substitutions; and in some embodiments, at least four, five, six, seven, eight, or nine substitutions at amino acid positions comprising 272, 274, 276, 322, 324, 326, 329, 330, and 331, according to the EU numbering scheme.

In some embodiments, a modified (e.g., BBB receptor-binding) Fc polypeptide present in a fusion protein described herein comprises at least one, two, or three substitutions; and in some embodiments, at least four, five, six, or seven substitutions at amino acid positions comprising 345, 346, 347, 349, 437, 438, 439, and 440, according to the EU numbering scheme.

In some embodiments, a modified (e.g., BBB receptor-binding) Fc polypeptide present in a fusion protein described herein comprises at least one, two, or three substitutions; and in some embodiments, at least four, five, six, seven, eight, or nine substitutions at amino acid positions 384, 386, 387, 388, 389, 390, 413, 416, and 421, according to the EU numbering scheme.

### FcRn Binding Sites

In certain aspects, modified (e.g., BBB receptor-binding) Fc polypeptides, or Fc polypeptides present in a fusion protein described herein that do not specifically bind to a BBB receptor, can also comprise an FcRn binding site. In some embodiments, the FcRn binding site is within the Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart modified Fc polypeptide having the wild-type residues at the mutated positions when assayed under the same conditions). In some embodiments, an FcRn binding site has one or more amino acid residues that are substituted at positions 250-256, 307, 380, 428, and 433-436, according to the EU numbering scheme.

In some embodiments, one or more residues at or near an FcRn binding site are mutated, relative to a native human IgG sequence, to extend serum half-life of the modified polypeptide. In some embodiments, mutations are introduced into one, two, or three of positions 252, 254, and 256. In some embodiments, the mutations are M252Y, S254T, and T256E. In some embodiments, a modified Fc polypeptide further comprises the mutations M252Y, S254T, and T256E. In some embodiments, a modified Fc polypeptide comprises a substitution at one, two, or all three of positions T307, E380, and N434, according to the EU numbering scheme. In some embodiments, the mutations are T307Q and N434A. In some embodiments, a modified Fc polypeptide comprises mutations T307A, E380A, and N434A. In some embodiments, a modified Fc polypeptide comprises substitutions at positions T250 and M428, according to the EU numbering scheme. In some embodiments, the modified Fc polypeptide comprises mutations T250Q and/or M428L. In some embodiments, a modified Fc polypeptide comprises substitutions at positions M428 and N434, according to the EU numbering scheme. In some embodiments, the modified Fc polypeptide comprises mutations M428L and N434S. In some embodiments, a modified Fc polypeptide comprises an N434S or N434A mutation.

### VI. Transferrin Receptor-Binding Fc Polypeptides

This section describes generation of modified Fc polypeptides in accordance with the disclosure that bind to transferrin receptor (TfR) and are capable of being transported across the blood-brain barrier (BBB).

### TfR-Binding Fc Polypeptides Comprising Mutations in the CH3 Domain

In some embodiments, a modified Fc polypeptide that specifically binds to TfR comprises substitutions in a CH3 domain. In some embodiments, a modified Fc polypeptide comprises a human Ig CH3 domain, such as an IgG CH3 domain, that is modified for TfR-binding activity. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20%/0 (e.g., less than about 19%, 18%, 17%, 16%, 15%, 14%, 13%, 12%, 11%, 10%, 9%, 8%, 7%, 6%, 5%, 4%, 3%, 2%, or 1%).

In some embodiments, a modified Fc polypeptide that specifically binds to TfR comprises at least two, three, four, five, six, seven, eight, or nine substitutions at positions 384, 386, 387, 388, 389, 390, 413, 416, and 421, according to the EU numbering scheme. Illustrative substitutions that may be introduced at these positions are shown in Tables 6 and 7 at the end of the Examples section. In some embodiments, the amino acid at position 388 and/or 421 is an aromatic amino acid, e.g., Trp, Phe, or Tyr. In some embodiments, the amino acid at position 388 is Trp. In some embodiments, the aromatic amino acid at position 421 is Trp or Phe.

In some embodiments, at least one position as follows is substituted: Leu, Tyr, Met, or Val at position 384: Leu, Thr, His, or Pro at position 386; Val, Pro, or an acidic amino acid at position 387; an aromatic amino acid, e.g., Trp at position 388; Val, Ser, or Ala at position 389; an acidic amino acid, Ala, Ser, Leu, Thr, or Pro at position 413; Thr or an acidic amino acid at position 416; or Trp, Tyr, His, or Phe at position 421. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 384, 386, and/or position 413. In some embodiments, the acidic amino acid at position one, two, or each of positions 387, 413, and 416 is Glu. In other embodiments, the acidic amino acid at one, two or each of positions 387, 413, and 416 is Asp. In some embodiments, two, three, four, five, six, seven, or all eight of positions 384, 386, 387, 388, 389, 413, 416, and 421 have an amino acid substitution as specified in this paragraph.

In some embodiments, an Fc polypeptide that is modified as described in the preceding two paragraphs comprises a native Asn at position 390. In some embodiments, the modified Fc polypeptide comprises Gly, His, Gln, Leu, Lys, Val, Phe, Ser, Ala, or Asp at position 390. In some embodiments, the modified Fc polypeptide further comprises one, two, three, or four substitutions at positions comprising 380, 391, 392, and 415, according to the EU numbering scheme. In some embodiments, Trp, Tyr, Leu, or Gln may be present at position 380. In some embodiments, Ser, Thr, Gln, or Phe may be present at position 391. In some embodiments, Gln, Phe, or His may be present at position 392. In some embodiments, Glu may be present at position 415.

In certain embodiments, the modified Fc polypeptide comprises two, three, four, five, six, seven, eight, nine, ten, or eleven positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421. In some embodiments, the modified Fc polypeptide comprises all eleven positions as follows: Trp, Leu, or Glu at position 380; Tyr or Phe at position 384; Thr at position 386; Glu at position 387; Trp at position 388: Ser, Ala, Val, or Asn at position 389; Ser or Asn at position 390; Thr or Ser at position 413; Glu or Ser at position 415; Glu at position 416; and/or Phe at position 421.

In certain embodiments, the modified Fc polypeptide comprises Leu or Met at position 384; Leu, His, or Pro at position 386; Val at position 387; Trp at position 388; Val or Ala at position 389; Pro at position 413; Thr at position 416; and/or Trp at position 421. In some embodiments, the modified Fc polypeptide further comprises Ser, Thr, Gln, or Phe at position 391. In some embodiments, the modified Fc polypeptide further comprises Trp, Tyr, Leu, or Gln at position 380 and/or Gln, Phe, or His at position 392. In some embodiments, Trp is present at position 380 and/or Gln is present at position 392. In some embodiments, the modified Fc polypeptide does not have a Trp at position 380.

In other embodiments, the modified Fc polypeptide comprises Tyr at position 384; Thr at position 386; Glu or Val and position 387; Trp at position 388; Ser at position 389; Ser or Thr at position 413; Glu at position 416; and/or Phe at position 421. In some embodiments, the modified Fc polypeptide comprises a native Asn at position 390. In certain embodiments, the modified Fc polypeptide further comprises Trp, Tyr, Leu, or Gln at position 380; and/or Glu at position 415. In some embodiments, the modified Fc polypeptide further comprises Tip at position 380 and/or Glu at position 415.

In additional embodiments, the modified Fc polypeptide further comprises one, two, or three substitutions at positions comprising 414, 424, and 426, according to the EU numbering scheme. In some embodiments, position 414 is Lys, Arg, Gly, or Pro; position 424 is Ser, Thr, Glu, or Lys; and/or position 426 is Ser, Trp, or Gly.

In some embodiments, the modified Fc polypeptide comprises one or more of the following substitutions: Trp at position 380; Thr at position 386; Trp at position 388; Val at position 389; Thr or Ser at position 413; Glu at position 415; and/or Phe at position 421, according to the EU numbering scheme.

In some embodiments, the modified Fc polypeptide has at least 70% identity, at least 75% identity, at least 80% identity, at least 85% identity, at least 90% identity, or at least 95% identity to amino acids 111-217 of any one of SEQ ID NOS:4-90, 93-96, and 101-104 (e.g., SEQ ID NOS:34-38, 58, and 60-90). In some embodiments, the modified Fc polypeptide has at least 70% identity, at least 75% identity, at least 80% identity, at least 85% identity, at least 90% identity, or at least 95% identity to any one of SEQ ID NOS:4-90, 93-96, and 101-104 (e.g., SEQ ID NOS:34-38, 58, and 60-90). In some embodiments, the modified Fc polypeptide comprises the amino acids at EU index positions 384-390 and/or 413-421 of any one of SEQ ID NOS:4-90, 93-96, and 101-104 (e.g., SEQ ID NOS:34-38, 58, and 60-90). In some embodiments, the modified Fc polypeptide comprises the amino acids at EU index positions 380-390 and/or 413-421 of any one of 4-90, 93-96, and 101-104 (e.g., SEQ ID NOS:34-38, 58, and 60-90). In some embodiments, the modified Fc polypeptide comprises the amino acids at EU index positions 380-392 and/or 413-426 of any one of SEQ ID NOS:4-90, 93-96, and 101-104 (e.g., SEQ ID NOS:34-38, 58, and 60-90).

In some embodiments, the modified Fc polypeptide has at least 75% identity, at least 80% identity, at least 85% identity, at least 90% identity, or at least 95% identity to any one of SEQ ID NOS:4-90, 93-96, and 101-104 (e.g., SEQ ID NOS:34-38, 58, and 60-90), and further comprises at least five, six, seven, eight, nine, ten, eleven, twelve, thirteen, fourteen, fifteen, or sixteen of the positions, numbered according to the EU index, as follows: Trp, Tyr, Leu, Gln, or Glu at position 380; Leu, Tyr, Met, or Val at position 384; Leu, Thr, His, or Pro at position 386; Val, Pro, or an acidic amino acid at position 387; an aromatic amino acid, e.g., Trp, at position 388; Val, Ser, or Ala at position 389; Ser or Asn at position 390; Ser, Thr, Gln, or Phe at position 391; Gln, Phe, or His at position 392; an acidic amino acid, Ala, Ser, Leu, Thr, or Pro at position 413; Lys, Arg, Gly or Pro at position 414; Glu or Ser at position 415; Thr or an acidic amino acid at position 416; Trp, Tyr, His or Phe at position 421; Ser, Thr, Glu or Lys at position 424; and Ser, Trp, or Gly at position 426.

In some embodiments, the modified Fc polypeptide comprises the amino acid sequence of any one of SEQ ID NOS:34-38, 58, and 60-90. In other embodiments, the modified Fc polypeptide comprises the amino acid sequence of any one of SEQ ID NOS:34-38, 58, and 60-90, but in which one, two, or three amino acids are substituted.

In some embodiments, the modified Fc polypeptide comprises additional mutations such as the mutations described in Section VII below, including, but not limited to, a knob mutation (e.g., T366W as numbered with reference to EU numbering), hole mutations (e.g., T366S, L368A, and Y407V as numbered with reference to EU numbering), mutations that modulate effector function (e.g., L234A, L235A, and/or P329G (e.g., L234A and L235A) as numbered with reference to EU numbering), and/or mutations that increase serum stability (e.g., M252Y, S254T, and T256E as numbered with reference to EU numbering). By way of illustration, SEQ ID NOS:136-210 provide non-limiting examples of modified Fc polypeptides with mutations in the CH3 domain (e.g., clones CH3C.35.21.17, CH3C.35.20.1, CH3C.35.23.2, CH3C.35.23.3, CH3C.35.23.4, CH3C.35.21.17.2, and CH3C.35.23) comprising one or more of these additional mutations.

In some embodiments, the modified Fc polypeptide comprises a knob mutation (e.g., T366W as numbered with reference to EU numbering) and has at least 85% identity, at least 90% identity, or at least 95% identity to the sequence of any one of SEQ ID NOS:136, 137, 149, 161, 173, 185, and 197. In some embodiments, the modified Fc polypeptide comprises the sequence of any one of SEQ ID NOS:136, 137, 149, 161, 173, 185, and 197. In some embodiments, the modified Fc polypeptide comprises the sequence of SEQ ID NO:136.

In some embodiments, the modified Fc polypeptide comprises a knob mutation (e.g., T366W as numbered with reference to EU numbering) and mutations that modulate effector function (e.g., L234A, L235A, and/or P329G (e.g., L234A and L235A) as numbered with reference to EU numbering), and has at least 85% identity, at least 90% identity, or at least 95% identity to the sequence of any one of SEQ ID NOS:138, 139, 150, 151, 162, 163, 174, 175, 186, 187, 198, 199, 209, and 210. In some embodiments, the modified Fc polypeptide comprises the sequence of any one of SEQ ID NOS:138, 139, 150, 151, 162, 163, 174, 175, 186, 187, 198, and 199. In some embodiments, the modified Fc polypeptide comprises the sequence of SEQ ID NO:150.

In some embodiments, the modified Fc polypeptide comprises a knob mutation (e.g., T366W as numbered with reference to EU numbering) and mutations that increase serum stability (e.g., M252Y, S254T, and T256E as numbered with reference to EU numbering), and has at least 85% identity, at least 90% identity, or at least 95% identity to the sequence of any one of SEQ ID NOS:140, 152, 164, 176, 188, and 200. In some embodiments, the modified Fc polypeptide comprises the sequence of any one of SEQ ID NOS: 140, 152, 164, 176, 188, and 200.

In some embodiments, the modified Fc polypeptide comprises a knob mutation (e.g., T366W as numbered with reference to EU numbering), mutations that modulate effector function (e.g., L234A, L235A, and/or P329G (e.g., L234A and L235A) as numbered with reference to EU numbering), and mutations that increase serum stability (e.g., M252Y, S254T, and T256E as numbered with reference to EU numbering), and has at least 85% identity, at least 90% identity, or at least 95% identity to the sequence of any one of SEQ ID NOS:141, 142, 153, 154, 165, 166, 177, 178, 189, 190, 201, and 202. In some embodiments, the modified Fc polypeptide comprises the sequence of any one of SEQ ID NOS:141, 142, 153, 154, 165, 166, 177, 178, 189, 190, 201, and 202.

In some embodiments, the modified Fc polypeptide comprises hole mutations (e.g., T366S, L368A, and Y407V as numbered with reference to EU numbering) and has at least 85% identity, at least 90% identity, or at least 95% identity to the sequence of any one of SEQ ID NOS:143, 155, 167, 179, 191, and 203. In some embodiments, the modified Fc polypeptide comprises the sequence of any one of SEQ ID NOS:143, 155, 167, 179, 191, and 203.

In some embodiments, the modified Fc polypeptide comprises hole mutations (e.g., T366S, L368A, and Y407V as numbered with reference to EU numbering) and mutations that modulate effector function (e.g., L234A, L235A, and/or P329G (e.g., L234A and L235A) as numbered with reference to EU numbering), and has at least 85% identity, at least 90% identity, or at least 95% identity to the sequence of any one of SEQ ID NOS:144, 145, 156, 157, 168, 169, 180, 181, 192, 193, 204, and 205. In some embodiments, the modified Fc polypeptide comprises the sequence of any one of SEQ ID NOS:144, 145, 156, 157, 168, 169, 180, 181, 192, 193, 204, and 205.

In some embodiments, the modified Fc polypeptide comprises hole mutations (e.g., T366S, L368A, and Y407V as numbered with reference to EU numbering) and mutations that increase serum stability (e.g., M252Y, S254T, and T256E as numbered with reference to EU numbering), and has at least 85% identity, at least 90% identity, or at least 95% identity to the sequence of any one of SEQ ID NOS:146, 158, 170, 182, 194, and 206. In some embodiments, the modified Fc polypeptide comprises the sequence of any one of SEQ ID NOS:146, 158, 170, 182, 194, and 206.

In some embodiments, the modified Fc polypeptide comprises hole mutations (e.g., T366S, L368A, and Y407V as numbered with reference to EU numbering), mutations that modulate effector function (e.g., L234A, L235A, and/or P329G (e.g., L234A and L235A) as numbered with reference to EU numbering), and mutations that increase serum stability (e.g., M252Y, S254T, and T256E as numbered with reference to EU numbering), and has at least 85% identity, at least 90% identity, or at least 95% identity to the sequence of any one of SEQ ID NOS:147, 148, 159, 160, 171, 172, 183, 184, 195, 196, 207, and 208. In some embodiments, the modified Fc polypeptide comprises the sequence of any one of SEQ ID NOS:147, 148, 159, 160, 171, 172, 183, 184, 195, 196, 207, and 208.

In some embodiments, a modified Fc polypeptide that specifically binds to TfR comprises at least two, three, four, five, six, seven, or eight substitutions at positions 345, 346, 347, 349, 437, 438, 439, and 440, according to the EU numbering scheme. Illustrative modified Fc polypeptides are provided in SEQ ID NOS: 111-115. In some embodiments, the modified Fc polypeptide comprises Gly at position 437; Phe at position 438; and/or Asp at position 440. In some embodiments, Glu is present at position 440. In certain embodiments, the modified Fc polypeptide comprises at least one substitution at a position as follows: Phe or Ile at position 345; Asp, Glu, Gly, Ala, or Lys at position 346; Tyr, Met, Leu, Ile, or Asp at position 347; Thr or Ala at position 349; Gly at position 437; Phe at position 438; His Tyr, Ser, or Phe at position 439; or Asp at position 440. In some embodiments, two, three, four, five, six, seven, or all eight of positions 345, 346, 347, 349, 437, 438, 439, and 440 and have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide has at least 70% identity, at least 75% identity, at least 80% identity, at least 85% identity, at least 90% identity, or at least 95% identity to amino acids 111-217 of any one of SEQ ID NOS:111-115. In some embodiments, the modified Fc polypeptide has at least 70% identity, at least 75% identity, at least 80% identity, at least 85% identity, at least 90% identity, or at least 95% identity to SEQ ID NOS: 111-115. In some embodiments, the modified Fc polypeptide comprises the amino acid sequence of any one of SEQ ID NOS: 111-115. In other embodiments, the modified Fc polypeptide comprises the amino acid sequence of any one of SEQ ID NOS:111-115, but in which one, two, or three amino acids are substituted.

### TfR-Binding Fc Polypeptides Comprising Mutations in the CH2 Domain

In some embodiments, a modified Fc polypeptide that specifically binds to TfR comprises substitutions in a CH2 domain. In some embodiments, a modified Fc polypeptide comprises a human Ig CH2 domain, such as an IgG CH2 domain, that is modified for TfR-binding activity. The CH2 domain can be of any IgG subtype, i.e., from IgG1, IgG2, IgG3, or IgG4. In the context of IgG antibodies, a CH2 domain refers to the segment of amino acids from about position 231 to about position 340 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2% a, or 1%).

In some embodiments, a modified Fc polypeptide that specifically binds to TfR comprises at least two, three, four, five, six, seven, eight, or nine substitutions at positions 274, 276, 283, 285, 286, 287, 288, and 290, according to the EU numbering scheme. Illustrative modified Fc polypeptides are provided in SEQ ID NOS:116-120. In some embodiments, the modified Fc polypeptide comprises Glu at position 287 and/or Trp at position 288. In some embodiments, the modified Fc polypeptide comprises at least one substitution at a position as follows: Glu, Gly, Gln, Ser, Ala, Asn, Tyr, or Trp at position 274; Ile, Val, Asp, Glu, Thr, Ala, or Tyr at position 276; Asp, Pro, Met, Leu, Ala, Asn, or Phe at position 283; Arg, Ser, Ala, or Gly at position 285; Tyr, Trp, Arg, or Val at position 286; Glu at position 287; Trp or Tyr at position 288; Gln, Tyr, His, lie, Phe, Val, or Asp at position 289; or Leu, Trp, Arg, Asn, Tyr, or Val at position 290. In some embodiments, two, three, four, five, six, seven, eight, or all nine of positions 274, 276, 283, 285, 286, 287, 288, and 290 have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises Glu, Gly, Gln, Ser, Ala, Asn, or Tyr at position 274; Ile, Val, Asp, Glu, Thr, Ala, or Tyr at position 276 Asp, Pro, Met, Leu, Ala, or Asn at position 283; Arg, Ser, or Ala at position 285; Tyr, Trp, Arg, or Val at position 286; Glu at position 287; Trp at position 288; Gln, Tyr, His, Ile, Phe, or Val at position 289; and/or Leu, Trp, Arg, Asn, or Tyr at position 290. In some embodiments, the modified Fc polypeptide comprises Arg at position 285; Tyr or Trp at position 286; Glu at position 287; Trp at position 288; and/or Arg or Trp at position 290.

In some embodiments, the modified Fc polypeptide has at least 70% identity, at least 75% identity, at least 80% identity, at least 85% identity, at least 90% identity, or at least 95% identity to amino acids 1-110 of any one of SEQ ID NOS:116-120. In some embodiments, the modified Fc polypeptide has at least 70% identity, at least 75% identity, at least 80% identity, at least 85% identity, at least 90% identity, or at least 95% identity to SEQ ID NOS:116-120. In some embodiments, the modified Fc polypeptide comprises the amino acid sequence of any one of SEQ ID NOS:116-120. In other embodiments, the modified Fc polypeptide comprises the amino acid sequence of any one of SEQ ID NOS: 116-120, but in which one, two, or three amino acids are substituted.

In some embodiments, a modified Fc polypeptide that specifically binds to TfR comprises at least two, three, four, five, six, seven, eight, nine, or ten substitutions at positions 266, 267, 268, 269, 270, 271, 295, 297, 298, and 299, according to the EU numbering scheme. Illustrative modified Fc polypeptides are provided in SEQ ID NOS:121-125. In some embodiments, the modified Fc polypeptide comprises Pro at position 270, Glu at position 295, and/or Tyr at position 297. In some embodiments, the modified Fc polypeptide comprises at least one substitution at a position as follows: Pro, Phe, Ala, Met, or Asp at position 266; Gln, Pro, Arg, Lys, Ala, Ile, Leu, Glu, Asp, or Tyr at position 267; Thr, Ser, Gly, Met, Val, Phe, Trp, or Leu at position 268; Pro, Val, Ala, Thr, or Asp at position 269; Pro, Val, or Phe at position 270; Trp, Gln, Thr, or Glu at position 271; Glu, Val, Thr, Leu, or Trp at position 295; Tyr, His, Val, or Asp at position 297; Thr, His, Gln, Arg, Asn, or Val at position 298; or Tyr, Asn, Asp, Ser, or Pro at position 299. In some embodiments, two, three, four, five, six, seven, eight, nine, or all ten of positions 266, 267, 268, 269, 270, 271, 295, 297, 298, and 299 have a substitution as specified in this paragraph. In some embodiments, a modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises Pro, Phe, or Ala at position 266; Gln, Pro, Arg, Lys, Ala, or Ile at position 267; Thr, Ser, Gly, Met, Val, Phe, or Trp at position 268; Pro, Val, or Ala at position 269; Pro at position 270; Trp or Gln at position 271; Glu at position 295; Tyr at position 297; Thr, His, or Gln at position 298; and/or Tyr, Asn, Asp, or Ser at position 299.

In some embodiments, the modified Fc polypeptide comprises Met at position 266; Leu or Glu at position 267; Trp at position 268; Pro at position 269; Val at position 270; Thr at position 271; Val or Thr at position 295; His at position 197; His, Arg, or Asn at position 198; and/or Pro at position 299.

In some embodiments, the modified Fc polypeptide comprises Asp at position 266; Asp at position 267; Leu at position 268; Thr at position 269; Phe at position 270; Gln at position 271; Val or Leu at position 295; Val at position 297; Thr at position 298; and/or Pro at position 299.

In some embodiments, the modified Fc polypeptide has at least 70% identity, at least 75% identity, at least 80% identity, at least 85% identity, at least 90% identity, or at least 95% identity to amino acids 1-110 of any one of SEQ ID NOS:121-125. In some embodiments, the modified Fc polypeptide has at least 70% identity, at least 75% identity, at least 80% identity, at least 85% identity, at least 90% identity, or at least 95% identity to SEQ ID NOS:121-125. In some embodiments, the modified Fc polypeptide comprises the amino acid sequence of any one of SEQ ID NOS:121-125. In other embodiments, the modified Fc polypeptide comprises the amino acid sequence of any one of SEQ ID NOS:121-125, but in which one, two, or three amino acids are substituted.

In some embodiments, a modified Fc polypeptide that specifically binds to TfR comprises at least two, three, four, five, six, seven, eight, nine, or ten substitutions at positions 268, 269, 270, 271, 272, 292, 293, 294, and 300, according to the EU numbering scheme. Illustrative modified Fc polypeptides are provided in SEQ ID NOS:126-130. In some embodiments, the modified Fc polypeptide comprises at least one substitution at a position as follows: Val or Asp at position 268; Pro, Met, or Asp at position 269; Pro or Trp at position 270; Arg, Trp, Glu, or Thr at position 271; Met, Tyr, or Trp at position 272; Leu or Trp at position 292; Thr, Val, Ile, or Lys at position 293; Ser, Lys, Ala, or Leu at position 294; His, Leu, or Pro at position 296; or Val or Trp at position 300. In some embodiments, two, three, four, five, six, seven, eight, nine, or all ten of positions 268, 269, 270, 271, 272, 292, 293, 294, and 300 have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises Val at position 268; Pro at position 269; Pro at position 270; Arg or Trp at position 271; Met at position 272; Leu at position 292; Thr at position 293; Ser at position 294; His at position 296; and/or Val at position 300.

In some embodiments, the modified Fc polypeptide comprises Asp at position 268; Met or Asp at position 269; Trp at position 270; Glu or Thr at position 271; Tyr or Trp at position 272; Trp at position 292; Val, Ile, or Lys at position 293; Lys, Ala, or Leu at position 294; Leu or Pro at position 296; and/or Trp at position 300.

In some embodiments, the modified Fc polypeptide has at least 70% identity, at least 75% identity, at least 80% identity, at least 85% identity, at least 90% identity, or at least 95% identity to amino acids 1-110 of any one of SEQ ID NOS:126-130. In some embodiments, the modified Fc polypeptide has at least 70% identity, at least 75% identity, at least 80% identity, at least 85% identity, at least 90% identity, or at least 95% identity to SEQ ID NOS:126-130. In some embodiments, the modified Fc polypeptide comprises the amino acid sequence of any one of SEQ ID NOS:126-130. In other embodiments, the modified Fc polypeptide comprises the amino acid sequence of any one of SEQ ID NOS:126-130, but in which one, two, or three amino acids are substituted.

In some embodiments, a modified Fc polypeptide that specifically binds to TfR has at least two, three, four, five, six, seven, eight, nine, or ten substitutions at positions 272, 274, 276, 322, 324, 326, 329, 330, and 331, according to the EU numbering scheme. Illustrative modified Fc polypeptides are provided in SEQ ID NOS:131-135. In some embodiments, the modified Fc polypeptide comprises Trp at position 330. In some embodiments, the modified Fc polypeptide comprises at least one substitution at a position as follows: Trp, Val, lie, or Ala at position 272; Trp or Gly at position 274; Tyr, Arg, or Glu at position 276; Ser, Arg, or Gln at position 322; Val, Ser, or Phe at position 324; Ile, Ser, or Trp at position 326; Trp, Thr, Ser, Arg, or Asp at position 329; Tip at position 330; or Ser, Lys, Arg, or Val at position 331. In some embodiments, two, three, four, five, six, seven, eight, or all nine of positions 272, 274, 276, 322, 324, 326, 329, 330, and 331 have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises two, three, four, five, six, seven, eight, or nine positions selected from the following: position 272 is Trp, Val, Ile, or Ala; position 274 is Trp or Gly; position 276 is Tyr, Arg, or Glu; position 322 is Ser, Arg, or Gln; position 324 is Val, Ser, or Phe; position 326 is lie, Ser, or Trp; position 329 is Trp, Thr, Ser, Arg, or Asp; position 330 is Trp; and position 331 is Ser, Lys, Arg, or Val. In some embodiments, the modified Fc polypeptide comprises Val or Ile at position 272; Gly at position 274; Arg at position 276; Arg at position 322; Ser at position 324; Ser at position 326; Thr, Ser, or Arg at position 329; Trp at position 330; and/or Lys or Arg at position 331.

In some embodiments, the modified Fc polypeptide has at least 70% identity, at least 75% identity, at least 80% identity, at least 85% identity, at least 90% identity, or at least 95% identity to amino acids 1-110 of any one of SEQ ID NOS:131-135. In some embodiments, the modified Fc polypeptide has at least 70% identity, at least 75% identity, at least 80% identity, at least 85% identity, at least 90% identity, or at least 95% identity to SEQ ID NOS:131-135. In some embodiments, the modified Fc polypeptide comprises the amino acid sequence of any one of SEQ ID NOS:131-135. In other embodiments, the modified Fc polypeptide comprises the amino acid sequence of any one of SEQ ID NOS:131-135, but in which one, two, or three amino acids are substituted.

### VII. Additional Fc Polypeptide Mutations

In some aspects, a fusion protein described herein comprises two Fc polypeptides that may each comprise independently selected modifications or may be a wild-type Fc polypeptide, e.g., a human IgG1 Fc polypeptide. In some embodiments, one or both Fc polypeptides contains one or more modifications that confer binding to a blood-brain barrier (BBB) receptor, e.g., transferrin receptor (TfR). Non-limiting examples of other mutations that can be introduced into one or both Fc polypeptides include, e.g., mutations to increase serum stability, to modulate effector function, to influence glycosylation, to reduce immunogenicity in humans, and/or to provide for knob and hole heterodimerization of the Fc polypeptides.

In some embodiments, the Fc polypeptides present in the fusion protein independently have an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide).

In some embodiments, the Fc polypeptides present in the fusion protein include knob and hole mutations to promote heterodimer formation and hinder homodimer formation. Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative effect on binding of the polypeptide to a BBB receptor, e.g., TfR.

In one illustrative embodiment of a knob and hole approach for dimerization, position 366 (numbered according to the EU numbering scheme) of one of the Fc polypeptides present in the fusion protein comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of a fusion protein described herein has the T366W knob mutation and the other Fc polypeptide has the Y407V mutation, which is typically accompanied by the T366S and L368A hole mutations.

In some embodiments, modifications to enhance serum half-life may be introduced. For example, in some embodiments, one or both Fc polypeptides present in a fusion protein described herein may comprise a tyrosine at position 252, a threonine at position 254, and a glutamic acid at position 256, as numbered according to the EU numbering scheme. Thus, one or both Fc polypeptides may have M252Y, S254T, and T256E substitutions. Alternatively, one or both Fc polypeptides may have M428L and N434S substitutions, as numbered according to the EU numbering scheme. Alternatively, one or both Fc polypeptides may have an N434S or N434A substitution.

In some embodiments, one or both Fc polypeptides present in a fusion protein described herein may comprise modifications that reduce effector function, i.e., having a reduced ability to induce certain biological functions upon binding to an Fc receptor expressed on an effector cell that mediates the effector function. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides present in a fusion protein described herein may also be engineered to contain other modifications for heterodimerization, e.g., electrostatic engineering of contact residues within a CH3-CH3 interface that are naturally charged or hydrophobic patch modifications.

In some embodiments, one or both Fc polypeptides present in a fusion protein described herein may include additional modifications that modulate effector function.

In some embodiments, one or both Fc polypeptides present in a fusion protein described herein may comprise modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (LALA) substitutions.

Additional Fc polypeptide mutations that modulate an effector function include, but are not limited to, the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

### Illustrative Fc Polypeptides Comprising Additional Mutations

By way of non-limiting example, one or both Fc polypeptides present in a fusion protein described herein may comprise additional mutations including a knob mutation (e.g., T366W as numbered according to the EU numbering scheme), hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), and/or mutations that increase serum stability (e.g., M252Y, S254T, and T256E as numbered according to the EU numbering scheme).

In some embodiments, an Fc polypeptide may have a knob mutation (e.g., T366W as numbered according to the EU numbering scheme) and at least 85% identity, at least 90% identity, or at least 95% identity to the sequence of any one of SEQ ID NOS:1, 4-90, and 111-135. In some embodiments, an Fc polypeptide having the sequence of any one of SEQ ID NOS:1, 4-90, and 111-135 may be modified to have a knob mutation.

In some embodiments, an Fc polypeptide may have a knob mutation (e.g., T366W as numbered according to the EU numbering scheme), mutations that modulate effector function (e.g., L234A, L235A, and/or P329G (e.g., L234A and L235A) as numbered according to the EU numbering scheme), and at least 85% identity, at least 900% identity, or at least 95% identity to the sequence of any one of SEQ ID NOS:1, 4-90, and 111-135. In some embodiments, an Fc polypeptide having the sequence of any one of SEQ ID NOS:1, 4-90, and 111-135 may be modified to have a knob mutation and mutations that modulate effector function.

In some embodiments, an Fc polypeptide may have a knob mutation (e.g., T366W as numbered according to the EU numbering scheme), mutations that increase serum stability (e.g., M252Y, S254T, and T256E as numbered according to the EU numbering scheme), and at least 85% identity, at least 90% identity, or at least 95% identity to the sequence of any one of SEQ ID NOS:1, 4-90, and 111-135. In some embodiments, an Fc polypeptide having the sequence of any one of SEQ ID NOS:1, 4-90, and 111-135 may be modified to have a knob mutation and mutations that increase serum stability.

In some embodiments, an Fc polypeptide may have a knob mutation (e.g., T366W as numbered according to the EU numbering scheme), mutations that modulate effector function (e.g., L234A, L235A, and/or P329G (e.g., L234A and L235A) as numbered according to the EU numbering scheme), mutations that increase serum stability (e.g., M252Y, S254T, and T256E as numbered according to the EU numbering scheme), and at least 85% identity, at least 90% identity, or at least 95% identity to the sequence of any one of SEQ ID NOS:1, 4-90, and 111-135. In some embodiments, an Fc polypeptide having the sequence of any one of SEQ ID NOS:1, 4-90, and 111-135 may be modified to have a knob mutation, mutations that modulate effector function, and mutations that increase serum stability.

In some embodiments, an Fc polypeptide may have hole mutations (e.g., T366S, L368A, and Y407V as numbered according to the EU numbering scheme) and at least 85% identity, at least 90% identity, or at least 95% identity to the sequence of any one of SEQ ID NOS:1, 4-90, and 111-135. In some embodiments, an Fc polypeptide having the sequence of any one of SEQ ID NOS:1, 4-90, and 111-135 may be modified to have hole mutations.

In some embodiments, an Fc polypeptide may have hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), and at least 85% identity, at least 90% identity, or at least 95% identity to the sequence of any one of SEQ ID NOS:1, 4-90, and 111-135. In some embodiments, an Fc polypeptide having the sequence of any one of SEQ ID NOS:1, 4-90, and 111-135 may be modified to have hole mutations and mutations that modulate effector function.

In some embodiments, an Fc polypeptide may have hole mutations (e.g., T366S, L368A, and Y407V as numbered according to the EU numbering scheme), mutations that increase serum stability (e.g., M252Y, S254T, and T256E as numbered according to the EU numbering scheme), and at least 85% identity, at least 90% identity, or at least 95% identity to the sequence of any one of SEQ ID NOS:1, 4-90, and 111-135. In some embodiments, an Fc polypeptide having sequence of any one of SEQ ID NOS:1, 4-90, and 111-135 may be modified to have hole mutations and mutations that increase serum stability.

In some embodiments, an Fc polypeptide may have hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), mutations that increase serum stability (e.g., M252Y, S254T, and T256E as numbered according to the EU numbering scheme), and at least 85% identity, at least 90% identity, or at least 95% identity to the sequence of any one of SEQ ID NOS:1, 4-90, and 111-135. In some embodiments, an Fc polypeptide having the sequence of any one of SEQ ID NOS:1, 4-90, and 111-135 may be modified to have hole mutations, mutations that modulate effector function, and mutations that increase serum stability.

### VIII. Illustrative Fusion Proteins Comprising a Progranulin Polypeptide

In some aspects, a fusion protein described herein comprises a first Fc polypeptide that is linked to a progranulin polypeptide or a variant thereof; and a second Fc polypeptide that forms an Fc dimer with the first Fc polypeptide. In some embodiments, the first Fc polypeptide and/or the second Fc polypeptide does not include an immunoglobulin heavy and/or light chain variable region sequence or an antigen-binding portion thereof. In some embodiments, the first Fc polypeptide is a modified Fc polypeptide and/or the second Fc polypeptide is a modified Fc polypeptide. In some embodiments, the second Fc polypeptide is a modified Fc polypeptide. In some embodiments, the modified Fc polypeptide contains one or more modifications that promote its heterodimerization to the other Fc polypeptide. In some embodiments, the modified Fc polypeptide contains one or more modifications that reduce effector function. In some embodiments, the modified Fc polypeptide contains one or more modifications that extend serum half-life. In some embodiments, the modified Fc polypeptide contains one or more modifications that confer binding to a blood-brain barrier (BBB) receptor, e.g., transferrin receptor (TfR).

In other aspects, a fusion protein described herein comprises a first polypeptide chain that comprises a modified Fc polypeptide that specifically binds to a BBB receptor, e.g., TfR, and a second polypeptide chain that comprises an Fc polypeptide which dimerizes with the modified Fc polypeptide to form an Fc dimer. A progranulin polypeptide may be linked to either the first or the second polypeptide chain. In some embodiments, the progranulin polypeptide is linked to the second polypeptide chain. In some embodiments, the protein comprises two progranulin polypeptides, each linked to one of the polypeptide chains. In some embodiments, the Fc polypeptide may be a BBB receptor-binding polypeptide that specifically binds to the same BBB receptor as the modified Fc polypeptide in the first polypeptide chain. In some embodiments, the Fc polypeptide does not specifically bind to a BBB receptor.

In some embodiments, a fusion protein described herein comprises a first polypeptide chain comprising a modified Fc polypeptide that specifically binds to TfR and a second polypeptide chain that comprises an Fc polypeptide, wherein the modified Fc polypeptide and the Fc polypeptide dimerize to from an Fc dimer. In some embodiments, the progranulin polypeptide is linked to the first polypeptide chain. In some embodiments, the progranulin polypeptide is linked to the second polypeptide chain. In some embodiments, the Fc polypeptide does not specifically bind to a BBB receptor, e.g., TfR.

In some embodiments, a fusion protein described herein comprises a first polypeptide chain that comprises a modified Fc polypeptide that binds to TfR and comprises a T366W (knob) substitution; and a second polypeptide chain that comprises an Fc polypeptide comprising T366S, L368A, and Y407V (hole) substitutions. In some embodiments, the modified Fc polypeptide and/or the Fc polypeptide further comprises L234A and L235A (LALA) substitutions. In some embodiments, the modified Fc polypeptide and/or the Fc polypeptide further comprises M252Y, S254T, and T256E (YTE) substitutions. In some embodiments, the modified Fc polypeptide and/or the Fc polypeptide further comprises L234A and L235A (LALA) substitutions and M252Y, S254T, and T256E (YTE) substitutions. In some embodiments, the modified Fc polypeptide and/or the Fc polypeptide comprises human IgG1 wild-type residues at positions 234, 235, 252, 254, 256, and 366.

In some embodiments, the modified Fc polypeptide comprises the knob, LALA, and YTE mutations as specified for any one of SEQ ID NOS:93-96, 136, 137-142, 149-154, 161-166, 173-178, 185-190, and 197-202, and has at least 85% identity, at least 90% identity, or at least 95% identity to the respective sequence; or comprises the sequence of any one of SEQ ID NOS:93-96, 136, 137-142, 149-154, 161-166, 173-178, 185-190, and 197-202. In some embodiments, the Fc polypeptide comprises the hole, LALA, and YTE mutations as specified for any one of SEQ ID NOS:97-100 and has at least 85% identity, at least 90% identity, or at least 95% identity to the respective sequence; or comprises the sequence of any one of SEQ ID NOS:97-100. In some embodiments, the modified Fc polypeptide comprises any one of SEQ ID NOS:93-96, 136, 137-142, 149-154, 161-166, 173-178, 185-190, and 197-202, and the Fc polypeptide comprises any one of SEQ ID NOS:97-100. In some embodiments, the N-terminus of the modified Fc polypeptide and/or the Fc polypeptide includes a portion of an IgG1 hinge region (e.g., DKTHTCPPCP; SEQ ID NO:109). In some embodiments, the modified Fc polypeptide has at least 85%, at least 90%, or at least 95% identity to any one of SEQ ID NOS: 110, 209, and 210, or comprises the sequence of any one of SEQ ID NOS:110, 209, and 210.

In some embodiments, a fusion protein described herein comprises a first polypeptide chain that comprises a modified Fc polypeptide that binds to TfR and comprises T366S, L368A, and Y407V (hole) substitutions; and a second polypeptide chain that comprises an Fc polypeptide comprising a T366W (knob) substitution. In some embodiments, the modified Fc polypeptide and/or the Fc polypeptide further comprises L234A and L235A (LALA) substitutions. In some embodiments, the modified Fc polypeptide and/or the Fc polypeptide further comprises M252Y, S254T, and T256E (YTE) substitutions. In some embodiments, the modified Fc polypeptide and/or the Fc polypeptide further comprises L234A and L235A (LALA) substitutions and M252Y, S254T, and T256E (YTE) substitutions. In some embodiments, the modified Fc polypeptide and/or the Fc polypeptide comprises human IgG1 wild-type residues at positions 234, 235, 252, 254, 256, and 366.

In some embodiments, the modified Fc polypeptide comprises the hole, LALA, and YTE mutations as specified for any one of SEQ ID NOS:101-104, 143-148, 155-160, 167-172, 179-184, 191-196, and 203-208, and has at least 85% identity, at least 90% identity, or at least 95% identity to the respective sequence; or comprises the sequence of any one of SEQ ID NOS:101-104, 143-148, 155-160, 167-172, 179-184, 191-196, and 203-208. In some embodiments, the Fc polypeptide comprises the knob, LALA, and YTE mutations as specified for any one of SEQ ID NOS:105-108 and has at least 85% identity, at least 90% identity, or at least 95% identity to the respective sequence; or comprises the sequence of any one of SEQ ID NOS:105-108. In some embodiments, the modified Fc polypeptide comprises any one of SEQ ID NOS:101-104, 143-148, 155-160, 167-172, 179-184, 191-196, and 203-208, and the Fc polypeptide comprises any one of SEQ ID NOS:105-108. In some embodiments, the N-terminus of the modified Fc polypeptide and/or the Fc polypeptide includes a portion of an IgG1 hinge region (e.g., DKTHTCPPCP; SEQ ID NO: 109).

In some embodiments, a progranulin polypeptide present in a fusion protein described herein is linked to a polypeptide chain that comprises an Fc polypeptide having at least 85%, at least 90%, or at least 95% identity to any one of SEQ ID NOS:97-100, or comprises the sequence of any one of SEQ ID NOS:97-100 (e.g., as a fusion polypeptide). In some embodiments, the progranulin polypeptide is linked to the Fc polypeptide by a linker, such as a flexible linker, and/or a hinge region or portion thereof (e.g., DKTHTCPPCP; SEQ ID NO: 109). In some embodiments, the fusion protein comprises a modified Fc polypeptide having at least 85%, at least 90%, or at least 95% identity to any one of SEQ ID NOS:93-96, 136, 137-142, 149-154, 161-166, 173-178, 185-190, and 197-202, or comprises the sequence of any one of SEQ ID NOS:93-96, 136, 137-142, 149-154, 161-166, 173-178, 185-190, and 197-202. In some embodiments, the N-terminus of the Fc polypeptide and/or the modified Fc polypeptide includes a portion of an IgG1 hinge region (e.g., DKTHTCPPCP; SEQ ID NO:109). In some embodiments, the progranulin polypeptide comprises a sequence having greater than 90%, or at least 95% identity to SEQ ID NO:212, or comprises the sequence of SEQ ID NO:212. In some embodiments, the modified Fc polypeptide has at least 85%, at least 90%, or at least 95% identity to any one of SEQ ID NOS: 110, 209, and 210, or comprises the sequence of any one of SEQ ID NOS: 110, 209, and 210.

In some embodiments, a progranulin polypeptide present in a fusion protein described herein is linked to a polypeptide chain that comprises an Fc polypeptide having at least 85%, at least 90%, or at least 95% identity to any one of SEQ ID NOS:105-108, or comprises the sequence of any one of SEQ ID NOS:105-108 (e.g., as a fusion polypeptide). In some embodiments, the progranulin polypeptide is linked to the Fc polypeptide by a linker, such as a flexible linker, and/or a hinge region or portion thereof (e.g., DKTHTCPPCP; SEQ ID NO:109). In some embodiments, the progranulin polypeptide comprises a sequence having greater than 90%, or at least 95% identity to SEQ ID NO:212, or comprises the sequence of SEQ ID NO:212. In some embodiments, the fusion protein comprises a modified Fc polypeptide having at least 85%, at least 90%, or at least 95% identity to any one of SEQ ID NOS:101-104, 143-148, 155-160, 167-172, 179-184, 191-196, and 203-208, or comprises the sequence of any one of SEQ ID NOS:101-104, 143-148, 155-160, 167-172, 179-184, 191-196, and 203-208. In some embodiments, the N-terminus of the Fc polypeptide and/or the modified Fc polypeptide includes a portion of an IgG1 hinge region (e.g., DKTHTCPPCP; SEQ ID NO:109).

In some embodiments, a progranulin polypeptide present in a fusion protein described herein is linked to a polypeptide chain that comprises a modified Fc polypeptide having at least 85%, at least 90%, or at least 95% identity to any one of SEQ ID NOS:93-96, 136, 137-142, 149-154, 161-166, 173-178, 185-190, and 197-202, or comprises the sequence of any one of SEQ ID NOS:93-96, 136, 137-142, 149-154, 161-166, 173-178, 185-190, and 197-202 (e.g., as a fusion polypeptide). In some embodiments, the progranulin polypeptide is linked to the modified Fc polypeptide by a linker, such as a flexible linker, and/or a hinge region or portion thereof (e.g., DKTHTCPPCP; SEQ ID NO:109). In some embodiments, the progranulin polypeptide comprises a sequence having greater than 90%, or at least 95% identity to SEQ ID NO:212, or comprises the sequence of SEQ ID NO:212. In some embodiments, the fusion protein comprises an Fc polypeptide having at least 85%, at least 90%, or at least 95% identity to any one of SEQ ID NOS:97-100, 149 and 150, or comprises the sequence of any one of SEQ ID NOS:97-100, 149 and 150. In some embodiments, the N-terminus of the modified Fc polypeptide and/or the Fc polypeptide includes a portion of an IgG1 hinge region (e.g., DKTHTCPPCP; SEQ ID NO:109).

In some embodiments, a progranulin polypeptide present in a fusion protein described herein is linked to a polypeptide chain that comprises a modified Fc polypeptide having at least 85%, at least 90%, or at least 95% identity to any one of SEQ ID NOS:101-104, 143-148, 155-160, 167-172, 179-184, 191-196, and 203-208, or comprises the sequence of any one of SEQ ID NOS:101-104, 143-148, 155-160, 167-172, 179-184, 191-196, and 203-208 (e.g., as a fusion polypeptide). In some embodiments, the progranulin polypeptide is linked to the modified Fc polypeptide by a linker, such as a flexible linker, and/or a hinge region or portion thereof (e.g., DKTHTCPPCP; SEQ ID NO:109). In some embodiments, the progranulin polypeptide comprises a sequence having greater than 90%, or at least 95% identity to SEQ ID NO:212, or comprises the sequence of SEQ ID NO:212. In some embodiments, the fusion protein comprises an Fc polypeptide having at least 85%, at least 90%, or at least 95% identity to any one of SEQ ID NOS:105-108, or comprises the sequence of any one of SEQ ID NOS:105-108. In some embodiments, the N-terminus of the modified Fc polypeptide and/or the Fc polypeptide includes a portion of an IgG1 hinge region (e.g., DKTHTCPPCP; SEQ ID NO:109).

### Fc Dimer:PGRN Fusion Proteins

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, according to EU numbering scheme, and (b) a second Fc polypeptide comprising knob mutation T366W, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:261.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and L234A and L235A (LALA) mutations, according to EU numbering scheme, and (b) a second Fc polypeptide comprising knob mutation T366W, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:215, 216, 227, or 228, and (b) comprises a sequence that has at least 85% identity, at least 90% identity, at least 95% identity, or 100% identity to the sequence of SEQ ID NO:261.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and L234A, L235A, and P329G (LALAPG) mutations, according to EU numbering scheme, and (b) a second Fc polypeptide comprising knob mutation T366W, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:217, 218, 229, or 230, and (b) comprises a sequence that has at least 85% identity, at least 90% identity, at least 95% identity, or 100% identity to the sequence of SEQ ID NO:261.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and M428L and N434S (LS) mutations, according to EU numbering scheme, and (b) a second Fc polypeptide comprising knob mutation T366W, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:261.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, L234A and L235A (LALA) mutations, and M428L and N434S (LS) mutations, according to EU numbering scheme, and (b) a second Fc polypeptide comprising knob mutation T366W, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:221, 222, 233, or 234, and (b) comprises a sequence that has at least 85% identity, at least 90% identity, at least 95% identity, or 100% identity to the sequence of SEQ ID NO:261.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, L234A, L235A, and P329G (LALAPG) mutations, and M428L and N434S (LS) mutations, according to EU numbering scheme, and (b) a second Fc polypeptide comprising knob mutation T366W, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:223, 224, 235, or 236, and (b) comprises a sequence that has at least 85% identity, at least 90% identity, at least 95% identity, or 100% identity to the sequence of SEQ ID NO:261.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, according to EU numbering scheme, and (b) a second Fc polypeptide comprising knob mutation T366W and L234A and L235A (LALA) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:262.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, according to EU numbering scheme, and (b) a second Fc polypeptide comprising knob mutation T366W and L234A, L235A, and P329G (LALAPG) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:263.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, according to EU numbering scheme, and (b) a second Fc polypeptide comprising knob mutation T366W and M428L and N434S (LS) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:264.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, according to EU numbering scheme, and (b) a second Fc polypeptide comprising knob mutation T366W, L234A and L235A (LALA) mutations, and M428L and N434S (LS) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 900% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:265.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, according to EU numbering scheme, and (b) a second Fc polypeptide comprising knob mutation T366W, L234A, L235A, and P329G (LALAPG) mutations, and M428L and N434S (LS) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:266.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and L234A and L235A (LALA) mutations, according to EU numbering scheme, and (b) a second Fc polypeptide comprising knob mutation T366W and L234A and L235A (LALA) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:215, 216, 227, or 228, and (b) comprises a sequence that has at least 85% identity, at least 90% identity, at least 95% identity, or 100% identity to the sequence of SEQ ID NO:262. In one embodiment, an Fc dimer:PGRN fusion protein comprises: (a) a sequence that has at least 85% identity, at least 90% c identity, at least 95% identity, or 100% identity to the sequence of SEQ ID NO:215, and (b) a sequence that has at least 85% identity, at least 90% identity, at least 95% identity, or 100% identity to the sequence of SEQ ID NO:210. In one embodiment, an Fc dimer:PGRN fusion protein comprises: (a) a sequence that has at least 85% identity, at least 90% identity, at least 95% identity, or 100% identity to the sequence of SEQ ID NO:227, and (b) a sequence that has at least 85% identity, at least 900% identity, at least 95% identity, or 100% identity to the sequence of SEQ ID NO:210. In one embodiment, an Fc dimer:PGRN fusion protein comprises: (a) a sequence that has at least 85% identity, at least 90% identity, at least 95% identity, or 100% identity to the sequence of SEQ ID NO:215, and (b) a sequence that has at least 85% identity, at least 90% identity, at least 95% identity, or 100% identity to the sequence of SEQ ID NO:291. In one embodiment, an Fc dimer:PGRN fusion protein comprises: (a) a sequence that has at least 85% identity, at least 90% identity, at least 95% identity, or 100% identity to the sequence of SEQ ID NO:227, and (b) a sequence that has at least 85% identity, at least 90% identity, at least 95% identity, or 100% identity to the sequence of SEQ ID NO:291.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and L234A, L235A, and P329G (LALAPG) mutations, according to EU numbering scheme, and (b) a second Fc polypeptide comprising knob mutation T366W and L234A, L235A, and P329G (LALAPG) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:217, 218, 229, or 230, and (b) comprises a sequence that has at least 85% identity, at least 90% identity, at least 95% identity, or 100% identity to the sequence of SEQ ID NO:263.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and M428L and N434S (LS) mutations, according to EU numbering scheme, and (b) a second Fc polypeptide comprising knob mutation T366W and M428L and N434S (LS) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:264.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and L234A and L235A (LALA) mutations, according to EU numbering scheme, and (b) a second Fc polypeptide comprising knob mutation T366W and M428L and N434S (LS) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:215, 216, 227, or 228, and (b) comprises a sequence that has at least 85% identity, at least 90% identity, at least 95% identity, or 100% identity to the sequence of SEQ ID NO:264.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and L234A, L235A, and P329G (LALAPG) mutations, according to EU numbering scheme, and (b) a second Fc polypeptide comprising knob mutation T366W and M428L and N434S (LS) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:217, 218, 229, or 230, and (b) comprises a sequence that has at least 85% identity, at least 90% identity, at least 95% identity, or 100% identity to the sequence of SEQ ID NO:264.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and M428L and N434S (LS) mutations, according to EU numbering scheme, and (b) a second Fc polypeptide comprising knob mutation T366W and L234A and L235A (LALA) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 900% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:262.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V and M428L and N434S (LS) mutations, according to EU numbering scheme, and (b) a second Fc polypeptide comprising knob mutation T366W and L234A, L235A, and P329G (LALAPG) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:263.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, L234A and L235A (LALA) mutations, and M428L and N434S (LS) mutations, according to EU numbering scheme, and (b) a second Fc polypeptide comprising knob mutation T366W, L234A and L235A (LALA) mutations, and M428L and N434S (LS) mutations according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:221, 222, 233, or 234, and (b) comprises a sequence that has at least 85% identity, at least 90% identity, at least 95% identity, or 100% identity to the sequence of SEQ ID NO:265.

In one embodiment, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, L234A, L235A, and P329G (LALAPG) mutations, and M428L and N434S (LS) mutations, according to EU numbering scheme, and (b) a second Fc polypeptide comprising knob mutation T366W, L234A, L235A, and P329G (LALAPG) mutations, and M428L and N434S (LS) mutations, according to EU numbering scheme. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, (a) comprises a sequence that has at least 85% identity, at least 90% identity, at least 95% identity, or 100% identity to the sequence of SEQ ID NO:223, 224, 235, or 236, and (b) comprises a sequence that has at least 85% identity, at least 90% identity, at least 95% identity, or 100% identity to the sequence of SEQ ID NO:266.

In any of the embodiments described above, an Fc dimer:PGRN fusion protein may comprise a first Fc polypeptide having the knob mutation T366W and the second Fc polypeptide having the hole mutations T366S, L368A, and Y407V, in which the first Fc polypeptide is linked to a progranulin polypeptide directly or through a polypeptide linker.

In any of the embodiments described above, a progranulin polypeptide may also be linked to the second Fc polypeptide, creating an Fc dimer:PGRN fusion protein comprising two progranulin polypeptides. For example, the C-terminus of the first and second progranulin polypeptides may be linked to the N-terminus of the first and second Fc polypeptides, respectively, directly or through a polypeptide linker. In another example, the N-terminus of the first and second progranulin polypeptides may be linked to the C-terminus of the first and second Fc polypeptides, respectively, directly or through a polypeptide linker. In another example, the C-terminus of the first progranulin polypeptide may be linked to the N-terminus of the first Fc polypeptide, and the N-terminus of the second progranulin polypeptide may be linked to the C-terminus of the second Fc polypeptide. In another example, the N-terminus of the first progranulin polypeptide may be linked to the C-terminus of the first Fc polypeptide, and the C-terminus of the second progranulin polypeptide may be linked to the N-terminus of the second Fc polypeptide. In some embodiments of the Fc dimer:PGRN fusion protein comprising two progranulin polypeptides, wherein each of the two progranulin polypeptides is linked to an Fc polypeptide through a polypeptide linker, the two polypeptide linkers in the fusion protein can be the same or different. For example, the two polypeptide linkers can each independently be a Gly₄-Ser linker (SEQ ID NO:277) or a (Gly₄-Ser)₂ linker (SEQ ID NO:276).

In any of the embodiments described above, the first Fc polypeptide or the second Fc polypeptide may comprise TfR-binding mutations. In some embodiments, the first Fc polypeptide may comprise TfR-binding mutations. The first Fc polypeptide may comprise a sequence of any one of SEQ ID NOS:101, 102, 143-145, 155-157, 167-169, 179-181, 191-193, and 203-205. In some embodiments, the second Fc polypeptide may comprise TfR-binding mutations. The second Fc polypeptide may comprise a sequence of any one of SEQ ID NOS:93, 94, 136-139, 149-151, 161-163, 173-175, 185-187, and 197-199. In some embodiments, both the first and second Fc polypeptides may comprise TfR-binding mutations.

In particular embodiments, an Fc dimer:PGRN fusion protein comprises: (a) a first Fc polypeptide linked to a progranulin polypeptide directly or through a polypeptide linker (e.g., GGGGSGGGGS (SEQ ID NO:276) or GGGGS (SEQ ID NO:277)), wherein the first Fc polypeptide comprises hole mutations T366S, L368A, and Y407V, according to EU numbering scheme, and (b) a second Fc polypeptide comprising knob mutation T366W, according to EU numbering scheme, and TfR-binding mutations. In some embodiments, the C-terminus of the progranulin polypeptide is linked to the N-terminus of the first Fc polypeptide directly or through a polypeptide linker. In some embodiments, the N-terminus of the progranulin polypeptide is linked to the C-terminus of the first Fc polypeptide directly or through a polypeptide linker. In some embodiments, the first and/or second Fc polypeptide may further be linked to an IgG1 hinge region or a portion thereof (e.g., SEQ ID NO: 91 or 109). In some embodiments, the first and/or second Fc polypeptide may comprise L234A and L235A with or without P329G mutations, and/or M428L and N434S (LS) mutations.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:281.

In certain embodiments of the Fc dimer:PGRN fusion protein, (a) comprises a sequence that has at least 85% identity, at least 90% identity, at least 95% identity, or 100% identity to the sequence of SEQ ID NO:215, 216, 227, or 228, and (b) comprises a sequence that has at least 85% identity, at least 90% identity, at least 95% identity, or 100% identity to the sequence of SEQ ID NO:281.

In certain embodiments of the Fc dimer:PGRN fusion protein, (a) comprises a sequence that has at least 85% identity, at least 90% identity, at least 95% identity, or 100% identity to the sequence of SEQ ID NO:217, 218, 229, or 230, and (b) comprises a sequence that has at least 85% identity, at least 90% identity, at least 95% identity, or 100% identity to the sequence of SEQ ID NO:281.

In certain embodiments of the Fc dimer:PGRN fusion protein, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:281.

In certain embodiments of the Fc dimer:PGRN fusion protein, (a) comprises a sequence that has at least 85% identity, at least 90% identity, at least 95% identity, or 100% identity to the sequence of SEQ ID NO:221, 222, 233, or 234, and (b) comprises a sequence that has at least 85% identity, at least 90% identity, at least 95% identity, or 100% identity to the sequence of SEQ ID NO:281.

In certain embodiments of the Fc dimer:PGRN fusion protein, (a) comprises a sequence that has at least 85% identity, at least 90% identity, at least 95% identity, or 100% identity to the sequence of SEQ ID NO:223, 224, 235, or 236, and (b) comprises a sequence that has at least 85% identity, at least 90% identity, at least 95% identity, or 100% identity to the sequence of SEQ ID NO:281.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:210.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:282.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:283.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:284.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:285.

In certain embodiments of the Fc dimer:PGRN fusion protein, (a) comprises a sequence that has at least 85% identity, at least 90% identity, at least 95% identity, or 100% identity to the sequence of SEQ ID NO:215, 216, 227, or 228, and (b) comprises a sequence that has at least 85% identity, at least 90% identity, at least 95% identity, or 100% identity to the sequence of SEQ ID NO:210.

In certain embodiments of the Fc dimer:PGRN fusion protein, (a) comprises a sequence that has at least 850% identity, at least 90% identity, at least 95% identity, or 100% identity to the sequence of SEQ ID NO:217, 218, 229, or 230, and (b) comprises a sequence that has at least 85% identity, at least 90% identity, at least 95% identity, or 100% identity to the sequence of SEQ ID NO:282.

In certain embodiments of the Fc dimer:PGRN fusion protein, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:283.

In certain embodiments of the Fc dimer:PGRN fusion protein, (a) comprises a sequence that has at least 85% identity, at least 90% identity, at least 95% identity, or 100% identity to the sequence of SEQ ID NO:215, 216, 227, or 228, and (b) comprises a sequence that has at least 85% identity, at least 90% identity, at least 95% identity, or 100% identity to the sequence of SEQ ID NO:283.

In certain embodiments of the Fc dimer:PGRN fusion protein, (a) comprises a sequence that has at least 85% identity, at least 90% identity, at least 95% identity, or 100% identity to the sequence of SEQ ID NO:217, 218, 229, or 230, and (b) comprises a sequence that has at least 85% identity, at least 90% identity, at least 95% identity, or 100% identity to the sequence of SEQ ID NO:283.

In certain embodiments of the Fc dimer:PGRN fusion protein, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:210.

In certain embodiments of the Fc dimer:PGRN fusion protein, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 900% identity, at least 95% identity, or 100% identity to the sequence of SEQ ID NO:282.

In certain embodiments of the Fc dimer:PGRN fusion protein, (a) comprises a sequence that has at least 85% identity, at least 90% identity, at least 95% identity, or 100% identity to the sequence of SEQ ID NO:221, 222, 233, or 234, and (b) comprises a sequence that has at least 85% identity, at least 90% identity, at least 95% identity, or 100% identity to the sequence of SEQ ID NO:284.

In certain embodiments of the Fc dimer:PGRN fusion protein, (a) comprises a sequence that has at least 85% identity, at least 90% identity, at least 95% identity, or 100% identity to the sequence of SEQ ID NO:223, 224, 235, or 236, and (b) comprises a sequence that has at least 85% identity, at least 90% identity, at least 95% identity, or 100% identity to the sequence of SEQ ID NO:285.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:286.

In certain embodiments of the Fc dimer:PGRN fusion protein, (a) comprises a sequence that has at least 85% identity, at least 90% identity, at least 95% identity, or 100% identity to the sequence of SEQ ID NO:215, 216, 227, or 228, and (b) comprises a sequence that has at least 85% identity, at least 90% identity, at least 95% identity, or 100% identity to the sequence of SEQ ID NO:286.

In certain embodiments of the Fc dimer:PGRN fusion protein, (a) comprises a sequence that has at least 85% identity, at least 90% identity, at least 95% identity, or 100% identity to the sequence of SEQ ID NO:217, 218, 229, or 230, and (b) comprises a sequence that has at least 85% identity, at least 90% identity, at least 95% identity, or 100% identity to the sequence of SEQ ID NO:286.

In certain embodiments of the Fc dimer:PGRN fusion protein, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 900% identity, at least 95% identity, or 100% identity to the sequence of SEQ ID NO:286.

In certain embodiments of the Fc dimer:PGRN fusion protein, (a) comprises a sequence that has at least 85% identity, at least 90% identity, at least 95% identity, or 100% identity to the sequence of SEQ ID NO:221, 222, 233, or 234, and (b) comprises a sequence that has at least 85% identity, at least 90% identity, at least 95% identity, or 100% identity to the sequence of SEQ ID NO:286.

In certain embodiments of the Fc dimer:PGRN fusion protein, (a) comprises a sequence that has at least 85% identity, at least 90% identity, at least 95% identity, or 100% identity to the sequence of SEQ ID NO:223, 224, 235, or 236, and (b) comprises a sequence that has at least 85% identity, at least 90% identity, at least 95% identity, or 100% identity to the sequence of SEQ ID NO:286.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:209.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:287.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:288.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:289.

In certain embodiments of the Fc dimer:PGRN fusion protein, (a) comprises a sequence that has at least 850% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:290.

In certain embodiments of the Fc dimer:PGRN fusion protein, (a) comprises a sequence that has at least 85% identity, at least 90% identity, at least 95% identity, or 100% identity to the sequence of SEQ ID NO:213, 214, 225, or 226, and (b) comprises a sequence that has at least 85% identity, at least 90% identity, at least 95% identity, or 100% identity to the sequence of SEQ ID NO:291.

In certain embodiments of the Fc dimer:PGRN fusion protein, (a) comprises a sequence that has at least 85% identity, at least 90% identity, at least 95% identity, or 100% identity to the sequence of SEQ ID NO:215, 216, 227, or 228, and (b) comprises a sequence that has at least 85% identity, at least 90% identity, at least 95% identity, or 100% identity to the sequence of SEQ ID NO:209.

In certain embodiments of the Fc dimer:PGRN fusion protein, (a) comprises a sequence that has at least 85% identity, at least 90% identity, at least 95% identity, or 100% identity to the sequence of SEQ ID NO:217, 218, 229, or 230, and (b) comprises a sequence that has at least 85% identity, at least 90% identity, at least 95% identity, or 100% identity to the sequence of SEQ ID NO:287.

In certain embodiments of the Fc dimer:PGRN fusion protein, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:288.

In certain embodiments of the Fc dimer:PGRN fusion protein, (a) comprises a sequence that has at least 85% identity, at least 90% identity, at least 95% identity, or 100% identity to the sequence of SEQ ID NO:215, 216, 227, or 228, and (b) comprises a sequence that has at least 85% identity, at least 90% identity, at least 95% identity, or 100% identity to the sequence of SEQ ID NO:288.

In certain embodiments of the Fc dimer:PGRN fusion protein, (a) comprises a sequence that has at least 850% identity, at least 90% identity, at least 95% identity, or 100% identity to the sequence of SEQ ID NO:217, 218, 229, or 230, and (b) comprises a sequence that has at least 85% identity, at least 90% identity, at least 95% identity, or 100% identity to the sequence of SEQ ID NO:288.

In certain embodiments of the Fc dimer:PGRN fusion protein, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:209.

In certain embodiments of the Fc dimer:PGRN fusion protein, (a) comprises a sequence that has at least 85% identity, at least 90% identity, at least 95% identity, or 100% identity to the sequence of SEQ ID NO:219, 220, 231, or 232, and (b) comprises a sequence that has at least 85% identity, at least 90% identity, at least 95% identity, or 100% identity to the sequence of SEQ ID NO:287.

In certain embodiments of the Fc dimer:PGRN fusion protein, (a) comprises a sequence that has at least 85% identity, at least 90% identity, at least 95% identity, or 100% identity to the sequence of SEQ ID NO:221, 222, 233, or 234, and (b) comprises a sequence that has at least 85% identity, at least 90% identity, at least 95% identity, or 100% identity to the sequence of SEQ ID NO:289.

In certain embodiments of the Fc dimer:PGRN fusion protein, (a) comprises a sequence that has at least 85% identity, at least 90% identity, at least 95% identity, or 100% identity to the sequence of SEQ ID NO:223, 224, 235, or 236, and (b) comprises a sequence that has at least 85% identity, at least 90% identity, at least 95% identity, or 100% identity to the sequence of SEQ ID NO:290.

In certain embodiments of the Fc dimer:PGRN fusion protein, (a) comprises a sequence that has at least 85% identity, at least 90% identity, at least 95% identity, or 100% identity to the sequence of SEQ ID NO:223, 224, 235, or 236, and (b) comprises a sequence that has at least 85% identity, at least 900% identity, at least 95% identity, or 100% identity to the sequence of SEQ ID NO:291.

In certain embodiments of the Fc dimer:PGRN fusion protein, (a) comprises a sequence that has at least 85% identity, at least 90% identity, at least 95% identity, or 100% identity to the sequence of SEQ ID NO:215, and (b) comprises a sequence that has at least 85% identity, at least 90% identity, at least 95% identity, or 100% identity to the sequence of SEQ ID NO:210.

In certain embodiments of the Fc dimer:PGRN fusion protein, (a) comprises a sequence that has at least 85% identity, at least 90% identity, at least 95% identity, or 100% identity to the sequence of SEQ ID NO:227, and (b) comprises a sequence that has at least 85% identity, at least 90% identity, at least 95% identity, or 100% identity to the sequence of SEQ ID NO:210.

In certain embodiments of the Fc dimer:PGRN fusion protein, (a) comprises a sequence that has at least 85% identity, at least 90% identity, at least 95% identity, or 100% identity to the sequence of SEQ ID NO:215, and (b) comprises a sequence that has at least 85% identity, at least 90% identity, at least 95% identity, or 100% identity to the sequence of SEQ ID NO:291.

In certain embodiments of the Fc dimer:PGRN fusion protein, (a) comprises a sequence that has at least 85% identity, at least 90% identity, at least 95% identity, or 100% identity to the sequence of SEQ ID NO:227, and (b) comprises a sequence that has at least 85% identity, at least 90% identity, at least 95% identity, or 100% identity to the sequence of SEQ ID NO:291.

In any of the embodiments described above, the partial hinge (DKTHTCPPCP (SEQ ID NO:109)) and/or the glycine-rich linker present in the sequence of any one of SEQ ID NOS:209, 210, 213-275, and 281-291 may be removed. In certain embodiments, the Fc dimer:PGRN fusion protein comprises a sequence of any one of SEQ ID NOS:209, 210, 213-275, and 281-291 without the partial hinge and/or the glycine-rich linker. In other embodiments, the partial hinge present in the sequence of any one of SEQ ID NOS:209, 210, 213-275, and 281-291 may be replaced by a full hinge sequence (e.g., EPKSCDKTHTCPPCP (SEQ ID NO:91)). In any of the embodiments described above, the partial hinge (DKTHTCPPCP (SEQ ID NO:109)) and/or the glycine-rich linker present in the sequence of SEQ ID NO:215 or 227 may be removed. In certain embodiments, the Fc dimer:PGRN fusion protein comprises a sequence of SEQ ID NO:215 or 227 without the partial hinge and/or the glycine-rich linker. In other embodiments, the partial hinge present in the sequence of SEQ ID NO:215 or 227 may be replaced by a full hinge sequence (e.g., EPKSCDKTHTCPPCP (SEQ ID NO:91)).

### IX. Binding Properties

Fusion proteins described herein may have a broad range of binding affinities. For example, in some embodiments, a protein has an affinity for a blood-brain barrier (BBB) receptor, e.g., transferrin receptor (TfR), ranging anywhere from 1 μM to 10 μM. In some embodiments, the affinity for TfR ranges from 1 nM to 5 μM, or from 10 nM to 1 μM.

Methods for analyzing binding affinity, binding kinetics, and cross-reactivity to analyze binding to a BBB receptor, e.g., TfR, are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet® (FortéBio, Inc., Menlo Park, Calif.)), and Western blot analysis. In some embodiments, ELISA is used to determine binding affinity and/or cross-reactivity. Methods for performing ELISA assays are known in the art and are also described in the Example section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

### X. Linkage Between Progranulin Polypeptides and Fc Polypeptides

In some embodiments, a fusion protein described herein comprises two Fc polypeptides as described herein and one or both of the Fc polypeptides may further comprise a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:91) or a portion thereof (e.g., DKTHTCPPCP; SEQ ID NO:109). In some embodiments, the hinge region is at the N-terminal region of the Fc polypeptide.

In some embodiments, the partial hinge (DKTHTCPPCP (SEQ ID NO:109)) present in the sequence of any one of SEQ ID NOS:213-275 may be removed. In other embodiments, the partial hinge (DKTHTCPPCP (SEQ ID NO:109)) present in the sequence of any one of SEQ ID NOS:213-275 may be replaced by the full hinge (EPKSCDKTHTCPPCP (SEQ ID NO:91)). In some embodiments, the partial hinge (DKTHTCPPCP (SEQ ID NO:109)) present in the sequence of SEQ ID NOS:215 or 227 may be removed. In other embodiments, the partial hinge (DKTHTCPPCP (SEQ ID NO:109)) present in the sequence of SEQ ID NO:215 or 227 may be replaced by the full hinge (EPKSCDKTHTCPPCP (SEQ ID NO:91)).

In some embodiments, an Fc polypeptide is joined to the progranulin polypeptide by a linker, e.g., a polypeptide linker. In some embodiments, the Fc polypeptide is joined to the progranulin polypeptide by a peptide bond or by a polypeptide linker, e.g., is a fusion polypeptide. The polypeptide linker may be configured such that it allows for the rotation of the progranulin polypeptide relative to the Fc polypeptide to which it is joined; and/or is resistant to digestion by proteases. Polypeptide linkers may contain natural amino acids, unnatural amino acids, or a combination thereof. In some embodiments, the polypeptide linker may be a flexible linker, e.g., containing amino acids such as Gly, Asn, Ser, Thr, Ala, and the like. Such linkers are designed using known parameters and may be of any length and contain any number of repeat units of any length (e.g., repeat units of Gly and Ser residues). For example, the linker may have repeats, such as two, three, four, five, or more Gly₄-Ser repeats or a single Gly₄-Ser. In some embodiments, the polypeptide linker may include a protease cleavage site, e.g., that is cleavable by an enzyme present in the central nervous system.

In some embodiments, the progranulin polypeptide is joined to the N-terminus of the Fc polypeptide, e.g., by a Gly₄-Ser linker (SEQ ID NO:277) or a (Gly₄-Ser)₂ linker (SEQ ID NO:276). In some embodiments, the Fc polypeptide may comprise a hinge sequence or partial hinge sequence at the N-terminus that is joined to the linker or directly joined to the progranulin polypeptide.

In some embodiments, the progranulin polypeptide is joined to the C-terminus of the Fc polypeptide, e.g., by a Gly₄-Ser linker (SEQ ID NO:277) or a (Gly₄-Ser)₂ linker (SEQ ID NO:276). In some embodiments, the C-terminus of the Fc polypeptide is directly joined to the progranulin polypeptide.

In some embodiments, the polypeptide linker between the Fc polypeptide and the progranulin polypeptide can have 3-200 (e.g., 3-180, 3-160, 3-140, 3-120, 3-100, 3-80, 3-60, 3-40, 3-20, 3-10, 3-5, 5-200, 10-200, 20-200, 40-200, 60-200, 80-200, 100-200, 120-200, 140-200, 160-200, 180-200, 3, 5, 7, 10, 20, 30, 40, 50, 60, 70, 80, 90, 100, 110, 120, 130, 140, 150, 160, 170, 180, 190, or 200) amino acids. Suitable polypeptide linkers are known in the art (e.g., as described in Chen et al. Adv. Drug Deliv Rev. 65(10):1357-1369, 2013), and include, for example, polypeptide linkers containing flexible amino acid residues such as glycine and serine. In certain embodiments, a polypeptide linker can be a polyglycine linker, e.g., (Gly)ₙ, in which n is an integer between 1 and 10. In certain embodiments, a polypeptide linker can contain motifs, e.g., multiple or repeating motifs, of (GS)ₙ, (GGS)ₙ, (GGGGS (SEQ ID NO:277))ₙ, (GGSG (SEQ ID NO:304))ₙ, or (SGGG (SEQ ID NO:305))ₙ, in which n is an integer between 1 and 10. In other embodiments, a polypeptide linker can also contain amino acids other than glycine and serine, e.g., KESGSVSSEQLAQFRSLD (SEQ ID NO:306), EGKSSGSGSESKST (SEQ ID NO:307), and GSAGSAAGSGEF (SEQ ID NO:308). In other embodiments, polypeptide linkers can also be rigid polypeptide linkers. In some embodiments, rigid polypeptide linkers can adopt an α-helical conformation, which can be stabilized by intra-segment hydrogen bonds and/or intra-segment salt bridges. Examples of rigid polypeptide linkers include, but are not limited to, A(EAAAK)ₙA (SEQ ID NO:309), in which n is an integer between 2 and 5, and (XP)ₙ, in which X is Ala, Lys, or Glu, and n is an integer between 1 and 10, as described in Chen et al. Adv. Drug Deliv Rev. 65(10):1357-1369, 2013.

In some embodiments, the progranulin polypeptide is joined to the Fc polypeptide by a chemical cross-linking agent. Such conjugates can be generated using well-known chemical cross-linking reagents and protocols. For example, there are a large number of chemical cross-linking agents that are known to those skilled in the art and useful for cross-linking the polypeptide with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including N-hydroxysuccinimide (NHS) or its water soluble analog N-hydroxysulfosuccinimide (sulfo-NHS), succinimidyl 4-(N-maleimidomethyl)cyclohexane-1-carboxylate (SMCC), m-maleimidobenzoyl-N-hydroxysuccinimide ester (MBS); N-succinimidyl (4-iodoacetyl) aminobenzoate (SLAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), and succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate.2HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers.

### XI. Evaluation of Effects of Fusion Proteins

Activity of fusion proteins described herein that comprise a progranulin polypeptide or a variant thereof, can be assessed using various assays, including assays that measure activity in vitro or in vivo. As described in the Examples, cellular uptake of the fusion proteins described herein may be assayed using bone marrow derived macrophages (BMDMs) and immunostaining with antibodies against human progranulin and human Fc. Proteolysis activity of the cells after the cells are treated with the fusion proteins described herein may be measured using the DQ-BSA assay described in Example 4. Cellular effects caused by GRN mutation (e.g., increased cathepsin D activity and elevated mRNA levels of lysosomal genes such as Cts1, Tmem/06b, and Psap) may be evaluated again after the cells are treated with the fusion proteins described herein (Examples 6 and 7). Fluorgenic probes and qPCR techniques may be used in these assays. Finally, pharmacokinetic properties and brain uptake of the fusion proteins described herein may be determined using wild-type and/or transgenic mice, as shown in Examples 9 and 10.

For cellular samples, the assay may include disrupting the cells and breaking open microvesicles. Disruption of cells may be achieved by using freeze-thawing and/or sonication. In some embodiments, a tissue sample is evaluated. A tissue sample can be evaluated using multiple free-thaw cycles, e.g., 2, 3, 4, 5, or more, which are performed before the sonication step to ensure that microvesicles are broken open.

Samples that can be evaluated by the assays described herein include, e.g., brain, liver, kidney, lung, spleen, plasma, serum, cerebrospinal fluid (CSF), and urine. In some embodiments, CSF samples from a patient receiving a fusion protein comprising a progranulin polypeptide or a variant thereof as described herein may be evaluated.

### XII. Bis(Monoacylglycero)Phosphate (Bmp)

Provided herein are methods of monitoring the levels of progranulin (e.g., in a sample, in a cell, in a tissue, and/or in a subject), wherein determining the level of progranulin comprises measuring the abundance of bis(monoacylglycero)phosphate (BMP) (e.g., in the sample, cell, tissue, and/or subject).

In some embodiments of methods of the present disclosure, the abundance of a single BMP species is measured. In some embodiments, the abundance of two or more BMP species is measured. In some embodiments, the abundance of at least two, three, four, five, or more of the BMP species in Table 3 is measured. When the abundance of two or more BMP species is measured, any combination of different BMP species can be used.

In some embodiments, the abundance of more than one BMP species can be summed, and the total abundance will be compared to a reference value. For example, the abundance of each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, or more BMP species (e.g., the BMP species listed in Table 3) can be summed, and the total abundance then compared to a reference value.

In some cases, one or more BMP species may be differentially expressed (e.g., more or less abundant) in one type of sample when compared to another, such as, for example, cell-based samples (e.g., cultured cells) versus tissue-based or blood samples. Accordingly, in some embodiments, the selection of the one or more BMP species (i.e., for the measurement of abundance) depends on the type of sample. In some embodiments, the one or more BMP species comprise BMP(18:1_18:1), e.g., when a sample (e.g., a test sample and/or a reference sample) is bone marrow-derived macrophage (BMDM). In other embodiments, the one or more BMP species comprise BMP(22:6_22:6), e.g., when a sample comprises tissue (e.g., brain tissue, liver tissue) or plasma, urine, or CSF.

In some embodiments, an internal BMP standard (e.g., BMP(14:0_14:0)) is used to measure the abundance of one or more BMP species in a sample and/or determine a reference value (e.g., measure the abundance of one or more BMP species in a reference sample). For example, a known amount of the internal BMP standard can be added to a sample (e.g., a test sample and/or a reference sample) to serve as a calibration point such that the amount of one or more BMP species that are present in the sample can be determined. In some embodiments, a reagent used in the extraction or isolation of BMP from a sample (e.g., methanol) is “spiked” with the internal BMP standard. Typically, the internal BMP standard will be one that does not naturally occur in the subject.

### XIII. Identification of Subjects Having, or at Risk of Having, a Progranulin-Associated Disorder

In some embodiments, a subject (e.g., a target subject) is determined to have a progranulin-associated disorder or a decreased level of progranulin when the abundance of at least one (e.g., a at least 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, or more) BMP species (e.g., the BMP species listed in Table 3) in a test sample is higher when the test sample is a BMDM or lower when the test sample is liver, brain, cerebrospinal fluid, plasma, or urine than a reference value of a corresponding cell, tissue, or fluid of a healthy control or a control not related to a progranulin-associated disorder.

In some embodiments, a subject (e.g., a target subject) is determined to have a progranulin-associated disorder or a decreased level of progranulin when the abundance of at least one (e.g., 1, 2, 3, 4, 5, 6, 7, 8, 9, 10, 11, 12, 13, 14, 15, 16, 17, 18, 19, 20, 21, 22, or all 23) of the BMP species selected from the group consisting of BMP(16:0_18:1), BMP(16:0_18:2), BMP(18:0_18:0), BMP(18:0_18:1), BMP(18:1_18:1), BMP(16:0_20:3), BMP(18:1_20:2), BMP(18:0_20:4), BMP(16:0_22:5), BMP(20:4_20:4), BMP(22:6_22:6), BMP(20:4_20:5), BMP(18:2_18:2), BMP(16:0_20:4), BMP(18:0_18:2), BMP(18:0e_22:6), BMP(18:1e_20:4), BMP(20:4_22:6), BMP(18:0e_20:4), BMP(18:2_20:4), BMP(18:1_22:6), BMP(18:1_20:4), BMP(18:0_22:6), and BMP(18:3_22:5) is elevated in BMDM or decreased in liver, brain, cerebrospinal fluid, plasma, or urine compared to a reference value of a corresponding cell, tissue, or fluid of a healthy control or a control not related to a progranulin-associated disorder.

In some embodiments, a subject (e.g., a target subject) is determined to have a progranulin-associated disorder or a decreased level of progranulin when the abundance of at least one (e.g., 1, 2, 3, 4, 5, 6, 7, or all 8) of the BMP species selected from the group consisting of BMP(18:1_18:1), BMP(18:0_20:4), BMP(20:4_20:4), BMP(22:6_22:6), BMP(20:4_22:6), BMP(18:1_22:6), BMP(18:1_20:4), BMP(18:0_22:6) and BMP(18:3_22:5) is elevated in BMDM or decreased in liver, brain, cerebrospinal fluid, plasma, or urine compared to a reference value of a corresponding cell, tissue, or fluid of a healthy control or a control not related to a progranulin-associated disorder.

In some embodiments, a subject is determined to have a progranulin-associated disorder or a decreased level of progranulin when BMP(18:1_18:1) levels are elevated in BMDM compared to a reference value of a healthy control or a control not related to a progranulin-associated disorder. In other embodiments, a subject is determined to have a progranulin-associated disorder or a decreased level of progranulin when BMP(22:6_22:6) are decreased in plasma, urine, cerebrospinal fluid (CSF), and/or brain or liver tissue compared to a reference value of a healthy control or a control not related to a progranulin-associated disorder. In other embodiments, a subject is determined to have a progranulin-associated disorder or a decreased level of progranulin when BMP(22:6_22:6) and/or BMP(18:3_22:5) levels are decreased in liver tissue. In other embodiments, a subject is determined to have a progranulin-associated disorder or a decreased level of progranulin when BMP(18:3_22:5) levels are decreased in microglia.

In some embodiments, a subject (e.g., a target subject) is determined to have a progranulin-associated disorder or a decreased level of progranulin when the abundance of at least one of the BMP species (e.g., measured in a test sample) is at least about 1.1-fold (e.g., about 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or more) higher in BMDM or lower in liver, brain, cerebrospinal fluid, plasma, or urine compared to a reference value of a corresponding cell, tissue, or fluid of a healthy control or a control not related to a progranulin-associated disorder.

In some embodiments, a subject (e.g., a target subject) is determined to have a progranulin-associated disorder or a decreased level of progranulin when the abundance of at least one of the BMP species (e.g., measured in a test sample) is at least about 1.2-fold to about 4-fold (e.g., at least about 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, or 4-fold) higher than a reference value (e.g., a corresponding reference value). In some embodiments, a subject is determined to have a progranulin-associated disorder or a decreased level of progranulin when the abundance of at least one of the BMP species is about 2-fold to about 3-fold (e.g., about 2-fold, 2.1-fold, 2.2-fold, 2.3-fold, 2.4-fold, 2.5-fold, 2.6-fold, 2.7-fold, 2.8-fold, 2.9-fold, or 3-fold) higher in BMDM or lower in liver, brain, cerebrospinal fluid, plasma, or urine compared to a reference value of a corresponding cell, tissue, or fluid of a healthy control or a control not related to a progranulin-associated disorder.

### XIV. Monitoring Response to Treatment

In one aspect, the present disclosure provides methods for monitoring progranulin levels in a subject (e.g., a target subject). In another aspects, provided are methods for monitoring a subject's response to a compound, pharmaceutical composition, or dosing regimen thereof or response to any therapy or therapeutic (e.g., response to a Fc dimer:PGRN fusion protein described herein) for treating a progranulin-associated disorder.

Typically, the abundance of each of the one or more BMP species in a test sample will be compared to one or more reference values (e.g., a corresponding reference value). In some embodiments, a BMP value is measured before treatment and at one or more time points after treatment. The abundance value taken at a later time point can be compared to the value prior to treatment as well as to a control value, such as that of a healthy or diseased control, to determine how the subject is responding to the therapy. The one or more reference values can be from different cells, tissues, or fluids corresponding to the cell, tissue, or fluid of the test sample.

In some embodiments, the reference value is the abundance of the one or more BMP species that is measured in a reference sample. The reference value can be a measured abundance value (e.g., abundance value measured in the reference sample), or can be derived or extrapolated from a measured abundance value. In some embodiments, the reference value is a range of values, e.g., when the reference values are obtained from a plurality of samples or a population of subjects. Furthermore, the reference value can be presented as a single value (e.g., a measured abundance value, a mean value, or a median value) or a range of values, with or without a standard deviation or standard of error.

When two or more test samples are obtained (e.g., from a subject), the time points at which they are obtained can be separated by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more minutes; about 1, 2, 3, 4, 5, 6, 7, 8, 9, 10, 11, 12, 13, 14, 15, 16, 17, 18, 19, 20, 21, 22, 23, 24, or more hours; about 1, 2, 3, 4, 5, 6, 7, or more days; about 1, 2, 3, 4, 5, 6, 7, 8, 9, 10, or more weeks; or even longer. When three or more test samples are obtained, the time intervals between when each test sample is obtained can all be the same, the intervals can all be different, or a combination thereof.

In some embodiments, both the first test sample and the second test sample are obtained from a subject (e.g., a target subject) after the subject has been treated, i.e., the first test sample is obtained from the subject at an earlier time point during treatment than the second test sample. In some embodiments, the first test sample is obtained before the subject has been treated for the disorder associated with a decreased level of progranulin (i.e., a pre-treatment test sample) and the second test sample is obtained after the subject has been treated for the disorder associated with a decreased level of progranulin (i.e., a post-treatment test sample). In some embodiments, more than one (e.g., 1, 2, 3, 4, 5, 6, 7, 8, 9, 10, or more) pre-treatment and/or post-treatment test samples are obtained from the subject. Furthermore, the number of pre-treatment and post-treatment test samples that are obtained need not be the same.

In some embodiments, it may be determined that the subject is not responding to the treatment when the abundance the BMP species measured is within about 1%, 2%, 3%, 4%, 5%, 6%, 7%, 8%, 9%, 10%, 11%, 12%, 13%, 14%, 15%, 16%, 17%, 18%, 19%, or 20% of the reference value.

When a subject (e.g., a target subject) is not responding to treatment (e.g., for a disorder associated with a decreased level of progranulin), in some embodiments, the dosage of one or more therapeutic agents (e.g., progranulin) is altered (e.g., increased) and/or the dosing interval is altered (e.g., the time between doses is decreased). In some embodiments, when a subject is not responding to treatment, a different therapeutic agent is selected. In some embodiments, when a subject is not responding to treatment, one or more therapeutic agents is discontinued.

### XV. Bmp Detection Techniques

In some embodiments, antibodies can be used to detect and/or measure the abundance of one or more BMP species. BMP species bound to the antibody can be detected such as by microscopy or enzyme-linked immunosorbent assay (ELISA).

In other embodiments, mass spectrometry (MS) is used to detect and/or measure the abundance of one or more BMP species according to methods of the present disclosure. Mass spectrometry is a technique in which compounds are ionized, and the resulting ions are sorted by their mass-to-charge ratios (abbreviated m/Q, m/q, m/Z, or m/z). A sample (e.g., comprising a BMP molecule), which can be present in gas, liquid, or solid form, is ionized, and the resulting ions are then accelerated through an electric and/or magnetic field, causing them to be separated by their mass-to-charge ratios. The ions ultimately strike an ion detector and a mass spectrogram is generated. The mass-to-charge ratios of the detected ions, together with their relative abundance, can be used to identify the parent compound(s), sometimes by correlating known masses (e.g., of entire or intact molecules) to the masses of the detected ions and/or by recognition of patterns that are detected in the mass spectrogram.

Mass spectrometers typically include at least four primary components: (1) a sample inlet device (e.g., a vaporizer), (2) an ionization device, (3) an ion path, and (4) an ion detector. In addition, mass spectrometers commonly comprise a device that converts samples into a form suitable for the inlet device and/or separates compounds that are present within the sample, which in some embodiments can be a chromatography device (e.g., liquid or gas chromatography) or a solid target for matrix-assisted laser desorption/ionization (MALDI) or another technique suitable for solid samples. Mass spectrometers also commonly comprise a device for signal processing of detector signals (e.g., an analog-digital converter (ADC)) and/or software for the processing, analysis, and display of detector signals.

The sample inlet device facilitates the transition of a solid or liquid specimen into the gaseous phase, which is required for subsequent processing and analysis. The ionization device can utilize, for example, hard ionization (e.g., electron ionization) or soft ionization (e.g., fast atom bombardment (FAB), chemical ionization (CI), electrospray ionization (ESI), MALDI, or atmospheric-pressure chemical ionization (APCI)). ESI methods comprise passing a solution through a length of capillary tube, to the end of which is applied a high positive or negative electric potential. Solution reaching the end of the tube is vaporized into a jet or spray comprising very small droplets of solution in solvent vapor. This spray of droplets flows through an evaporation chamber that is heated slightly to prevent condensation and to evaporate solvent. As the droplets get smaller, the electrical surface charge density increases until the natural repulsion between like charges causes ions as well as neutral molecules to be released.

In the ion path, ions transition from a near atmospheric pressure environment to the low pressure (e.g., high vacuum) environment of the mass analyzer, which separates the ions according to their mass-to-charge ratios, and are moved towards the ion detector. The ion detector is commonly an electron multiplier or a microchannel plate that releases a cascade of electrons in response to being struck by an ion.

Different types of mass analyzers can be used, examples of which include sector field mass analyzers, time-of-flight (TOF) mass analyzers, and quadrupole mass analyzers. Sector field mass analyzers use a static electric and/or magnetic field to modify the ion path and/or velocity, effectively bending the trajectories of the ions according to their mass-to-charge ratios. Ions with higher charges and/or lower masses will be deflected more than ions with lower charges and/or higher masses. A TOF mass analyzer uses an electric field to accelerate the ions through a specified potential, and the time that an ion takes to strike the detector is measured. If all of the ions have the same charge, then their velocities will only differ as a function of their masses, and ions with lower masses will strike the detector first. Quadrupole mass analyzers employ one or more sets of four parallel rods (a set of four parallel rods being known as a quadrupole) to generate oscillating electrical fields that stabilize or destabilize the paths of ions as they pass through an electric quadrupole field (e.g., radio frequency (RF) quadrupole file) that is created between the four rods. In particular, only ions within a specific range of mass-to-charge ratios are allowed to pass though the mass analyzer at any given time. However, by varying the potentials on the rods, a wide range of mass-to-charge ratios can be swept rapidly. Mass-to-charge ratios can be swept either continuously or by specifying discrete jumps.

As opposed to single mass spectrometry (MS) that uses a single mass analyzer (e.g., quadrupole), tandem mass spectrometry (MS/MS) uses a series of mass analyzers (e.g., three mass analyzers) to perform multiple rounds of mass spectrometry, typically having a molecule fragmentation step in between. As a non-limiting example, MS/MS instruments commonly employ three quadrupole mass analyzers. The first quadrupole (Q1) can act as a first mass filter, separating a species or protein of interest from a larger heterogeneous population. The second quadruple (Q2) can act as a collision chamber that stabilizes the ions that have passed through Q1 and can be filled with a low-pressure gas, with which the ions collide, causing them to fragment (collision-induced-fragmentation (CID)). The third quadrupole (Q3) can act as a second mass filter that separates the fragments produced in Q2 and passes them along to the detector. Alternatively, instead of performing tandem mass spectrometry in space, tandem mass spectrometry can be performed over time using a single mass analyzer, such as when a quadrupole ion trap is used and the field is varied over time. Briefly, a quadrupole ion trap works based on the same physical principles as a quadrupole mass analyzer, but the ions are trapped within the quadrupole (i.e., the electric field changes faster than the time required for the ions to escape) and are selectively ejected over time by varying the field generated by the quadrupole.

Several methods can be used for fragmentation, including but not limited to CID, electron capture dissociation (ECD), electron transfer dissociation (ETD), infrared multiphoton dissociation (IRMPD), blackbody infrared radiative dissociation (BIRD), electron-detachment dissociation (EDD), and surface-induced dissociation (SID).

Tandem mass spectrometers can be used to run different types of experiments, including full scans, product ion scans, precursor ion scans, neutral loss scans, and selective (or multiple) reaction monitoring (SRM or MRM) scans. In a full scan experiment, the entire mass range or a portion thereof) of both mass analyzers (e.g., Q1 and Q3) are scanned and the second mass analyzer (e.g., Q2) does not contain any collision gas. This allows all ions contained in a sample to be detected. In a product ion scan experiment, a specific mass-to-charge ratio is selected for the first mass analyzer (e.g., Q1), the second mass analyzer (e.g., Q2) is filled with a collision gas to fragment ions having the selected mass-to-charge ratio, and then the entire mass range (or a portion thereof) of the third mass analyzer (e.g., Q3) is scanned. This allows all fragment ions of a selected precursor ion to be detected. In a precursor ion scan experiment, the entire mass range (or a portion thereof) of the first mass analyzer (e.g., Q1) is scanned, the second mass analyzer (e.g., Q2) is filled with collision gas to fragment ions falling within the scan range, and a specific mass-to-charge ratio is selected for the third mass analyzer (e.g., Q3). By correlating the time between detection of a product ion and the particular mass-to-charge ratio that was selected just prior to its detection, this type of experiment can allow a user to determine which precursor ion(s) may have generated the product ion of interest. In a neutral loss scan experiment, the entire mass range (or a portion thereof) of the first mass analyzer (e.g., Q1) is scanned, the second mass analyzer (e.g., Q2) is filled with collision gas to fragment all ions within the scan range, and the third mass analyzer (e.g., Q3) is scanned across a specified range that corresponds to the fragmentation-induced loss of a single specific mass that has occurred for every potential ion in the precursor scan range. This type of experiment permits the identification of all precursors that have lost a particular chemical group of interest (e.g., a methyl group) in common. In an MRM experiment, one specific mass-to-charge ratio is selected for the first mass analyzer (e.g., Q1), the second mass analyzer (e.g., Q2) is filled with collision gas, and the third mass analyzer (e.g., Q3) is set for another specific mass-to-charge ratio. This type of experiment permits the highly specific detection of molecules that are known to fragment into the products that are selected for in the third mass analyzer. MS and MS/MS methods are described further in Grebe et al. Clin. Biochem. Rev. (2011) 32:5-31, hereby incorporated by reference in its entirety for all purposes.

Furthermore, MS and MS/MS techniques can be coupled with liquid chromatography (LC) or gas chromatography (GC) techniques. Such liquid chromatography-mass spectrometry (LC-MS), liquid chromatography-tandem mass spectrometry (LC-MS/MS), gas chromatography-mass spectrometry (GC-MS), and gas chromatography-tandem mass spectrometry (GC-MS/MS) methods allow for enhanced mass resolving and mass determining over what is typically possible with MS or MS/MS alone.

Liquid chromatography refers to a process in which one or more components of a fluid solution are selectively retarded as the fluid uniformly percolates through a column of a finely divided substance, or through capillary passageways. The retardation results from the distribution of the components of the mixture between one or more stationary phases and the bulk fluid (i.e., mobile phase), as the fluid moves relative to the stationary phase(s). High performance liquid chromatography (HPLC), also sometimes known as “high pressure liquid chromatography,” is a variant of LC in which the degree of separation is increased by forcing the mobile phase under pressure through a stationary phase, typically a densely packed column.

Furthermore, ultra high performance liquid chromatography (UHPLC), also known as “ultra high pressure liquid chromatography,” or “ultra performance liquid chromatography (UPLC),” is a variant of HPLC that is performed using much higher pressures than traditional HPLC techniques.

In some embodiments, the size of particles in the column is less than about 2.0 μm (e.g., about 1.9 μm, 1.8 μm, 1.7 μm, 1.6 μm, 1.5 μm, or smaller). In some embodiments, the particle size is about 1.7 μm.

In some embodiments, the working pressure on the column is about 400 bar to about 1,000 bar (e.g., about 400, 450, 500, 550, 600, 650, 700, 750, 800, 850, 900, 950, or 1,000 bar).

In some embodiments, during chromatography the temperature of the column is maintained at a temperature between about 40° C. and about 60° C. (e.g., about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C.). In some embodiments, the column is maintained at a temperature of about 55° C.

In some embodiments, the flow rate of the column is between about 0.10 mL/minute and about 0.50 mL/minute (e.g., about 0.10 mL/minute, 0.11 mL/minute, 0.12 mL/minute, 0.13 mL/minute, 0.14 mL/minute, 0.15 mL/minute, 0.16 mL/minute, 0.17 mL/minute, 0.18 mL/minute, 0.19 mL/minute, 0.20 mL/minute, 0.21 mL/minute, 0.22 mL/minute, 0.23 mL/minute, 0.24 mL/minute, 0.25 mL/minute, 0.26 mL/minute, 0.27 mL/minute, 0.28 mL/minute, 0.29 mL/minute, 0.30 mL/minute, 0.31 mb/minute, 0.32 mL/minute, 0.33 mL/minute, 0.34 mL/minute, 0.35 mL/minute, 0.36 mL/minute, 0.37 mL/minute, 0.38 mL/minute, 0.39 mL/minute, 0.40 mb/minute, 0.41 mb/minute, 0.42 mL/minute, 0.43 mL/minute, 0.44 mL/minute, 0.45 mL/minute, 0.46 mb/minute, 0.47 mL/minute, 0.48 mL/minute, 0.49 mL/minute, or 0.50 mb/minute). In some embodiments, the flow rate is about 0.40 mL/minute.

The gradient elution can be made using two solvents (e.g., A and B solvents). In some embodiments, the A solvent is 10 mM ammonium formate+0.1% formic acid in water and the B solvent is acetonitrile with 0.1% formic acid. In some embodiments, the gradient is produced by the following method: 5% A and 95% B for 1 minute, changed to 50% A and 50% B over 6 minutes, changed to 5% A and 95% B over 0.1 minutes, then maintained at 5% A and 95% B for 4.9 minutes. Once the compounds are separated by LC, HPLC, or UHPLC, they may be introduced into the mass spectrometer.

Gas chromatography refers to a method for separating and/or analyzing compounds that can be vaporized without being decomposed. The mobile phase is a carrier gas that is typically an inert gas (e.g., helium) or an unreactive gas (e.g., nitrogen), and the stationary phase is typically a microscopic liquid or polymer layer positioned on an inert solid support inside glass or metal tubing that serves as the “column.” As the gaseous compounds of interest interact with the stationary phase within the column, they are differentially retarded and eluted from the column at different times. The separated compounds can then be introduced into the mass spectrometer.

In some embodiments, antibody-based methods are used to detect and/or measure the abundance of one or more BMP species. Non-limiting examples of suitable methods include enzyme-linked immunosorbent assay (ELISA), immunofluorescence, and radioimmunoassay (RIA) techniques. Methods for performing ELISA, immunofluorescence, and RIA techniques are known in the art.

Any number of sample types can be used as a test sample and/or reference sample in methods of the present disclosure so long as the sample comprises BMP in an amount sufficient for detection such that the abundance can be measured. Non-limiting examples include cells, tissues, blood (e.g., whole blood, plasma, serum), fluids (e.g., cerebrospinal fluid, urine, bronchioalveolar lavage fluid, lymph, semen, breast milk, amniotic fluid), feces, sputum, or any combination thereof. Non-limiting examples of suitable cell types include bone marrow-derived macrophages (BMDMs), blood cells (e.g., peripheral blood mononuclear cells (PBMCs), erythrocytes, leukocytes), neural cells (e.g., brain cells, cerebral cortex cells, spinal cord cells), bone marrow cells, liver cells, kidney cells, splenic cells, lung cells, eye cells (e.g., retinal cells such as retinal pigmented epithelial (RPE) cells), chorionic villus cells, muscle cells, skin cells, fibroblasts, heart cells, lymph node cells, or a combination thereof. In some embodiments, the sample comprises a portion of a cell. In some embodiments, the sample is purified from a cell or a tissue. Non-limiting examples of purified samples include endosomes, lysosomes, extracellular vesicles (e.g., exosomes, microvesicles), and combinations thereof.

In some embodiments, the sample (e.g., test sample and/or reference sample) comprises a cell that is a cultured cell. Non-limiting examples include BMDMs and RPE cells. BMDMs can be obtained, for example, by procuring a sample comprising PBMCs and culturing the monocytes contained therein.

Non-limiting examples of suitable tissue sample types include neural tissue (e.g., brain tissue, cerebral cortex tissue, spinal cord tissue), liver tissue, kidney tissue, muscle tissue, heart tissue, eye tissue (e.g., retinal tissue), lymph nodes, bone marrow, skin tissue, blood vessel tissue, lung tissue, spleen tissue, valvular tissue, and a combination thereof. In some embodiments, a test sample and/or a reference sample comprises brain tissue or liver tissue. In some embodiments, a test and/or a reference sample comprises plasma.

### XVI. Nucleic Acids, Vectors, and Host Cells

Polypeptide chains contained in the fusion proteins as described herein are typically prepared using recombinant methods. Accordingly, in some aspects, the disclosure provides isolated nucleic acids comprising a nucleic acid sequence encoding any of the polypeptide chains comprising Fc polypeptides as described herein, and host cells into which the nucleic acids are introduced that are used to replicate the polypeptide-encoding nucleic acids and/or to express the polypeptides. In some embodiments, the host cell is eukaryotic, e.g., a human cell.

In another aspect, polynucleotides are provided that comprise a nucleotide sequence that encodes the polypeptide chains described herein. The polynucleotides may be single-stranded or double-stranded. In some embodiments, the polynucleotide is DNA. In particular embodiments, the polynucleotide is cDNA. In some embodiments, the polynucleotide is RNA.

The disclosure provides an isolated nucleic acid comprising a nucleic acid sequence encoding a polypeptide having the sequence of any one of SEQ ID NOS: 110, 210, 213-215, 225, 227, 261, 273-275, 282, 284, 285, and 291.

The disclosure provides an isolated nucleic acid comprising a nucleic acid sequence encoding a polypeptide having the sequence of SEQ ID NO:215.

The disclosure provides an isolated nucleic acid comprising a nucleic acid sequence encoding a polypeptide having the sequence of SEQ ID NO:210.

The disclosure provides an isolated nucleic acid comprising a nucleic acid sequence encoding a polypeptide having the sequence of SEQ ID NO:227.

The disclosure provides an isolated nucleic acid comprising a nucleic acid sequence encoding a polypeptide having the sequence of SEQ ID NO:291.

The disclosure provides isolated nucleic acids comprising (a) a nucleic acid sequence encoding a polypeptide having the sequence of SEQ ID NO:215, and (b) a nucleic acid sequence encoding a polypeptide having the sequence of SEQ ID NO:210.

The disclosure provides isolated nucleic acids comprising (a) a nucleic acid sequence encoding a polypeptide having the sequence of SEQ ID NO:227, and (b) a nucleic acid sequence encoding a polypeptide having the sequence of SEQ ID NO:210.

The disclosure provides isolated nucleic acids comprising (a) a nucleic acid sequence encoding a polypeptide having the sequence of SEQ ID NO:215, and (b) a nucleic acid sequence encoding a polypeptide having the sequence of SEQ ID NO:291.

The disclosure provides isolated nucleic acids comprising (a) a nucleic acid sequence encoding a polypeptide having the sequence of SEQ ID NO:227, and (b) a nucleic acid sequence encoding a polypeptide having the sequence of SEQ ID NO:291.

In some embodiments, the polynucleotide is included within a nucleic acid construct. In some embodiments, the construct is a replicable vector. In some embodiments, the vector is selected from a plasmid, a viral vector, a phagemid, a yeast chromosomal vector, and a non-episomal mammalian vector.

In some embodiments, the polynucleotide is operably linked to one or more regulatory nucleotide sequences in an expression construct. In one series of embodiments, the nucleic acid expression constructs are adapted for use as a surface expression library. In some embodiments, the library is adapted for surface expression in yeast. In some embodiments, the library is adapted for surface expression in phage. In another series of embodiments, the nucleic acid expression constructs are adapted for expression of the polypeptide in a system that permits isolation of the polypeptide in milligram or gram quantities. In some embodiments, the system is a mammalian cell expression system. In some embodiments, the system is a yeast cell expression system.

Expression vehicles for production of a recombinant polypeptide include plasmids and other vectors. For instance, suitable vectors include plasmids of the following types: pBR322-derived plasmids, pEMBL-derived plasmids, pEX-derived plasmids, pBTac-derived plasmids, and pUC-derived plasmids for expression in prokaryotic cells, such as E. coli. The pcDNAI/amp, pcDNAI/neo, pRc/CMV, pSV2gpt, pSV2neo, pSV2-dhfr, pTk2, pRSVneo, pMSG, pSVT7, pko-neo, and pHyg-derived vectors are examples of mammalian expression vectors suitable for transfection of eukaryotic cells. Alternatively, derivatives of viruses such as the bovine papilloma virus (BPV-1), or Epstein-Ban-virus (pHEBo, pREP-derived, and p205) can be used for transient expression of polypeptides in eukaryotic cells. In some embodiments, it may be desirable to express the recombinant polypeptide by the use of a baculovirus expression system. Examples of such baculovirus expression systems include pVL-derived vectors (such as pVL1392, pVL1393, and pVL941), pAcUW-derived vectors (such as pAcUW1), and pBlueBac-derived vectors. Additional expression systems include adenoviral, adeno-associated virus, and other viral expression systems.

Vectors may be transformed into any suitable host cell. In some embodiments, the host cells, e.g., bacteria or yeast cells, may be adapted for use as a surface expression library. In some cells, the vectors are expressed in host cells to express relatively large quantities of the polypeptide. Such host cells include mammalian cells, yeast cells, insect cells, and prokaryotic cells. In some embodiments, the cells are mammalian cells, such as Chinese Hamster Ovary (CHO) cell, baby hamster kidney (BHK) cell, NS0 cell, Y0 cell, HEK293 cell, COS cell, Vero cell, or HeLa cell.

A host cell transfected with an expression vector encoding one or more Fc polypeptide chains as described herein can be cultured under appropriate conditions to allow expression of the one or more polypeptides to occur. The polypeptides may be secreted and isolated from a mixture of cells and medium containing the polypeptides. Alternatively, the polypeptides may be retained in the cytoplasm or in a membrane fraction and the cells harvested, lysed, and the polypeptide isolated using a desired method.

### XVII. Therapeutic Methods

A fusion protein in accordance with the disclosure may be used therapeutically to treat progranulin-associated disorders (e.g., a neurodegenerative disease (e.g., FTD, NCL, NPA, NPB, NPC, C9ORF72-associated ALS/FTD, sporadic ALS, AD, Gaucher's disease (e.g., Gaucher's disease types 2 and 3), and Parkinson's disease), atherosclerosis, a disorder associated with TDP-43, and AMD).

A fusion protein described herein that comprises a progranulin polypeptide or a variant thereof may be administered to a subject at a therapeutically effective amount or dose. Illustrative dosages include a dose range of about 1 mg/kg to about 100 mg/kg, or about 10 mg/kg to about 50 mg/kg, can be used. The dosages, however, may be varied according to several factors, including the dose frequency, the chosen route of administration, the formulation of the composition, patient response, the severity of the condition, the subject's weight, and the judgment of the prescribing physician. The dosage can be increased or decreased over time, as required by an individual patient. In some embodiments, a patient initially is given a low dose, which is then increased to an efficacious dosage tolerable to the patient.

In various embodiments, a fusion protein described herein is administered parenterally. In some embodiments, the protein is administered intravenously. Intravenous administration can be by infusion, e.g., over a period of from about 10 to about 30 minutes, or over a period of at least 1 hour, 2 hours, or 3 hours. In some embodiments, the protein is administered as an intravenous bolus. Combinations of infusion and bolus administration may also be used.

In some parenteral embodiments, a fusion protein is administered intraperitoneally, subcutaneously, intradermally, or intramuscularly. In some embodiments, the protein is administered intradermally or intramuscularly. In some embodiments, the protein is administered intrathecally, such as by epidural administration, or intracerebroventricularly.

In other embodiments, a fusion protein may be administered orally, by pulmonary administration, intranasal administration, intraocular administration, or by topical administration. Pulmonary administration can also be employed, e.g., by use of an inhaler or nebulizer, and formulation with an aerosolizing agent.

### XVIII. Pharmaceutical Compositions and Kits

In other aspects, pharmaceutical compositions and kits comprising a fusion protein in accordance with the disclosure are provided.

### Pharmaceutical Compositions

Guidance for preparing formulations for use in the disclosure can be found in any number of handbooks for pharmaceutical preparation and formulation that are known to those of skill in the art.

In some embodiments, a pharmaceutical composition comprises a fusion protein as described herein and further comprises one or more pharmaceutically acceptable carriers and/or excipients. A pharmaceutically acceptable carrier includes any solvents, dispersion media, or coatings that are physiologically compatible and that do not interfere with or otherwise inhibit the activity of the active agent.

In some embodiments, the carrier is suitable for intravenous, intrathecal, intraocular, intracerebroventricular, intramuscular, oral, intraperitoneal, transdermal, topical, or subcutaneous administration. Pharmaceutically acceptable carriers can contain one or more physiologically acceptable compounds that act, for example, to stabilize the composition or to increase or decrease the absorption of the polypeptide. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are also available in the art.

The pharmaceutical compositions described herein can be manufactured, e.g., by means of conventional mixing, dissolving, granulating, dragee-making, emulsifying, encapsulating, entrapping, or lyophilizing processes. The following methods and excipients are exemplary.

For oral administration, a fusion protein as described herein can be formulated by combining it with pharmaceutically acceptable carriers that are well known in the art. Such carriers enable the compounds (e.g., fusion proteins as described herein) to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the fusion proteins with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents can be added, such as a cross-linked polyvinyl pyrrolidone, agar, or alginic acid or a salt thereof such as sodium alginate.

As disclosed above, a fusion protein as described herein can be formulated for parenteral administration by injection, e.g., by bolus injection or continuous infusion. For injection, the fusion protein can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. In some embodiments, the fusion protein can be formulated in aqueous solutions, such as physiologically compatible buffers, non-limiting examples of which include Hanks's solution, Ringer's solution, and physiological saline buffer. Formulations for injection can be presented in unit dosage form, e.g., in ampules or in multi-dose containers, with an added preservative. The compositions can take such forms as suspensions, solutions, or emulsions in oily or aqueous vehicles, and can contain formulatory agents such as suspending, stabilizing, and/or dispersing agents.

In some embodiments, a fusion protein as described herein is prepared for delivery in a sustained-release, controlled release, extended-release, timed-release, or delayed-release formulation, for example, in semi-permeable matrices of solid hydrophobic polymers containing the active agent. Various types of sustained-release materials have been established and are well known by those skilled in the art. Extended-release formulations include film-coated tablets, multiparticulate or pellet systems, matrix technologies using hydrophilic or lipophilic materials and wax-based tablets with pore-forming excipients. Usually, sustained release formulations can be prepared using naturally-occurring or synthetic polymers, for instance, polymeric vinyl pyrrolidones, such as polyvinyl pyrrolidone; carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions described herein may vary depending on the particular use envisioned. Suitable dosages are also described in Section XVII above.

### Kits

In some embodiments, a kit for use in treating a progranulin-associated disorder (e.g., a neurodegenerative disease (e.g., FTD, NCL, NPA, NPB, NPC, C9ORF72-associated ALS/FTD, sporadic ALS, AD, Gaucher's disease (e.g., Gaucher's disease types 2 and 3), and Parkinson's disease), atherosclerosis, a disorder associated with TDP-43, and AMD) comprising a fusion protein as described herein is provided.

In some embodiments, the kit further comprises one or more additional therapeutic agents. For example, in some embodiments, the kit comprises a fusion protein as described herein and further comprises one or more additional therapeutic agents for use in the treatment of progranulin-associated disorders (e.g., a neurodegenerative disease (e.g., FTD)). In some embodiments, the kit further comprises instructional materials containing directions (i.e., protocols) for the practice of the methods described herein (e.g., instructions for using the kit for administering a fusion protein comprising the progranulin polypeptide across the blood-brain barrier). While the instructional materials typically comprise written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. Such media may include addresses to internet sites that provide such instructional materials.

### XIX. Transgenic Animals

Further, the disclosure also provides non-human transgenic animals that comprise (a) a nucleic acid that encodes a chimeric TfR polypeptide comprising: (i) an apical domain having at least 90% identity to SEQ ID NO:296 and (ii) the transferrin binding site of the native TfR polypeptide of the animal, and (b) a knockout of the GRN gene, and wherein the chimeric TfR polypeptide is expressed in the brain of the animal. The chimeric forms of the transferrin receptor include a non-human (e.g., mouse) mammalian transferrin binding site and an apical domain that is heterologous to the domain containing the transferrin binding site. These chimeric receptors can be expressed in transgenic animals, particularly where the transferrin binding site is derived from the transgenic animal species and where the apical domain is derived from a primate (e.g., human or monkey). The chimeric TfR polypeptide can comprise an amino acid sequence having at least 95% (e.g., 97%, 98%, or 990%) identity to SEQ ID NO:300. Also described herein is a polynucleotide encoding a chimeric transferrin receptor that comprises a non-human mammalian transferrin binding site and an apical domain having an amino acid sequence at least 80%, 90%, 95%, or 98% identical to SEQ ID NO:296. The nucleic acid sequence encoding the apical domain can comprise a nucleic acid sequence having at least 95% (e.g., 97%, 98%, or 99%) identity to SEQ ID NO:301. The transgenic animal can be homozygous or heterozygous for the nucleic acid encoding the chimeric TfR polypeptide. Further, the knockout of the GRN gene can comprise a deletion of exons 1-4 of the GRNgene.

Methods for generating transgenic knock-in mice have been published in the literature and are well known to those with skill in the art. A method to generate a human TfR knock-in mouse includes pronuclear injection into single cell embryos, in for example C57B16 mice, followed by embryo transfer to pseudo pregnant females. More specifically, Cas9, sgRNAs, and a donor DNA, are introduced into the embryos. The donor DNA encodes the human apical domain coding sequence that has been codon optimized for expression in mouse. The apical domain coding sequence can be flanked with a left and right homology arm. The donor sequence is designed in this manner such that the apical domain is to be inserted after the fourth mouse exon, and is immediately flanked at the 3′ end by the ninth mouse exon. A founder male from the progeny of the female that received the embryos can then be bred to wild-type females to generate F1 heterozygous mice. Homozygous mice can be subsequently generated from breeding of F1 generation heterozygous mice.

The disclosure also provides a non-human, for example, non-primate, transgenic animal (e.g., a mouse or a rat) expressing such chimeric TfRs and a knockout of the GRNgene and the use of the non-human transgenic animal to screen for polypeptides that can cross the BBB by binding to human transferrin receptor (huTfR) in vivo. In some embodiments, the non-human transgenic animal contains a native transferrin receptor (such as a mouse transferrin receptor (mTfR)), in which the apical domain is replaced with an orthologous apical domain having an amino acid sequence at least 80%, 90%, 95%, or 98% identical to SEQ ID NO:296, thereby leaving the native transferrin binding site and the majority, e.g., at least 70%, or at least 75%, of the sequence encoding the transferrin receptor intact. This non-human transgenic animal thus maximally retains the transferrin-binding functionality of the endogenous transferrin receptor of the non-human animal, including the ability to maintain proper iron homeostasis as well as bind and transport transferrin. As a result, the transgenic animal is healthy and suitable for use in discovery and development of therapeutics for treating brain diseases.

### XX. Examples

The present disclosure will be described in greater detail byway of specific examples. The following examples are offered for illustrative purposes only, and are not intended to limit the disclosure in any manner. Those of skill in the art will readily recognize a variety of noncritical parameters which can be changed or modified to yield essentially the same results. Efforts have been made to ensure accuracy with respect to numbers used (e.g., amounts, temperatures, etc.), but some experimental error and deviation may be present. The practice of the present disclosure will employ, unless otherwise indicated, conventional methods of protein chemistry, biochemistry, recombinant DNA techniques and pharmacology, within the skill of the art. Such techniques are explained fully in the literature. Additionally, it should be apparent to one of skill in the art that the methods for engineering as applied to certain libraries can also be applied to other libraries described herein.

### Example 1. Recombinant Fc Dimer:PGRN Fusion Protein Expression and Purification

To express the recombinant Fc dimer:PGRN fusion proteins in Expi293 (Thermo-Fisher), cells were transfected at 2×10⁶ cells/mL density with Expifectamine™ 293/plasmid DNA complex according to manufacturer's instructions (Thermo-Fisher). After transfection, cells were incubated at 37° C. with a humidified atmosphere of 6-8% C02 in an orbital shaker (Infors HT Multitron). On day one post-transfection, Expifectamine™ transfection enhancer 1 and 2 were added to the culture. Media supernatant was harvested by centrifugation after 96-hour post-transfection. The clarified supernatant was supplemented with EDTA-free protease inhibitor (Roche) and was stored at −80° C.

For recombinant fusion protein isolation, clarified media supernatant was loaded on HiTrap MabSelect SuRe Protein A affinity column (GE Healthcare Life Sciences) and washed with wash buffer I (PBS buffer pH 7.4) and wash buffer II (PBS buffer pH 7.4 and 150 mM NaCl). The fusion protein was eluted in 50 mM QB citrate buffer pH 3.0 with 150 mM NaCl. Immediately after elution, the arginine-succinate buffer (1 M arginine, 685 mM succinic acid pH 5.0) was added to adjust the pH. Protein aggregates were separated from monodispersed fusion proteins by size exclusion chromatography (SEC) on Superdex 200 increase 16/60 GL column (GE Healthcare Life Sciences). The SEC mobile phase was kept in arginine-succinate pH 5.0 buffer. All chromatography steps were performed on AKTA pure or AKTA Avant systems (GE Healthcare Life Sciences).

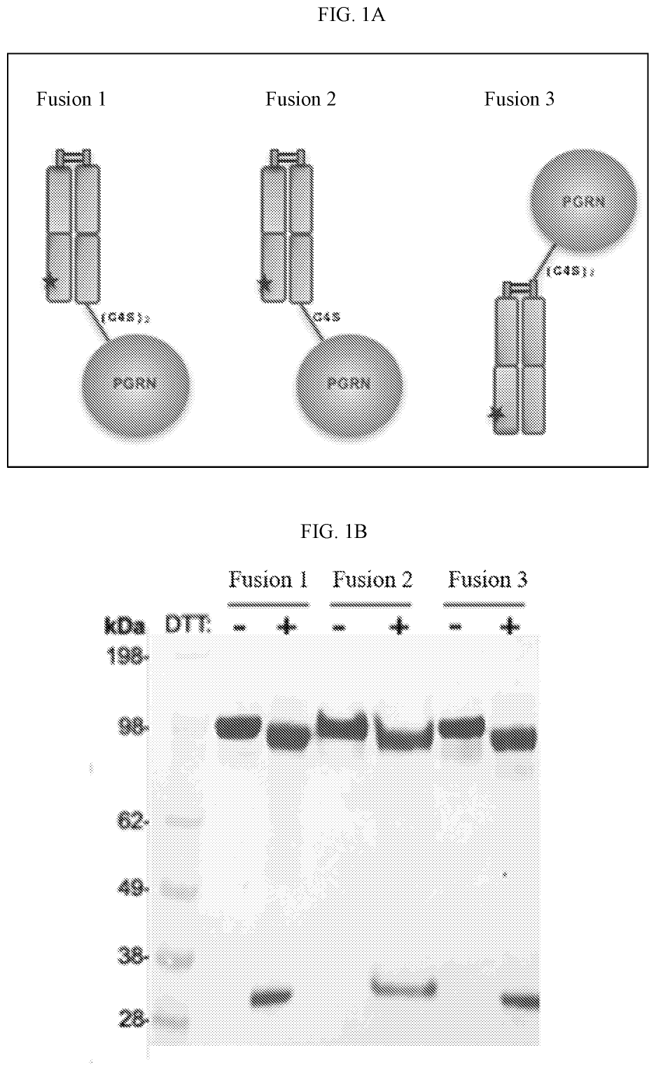

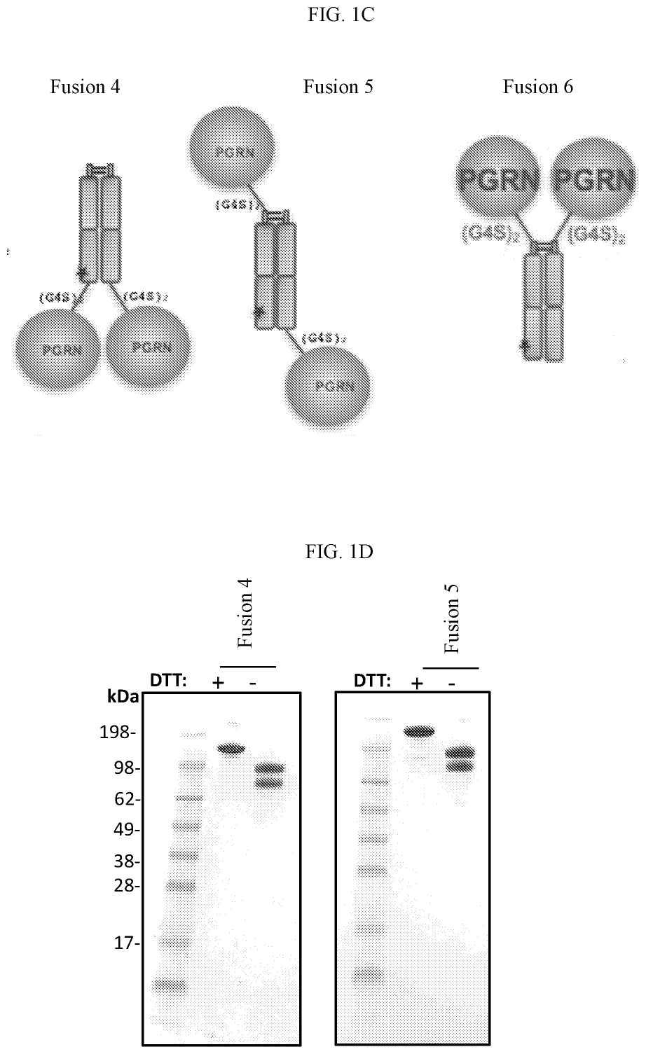

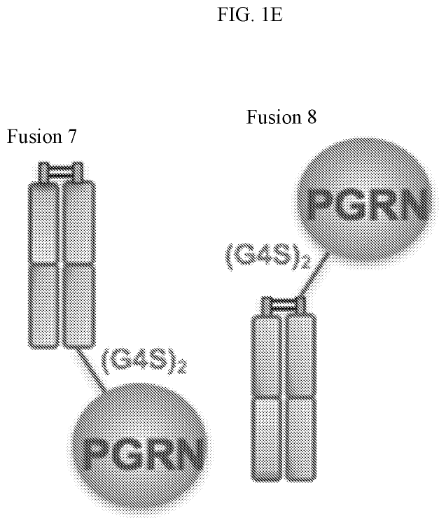

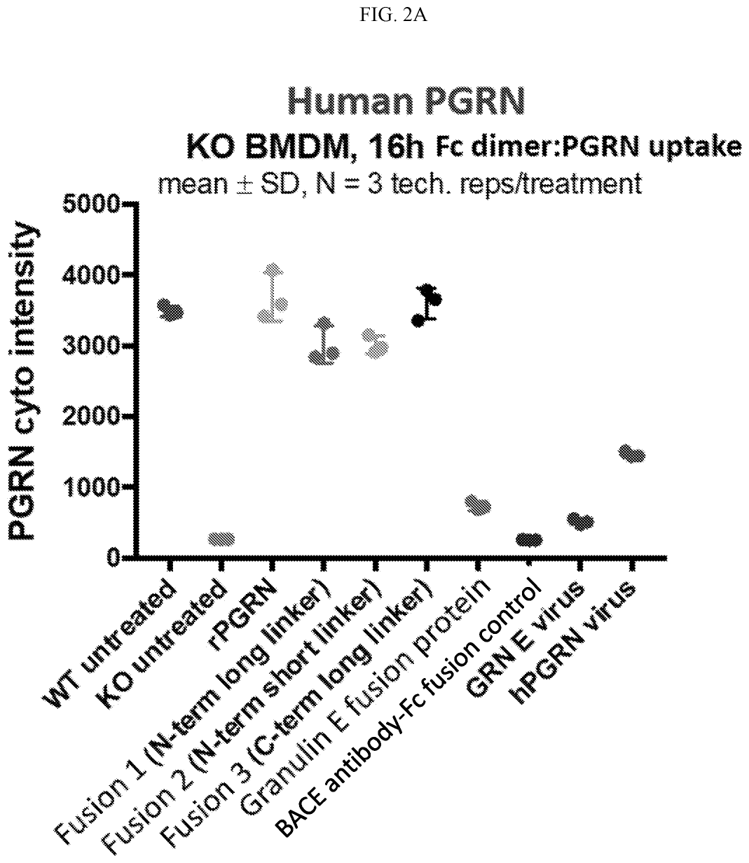

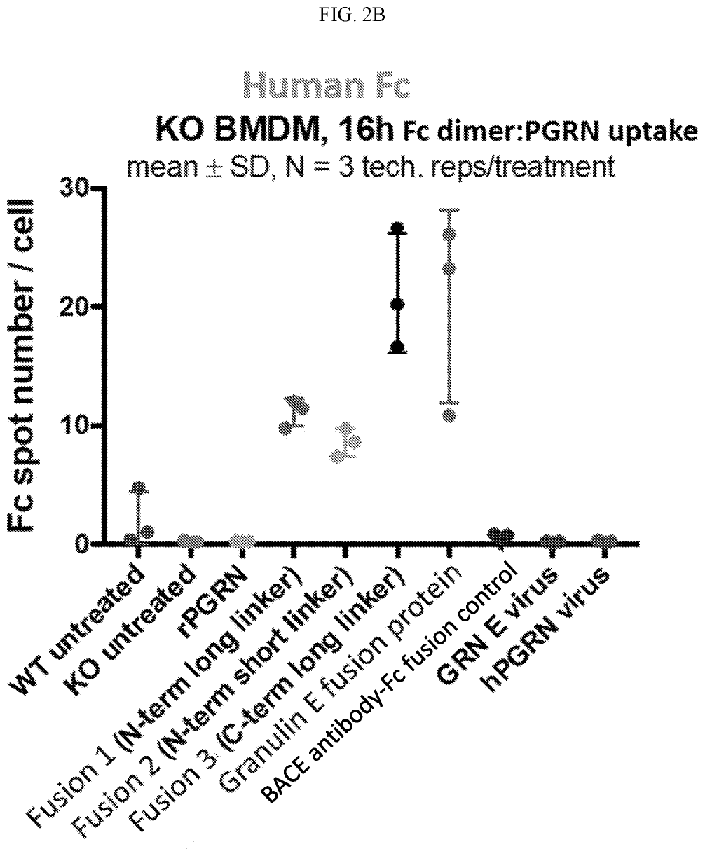

FIG. 1A is a schematic drawing showing three Fc dimer:PGRN fusion proteins Fusion 1, Fusion 2, and Fusion 3. In Fusion 1 and Fusion 2, the N-terminus of PGRN is fused to the C-terminus of the Fc polypeptide that does not contain TfR-binding mutations (indicated by star) by way of a (G₄S)₂ linker (SEQ ID NO:276) and a G₄S linker (SEQ ID NO:277), respectively. In Fusion 3, the C-terminus of PGRN is fused to the N-terminus of the Fc polypeptide that does not contain TfR-binding mutations (indicated by star) by way of a (G₄S)₂ linker. FIG. 1B shows that Fusion 1, Fusion 2, and Fusion 3 each containing one PGRN molecule were purified to greater than 85% purity.

FIG. 1C is a schematic drawing showing the Fc dimer:PGRN fusion protein Fusion 4, Fusion 5, and Fusion 6. In Fusion 4, each of the two PGRN molecules is fused to the C-terminus of an Fc polypeptide by way of the linker (G₄S)₂. One PGRN molecule is fused to C-terminus of the Fc polypeptide containing TfR-binding mutations (indicated by star), while the other PGRN molecule is fused to C-terminus of the Fc polypeptide without the TfR-binding III mutations. In Fusion 5, one PGRN molecule is fused to the N-terminus of the Fc polypeptide containing TfR-binding mutations (indicated by star) by way of the linker (G₄S)₂, while the other PGRN molecule is fused to the C-terminus of the other Fc polypeptide without the TfR-binding mutations. In Fusion 6, each of the two PGRN molecules is fused to the N-terminus of an Fc polypeptide by way of the linker (G₄S)₂. One PGRN molecule is fused to N-terminus of the Fc polypeptide containing TfR-binding mutations (indicated by star), while the other PGRN molecule is fused to N-terminus of the Fc polypeptide without the TfR-binding mutations. FIG. 1D shows that fusion proteins Fusion 4 and Fusion 5 each containing two PGRN molecules were purified to greater than 85% purity.

Other Fc dimer:PGRN fusion proteins include Fusion 7 and Fusion 8 (FIG. 1E). Both fusion proteins Fusion 7 and Fusion 8 contain Fc polypeptides that do not contain TfR-binding mutations. In Fusion 7, the N-terminus of PGRN is fused to the C-terminus of an Fc polypeptide by way of the linker (G₄S)₂. In Fusion 8, the C-terminus of PGRN is fused to the N-terminus of an Fc polypeptide by way of the linker (G₄S)₂. Additional Fc dimer:PGRN fusion proteins are described in Table 1 below, which lists the sequences for each fusion protein.

TABLE 1
Sequences of Fc Dimer: PGRN

| Fc | | Fc Polypeptide or Fc Polypeptide and |
|---|---|---|
| Dimer: PGRN | Fc Polypeptide and PGRN | PGRN |
| Fusion 1 | Partial hinge-Fc polypeptide with hole | Partial hinge-Fc polypeptide with TfR- |
| | mutations-(G₄S)₂-PGRN: SEQ ID NO: 213 | binding (CH3C.35.21.17) and knob |
| | | mutation: SEQ ID NO: 273 |
| Fusion 2 | Partial hinge-Fc polypeptide with hole | Partial hinge-Fc polypeptide with TfR- |
| | mutations-G₄S-PGRN: SEQ ID NO: 214 | binding (CH3C.35.21.17) and knob |
| | | mutation: SEQ ID NO: 273 |
| Fusion 3 | PGRN-(G₄S)₂-Partial hinge-Fc polypeptide | Partial hinge-Fc polypeptide with TfR- |
| | with hole mutations: SEQ ID NO: 225 | binding (CH3C.35.21.17) and knob |
| | | mutation: SEQ ID NO: 273 |
| Fusion 4 | Partial hinge-Fc polypeptide with hole | Partial hinge-Fc polypeptide with TfR- |
| | mutations-(G₄S)₂-PGRN: SEQ ID NO: 213 | binding (CH3C.35.21.17) and knob |
| | | mutation-(G₄S)₂-PGRN: SEQ ID NO: 274 |
| Fusion 5 | Partial hinge-Fc polypeptide with hole | PGRN-(G₄S)₂-Partial hinge-Fc polypeptide |
| | mutations-(G₄S)₂-PGRN: SEQ ID NO: 213 | with TfR-binding (CH3C35.21.17) and |
| | | knob mutation: SEQ ID NO: 275 |
| Fusion 6 | PGRN-(G₄S)₂-Partial hinge-Fc polypeptide | PGRN-(G₄S)₂-Partial hinge-Fc polypeptide |
| | with hole mutations: SEQ ID NO: 225 | with TfR-binding (CH3C35.21.17) and |
| | | knob mutation: SEQ ID NO: 275 |
| Fusion 7 | Partial hinge-Fc polypeptide with hole | Partial hinge-Fc polypeptide with knob |
| | mutations-(G₄S)₂-PGRN: SEQ ID NO: 213 | mutation: SEQ ID NO: 261 |
| Fusion 8 | PGRN-(G₄S)₂-Partial hinge-Fc polypeptide | Partial hinge-Fc polypeptide with knob |
| | with hole mutations: SEQ ID NO: 225 | mutation: SEQ ID NO: 261 |
| Fusion 9 | Partial hinge-Fc polypeptide with hole and | Partial hinge-Fc polypeptide with TfR- |
| | LALA mutations-(G₄S)₂-PGRN: SEQ ID | binding (CH3C.35.21) and knob and |
| | NO: 215 | LALA mutations: SEQ ID NO: 110. |
| Fusion 10 | PGRN-(G₄S)₂-Partial hinge-Fc polypeptide | Partial hinge-Fc polypeptide with TfR- |
| | with hole and LALA mutations: SEQ ID | binding (CH3C.35.21) and knob and |
| | NO: 227 | LALA mutations: SEQ ID NO: 110. |
| Fusion 11 | Partial hinge-Fc polypeptide with hole and | Partial hinge-Fc polypeptide with TfR- |
| | LALA mutations-(G₄S)₂-PGRN: SEQ ID | binding (CH3C35.21.17) and knob and |
| | NO: 215 | LALA mutations: SEQ ID NO: 291 |
| Fusion 12 | PGRN-(G₄S)₂-Partial hinge-Fc polypeptide | Partial hinge-Fc polypeptide with TfR- |
| | with hole and LALA mutations: SEQ ID | binding (CH3C35.21.17) and knob and |
| | NO: 227 | LALA mutations: SEQ ID NO: 291 |
| Fusion 13 | Partial hinge-Fc polypeptide with hole and | Partial hinge-Fc polypeptide with TfR- |
| | LALA mutations-(G₄S)₂-PGRN: SEQ ID | binding (CH3C.35.21.17) and knob |
| | NO: 215 | mutation: SEQ ID NO: 273 |
| Fusion 14 | PGRN-(G₄S)₂-Partial hinge-Fc polypeptide | Partial hinge-Fc polypeptide with TfR- |
| | with hole and LALA mutations: SEQ ID | binding (CH3C.35.21.17) and knob |
| | NO: 227 | mutation: SEQ ID NO: 273 |
| Fusion 15 | Partial hinge-Fc polypeptide with hole and | Partial hinge-Fc polypeptide with TfR- |
| | LALA mutations-(G₄S)₂-PGRN: SEQ ID | binding (CH3C35.23.2) and knob and |
| | NO: 215 | LALA mutations: SEQ ID NO: 210 |
| Fusion 16 | PGRN-(G₄S)₂-Partial hinge-Fc polypeptide | Partial hinge-Fc polypeptide with TfR- |
| | with hole and LALA mutations: SEQ ID | binding (CH3C35.23.2) and knob and |
| | NO: 227 | LALA mutations: SEQ ID NO: 210 |
| Fusion 17 | Partial hinge-Fc polypeptide with hole and | Partial hinge-Fc polypeptide with TfR- |
| | LALA mutations-(G₄S)₂-PGRN: SEQ ID | binding (CH3C35.23.2) and knob and |
| | NO: 215 | LALAPG mutations: SEQ ID NO: 282 |
| Fusion 18 | PGRN-(G₄S)₂-Partial hinge-Fc polypeptide | Partial hinge-Fc polypeptide with TfR- |
| | with hole and LALA mutations: SEQ ID | binding (CH3C35.23.2) and knob and |
| | NO: 227 | LALAPG mutations: SEQ ID NO: 282 |
| Fusion 19 | Partial hinge-Fc polypeptide with hole and | Partial hinge-Fc polypeptide with TfR- |
| | LALA mutations-(G₄S)₂-PGRN: SEQ ID | binding (CH3C35.23.2) and knob, LALA, |
| | NO: 215 | and LS mutations: SEQ ID NO: 284 |
| Fusion 20 | PGRN-(G₄S)₂-Partial hinge-Fc polypeptide | Partial hinge-Fc polypeptide with TfR- |
| | with hole and LALA mutations: SEQ ID | binding (CH3C35.23.2) and knob, LALA, |
| | NO: 227 | and LS mutations: SEQ ID NO: 284 |
| Fusion 21 | Partial hinge-Fc polypeptide with hole and | Partial hinge-Fc polypeptide with TfR- |
| | LALA mutations-(G₄S)₂-PGRN: SEQ ID | binding (CH3C35.23.2) and knob, |
| | NO: 215 | LALAPG, and LS mutations: SEQ ID |
| | | NO: 285 |
| Fusion 22 | PGRN-(G₄S)₂-Partial hinge-Fc polypeptide | Partial hinge-Fc polypeptide with TfR- |
| | with hole and LALA mutations: SEQ ID | binding (CH3C35.23.2) and knob, |
| | NO: 227 | LALAPG, and LS mutations: SEQ ID |
| | | NO: 285 |

### Example 2. Recombinant Fc Dimer:PGRN Fusion Protein Binding to MTR and Sortilin

All surface plasmon resonance (SPR) experiments were performed on a GE Healthcare Biacore 8K instrument with Series S Sensor Chip CM5 and HBS-EP+ running buffer at 25° C. To measure the binding affinity of the fusion proteins for hMR, the sensor chip was immobilized with streptavidin and biotinylated-AviTag-hTfR was captured. Single-cycle kinetics was used with a 3-fold concentration series of fusion protein analyte ranging from 25 nM-2 μM, allowing for 80 seconds of contact time, 180 seconds of dissociation time, and a flow rate of 30 μL/min. A steady-state affinity model was used to demonstrate that the fusion proteins are capable of binding hTfR.

To measure the binding affinity for sortilin, the fusion proteins were captured using a sensor chip that was immobilized with a GE Healthcare human antibody capture kit. Multi-cycle kinetics was used with a 3-fold concentration series of sortilin analyte ranging from 0.4 nM-100 nM, allowing for 300 seconds of contact time, 600 seconds of dissociation time, and a flow rate of 30 μL/min. A 1:1 kinetics model was used to evaluate the binding kinetics of sortilin binding. The binding affinities of two Fc dimer:PGRN fusion proteins to sortilin are as follows: Fusion 1: 19 nM and Fusion 2: 19 nM, which are similar to the sortilin binding affinity for PGRN reported in literature (about 18 nM). Fusion 3 did not appear to bind sortilin. Fc fusion at C-terminus of PGRN might block the sortilin binding site of PGRN. Alternatively, immobilized Fc used in the Biacore assay might cause steric hinderance for sortilin to access the C-terminus of PGRN.

### Example 3. Cellular Uptake

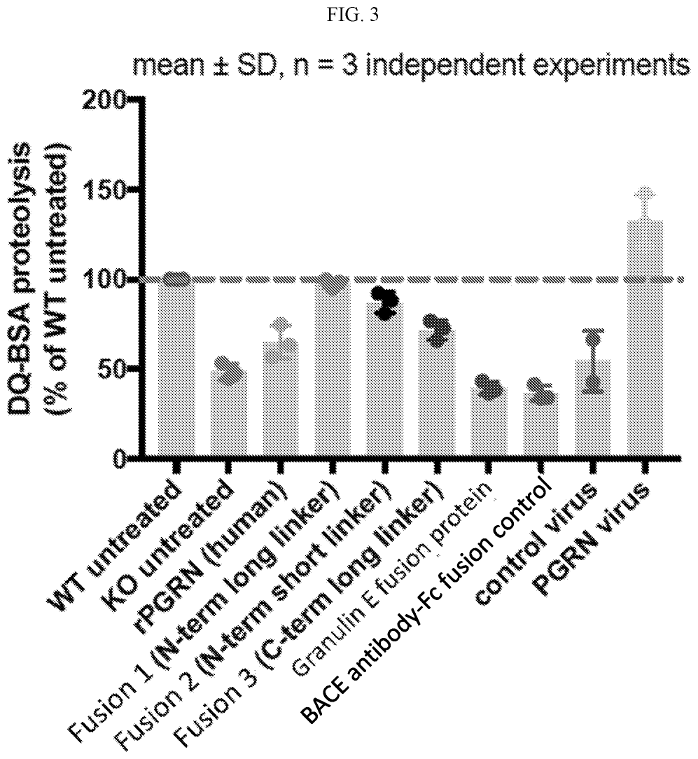

Bone marrow derived macrophages (BMDMs) isolated from GRN WT and KO mice were treated for 16 h with 50 nM recombinant progranulin (PGRN) (Adipogen), 50 nM Fc dimer:PGRN fusion proteins, or human PGRN lentivirus. Fixed BMDMs were immunostained with antibodies against human PGRN (R&D Systems) and human Fc (Thermo Fisher Scientific). Cellular uptake was quantified with an Opera Phenix high-content imaging platform (PerkinElmer) in confocal mode. Treatment with recombinant PGRN and full-length Fc dimer:PGRN fusion proteins (Fusion 1, Fusion 2, and Fusion 3) led to robust increase in cellular PGRN, as shown by both PGRN and Fc stainings (FIGS. 2A and 2B), indicating efficient uptake. The granulin E fusion protein (SEQ ID NO:280 (Partial hinge-Fc polypeptide with hole mutations-(G₄S)₂-Granulin E fusion (amino acids 497-593 of SEQ ID NO:211)) dimerized with Fc polypeptide with knob mutations) was used as a negative control.

### Example 4. Proteolysis Assay

### DQ-BSA Assay

DQ-BSA Red is BSA protein heavily conjugated with BODIPY Red dye. DQ-BSA Red is a fluorogenic substrate (max excitation at 590 nm, emission at 615 nm) for resident proteases of the endo-lysosomal system. Once DQ-BSA is hydrolyzed to smaller dye-labeled peptides inside lysosomes, the strong self-quenching effect conferred by heavy labeling of Bodipy dye is relieved, producing a large increase in fluorescence that is amenable to quantitation by confocal microscopy.

In experiments, BMDMs isolated from GRN WT and KO mice were treated for 6 h with a final concentration of 10 μg/mL DQ-BSA Red in complete media. This time period allowed for DQ-BSA to be passively loaded into cells by fluid phase endocytosis, leading to its distribution throughout the endo-lysosomal network, and ultimately to its proteolysis in lysosomes.

After the 6 h treatment was completed, BMDMs were washed three times with PBS and fixed with 4% PFA in PBS for 15 min. To label cellular nuclei for confocal microscopy, fixed BMDMs were treated with 1 μg/mLDAPI (Thermo-Fisher) in PBS for 10 min and imaged on an Opera-Phenix high content imaging platform (PerkinElmer). Unquenched Bodipy-Red signal was measured by excitation at 568 nm and endo-lysosomal proteolysis was quantified by calculating the integrated Bodipy spot area per cell using an automated analysis module built in Harmony software (PerkinElmer).

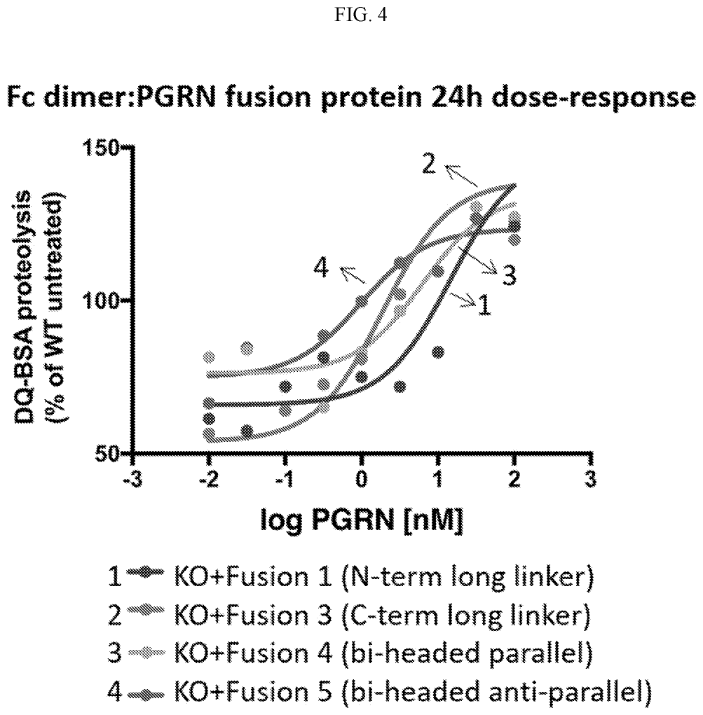

GRN WT and KO BMDMs were pre-treated with 50 nM recombinant PGRN (Adipogen), 50 nM Fc dimer:PGRN fusion proteins, or human PGRN lentivirus for 48 h. A quenched fluorogenic Bodipy-BSA conjugate (DQ-BSA Red, Thermo Fisher Scientific) was added to pre-treated BMDMs at a final concentration of 10 μg/mL for 6 h. Unquenched Bodipy-Red signal was measured by excitation at 568 nm using an Opera Phenix high-content imaging platform (PerkinElmer) in confocal mode. Endo-lysosomal proteolysis was quantified by calculating the integrated Bodipy spot area per cell using an automated analysis module built in Harmony software (PerkinElmer). Fc dimer:PGRN fusion proteins showed either complete (Fusion 1) or partial (Fusion 2 and Fusion 3) rescue of impairment of proteolytic deficits in KO BMDMs (FIG. 3). As shown in FIG. 3, the granulin E fusion protein (SEQ ID NO:280 (Partial hinge-Fc polypeptide with hole mutations-(G₄S)₂-Granulin E fusion (amino acids 497-593 of SEQ ID NO:211)) dimerized with Fc polypeptide with knob mutations) failed to rescue proteolysis.

### Example 5. Bodipy-BSA Conjugate Dose-Response

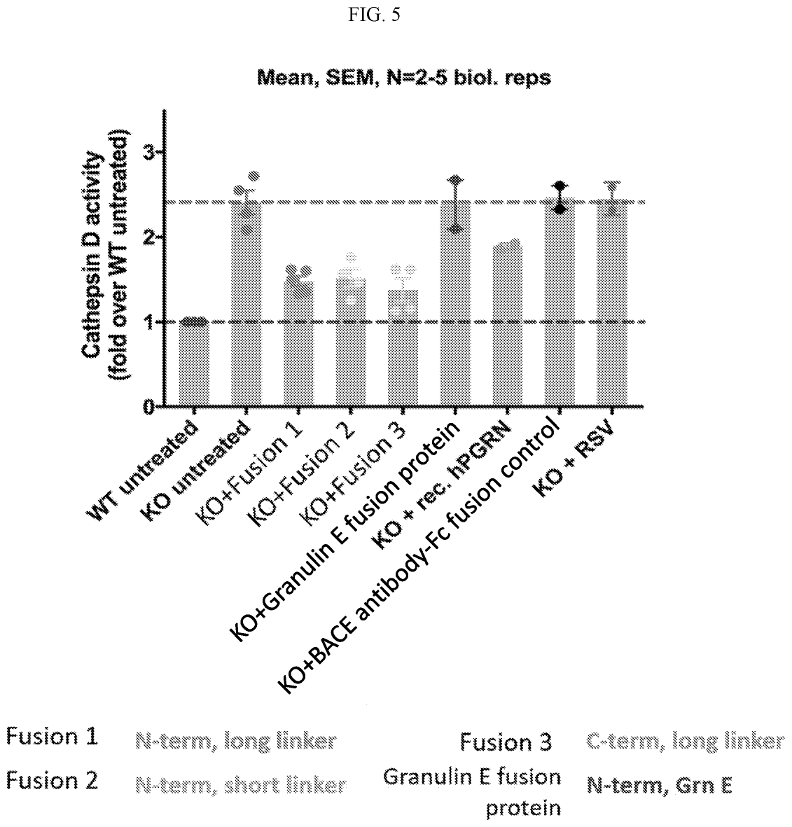

WT and KO GRN BMDMs were treated for 24 h in a Bodipy-BSA conjugate (DQ-BSA) assay with semi-log dose-titrations (100 nM down to 10 μM) of Fc dimer:PGRN fusion proteins (Fusion 1, Fusion 3, Fusion 4, and Fusion 5). Endo-lysosomal proteolysis was determined as described previously. Fusion 5 showed evidence of enhanced potency in the DQ-BSA (FIG. 4).

### Example 6. Cathepsin D Assay

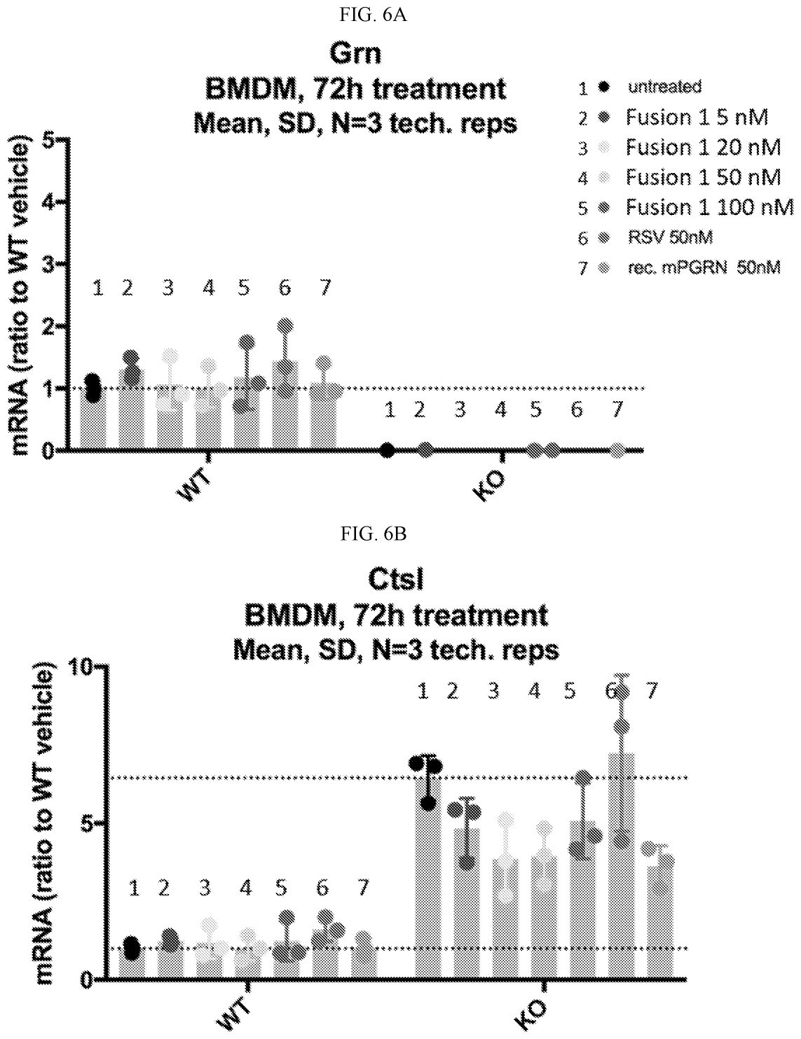

A fluorogenic probe was used to measure cathepsin D activity in cellular lysates. GRN WT and KO BMDMs were treated for 72 h with Fc dimer:PGRN fusion proteins at 50 nM, then the cells were lysed in CST lysis buffer. Cell lysate was diluted into the low pH assay buffer and mixed with the fluorogenic probe. Cathepsin D activity was read on a plate reader and calculated as fold over WT untreated. Three fusion proteins (Fusion 1, Fusion 2, and Fusion 3) showed partial rescue of the elevated cathepsin D activity observed in the GRN KO BMDMs (FIG. 5).

### Example 7. Lysosomal Gene Dysregulation

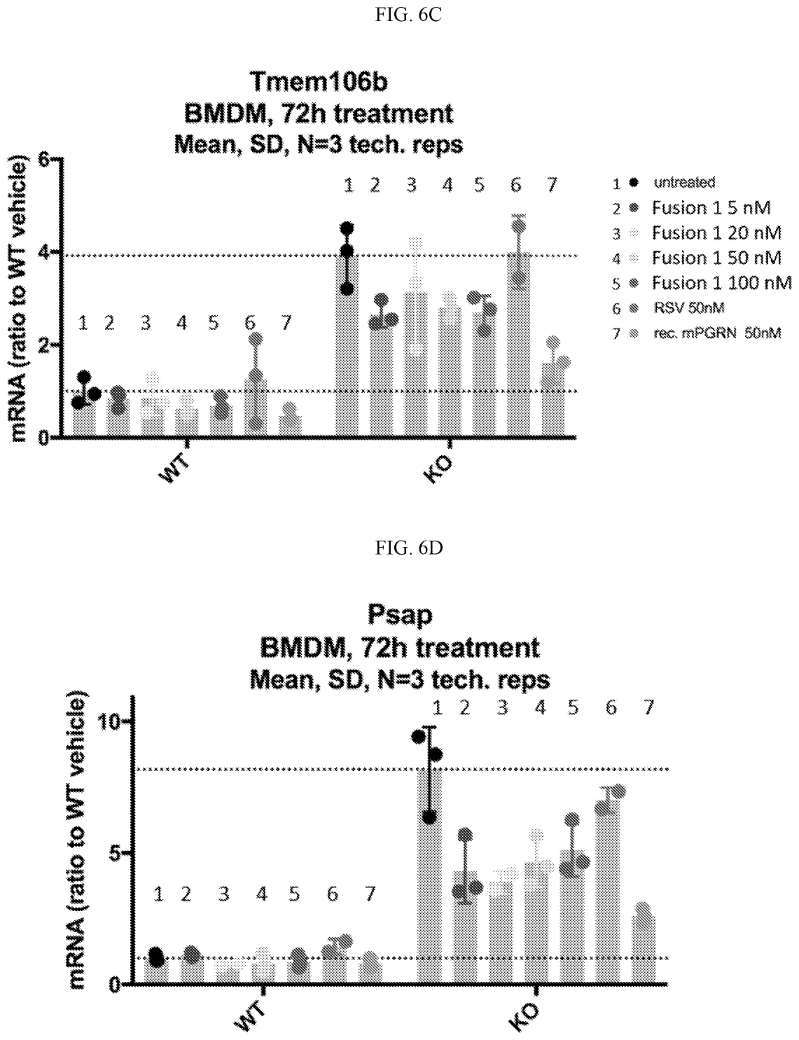

GRN WT and KO BMDMs were treated for 72 h with Fc dimer:PGRN fusion protein Fusion 1 between 5 nM and 50 nM. Cells were lysed and RNA was extracted using Cell-to-CT kit (Fisher Scientific). mRNA levels of lysosomal genes Cts1, Tmem106b, and Psap were measured by qPCR. Treatment with Fc dimer:PGRN fusion protein Fusion 1 showed partial rescue of the elevation of the lysosomal genes in the GRNKO BMDMs (FIGS. 6B-6D).

### Example 8. Pharmacokinetic Properties of Fusion Proteins in Plasma and Liver

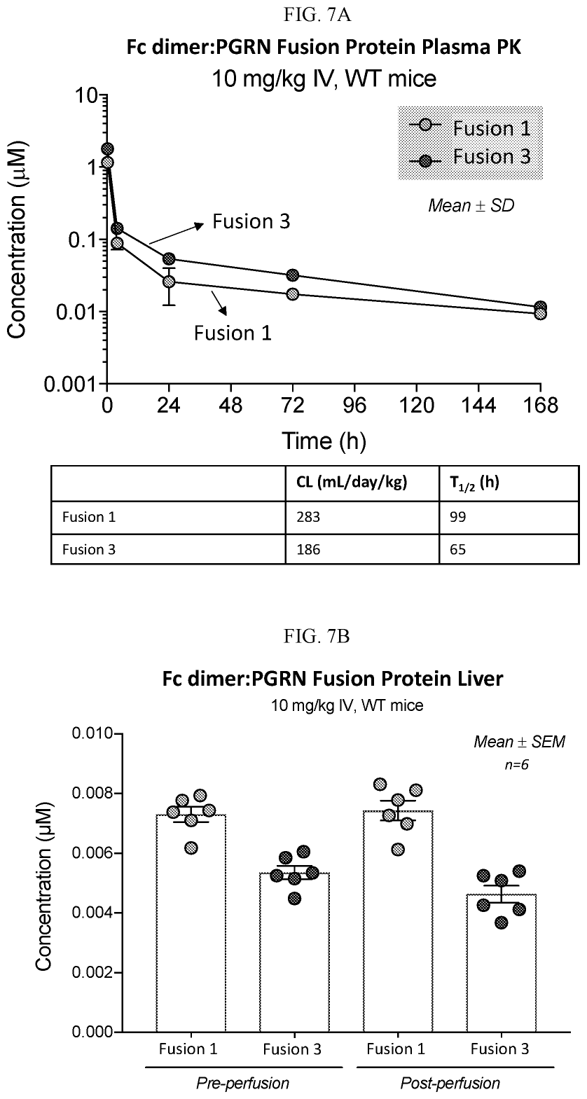

WT mice were dosed once at 10 mg/kg of Fc dimer:PGRN fusion protein Fusion 1 or Fusion 3, and plasma samples were obtained at the timepoints indicated. Liver was homogenized in CST lysis buffer using bead homogenization. The concentration of each Fc dimer:PGRN fusion protein was measured by ELISA in both plasma and terminal liver samples using an Fc-capture-PGRN-detection architecture. PK profiles indicate similar clearance and half-lives of the two fusion proteins (FIGS. 7A and 7B).

### Example 9. Brain Uptake of Fusion Proteins in hTfR Knock-in Mice

This study aimed to determine whether the Fc dimer:PGRN fusion proteins (Fusion 1 and Fusion 3) can be detected in the brain of mice expressing hTfR (for a description of the mice, see, e.g., U.S. Pat. No. 10,143,187). The fusion proteins were injected via the tail vein into hTfR knock-in (hTfR.KI)(homozygous) and non-transgenic mice in an effort to determine if human progranulin (hPGRN) can be detected in the brain of hTfR.KI mice and if so, what is the fold increase of hPGRN in the hTfR.KI mice over that in the non-transgenic injected mice lacking the hTfR.

### Materials and Methods

Animals: The mice used for this study were obtained from JAX Laboratories and consisted of 16 hTfR.KI (homozygous) mice and 10 non-transgenic C57BL/6 males at 8 weeks of age. Animals were housed in standard conditions in the vivarium with ad libitum access to food and water at least 7 days prior to the initiation of the study.

TABLE 2
Study Design/Experimental Groups

| | | Dose | Time points | | Tissue/fluids to be | Material |
|---|---|---|---|---|---|---|
| Genotype | Protein | (mg/kg) | (h) | n/group | collected | (mg) |
| hTfR.KI | Vehicle | — | 15, 4 | 4 | Plasma, brain, liver | NA |
| (homozygous) | Fusion 1 | 50 | 15, 4 | 5 | Plasma, brain, liver | 7.5 mg |
| | Fusion 3 | 50 | 15, 4 | 6 | Plasma, brain, liver | 7.5 mg |
| WT | Fusion 1 | 50 | 15, 4 | 5 | Plasma, brain, liver | 7.5 mg |
| | Fusion 3 | 50 | 15, 4 | 5 | Plasma, brain, liver | 7.5 mg |

Animal allocation to experimental groups: All animals used in this study were males but were distributed equally in each experimental group to account for differences across litters.

Formulation: Fc dimer:PGRN fusion proteins Fusion 1 and Fusion 3 were used at 5.87 mg/mL and 4.98 mg/mL, respectively.

Overall procedures: Experimental conditions were alternated when collecting tissues. Animal groups and sample collections were randomized.

In-life procedures: Submandibular bleeds were performed using 3 mm lancets (GoldenRod animal lancets). Plasma collection: Blood was collected in EDTA tubes (Sarstedt Microvette 500 K3E, Ref #201341102) and slowly inverted 10 times. For small volumes of blood (<100 μL), blood was collected in EDTA tubes with capillary tube (Sarstedt Microvette 100 K3E, Ref #201278100). EDTA tubes were immediately stored in the fridge until plasma preparation. The time between storage and preparation did not exceed 1 hour. The time of collection and time of processing were followed consistently. The tubes were centrifuged at 12,700 rpm for 7 minutes at 4° C. Plasma (top layer) was transferred to 0.6 mL Matrix tubes with rubber seal. Matrix tubes were snap frozen on dry ice before transferring to −80° C.

Terminal fluids/tissue collection procedures: Animals were deeply anesthetized via intraperitoneal (i.p.) injection of 2.5% Avertin and then tissues were collected in the order as described below:

Samples collected before intracardiac perfusion: Plasma collection: Blood was collected via cardiac puncture using a 1 mL Terumo tuberculin syringe attached to a 25 gauge needle (Ref #SS-01T2516) (Not pre-conditioned with EDTA). The needle was then detached and the blood was transferred to EDTA tubes (Sarstedt Microvette 500 K3E, Ref #201341102) and slowly inverted 10 times. Following this procedure, post collection methods were continued as described above in In-Life Procedures.

For samples collected after intracardiac perfusion, animals were transcardially perfused for 5 minutes with ice cold PBS using a peristaltic pump (Gilson Inc. Minipuls Evolution F110701) at a flow rate of 5 mL/minute. For tissues that can be dissected and pre-weighted during collection, the samples were placed directly into 1.5 mL Eppendorf tubes. Tissues were collected in the following order: Liver (post-perfusion): about 100 mg of liver was collected into 1.5 mL Eppendorf tubes. Eppendorf tubes were snap frozen on dry ice before transferring to −80° C. Brain: Right hemisphere was dissected into PK and other brain pieces. Both 50 mg PK and remaining brain pieces were collected into 1.5 mL Eppendorf tubes. Eppendorf tubes were snap frozen on dry ice before transferring to −80° C. Left hemisphere was placed in 4 mL of freshly made 4% paraformaldehyde at 4° C. for 72 hours and then transferred to 30% sucrose for 72 hours before being cut on the freezing microtome coronally at 30 μm/section.

### Protocol for Homogenization

Homogenization buffer was made by making a 1× of the Cell Signaling Technology 10× Cell lysis buffer (Cat No: 9803) (1× buffer: 20 mM Tris-HCl (pH 7.5), 150 mM NaCl, 1 mM Na₂EDTA, 1 mM EGTA, 1% Triton, 2.5 mM sodium pyrophosphate, 1 mM s-glycerophosphate, 1 mM Na₃VO₄, 1 μg/mL leupeptin) and supplementing with 1× protease inhibitor (Complete, Mini Protease Inhibitor cocktail tablets, Roche Cat. No. 04693124001) and 1× phosphatase inhibitor (PhosSTOP, Roche, Cat. No. 04906837001). Lysis buffer was added at about 10× volume of tissue weight for brain and liver samples in 1.5 mL tubes. A 3 mm tungsten carbide bead (Qiagen, Cat. No. 69997) was added to each tube and the tubes loaded into TissueLyser Cassetts. Samples were homogenized on the TissueLyser II (Qiagen Cat. No. 85300) at 29 Hz for 6 minutes (2×3 minute runs). Samples were then removed and run at max speed (18,000×g) on the tabletop centrifuge for 30 minutes at 4° C. The supernatant was transferred to a new 1.5 mL Eppendorf tube and protein concentration was measured using BCA.

Protocol for Fc Capture of hPGRN in ELISA

For the ELISA, 384-well clear microplates (Thermo Fisher Scientific 464718) were coated with a donkey polyclonal antibody specific for human Fc (Jackson ImmunoResearch #709-006-098). The capture antibody was diluted to a final concentration of 1 μg/mL in a sodium biocarbonate buffer. 25 μL/well of the capture coating solution was added to each assay plate, and the plates were incubated overnight at 4° C.

On the day of the assay, the assay plates were washed 3 times with PBST buffer using an automated plate washer (Biotek), after which 80 μL/well of a blocking solution (5% BSA in PBST buffer) was added. The assay plates were incubated in the blocking solution for at least 1 hour at room temperature. During the blocking, dilutions of the plasma, liver lysate, and brain lysate samples were prepared in assay diluent buffer (1% BSA in PBST) in 96-well polypropylene V-bottom plates (Greiner Bio-One 651201). For plasma, serial dilutions of 1:10, 1:100, 1:1000, 1:10,000, 1:100,000, and 1:1,000,000 were prepared. For liver, serial dilutions of 1:20, 1:100, 1:200, 1:400, and 1:800 were prepared. For brain, serial dilutions of 1:10 and 1:40 were prepared. In addition, standard curve samples ranging from 2 nM to 0 nM in concentration were prepared for both Fusion 1 and Fusion 3 using the same source material as was used for the dosing. For all samples and standards, a minimum volume of 100 μL was prepared in the 96-well plates.

After blocking, assay plates were washed 3 times with PBST using the plate washer, and then a liquid transfer robot (Hamilton) was used to transfer 25 μL of each standard or sample (in duplicate) to the assay plates. Following sample transfer, assay plates were covered and incubated for 2 hours at room temperature.

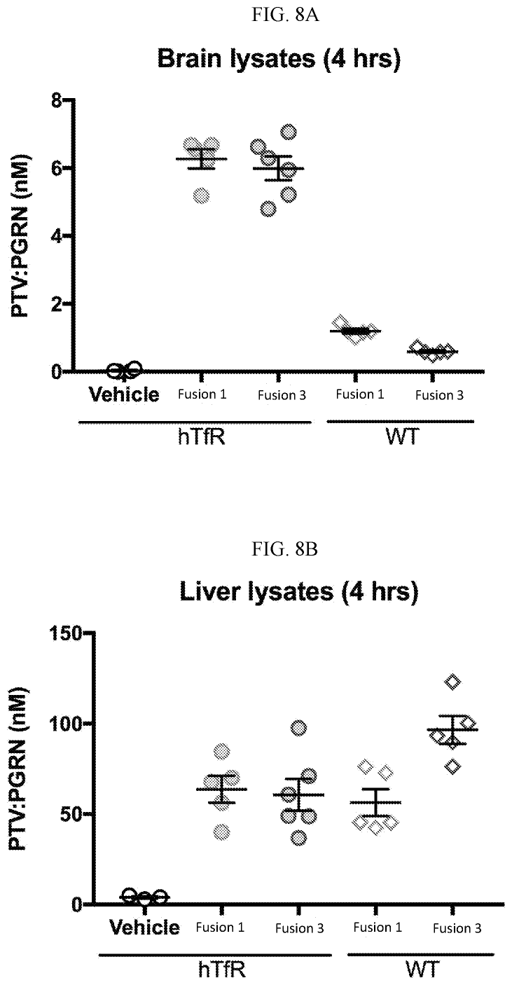

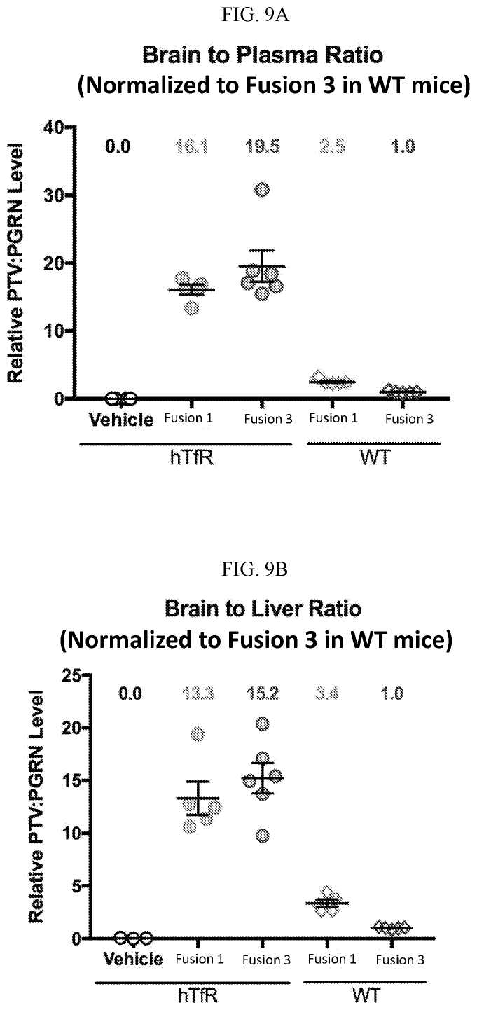

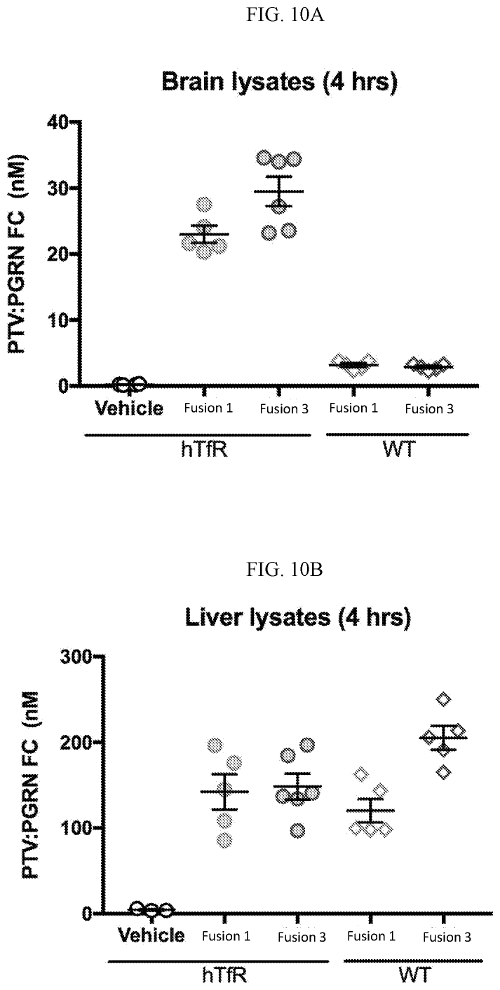

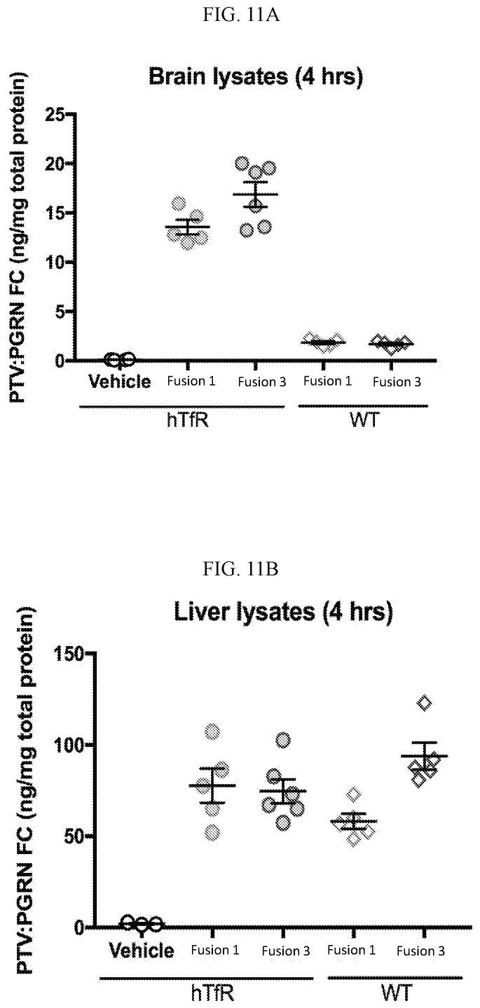

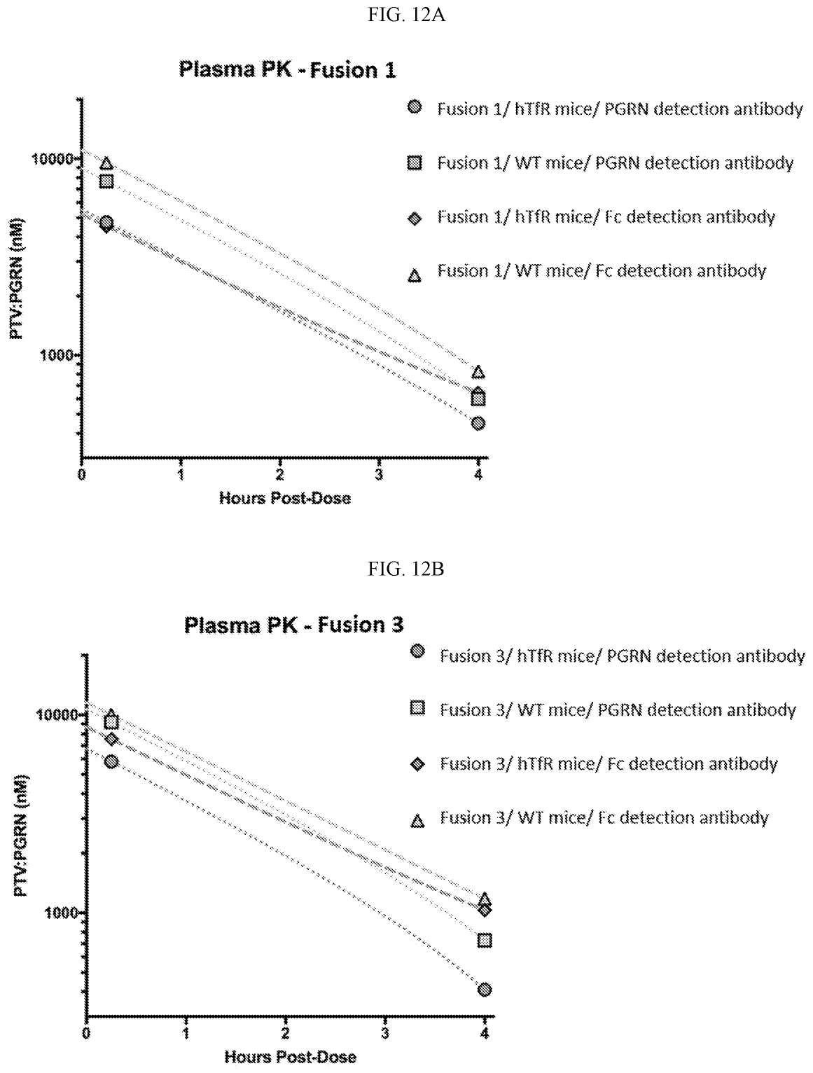

A detection antibody solution was prepared by dilution of a biotinylated goat polyclonal human progranulin detection antibody (R&D #BAF2420; FIGS. 8A, 8B, 9A, and 9B) or another detection antibody targeting a site in Fc (FIGS. 10A, 10B, 11A, and 11B) to a final concentration of 0.5 μg/mL in the assay diluent buffer. After sample incubation, plates were washed 6 times with PBST using the plate washer (rotating the plates 180 degrees after the first 3 washes), and 25 μl/well of the detection solution was added to the plates. Assay plates were incubated for 1 hour at room temperature with the detection antibody, after which they were washed 3 times with PBST on the plate washer. A working solution of streptavidin-HRP (Jackson Immunoresearch #016-030-084) was prepared by diluting the streptavidin-HRP stock 1:50,000 in assay diluent buffer. 25 μL/well of this solution was added to each well and plates were incubated 1 hour at room temperature.

After streptavidin-HRP incubation, plates were washed 3 times with PBST on the plate washer, and 25 μL/well of 1-Step Ultra-TMB substrate solution (Thermo Fisher #34029) was added to the assay plates. Assay plates were incubated at room temperature for approximately 10 minutes to allow color to develop, after which 25 μL/well of BioFX 450 nm Liquid Stop Solution (Surmodics #LSTP-1000-01) was added. After stop solution addition, plates were read using the absorbance mode of a plate reader (BioTek).

Raw absorbance data from the ELISA was analyzed by first subtracting the background absorbance signal (from wells containing no sample or standard in the assay diluent) from all assay wells. The means of the standard curve samples were fit to a four parameter logistic model equation using GraphPad Prism software, and the fit was used to calculate the concentration of Fusion 1 and Fusion 3 in each plasma or tissue lysate sample (after correcting for the sample dilution). For the brain and liver lysates, the concentrations were multiplied by 10 to adjust for the dilution of tissue during lysate preparation to obtain the concentration value in the original tissue sample.

### Results

The brain and liver levels of Fc dimer:PGRN fusion proteins in hTfR and WT mice are shown in FIGS. 8A and 8B (generated using the biotinylated goat polyclonal human progranulin detection antibody (R&D #BAF2420)). hTfR mice showed significant increase in Fc dimer:PGRN fusion proteins uptake compared to that of WT mice. FIGS. 9A and 9B show the brain:plasma ratios and brain:liver ratios, respectively, of Fc dimer:PGRN fusion proteins Fusion 1 and Fusion 3 normalized to Fusion 3 in WT. Further, FIGS. 10A, 10B, 11A, and 11B (generated using a different detection antibody that targets a site in Fc) also show that the hTfR mice had more Fc dimer:PGRN fusion proteins uptake in the brain compared to that of WT mice.

### Example 10. Pharmacokinetic Properties of Fusion Proteins in Plasma

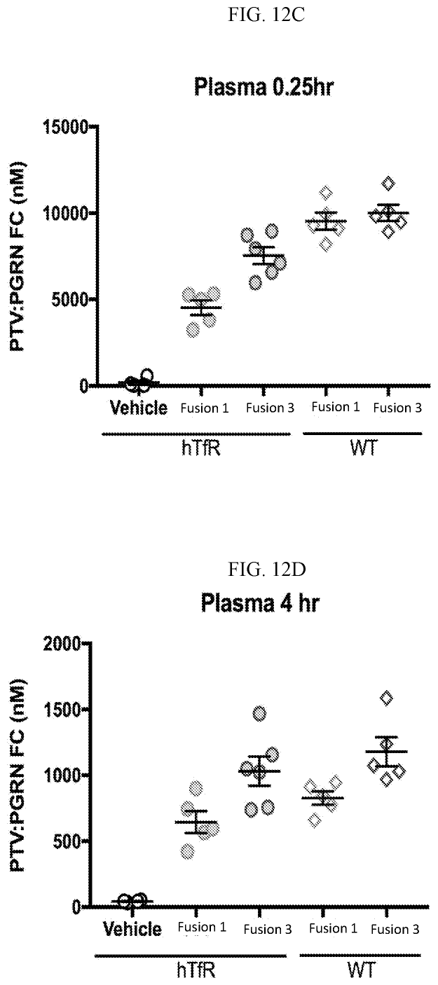

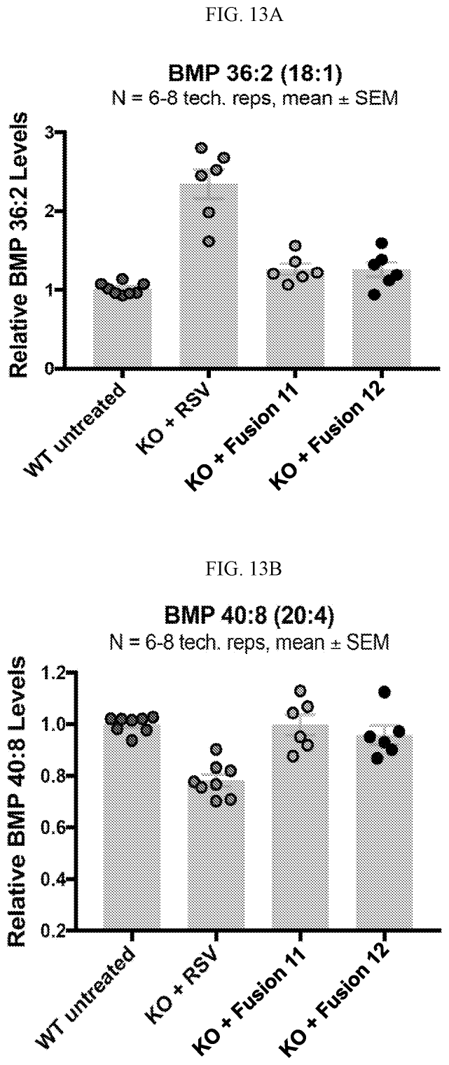

WT mice were dosed once at 10 mg/kg of Fc dimer:PGRN fusion protein Fusion 1 or Fusion 3, and plasma samples were obtained at the timepoints indicated. The concentration of each Fc dimer:PGRN fusion protein was measured by ELISA in plasma samples using an Fc-capture-Fc-detection architecture. The detection antibody used detects a site in Fc of the fusion proteins. FIGS. 12A and 12B indicate mean plasma concentrations of Fusion 1 and Fusion 3. FIGS. 12C and 12D indicate the plasma concentrations of Fusion 1 and Fusion 3 at 0.25 hr and 4 hr post dosing.

### Example 11. Modified Fc Polypeptides that Bind to TfR

This example describes modifications to Fc polypeptides to confer transferrin receptor (TfR) binding and transport across the blood-brain barrier (BBB).

Unless otherwise indicated, the positions of amino acid residues in this section are numbered based on EU index numbering for a human IgG1 wild-type Fc region.

### Generation and Characterization of Fc Polypeptides Comprising Modifications at Positions 384, 386, 387, 388, 389, 390, 413, 416, and 421 (CH3C Clones)

Yeast libraries containing Fc regions having modifications introduced into positions including amino acid positions 384, 386, 387, 388, 389, 390, 413, 416, and 421 were generated as described below. Illustrative clones that bind to TfR are shown in Tables 6 and 7 at the end of the Examples section.

After an additional two rounds of sorting, single clones were sequenced and four unique sequences were identified. These sequences had a conserved Trp at position 388, and all had an aromatic residue (i.e., Trp, Tyr, or His) at position 421. There was a great deal of diversity at other positions.

The four clones selected from the library were expressed as Fc fusions to Fab fragments in CHO or 293 cells, and purified by Protein A and size-exclusion chromatography, and then screened for binding to human TfR in the presence or absence of holo-Tf by ELISA. The clones all bound to human TfR and the binding was not affected by the addition of excess (5 μM) holo-Tf. Clones were also tested for binding to 293F cells, which endogenously express human TfR. The clones bound to 293F cells, although the overall binding was substantially weaker than the high-affinity positive control.

Next, it was tested whether clones could internalize in TfR-expressing cells using clone CH3C.3 as a test clone. Adherent HEK 293 cells were grown in 96-well plates to about 80% confluence, media was removed, and samples were added at 1 μM concentrations: clone CH3C.3, anti-TfR benchmark positive control antibody (Ab204), anti-BACE1 benchmark negative control antibody (Ab107), and human IgG isotype control (obtained from Jackson Immunoresearch). The cells were incubated at 37° C. and 8% C02 concentration for 30 minutes, then washed, permeabilized with 0.1% Triton™ X-100, and stained with anti-human-IgG-Alexa Fluor® 488 secondary antibody. After additional washing, the cells were imaged under a high content fluorescence microscope (i.e., an Opera Phenix™ system), and the number of puncta per cell was quantified. At 1 μM, clone CH3C.3 showed a similar propensity for internalization to the positive anti-TfR control, while the negative controls showed no internalization.

### Further Engineering of Clones

Additional libraries were generated to improve the affinity of the initial hits against human TfR using a soft randomization approach, wherein DNA oligos were generated to introduce soft mutagenesis based on each of the original four hits. Additional clones were identified that bound TfR and were selected. The selected clones fell into two general sequence groups. Group 1 clones (i.e., clones CH3C.18, CH3C.21, CH3C.25, and CH3C.34) had a semi-conserved Leu at position 384, a Leu or His at position 386, a conserved and a semi-conserved Val at positions 387 and 389, respectively, and a semi-conserved P-T-W motif at positions 413, 416, and 421, respectively. Group 2 clones had a conserved Tyr at position 384, the motif TXWSX at positions 386-390, and the conserved motif S/T-E-F at positions 413, 416, and 421, respectively. Clones CH3C.18 and CH3C.35 were used in additional studies as representative members of each sequence group.

### Epitope Mapping

To determine whether the engineered Fc regions bound to the apical domain of TfR, TfR apical domain was expressed on the surface of phage. To properly fold and display the apical domain, one of the loops had to be truncated and the sequence needed to be circularly permuted. Clones CH3C.18 and CH3C.35 were coated on ELISA plates and a phage ELISA protocol was followed. Briefly, after washing and blocking with 1% PBSA, dilutions of phage displaying were added and incubated at room temperature for 1 hour. The plates were subsequently washed and anti-M13-HRP was added, and after additional washing the plates were developed with TMB substrate and quenched with 2N H₂SO₄. Both clones CH3C.18 and CH3C.35 bound to the apical domain in this assay.

### Paratope Mapping

To understand which residues in the Fc domain were most important for TfR binding, a series of mutant clone CH3C.18 and clone CH3C.35 Fc regions was created in which each mutant had a single position in the TfR binding register mutated back to wild-type. The resulting variants were expressed recombinantly as Fc-Fab fusions and tested for binding to human or cyno TfR. For clone CH3C.35, positions 388 and 421 were important for binding; reversion of either of these to wild-type completely ablated binding to human TfR.

### Binding Characterization of Maturation Clones

Binding ELISAs were conducted with purified Fc-Fab fusion variants with human or cyno TfR coated on the plate, as described above. The variants from the clone CH3C.18 maturation library, clone CH3C.3.2-1, clone CH3C.3.2-5, and clone CH3C.3.2-19, bound human and cyno TfR with approximately equivalent EC₅₀ values, whereas the parent clones CH3C.18 and CH3C.35 had greater than 10-fold better binding to human versus cyno TfR.

Next, it was tested whether the modified Fc polypeptides internalized in human and monkey cells. Using the protocol described above, internalization in human HEK 293 cells and rhesus LLC-MK2 cells was tested. The variants that similarly bound human and cyno TfR, clones CH3C.3.2-5 and CH3C.3.2-19, had significantly improved internalization in LLC-MK2 cells as compared with clone CH3C.35.

### Additional Engineering of Clones

Additional engineering to further affinity mature clones CH3C.18 and CH3C.35 involved adding additional mutations to the positions that enhanced binding through direct interactions, second-shell interactions, or structure stabilization. This was achieved via generation and selection from an “NNK walk” or “NNK patch” library. The NNK walk library involved making one-by-one NNK mutations of residues that are near to the paratope. By looking at the structure of Fc bound to FcγRI (PDB ID: 4W40), 44 residues near the original modification positions were identified as candidates for interrogation. Specifically, the following residues were targeted for NNK mutagenesis: K248, R255, Q342, R344, E345, Q347, T359, K360, N361, Q362, S364, K370, E380, E382, S383, G385, Y391, K392, T393, D399, S400, D401, S403, K409, L410, T411, V412, K414, S415, Q418, Q419, G420, V422, F423, S424, S426, Q438, S440, S442, L443, S444, P4458, G446, and K447. The 44 single point NNK libraries were generated using Kunkel mutagenesis, and the products were pooled and introduced to yeast via electroporation, as described above for other yeast libraries.

The combination of these mini-libraries (each of which had one position mutated, resulting in 20 variants) generated a small library that was selected using yeast surface display for any positions that lead to higher affinity binding. Selections were performed as described above, using TfR apical domain proteins. After three rounds of sorting, clones from the enriched yeast library were sequenced, and several “hot-spot” positions were identified where certain point mutations significantly improved the binding to apical domain proteins. For clone CH3C.35, these mutations included E380 (mutated to Trp, Tyr, Leu, or Gln) and S415 (mutated to Glu). The sequences of the clone CH3C.35 single and combination mutants are set forth in SEQ ID NOS:27-38. For clone CH3C.18, these mutations included E380 (mutated to Trp, Tyr, or Leu) and K392 (mutated to Gln, Phe, or His). The sequences of the clone CH3C.18 single mutants are set forth in SEQ ID NOS:21-26.

### Additional Maturation Libraries to Improve Clone CH3C.35 Affinity

An additional library to identify combinations of mutations from the NNK walk library, while adding several additional positions on the periphery of these, was generated as described for previous yeast libraries. In this library, the YxTEWSS (SEQ ID NO:302) and TxxExxxxF motifs were kept constant, and six positions were completely randomized: E380, K392, K414, S415, S424, and S426. Positions E380 and S415 were included because they were “hot spots” in the NNK walk library. Positions K392, S424, and S426 were included because they make up part of the core that may position the binding region, while K414 was selected due to its adjacency to position 415.

This library was sorted, as previously described, with the cyno TfR apical domain only. The enriched pool was sequenced after five rounds, and the sequences of the modified regions of the identified unique clones are set forth in SEQ ID NOS:42-59.

The next libraries were designed to further explore acceptable diversity in the main binding paratope. Each of the original positions (384, 386, 387, 388, 389, 390, 413, 416, and 421) plus the two hot spots (380 and 415) were individually randomized with NNK codons to generate a series of single-position saturation mutagenesis libraries on yeast. In addition, each position was individually reverted to the wild-type residue, and these individual clones were displayed on yeast. It was noted that positions 380, 389, 390, and 415 were the only positions that retained substantial binding to TfR upon reversion to the wild-type residue (some residual but greatly diminished binding was observed for reversion of 413 to wild-type).

The single-position NNK libraries were sorted for three rounds against the human TfR apical domain to collect the top ˜5% of binders, and then at least 16 clones were sequenced from each library. The results indicate what amino acids at each position can be tolerated without significantly reducing binding to human TfR, in the context of clone CH3C.35. A summary is below:

### Position 380: Trp, Leu, or Glu;

### Position 384: Tyr or Phe;

Position 386: Thr only;
Position 387: Glu only;
Position 388: Trp only;
Position 389: Ser, Ala, or Val (although the wild type Asn residue seems to retain some binding, it did not appear following library sorting);

### Position 390: Ser or Asn;

### Position 413: Thr or Ser;

### Position 415: Glu or Ser;

Position 416: Glu only; and
Position 421: Phe only.

The above residues, when substituted into clone CH3C.35 as single changes or in combinations, represent paratope diversity that retains binding to TfR apical domain. Clones having mutations at these positions include those shown in Table 7, and the sequences of the CH3 domains of these clones are set forth in SEQ ID NOS:34-38, 58, and 60-90.

### Example 12. Additional Fc Positions that can be Modified to Confer TfR Binding

Additional modified Fc polypeptides that bind to transferrin receptor (TfR) were generated having modifications at alternative sites in the Fc region, e.g., at the following positions:

positions 274, 276, 283, 285, 286, 287, 288, and 290 (CH2A2 clones);

positions 266, 267, 268, 269, 270, 271, 295, 297, 298, and 299 (CH2C clones);

positions 268, 269, 270, 271, 272, 292, 293, 294, and 300 (CH2D clones);

positions 272, 274, 276, 322, 324, 326, 329, 330, and 331 (CH2E3 clones); or

positions 345, 346, 347, 349, 437, 438, 439, and 440 (CH3B clones).

Illustrative CH3B clones that bind to TfR are set forth in SEQ ID NOS:111-115. Illustrative CH2A2 clones that bind to TfR are set forth in SEQ ID NOS: 116-120. Illustrative CH2C clones that bind to TfR are set forth in SEQ ID NOS:121-125. Illustrative CH2D clones that bind to TfR are set forth in SEQ ID NOS:126-130. Illustrative CH2E3 clones that bind to TfR are set forth in SEQ ID NOS:131-135.

### Example 13. Methods

### Generation of Phage-Display Libraries

A DNA template coding for the wild-type human Fc sequence was synthesized and incorporated into a phagemid vector. The phagemid vector contained an ompA or pelB leader sequence, the Fc insert fused to c-Myc and 6×His (SEQ ID NO:303) epitope tags, and an amber stop codon followed by M13 coat protein pIII.

Primers containing “NNK” tricodons at the desired positions for modifications were generated, where N is any DNA base (i.e., A, C, G, or T) and K is either G or T. Alternatively, primers for “soft” randomization were used, where a mix of bases corresponding to 70% wild-type base and 10% of each of the other three bases was used for each randomization position. Libraries were generated by performing PCR amplification of fragments of the Fc region corresponding to regions of randomization and then assembled using end primers containing SfI restriction sites, then digested with SfI and ligated into the phagemid vectors. Alternatively, the primers were used to conduct Kunkel mutagenesis. The ligated products or Kunkel products were transformed into electrocompetent E. coli cells of strain TG1 (obtained from Lucigen®). The E. coli cells were infected with M13K07 helper phage after recovery and grown overnight, after which library phage were precipitated with 5% PEG/NaCl, resuspended in 15% glycerol in PBS, and frozen until use. Typical library sizes ranged from about 10⁹ to about 10¹¹ transformants. Fc-dimers were displayed on phage via pairing between pIII-fused Fc and soluble Fc not attached to pIII (the latter being generated due to the amber stop codon before pIII).

### Generation of Yeast-Display Libraries

A DNA template coding for the wild-type human Fc sequence was synthesized and incorporated into a yeast display vector. For CH2 and CH3 libraries, the Fc polypeptides were displayed on the Aga2p cell wall protein. Both vectors contained prepro leader peptides with a Kex2 cleavage sequence, and a c-Myc epitope tag fused to the terminus of the Fc.

Yeast display libraries were assembled using methods similar to those described for the phage libraries, except that amplification of fragments was performed with primers containing homologous ends for the vector. Freshly prepared electrocompetent yeast (i.e., strain EBY100) were electroporated with linearized vector and assembled library inserts. Electroporation methods will be known to one of skill in the art. After recovery in selective SD-CAA media, the yeast were grown to confluence and split twice, then induced for protein expression by transferring to SG-CAA media. Typical library sizes ranged from about 10⁷ to about 10¹¹ transformants. Fc-dimers were formed by pairing of adjacently displayed Fc monomers.

### General Methods for Phaize Selection

Phage methods were adapted from Phage Display: A Laboratory Manual (Barbas, 2001). Additional protocol details can be obtained from this reference.

### Plate Sorting Methods

Antigen was coated on MaxiSorp® microtiter plates (typically 1-10 μg/mL) overnight at 4° C. The phage libraries were added into each well and incubated overnight for binding. Microtiter wells were washed extensively with PBS containing 0.05% Tween® 20 (PBST) and bound phage were eluted by incubating the wells with acid (typically 50 mM HCl with 500 mM KCl, or 100 mM glycine, pH 2.7) for 30 minutes. Eluted phage were neutralized with 1 M Tris (pH 8) and amplified using TG1 cells and M13/KO7 helper phage and grown overnight at 37° C. in 2YT media containing 50 μg/mL carbenacillin and 50 ug/mL Kanamycin. The titers of phage eluted from a target-containing well were compared to titers of phage recovered from a non-target-containing well to assess enrichment. Selection stringency was increased by subsequently decreasing the incubation time during binding and increasing washing time and number of washes.

### Bead Sorting Methods

Antigen was biotinylated through free amines using NHS-PEG4-Biotin (obtained from Pierce™). For biotinylation reactions, a 3- to 5-fold molar excess of biotin reagent was used in PBS. Reactions were quenched with Tris followed by extensive dialysis in PBS. The biotinylated antigen was immobilized on streptavidin-coated magnetic beads, (i.e., M280-streptavidin beads obtained Thermo Fisher). The phage display libraries were incubated with the antigen-coated beads at room temperature for 1 hour. The unbound phage were then removed and beads were washed with PBST. The bound phage were eluted by incubating with 50 mM HCl containing 500 mM KCl (or 0.1 M glycine, pH 2.7) for 30 minutes, and then neutralized and propagated as described above for plate sorting.

After three to five rounds of panning, single clones were screened by either expressing Fc on phage or solubly in the E. coli periplasm. Such expression methods will be known to one of skill in the art. Individual phage supernatants or periplasmic extracts were exposed to blocked ELISA plates coated with antigen or a negative control and were subsequently detected using HRP-conjugated goat anti-Fc (obtained from Jackson Immunoresearch) for periplasmic extracts or anti-M13 (GE Healthcare) for phage, and then developed with TMB reagent (obtained from Thermo Fisher). Wells with OD₄₅₀ values greater than around 5-fold over background were considered positive clones and sequenced, after which some clones were expressed either as a soluble Fc fragment or fused to Fab fragments

### General Methods for Yeast Selection

### Bead Sorting (Magnetic-Assisted Cell Sorting (MAC's)) Methods

MACS and FACS selections were performed similarly to as described in Ackerman, et al. 2009 Biotechnol. Prog. 25(3), 774. Streptavidin magnetic beads (e.g., M-280 streptavidin beads from ThermoFisher) were labeled with biotinylated antigen and incubated with yeast (typically 5-10× library diversity). Unbound yeast were removed, the beads were washed, and bound yeast were grown in selective media and induced for subsequent rounds of selection.

### Fluorescence-Activated Cell Sorting (FACS) Methods

Yeast were labeled with anti-c-Myc antibody to monitor expression and biotinylated antigen (concentration varied depending on the sorting round). In some experiments, the antigen was pre-mixed with streptavidin-Alexa Fluor® 647 in order to enhance the avidity of the interaction. In other experiments, the biotinylated antigen was detected after binding and washing with streptavidin-Alexa Fluor® 647. Singlet yeast with binding were sorted using a FACS Aria III cell sorter. The sorted yeast were grown in selective media then induced for subsequent selection rounds.

After an enriched yeast population was achieved, yeast were plated on SD-CAA agar plates and single colonies were grown and induced for expression, then labeled as described above to determine their propensity to bind to the target. Positive single clones were subsequently sequenced for binding antigen, after which some clones were expressed either as a soluble Fc fragment or as fused to Fab fragments.

### General Methods for Screening

### Screening by ELISA

Clones were selected from panning outputs and grown in individual wells of 96-well deep-well plates. The clones were either induced for periplasmic expression using autoinduction media (obtained from EMD Millipore) or infected with helper phage for phage-display of the individual Fc variants on phage. ELISA plates were coated with antigen, typically at 0.5 mg/mL overnight, then blocked with 1% BSA before addition of phage or periplasmic extracts. After a 1-hour incubation and washing off unbound protein, HRP-conjugated secondary antibody was added (i.e., anti-Fc or anti-M13 for soluble Fc or phage-displayed Fc, respectively) and incubated for 30 minutes. The plates were washed again, and then developed with TMB reagent and quenched with 2N sulfuric acid. Absorbance at 450 nm was quantified using a plate reader (BioTek*) and binding curves were polotted using Prism software where applicable. In some assays, soluble transferrin or other competitor was added during the binding step, typically at significant molar excess.

### Screening by Flow Cytometry

Fc variant polypeptides (expressed either on phage, in periplasmic extracts, or solubly as fusions to Fab fragments) were added to cells in 96-well V-bottom plates (about 100,000 cells per well in PBS+1% BSA (PBSA)), and incubated at 4° C. for 1 hour. The plates were subsequently spun and the media was removed, and then the cells were washed once with PBSA. The cells were resuspended in PBSA containing secondary antibody (typically goat anti-human-IgG-Alexa Fluor® 647 (obtained from Thermo Fisher)). After 30 minutes, the plates were spun and the media was removed, the cells were washed 1-2 times with PBSA, and then the plates were read on a flow cytometer (i.e., a FACSCanto™ II flow cytometer). Median fluorescence values were calculated for each condition using FlowJo software and binding curves were plotted with Prism software.

### Example 14. Lipidomics and Mass Spectrometry Methods

### Mass Spectrometry Sample Preparation

Cells (e.g., bone marrow-derived macrophages (BMDMs)) were washed thoroughly with PBS, and BMP species were extracted with methanol spiked with BMP(14:0_14:0) as an internal standard. Following extraction of BMP species with methanol, samples were vortex mixed and centrifuged at 14,000 rpm and 4° C. for 20 minutes. Supernatants were then transferred to liquid chromatography-mass spectrometry vials for further analysis.

Tissue samples were weighed (e.g., 20 mg) and then homogenized in methanol (200 μL) spiked with BMP(14:0_14:0) using a TissueLyser homogenizer (Qiagen, Valencia, Calif., USA). Homogenates were spun at 14,000 rpm for 20 minutes at 4° C. Supernatants were then transferred to liquid chromatography-mass spectrometry vials for further analysis.

Biofluids (10 μL) were protein-precipitated with methanol (100 μL) containing BMP(14:0_14:0) and spun at 14,000 rpm for 20 min, 4° C. Supernatants were then transferred to liquid chromatography-mass spectrometry vials for further analysis.

### Lipidomics Procedures

Overall procedures: Animal groups and sample collection were randomized during lipid extraction.

Extraction of lipids and metabolites from urine: Urine was collected into Thermo Matrix Tubes and stored at −80° C. Urine was thawed on ice and centrifuged at 1,000×g for 10 minutes at 4° C. to remove particulates. 10 μL of urine was transferred into 2.0 mL Safe-Lock Eppendorf tube (Eppendorf Cat #022600044) and 200 μL of ice-cold MS-grade methanol containing internal standard mix (2 μL per sample). Samples were vortexed for 5 minutes at 2,500 rpm. Samples were centrifuged for 20 minutes at 21,000×g at 4° C. Methanol supernatant was transferred to LCMS glass vials in 96 well plate. Samples were stored at −80° C. until run on LCMS.

Extraction of lipids and metabolites from plasma: Plasma samples were thawed on ice. Plasma/serum (10 μL) or urine (20 μL) were transferred into a 2 mL Safe-Lock Eppendorf tube (Eppendorf Cat #022600044). Ice cold MS-grade methanol (200 μL) containing internal standard mix (2 μL per sample) was vortexed for 5 min and then centrifuged for 20 min at 21,000×g at 4° C. Methanol supernatant was transferred into LCMS glass vials in 96 well-plates for lipidomics and metabolomics analysis. Samples were stored at −80° C. until run on LCMS.

Extraction of lipids and metabolites from brain: Frontal cortex of mouse brain (18-20 mg) was transferred into 2 mL Safe-Lock Eppendorf tube (Eppendorf Cat #022600044) that was kept in dry ice containing a 5 mm stainless steel bead (QIAGEN Cat #69989). MS-grade methanol (400 μL) containing internal standard mix (2 μL per sample) was added. Tissues were homogenized with Tissuelyser for 30 sec at 25 Hz in the cold room and then centrifuged for 20 min at 21,000×g at 4° C. (bead left in the tube). Methanol supernatant was transferred into new 1.5 mL Eppendorf vials and left at −20° C. for 1 hour to allow further precipitation of proteins. Vials were centrifuged for 20 min at 21,000×g at 4° C. and the methanol supernatant was transferred in LCMS glass vials for lipidomics and metabolomics analysis. Samples stored at −80° C. until run on LCMS.

Extraction of lipids and metabolites from CSF: CSF was collected from mouse with the pipet method. CSF (5 μL) was added to ice-cold MS-grad MeOH (100 μL) containing internal standard mix (0.2 μL per sample) directly in glass vials. Glass vials with CSF and MeOH were vortexed for 5 minutes and directly run on LCMS.

Extraction of lipids and metabolites from liver: liver (20 mg) was transferred into a 2 mL Safe-Lock Eppendorf tube (Eppendorf Cat #022600044) kept in dry ice containing a 5 mm stainless steel bead (QIAGEN Cat #69989). MS-grade methanol (400 μL) containing internal standard mix (2 μL per sample) was then added. Tissues were homogenized with Tissuelyser for 30 sec at 25 Hz (in the cold room) and centrifuged for 20 min at 21,000×g at 4° C. (bead left in the tube). The methanol supernatant was transferred to new 1.5 mL Eppendorf vials and left at −20° C. for three hours to allow further precipitation of proteins. Vials were centrifuged for 20 min at 21,000×g at 4° C., and the methanol supernatant was transferred to LCMS glass vials for lipidomics and metabolomics analysis. Samples stored −80° C. until run on LCMS.

### Liquid Chromatography-Mass Spectrometry

BMP analyses were performed by liquid chromatography (Shimadzu Nexera X2 system, Shimadzu Scientific Instrument, Columbia, Md., USA) coupled to electrospray mass spectrometry (Sciex 6500+ QTRAP, Sciex, Framingham, Mass., USA). For each analysis, 5 μL of sample was injected onto a BEH amide 1.7 μm, 2.1×150 mm column (Waters Corporation, Milford, Mass., USA) using a flow rate of 0.40 mL/min. at 55° C. Mobile phase A consisted of water with 10 mM ammonium formate+0.1% formic acid. Mobile phase B consisted of acetonitrile with 0.1% formic acid. The gradient was programmed as follows: 0.0-1.0 min. at 95% B; 1.0-7.0 min. to 50% B; 7.0-7.1 min. to 95% B; and 7.1-12.0 min. at 95% B. Electrospray ionization was performed in the negative-ion mode using the following settings: curtain gas at 25; collision gas was set at medium; ion spray voltage at −4500; temperature at 600; ion source gas 1 at 50; ion source gas 2 at 60; collision energy at −50, CXP at −15; DP at −60; EP at −10; dwell time at 20 ms. Data acquisition was performed using Analyst 1.6.3 (Sciex) in multiple reaction monitoring mode (MRM). BMP species were detected using the MRM transition parameters reported in Table 3. BMP species were quantified using BMP(14:0_14:0) as the internal standard. BMP species were identified based on their retention times and MRM properties. Quantification was performed using MultiQuant 3.02 (Sciex) after correction for isotopic overlap. BMP species were normalized to either total protein amount, tissue weight or biofluid volume. Protein concentration was measured using the bicinchoninic acid (BCA) assay (Pierce, Rockford, Ill., USA).

Precursor (Q1) [M−H]⁻ and product ion (Q3) m/z transitions were used to measure BMP species. The BMP species were identified from the Q1 and Q3 values according to Table 3. Abbreviations are used herein to refer to species with two side-chains, where the structures of the fatty acid side chains are indicated within parentheses in the BMP format (e.g., BMP(18:1_18:1)). The numerals follow the standard fatty acid notation format of number of fatty acid carbon atoms: number of double bonds. The “e-” prefix is used to indicate the presence of an alkyl ether substituent (e.g., BMP (16:0e_18:0)) where the carbonyl oxygen of the fatty acid side chain is replaced with two hydrogen atoms. Alternatively the BMP species can be referred to generically according to the total number of carbon atoms: total number of double bonds; species having similar values can be distinguished by their Q1 and Q3 values.

TABLE 3
BMP Species and MRM Transition Parameters

| | Total carbon | | |
|---|---|---|---|
| | atoms:total | | |
| Name | unsaturations | Q1 | Q3 |
| BMP(14:0_14:0) | BMP(28:0) | 665.5 | 227.2 |
| BMP(14:0_16:0) | BMP(30:0) | 693.6 | 255.2 |
| BMP(14:0_16:1) | BMP(30:1) | 691.6 | 253.2 |
| BMP(14:0_18:0) | BMP(32:0) | 721.6 | 283.2 |
| BMP(14:0_18:1) | BMP(32:1) | 719.6 | 281.2 |
| BMP(14:0_18:2) | BMP(32:2) | 717.6 | 279.2 |
| BMP(14:0_18:3) | BMP(32:3) | 715.6 | 277.2 |
| BMP(14:0_20:1) | BMP(34:1) | 747.6 | 309.2 |
| BMP(14:0_20:2) | BMP(34:2) | 745.6 | 307.2 |
| BMP(14:0_20:3) | BMP(34:3) | 743.6 | 305.2 |
| BMP(14:0_20:4) | BMP(34:4) | 741.6 | 303.2 |
| BMP(14:0_20:5) | BMP(34:5) | 739.6 | 301.2 |
| BMP(14:0_22:4) | BMP(36:4) | 769.6 | 331.2 |
| BMP(14:0_22:5) | BMP(36:5) | 767.6 | 329.2 |
| BMP(14:0_22:6) | BMP(36:6) | 765.6 | 327.2 |
| BMP(16:0_16:0) | BMP(32:0) | 721.6 | 255.2 |
| BMP(16:0_16:1) | BMP(32:1) | 719.6 | 253.2 |
| BMP(16:0_18:0) | BMP(34:0) | 749.6 | 283.2 |
| BMP(16:0_18:1) | BMP(34:1) | 747.6 | 281.2 |
| BMP(16:0_18:2) | BMP(34:2) | 745.6 | 279.2 |
| BMP(16:0_18:3) | BMP(34:3) | 743.6 | 277.2 |
| BMP(16:0_20:1) | BMP(36:1) | 775.6 | 309.2 |
| BMP(16:0_20:2) | BMP(36:2) | 773.6 | 307.2 |
| BMP(16:0_20:3) | BMP(36:3) | 771.6 | 305.2 |
| BMP(16:0_20:4) | BMP(36:4) | 769.6 | 303.2 |
| BMP(16:0_20:5) | BMP(36:5) | 767.6 | 301.2 |
| BMP(16:0_22:4) | BMP(38:4) | 797.6 | 331.2 |
| BMP(16:0_22:5) | BMP(38:5) | 795.6 | 329.2 |
| BMP(16:0_22:6) | BMP(38:6) | 793.6 | 327.2 |
| BMP(16:1_16:1) | BMP(32:2) | 717.6 | 253.2 |
| BMP(16:1_18:0) | BMP(34:1) | 747.6 | 283.2 |
| BMP(16:1_18:1) | BMP(34:2) | 745.6 | 281.2 |
| BMP(16:1_18:2) | BMP(34:3) | 743.6 | 279.2 |
| BMP(16:1_18:3) | BMP(34:4) | 741.6 | 277.2 |
| BMP(16:1_20:1) | BMP(36:2) | 773.6 | 309.2 |
| BMP(16:1_20:2) | BMP(36:3) | 771.6 | 307.2 |
| BMP(16:1_20:3) | BMP(36:4) | 769.6 | 305.2 |
| BMP(16:1_20:4) | BMP(36:5) | 767.6 | 303.2 |
| BMP(16:1_20:5) | BMP(36:6) | 765.6 | 301.2 |
| BMP(16:1_22:4) | BMP(38:5) | 795.6 | 331.2 |
| BMP(16:1_22:5) | BMP(38:6) | 793.6 | 329.2 |
| BMP(16:1_22:6) | BMP(38:7) | 791.6 | 327.2 |
| BMP(16:0e_14:0) | BMP(40:0) | 679.5 | 227.2 |
| BMP(16:0e_16:0) | BMP(32:0) | 707.6 | 255.2 |
| BMP(16:0e_18:0) | BMP(34:0) | 735.6 | 283.2 |
| BMP(16:0e_18:1) | BMP(34:1) | 733.6 | 281.2 |
| BMP(16:0e_18:2) | BMP(34:2) | 731.6 | 279.2 |
| BMP(16:0e_18:3) | BMP(34:3) | 729.6 | 277.2 |
| BMP(16:0e_20:3) | BMP(36:3) | 757.6 | 305.2 |
| BMP(16:0e_20:4) | BMP(36:4) | 755.6 | 303.2 |
| BMP(16:0e_20:5) | BMP(36:5) | 753.6 | 301.2 |
| BMP(16:0e_22:4) | BMP(38:4) | 783.6 | 331.2 |
| BMP(16:0e_22:6) | BMP(38:6) | 779.6 | 327.2 |
| BMP(16:1e_14:0) | BMP(30:1) | 677.5 | 227.2 |
| BMP(16:1e_16:0) | BMP(32:1) | 705.6 | 255.2 |
| BMP(16:1e_18:0) | BMP(34:1) | 733.6 | 283.2 |
| BMP(16:1e_18:1) | BMP(34:2) | 731.6 | 281.2 |
| BMP(16:1e_18:2) | BMP(34:3) | 729.6 | 279.2 |
| BMP(16:1e_18:3) | BMP(34:4) | 727.6 | 277.2 |
| BMP(16:1e_20:3) | BMP(36:4) | 755.6 | 305.2 |
| BMP(16:1e_20:4) | BMP(36:5) | 753.6 | 303.2 |
| BMP(16:1e_20:5) | BMP(36:6) | 751.6 | 301.2 |
| BMP(16:1e_22:4) | BMP(38:5) | 781.6 | 331.2 |
| BMP(16:1e_22:6) | BMP(38:7) | 777.6 | 327.2 |
| BMP(18:0_18:0) | BMP(36:0) | 777.6 | 283.2 |
| BMP(18:0_18:1) | BMP(36:1) | 775.6 | 281.2 |
| BMP(18:0_18:2) | BMP(36:2) | 773.6 | 279.2 |
| BMP(18:0_18:3) | BMP(36:3) | 771.6 | 277.2 |
| BMP(18:0_20:1) | BMP(38:1) | 803.6 | 309.2 |
| BMP(18:0_20:2) | BMP(38:2) | 801.6 | 307.2 |
| BMP(18:0_20:3) | BMP(38:3) | 799.6 | 305.2 |
| BMP(18:0_20:4) | BMP(38:4) | 797.6 | 303.2 |
| BMP(18:0_20:5) | BMP(38:5) | 795.6 | 301.2 |
| BMP(18:0_22:4) | BMP(40:4) | 825.6 | 331.2 |
| BMP(18:0_22:5) | BMP(40:5) | 823.6 | 329.2 |
| BMP(18:0_22:6) | BMP(40:6) | 821.6 | 327.2 |
| BMP(18:1_18:1) | BMP(36:2) | 773.6 | 281.2 |
| BMP(18:1_18:2) | BMP(36:3) | 771.6 | 279.2 |
| BMP(18:1_18:3) | BMP(36:4) | 769.6 | 277.2 |
| BMP(18:1_20:1) | BMP(38:2) | 801.6 | 309.2 |
| BMP(18:1_20:2) | BMP(38:3) | 799.6 | 307.2 |
| BMP(18:1_20:3) | BMP(38:4) | 797.6 | 305.2 |
| BMP(18:1_20:4) | BMP(38:5) | 795.6 | 303.2 |
| BMP(18:1_20:5) | BMP(38:6) | 793.6 | 301.2 |
| BMP(18:1_22:4) | BMP(40:5) | 823.6 | 331.2 |
| BMP(18:1_22:5) | BMP(40:6) | 821.6 | 329.2 |
| BMP(18:1_22:6) | BMP(40:7) | 819.6 | 327.2 |
| BMP(18:2_18:2) | BMP(36:4) | 769.6 | 279.2 |
| BMP(18:2_18:3) | BMP(36:5) | 767.6 | 277.2 |
| BMP(18:2_20:1) | BMP(38:3) | 799.6 | 309.2 |
| BMP(18:2_20:2) | BMP(38:4) | 797.6 | 307.2 |
| BMP(18:2_20:3) | BMP(38:5) | 795.6 | 305.2 |
| BMP(18:2_20:4) | BMP(38:6) | 793.6 | 303.2 |
| BMP(18:2_20:5) | BMP(38:7) | 791.6 | 301.2 |
| BMP(18:2_22:4) | BMP(40:6) | 821.6 | 331.2 |
| BMP(18:2_22:5) | BMP(40:7) | 819.6 | 329.2 |
| BMP(18:2_22:6) | BMP(40:8) | 817.6 | 327.2 |
| BMP(18:3_18:3) | BMP(36:6) | 765.6 | 277.2 |
| BMP(18:3_20:1) | BMP(38:4) | 797.6 | 309.2 |
| BMP(18:3_20:2) | BMP(38:5) | 795.6 | 307.2 |
| BMP(18:3_20:3) | BMP(38:6) | 793.6 | 305.2 |
| BMP(18:3_20:4) | BMP(38:7) | 791.6 | 303.2 |
| BMP(18:3_20:5) | BMP(38:8) | 789.6 | 301.2 |
| BMP(18:3_22:4) | BMP(40:7) | 819.6 | 331.2 |
| BMP(18:3_22:5) | BMP(40:8) | 817.6 | 329.2 |
| BMP(18:3_22:6) | BMP(40:9) | 815.6 | 327.2 |
| BMP(18:0e_14:0) | BMP(32:0) | 707.5 | 227.2 |
| BMP(18:0e_16:0) | BMP(34:0) | 735.6 | 255.2 |
| BMP(18:0e_18:0) | BMP(36:0) | 763.6 | 283.2 |
| BMP(18:0e_18:1) | BMP(36:1) | 761.6 | 281.2 |
| BMP(18:0e_18:2) | BMP(36:2) | 759.6 | 279.3 |
| BMP(18:0e_18:3) | BMP(36:3) | 757.6 | 277.2 |
| BMP(18:0e_20:3) | BMP(38:3) | 785.6 | 305.2 |
| BMP(18:0e_20:4) | BMP(38:4) | 783.6 | 303.2 |
| BMP(18:0e_20:5) | BMP(38:5) | 781.6 | 301.2 |
| BMP(18:0e_22:4) | BMP(40:4) | 811.6 | 331.3 |
| BMP(18:0e_22:6) | BMP(40:6) | 807.6 | 327.3 |
| BMP(18:1e_14:0) | BMP(32:1) | 705.5 | 227.2 |
| BMP(18:1e_16:0) | BMP(34:1) | 733.6 | 255.2 |
| BMP(18:1e_18:0) | BMP(36:1) | 761.6 | 283.2 |
| BMP(18:1e_18:1) | BMP(36:2) | 759.6 | 281.2 |
| BMP(18:1e_18:2) | BMP(36:3) | 757.6 | 279.3 |
| BMP(18:1e_18:3) | BMP(36:4) | 755.6 | 277.2 |
| BMP(18:1e_20:3) | BMP(38:4) | 783.6 | 305.2 |
| BMP(18:1e_20:4) | BMP(38:5) | 781.6 | 303.2 |
| BMP(18:1e_20:5) | BMP(38:6) | 779.6 | 301.2 |
| BMP(18:1e_22:4) | BMP(40:5) | 809.6 | 331.3 |
| BMP(18:1e_22:6) | BMP(40:7) | 805.6 | 327.3 |
| BMP(20:3_20:3) | BMP(40:6) | 821.6 | 305.2 |
| BMP(20:3_20:4) | BMP(40:7) | 819.6 | 303.2 |
| BMP(20:3_20:5) | BMP(40:8) | 817.6 | 301.2 |
| BMP(20:3_22:4) | BMP(42:7) | 847.6 | 331.3 |
| BMP(20:3_22:5) | BMP(42:8) | 845.6 | 329.3 |
| BMP(20:3_22:6) | BMP(42:9) | 843.6 | 327.3 |
| BMP(20:4_20:4) | BMP(40:8) | 817.6 | 303.2 |
| BMP(20:4_20:5) | BMP(40:9) | 815.6 | 301.2 |
| BMP(20:4_22:4) | BMP(42:8) | 845.6 | 331.3 |
| BMP(20:4_22:5) | BMP(42:9) | 843.6 | 329.3 |
| BMP(20:4_22:6) | BMP(42:10) | 841.6 | 327.3 |
| BMP(20:5_20:5) | BMP(40:10) | 813.6 | 301.2 |
| BMP(20:5_22:4) | BMP(42:9) | 843.6 | 331.3 |
| BMP(20:5_22:5) | BMP(42:10) | 841.6 | 329.3 |
| BMP(20:5_22:6) | BMP(42:11) | 839.6 | 327.3 |
| BMP(22:4_22:4) | BMP(44:8) | 873.6 | 331.3 |
| BMP(22:4_22:5) | BMP(44:9) | 871.6 | 329.3 |
| BMP(22:4_22:6) | BMP(44:10) | 869.6 | 327.3 |
| BMP(22:6_22:6) | BMP(44:12) | 865.6 | 327.2 |

### Example 15. Treatment of BMDMS from GRNKnockout Mice

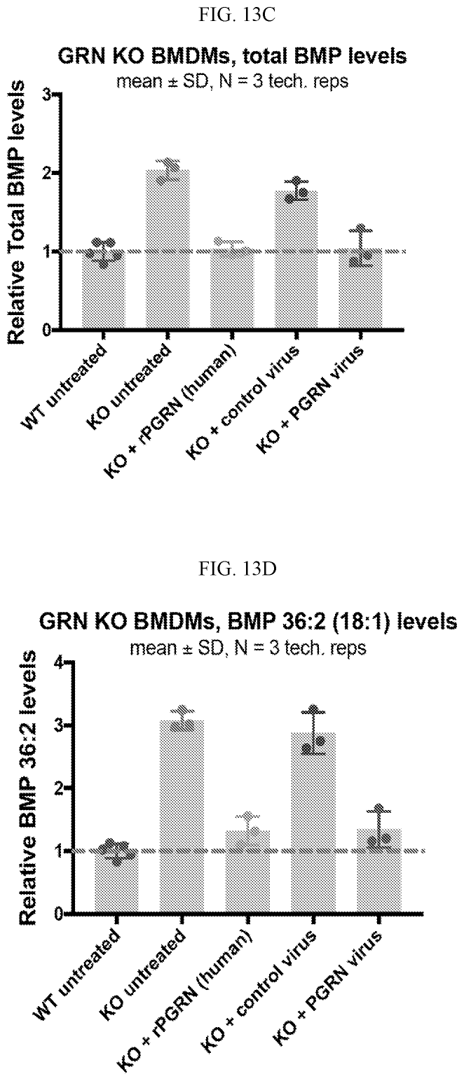

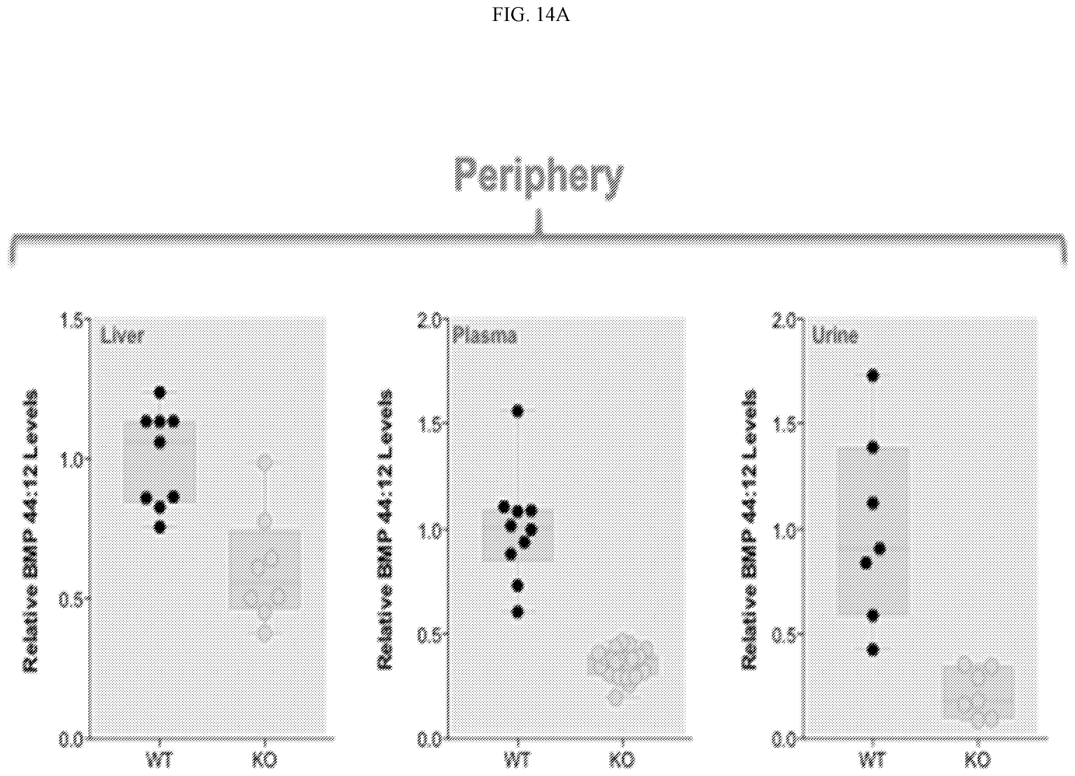

BMDMs were derived in vitro from bone marrow of wild-type and GRN knockout mice using a similar method as in Trouplin et al. J. Vis. Exp. 2013 (81) 50966, but recombinant M-CSF was added directly to the cell growth media to induce differentiation. The BMDMs were treated for 72 hours with 50 nM Fc dimer:PGRN fusion proteins Fusion 11 and Fusion 12 of Table 1 or RSV (respiratory syncytial virus) control. Cellular lipids were extracted via addition of methanol containing an internal standard mixture and BMP abundance was measured by liquid chromatography-mass spectrometry (LC-MS/MS) on a Q-trap 6500 (SCIEX). As shown in FIGS. 13A and 13B, the abundance of BMP(18:1_18:1) and BMP(20:4_20:4) was increased in BMDMs from untreated GRN knockout mice compared to the wild-type control. Furthermore in separate experiments (three technical repetitions) as shown in FIGS. 13C and 13D, total BMP and BMP(18:1_18:1) abundance decreased in the GRN knockout BMDMs that were treated with either recombinant progranulin (Adipogen) or progranulin expressed by lentivirus.

BMP species that were found to have increased abundance in BMDMs derived from granulin (GRI) knockout animals are shown in Table 4. Species that exhibited particularly marked increases in abundance are marked with an asterisk.

TABLE 4
Elevated BMP Species in BMDM of GRN KO Mice
BMP

BMP(16:0_18:1)
BMP(16:0_18:2)
BMP(18:0_18:0)
BMP(18:0_18:1)
BMP(18:1_18:1)*
BMP(16:0_20:3)
BMP(18:1_20:2)
BMP(18:0_20:4)*
BMP(16:0_22:5)
BMP(20:4_20:4)*
BMP(22:6_22:6)*
BMP(20:4_20:5)
BMP(18:2_18:2)
BMP(16:0_20:4)
BMP(18:0_18:2)
BMP(18:0e_22:6)
BMP(18:1e_20:4)
BMP(20:4_22:6)*
BMP(18:0e_20:4)
BMP(18:2_20:4)
BMP(18:1_22:6)*
BMP(18:1_20:4)*
BMP(18:0_22:6)*

### Example 16. Treatment of GRN KO Mice to Rescue Phenotypes

Fusion 11 and Fusion 12 of Table 1 were injected via the tail vein into GRN WT and GRN KO mice to determine whether peripheral and CNS GRN KO phenotypes can be rescued following a single 50 mg/kg injection.

### Materials and Methods

Animals: The mice used for this study were obtained from JAX Laboratories and consisted of 21 GRN KO mice (n=12 males, n=9 females) and 10 GRN WT mice (n=5 males, n=5 females) age 3-5 months (Table 5). Animals were housed in standard conditions in the vivarium with ad libitum access to food and water at least 7 days prior to the initiation of the study.

TABLE 5
Study Design/Experimental Groups

| Genotype | Protein | Time points (h) | n/group |
|---|---|---|---|
| GRN KO | Vehicle | 0.5, 24, 48, 96 | 4 |
| | Vehicle | 0.5, 24 | 4 |
| | Fusion 11 | 0.5, 24, 48, 96 | 5 |
| | Fusion 11 | 0.5, 24 | 4 |
| | Fusion 12 | 0.5, 24, 48, 96 | 4 |
| WT | Vehicle | 0.5, 24, 48, 96 | 5 |
| | Vehicle | 0.5, 24 | 5 |

Animal allocation to experimental groups: Mice were distributed equally in each experimental group to account for differences across litters, gender, and age.

Formulation: Fc dimer:PGRN fusion proteins Fusion 11 and Fusion 12 were used at 5.05 mg/mL and 4.90 mg/mL saline, respectively.

Overall procedures: Experimental conditions were alternated when collecting tissues. Animal groups and sample collections were randomized.

In-life procedures: Submandibular bleeds were performed using 3 mm lancets (GoldenRod animal lancets). Plasma collection: Blood was collected in EDTA tubes (Sarstedt Microvette 500 K3E, Ref #201341102) and slowly inverted 10 times. For small volumes of blood (<100 μL), blood was collected in EDTA tubes with capillary tube (Sarstedt Microvette 100 K3E, Ref #201278100). EDTA tubes were immediately stored in the fridge until plasma preparation. The time between storage and preparation did not exceed 1 hour. The time of collection and time of processing were followed consistently. The tubes were centrifuged at 12,700 rpm for 7 minutes at 4° C. Plasma (top layer) was transferred to 0.6 mL Matrix tubes with rubber seal. Matrix tubes were snap frozen on dry ice before transferring to −80° C. Urine collection: Urine was collected by restraining mice in a plastic weigh boat, causing most of them to expel urine into the weigh boat. The urine was collected with a p200 pipet, transferred to 0.6 mL matrix tubes with rubber seal. Matrix tubes were snap frozen on dry ice before transferring to −80° C.

Terminal fluids/tissue collection procedures: Animals were deeply anesthetized via intraperitoneal (i.p.) injection of 2.5% Avertin and then tissues were collected in the order as described below:

Samples collected before intracardiac perfusion: Plasma collection: Blood was collected via cardiac puncture using a 1 mL Terumo tuberculin syringe attached to a 25 gauge needle (Ref #SS-01T2516) (Not pre-conditioned with EDTA). The needle was then detached and the blood was transferred to EDTA tubes (Sarstedt Microvette 500 K3E, Ref #201341102) and slowly inverted 10 times. Following this procedure, post collection methods were continued as described above in In-Life Procedures. Cerebrospinal fluid collection: The three muscle layers on the back of the neck were peeled back with small scissors and forceps and the area surrounding the revealed cisterna magna was cauterized to prevent contamination from blood, and the membrane was cleaned with a Q-tip and PBS. The membrane was dried and the cisterna magna was punctured with the tip of a 28% gauge insulin syringe needle. A p20 pipet was then quickly placed over the newly punctured hole and CSF drawn up into the pipet tip. CSF was placed in a 0.5 mL Lo-bind Eppendorf tube and spun at 12,700 rpm for 7 minutes at 4° C. The CSF supernatant was transferred to a 0.5 mL Lo-bind Eppendorf tube and snap frozen on dry ice before transferring to −80° C.

For samples collected after intracardiac perfusion, animals were transcardially perfused for 5 minutes with ice cold PBS using a peristaltic pump (Gilson Inc. Minipuls Evolution F110701) at a flow rate of 5 mL/minute. For tissues that could be dissected and pre-weighed during collection, the samples were placed directly into 1.5 mL Eppendorf tubes. Tissues were collected in the following order: Liver (post-perfusion): about 150 mg of liver was collected into 1.5 mL Eppendorf tubes. Eppendorf tubes were snap frozen on dry ice before transferring to −80° C. Eye: both eyes were removed, muscle and optic nerve removed, and eyes placed in a single 1.5 mL Eppendorf tube. Eppendorf tubes were snap frozen on dry ice before transferring to −80° C. Brain: right hemisphere without olfactory bulb and cerebellum was collected and weighed before placing in 1.5 mL Eppendorf tubes. Eppendorf tubes were snap frozen on dry ice before transferring to −80° C.

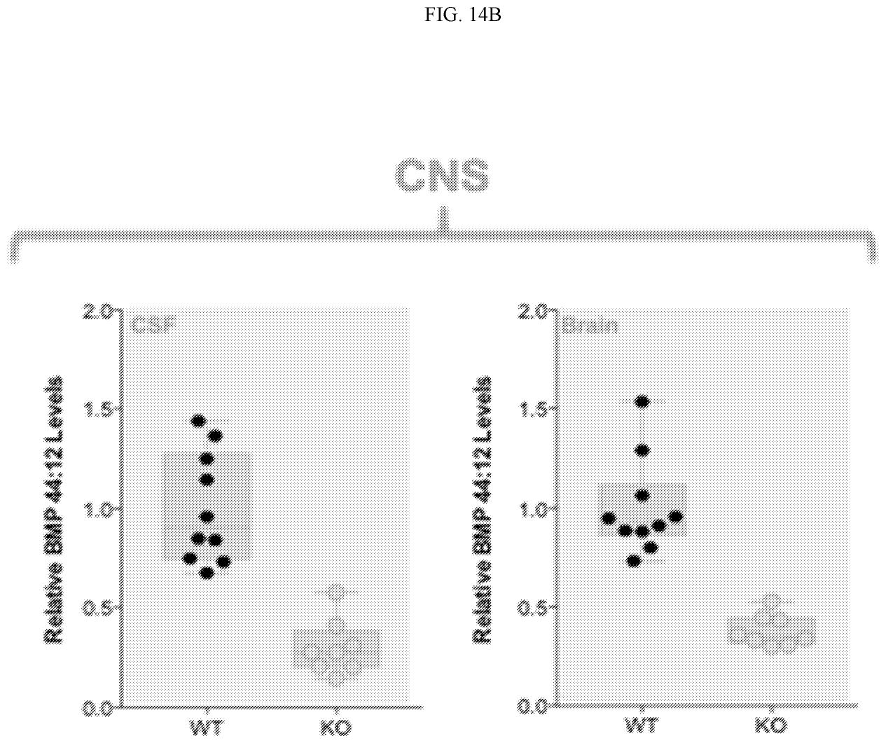

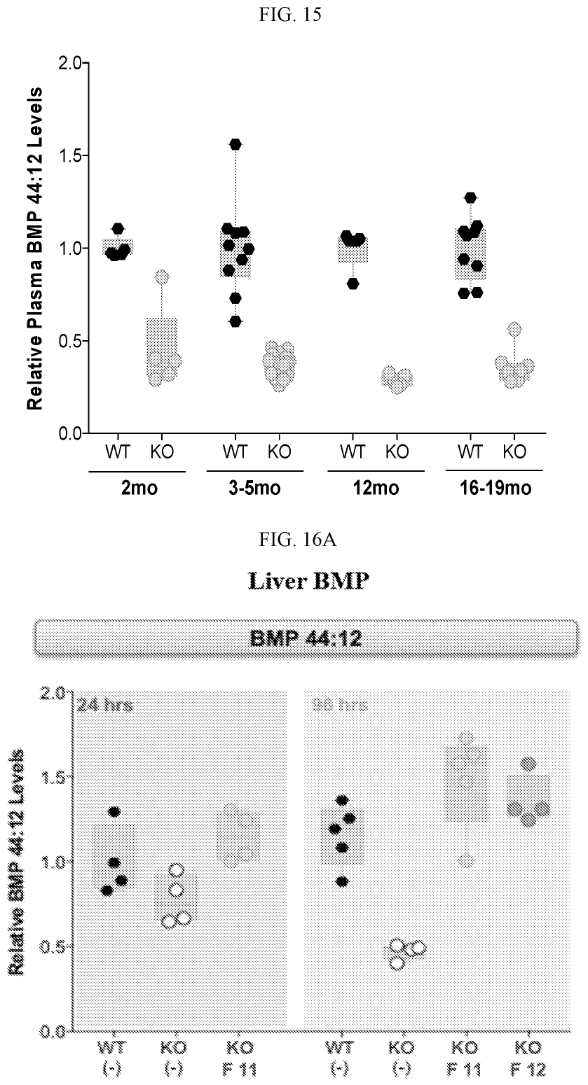

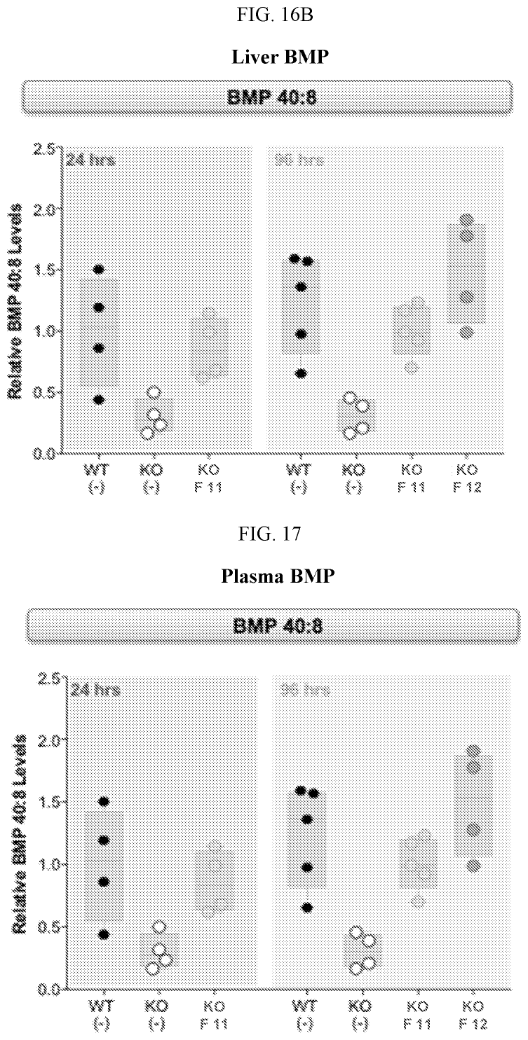

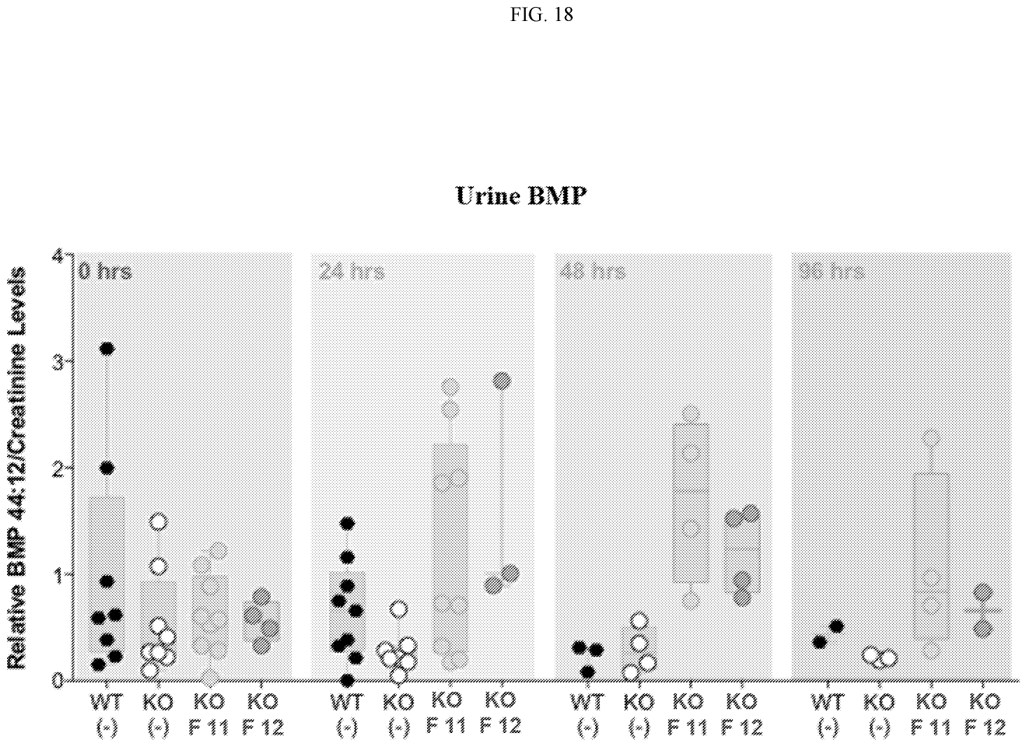

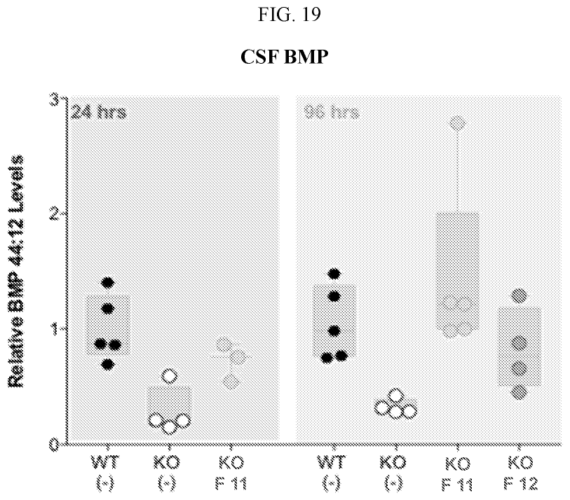

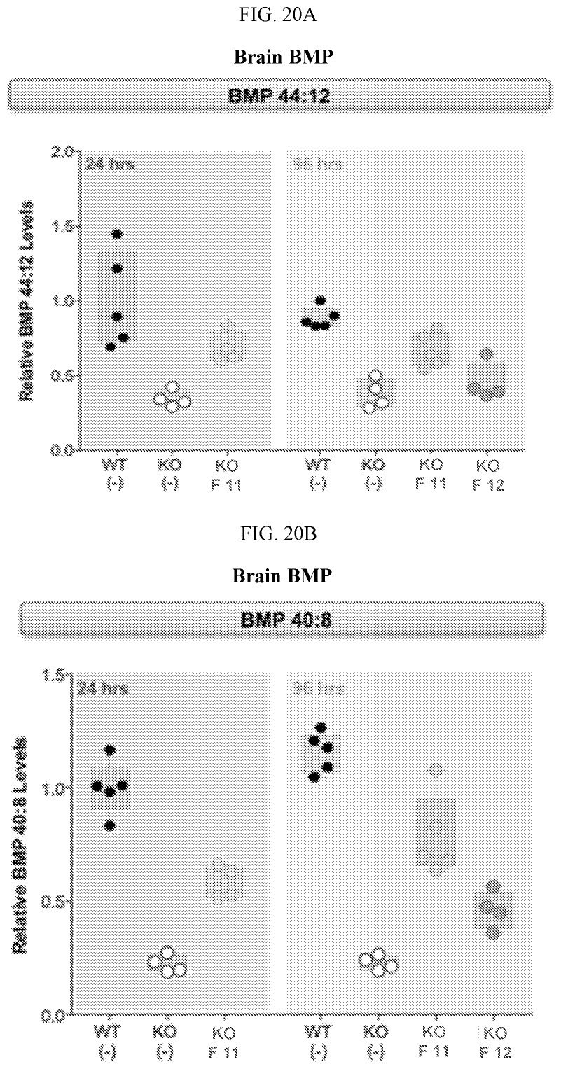

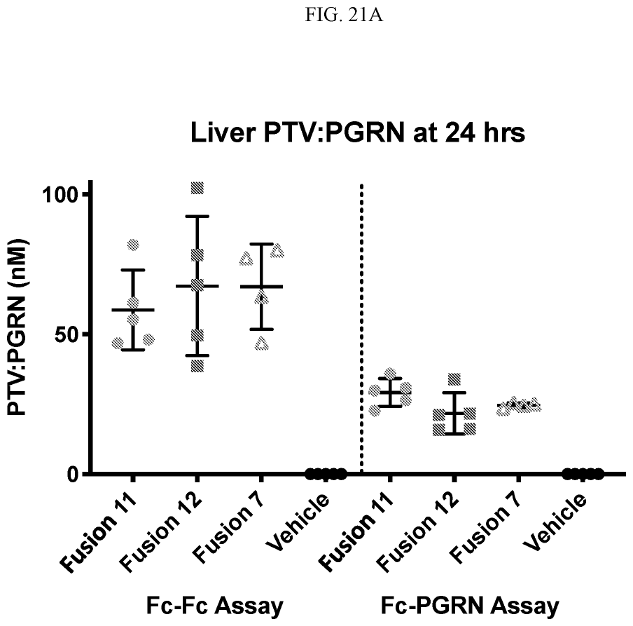

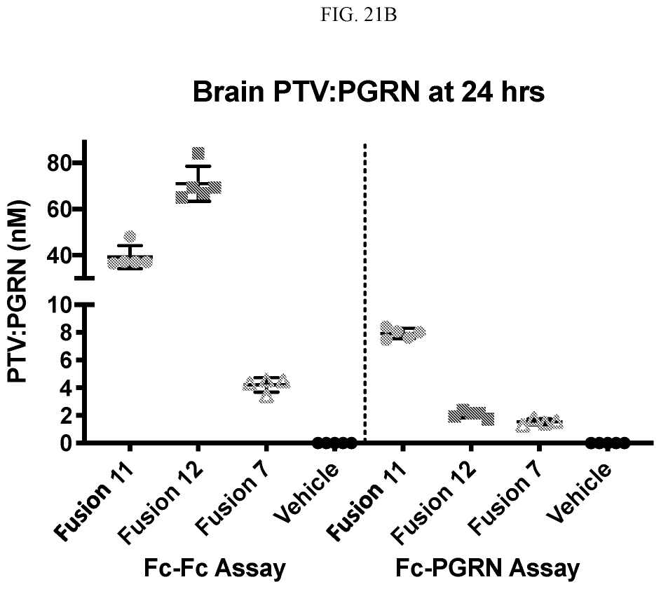

As shown in FIGS. 14-20, administration of Fc dimer:PGRN fusion proteins Fusion 11 and Fusion 12 was found to increase and restore BMP levels in the liver, plasma, urine, CSF, and brain.

### Example 17. Fusion Proteins Crossing the BBB

Fusion 11 and Fusion 12 of Table 1 were injected via the tail vein into GRNKO mice (Jackson Laboratory, Stock No. 013175) crossed with hTfR KI mice (GRN KO/hTfR.KI mice) to test their ability to cross the BBB. A description of the hTfR KI mice can be found in International Patent Publication No. WO2018152285. To generate GRN KO/hTfR.KI mice, in the first round of breeding, GRN heterozygous (GRN HET) mice were crossed to the TfR^{ms/hu} KI homozygous (TfR^{ms/hu}.KI HOM) mice to generate GRN HET x TfR^{ms/hu}.KI HET progeny. The GRN HET x TfR^{ms/hu}.KI HET mice were then crossed to the TfR^{ms/hu}.KI HOM mice to get GRN HET x TfR^{ms/hu}.KI HOM progeny in this second round. In the third and final round of breeding, GRN HET x TfR^{ms/hu}.KI HOM mice were crossed to GRN HET x TfR^{ms/hu}.KI HOM mice to generate the final GRNKO x TfR^{ms/hu}.KI HOM mice that were used in this study.

2-3 months old GRN KO/hTfR.KI mice were dosed with 50 mg/kg intravenously via the tail vein with either sterile saline (vehicle), Fusion 7, Fusion 11, or Fusion 12 (Table 8). Mice were sedated with avertine 24 hours after the treatment and a cardiac puncture was performed to collect whole blood for plasma isolation. Animals were transcardially perfused with chilled 1×PBS at a rate of 5 mL/minute for 5-8 minutes, or until the livers were cleared of blood. A 100 mg portion of the liver was collected and the left hemisphere of the brain was collected and immediately snapped frozen on dry ice.

TABLE 8
Study Design/Experimental Groups

| | | | Dose | Time | | Material |
|---|---|---|---|---|---|---|
| Molecule | Cell Line | Genotype | (mg/kg) | point | N/group | (mg) |
| Saline | N/A | TfR.KI | N/A | −3 d, | 6 | N/A |
| | | | | 1 d* | | |
| Saline | N/A | GRN | N/A | −3 d, | 5 | N/A |
| | | KO/TfR.KI | | 1 d* | | |
| Fusion 11 | HEK | GRN | 50 | −3 d, | 5 | 18 |
| | | KO/TfR.KI | | 1 d* | | |
| Fusion 12 | HEK | GRN | 50 | −3 d, | 5 | 18 |
| | | KO/TfR.KI | | 1 d* | | |
| Fusion 7 | HEK | GRN | 50 | −3 d, | 4 | 18 |
| | | KO/TfR.KI | | 1 d* | | |

Tissue samples were weighed and were homogenized in 10× volume by weight cell lysis buffer (Cell Signaling Technologies; 20 mM Tris-HCl pH 7.5, 150 mM NaCl, 1 mM Na₂EDTA, 1 mM EGTA, 1% Triton, 2.5 mM sodium pyrophosphate, 1 mM beta-glycerophosphate, 1 mM Na₃VO₄, and 1 μg/mL leupeptin) supplemented with 1× protease inhibitor (Roche) and 1× phosphatase inhibitor (Roche). Samples were homogenized using the TissueLyzer with 3 mm metal beads for 2×3 min at 29 Hz. Following homogenization, samples were spun at maximum speed on the tabletop centrifuge for 20 minutes at 4° C. Supernatant was transferred to new tubes and the Fc-PGRN ELISA assay (Fc capture and PGRN detection ELISA) and Fc-Fc ELISA assay (Fc capture and Fc detection ELISA) were run using those samples.

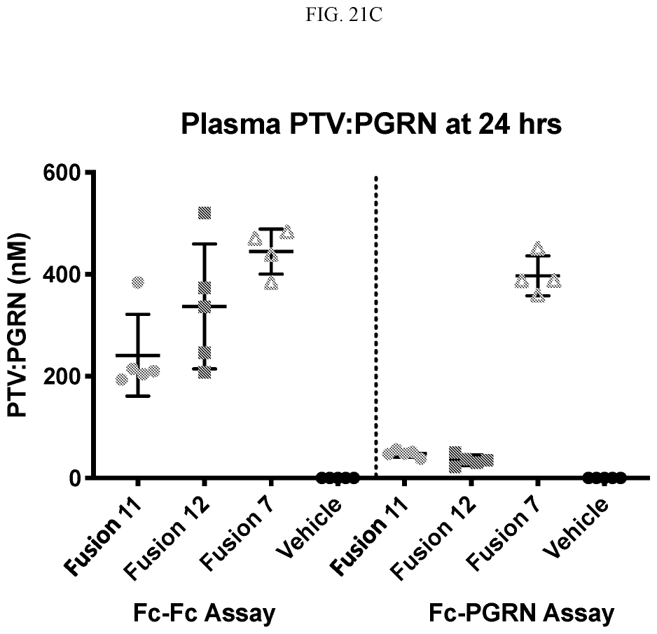

The data in FIGS. 21A-21C shows that Fusion 11 and Fusion 12 were able to cross the BBB in the brain of GRN KO/hTfR.KI mice.

TABLE 6
CH3 Domain Modifications

| Clone name | Group | 384 | 385 | 386 | 387 | 388 | 389 | 390 | 391 | . . . | 413 | 414 | 415 | 416 | 417 | 418 | 419 | 420 | 421 |
|---|---|---|---|---|---|---|---|---|---|---|---|---|---|---|---|---|---|---|---|
| Wild-type | n/a | N | G | Q | P | E | N | N | Y | . . . | D | K | S | R | W | Q | Q | G | N |
| 1 | | L | G | L | V | W | V | G | Y | . . . | A | K | S | T | W | Q | Q | G | W |
| 2 | | Y | G | Y | V | W | S | H | Y | . . . | S | K | S | E | W | Q | Q | G | Y |
| 3 | | Y | G | T | E | W | S | Q | Y | . . . | E | K | S | D | W | Q | Q | G | H |
| 4 | | V | G | T | P | W | A | L | Y | . . . | L | K | S | E | W | Q | Q | G | W |
| 17 | 2 | Y | G | T | V | W | S | K | Y | . . . | S | K | S | E | W | Q | Q | G | F |
| 18 | 1 | L | G | H | V | W | A | V | Y | . . . | P | K | S | T | W | Q | Q | G | W |
| 21 | 1 | L | G | L | V | W | V | G | Y | . . . | P | K | S | T | W | Q | Q | G | W |
| 25 | 1 | M | G | H | V | W | V | G | Y | . . . | D | K | S | T | W | Q | Q | G | W |
| 34 | 1 | L | G | L | V | W | V | F | S | . . . | P | K | S | T | W | Q | Q | G | W |
| 35 | 2 | Y | G | T | E | W | S | S | Y | . . . | T | K | S | E | W | Q | Q | G | F |
| 44 | 2 | Y | G | T | E | W | S | N | Y | . . . | S | K | S | E | W | Q | Q | G | F |
| 51 | ½ | L | G | H | V | W | V | G | Y | . . . | S | K | S | E | W | Q | Q | G | W |
| 3.1-3 | 1 | L | G | H | V | W | V | A | T | . . . | P | K | S | T | W | Q | Q | G | W |
| 3.1-9 | 1 | L | G | P | V | W | V | H | T | . . . | P | K | S | T | W | Q | Q | G | W |
| 3.2-5 | 1 | L | G | H | V | W | V | D | Q | . . . | P | K | S | T | W | Q | Q | G | W |
| 3.2-19 | 1 | L | G | H | V | W | V | N | Q | . . . | P | K | S | T | W | Q | Q | G | W |
| 3.2-1 | 1 | L | G | H | V | W | V | N | F | . . . | P | K | S | T | W | Q | Q | G | W |

TABLE 7
Additional CH3 Domain Modifications

| Clone name | 378 | 379 | 380 | 381 | 382 | 383 | 384 | 385 | 386 | 387 | 388 | 389 | 390 | 391 | 392 | 411 | 412 | 413 | 414 | 415 | 416 | 417 | 418 | 419 | 420 | 421 | 422 | 423 |
|---|---|---|---|---|---|---|---|---|---|---|---|---|---|---|---|---|---|---|---|---|---|---|---|---|---|---|---|---|
| Wild-type | A | V | E | W | E | S | N | G | Q | P | E | N | N | Y | K | T | V | D | K | S | R | W | Q | Q | G | N | V | F |
| 35.20.1 | . | . | . | . | . | . | F | . | T | E | W | S | S | . | . | . | . | T | . | E | E | . | . | . | . | F | . | . |
| 35.20.2 | . | . | . | . | . | . | Y | . | T | E | W | A | S | . | . | . | . | T | . | E | E | . | . | . | . | F | . | . |
| 35.20.3 | . | . | . | . | . | . | Y | . | T | E | W | V | S | . | . | . | . | T | . | E | E | . | . | . | . | F | . | . |
| 35.20.4 | . | . | . | . | . | . | Y | . | T | E | W | S | S | . | . | . | . | S | . | E | E | . | . | . | . | F | . | . |
| 35.20.5 | . | . | . | . | . | . | F | . | T | E | W | A | S | . | . | . | . | T | . | E | E | . | . | . | . | F | . | . |
| 35.20.6 | . | . | . | . | . | . | F | . | T | E | W | V | S | . | . | . | . | T | . | E | E | . | . | . | . | F | . | . |
| 35.21.a.1 | . | . | W | . | . | . | F | . | T | E | W | S | S | . | . | . | . | T | . | E | E | . | . | . | . | F | . | . |
| 35.21.a.2 | . | . | W | . | . | . | Y | . | T | E | W | A | S | . | . | . | . | T | . | E | E | . | . | . | . | F | . | . |
| 35.21.a.3 | . | . | W | . | . | . | Y | . | T | E | W | V | S | . | . | . | . | T | . | E | E | . | . | . | . | F | . | . |
| 35.21.a.4 | . | . | W | . | . | . | Y | . | I | E | W | S | S | . | . | . | . | S | . | E | E | . | . | . | . | F | . | . |
| 35.21.a.5 | . | . | W | . | . | . | F | . | I | E | W | A | S | . | . | . | . | T | . | E | E | . | . | . | . | F | . | . |
| 35.21.a.6 | . | . | W | . | . | . | F | . | I | E | W | V | S | . | . | . | . | T | . | E | E | . | . | . | . | F | . | . |
| 35.23.1 | . | . | . | . | . | . | F | . | I | E | W | S | . | . | . | . | . | T | . | E | E | . | . | . | . | F | . | . |
| 35.23.2 | . | . | . | . | . | . | Y | . | T | E | W | A | . | . | . | . | . | T | . | E | E | . | . | . | . | F | . | . |
| 35.23.3 | . | . | . | . | . | . | Y | . | T | E | W | V | . | . | . | . | . | T | . | E | E | . | . | . | . | F | . | . |
| 35.23.4 | . | . | . | . | . | . | Y | . | T | E | W | S | . | . | . | . | . | S | . | E | E | . | . | . | . | F | . | . |
| 35.23.5 | . | . | . | . | . | . | F | . | T | E | W | A | . | . | . | . | . | T | . | E | E | . | . | . | . | F | . | . |
| 35.23.6 | . | . | . | . | . | . | F | . | T | E | W | V | . | . | . | . | . | T | . | E | E | . | . | . | . | F | . | . |
| 35.24.1 | . | . | W | . | . | . | F | . | T | E | W | S | . | . | . | . | . | T | . | E | E | . | . | . | . | F | . | . |
| 35.24.2 | . | . | W | . | . | . | Y | . | T | E | W | A | . | . | . | . | . | T | . | E | E | . | . | . | . | F | . | . |
| 35.24.3 | . | . | W | . | . | . | Y | . | T | E | W | V | . | . | . | . | . | T | . | E | E | . | . | . | . | F | . | . |
| 35.24.4 | . | . | W | . | . | . | Y | . | T | E | W | S | . | . | . | . | . | S | . | E | E | . | . | . | . | F | . | . |
| 35.24.5 | . | . | W | . | . | . | F | . | T | E | W | A | . | . | . | . | . | T | . | E | E | . | . | . | . | F | . | . |
| 35.24.6 | . | . | W | . | . | . | F | . | T | E | W | V | . | . | . | . | . | T | . | E | E | . | . | . | . | F | . | . |
| 35.21.17.1 | . | . | L | . | . | . | F | . | T | E | W | S | S | . | . | . | . | T | . | E | E | . | . | . | . | F | . | . |
| 35.21.17.2 | . | . | L | . | . | . | Y | . | T | E | W | A | S | . | . | . | . | T | . | E | E | . | . | . | . | F | . | . |
| 35.21.17.3 | . | . | L | . | . | . | Y | . | T | E | W | V | S | . | . | . | . | T | . | E | E | . | . | . | . | F | . | . |
| 35.21.17.4 | . | . | L | . | . | . | Y | . | T | E | W | S | S | . | . | . | . | S | . | E | E | . | . | . | . | F | . | . |
| 35.21.17.5 | . | . | L | . | . | . | F | . | T | E | W | A | S | . | . | . | . | T | . | E | E | . | . | . | . | F | . | . |
| 35.21.17.6 | . | . | L | . | . | . | F | . | T | E | W | V | S | . | . | . | . | T | . | E | E | . | . | . | . | F | . | . |
| 35.20 | . | . | . | . | . | . | Y | . | T | E | W | S | S | . | . | . | . | T | . | E | E | . | . | . | . | F | . | . |
| 35.21 | . | . | W | . | . | . | Y | . | T | E | W | S | S | . | . | . | . | T | . | E | E | . | . | . | . | F | . | . |
| 35.22 | . | . | W | . | . | . | Y | . | T | E | W | S | . | . | . | . | . | T | . | . | E | . | . | . | . | F | . | . |
| 35.23 | . | . | . | . | . | . | Y | . | T | E | W | S | . | . | . | . | . | T | . | E | E | . | . | . | . | F | . | . |
| 35.24 | . | . | W | . | . | . | Y | . | T | E | W | S | . | . | . | . | . | T | . | E | E | . | . | . | . | F | . | . |
| 35.21.17 | . | . | L | . | . | . | Y | . | T | E | W | S | S | . | . | . | . | T | . | E | E | . | . | . | . | F | . | . |
| 35.N390 | . | . | . | . | . | . | Y | . | T | E | W | S | . | . | . | . | . | T | . | . | E | . | . | . | . | F | . | . |

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

| SEQ ID | | |
|---|---|---|
| NO: | Sequence | Description |
| 1 | APELLGGPSVFLFPPKPKDTLMISRTPEVTCVVVDVSHEDPEVKFN | Wild-type human Fc |
| | WYVDGVEVHNAKTKPREEQYNSTYRVVSVLTVLHQDWLNGKEY | sequence |
| | KCKVSNKALPAPIEKTISKAKGQPREPQVYTLPPSRDELTKNQVSLT | positions 231-447 EU |
| | CLVKGFYPSDIAVEWESNGQPENNYKTTPPVLDSDGSFFLYSKLTV | index numbering |
| | DKSRWQQGNVFSCSVMHEALHNHYTQKSLSLSPGK | |
| 2 | APELLGGPSVFLFPPKPKDTLMISRTPEVTCVVVDVSHEDPEVKFN | CH2 domain sequence |
| | WYVDGVEVHNAKTKPREEQYNSTYRVVSVLTVLHQDWLNGKEY | positions 231-340 EU |
| | KCKVSNKALPAPIEKTISKAK | index numbering |
| 3 | GQPREPQVYTLPPSRDELTKNQVSLTCLVKGFYPSDIAVEWESNGQ | CH3 domain sequence |
| | PENNYKTTPPVLDSDGSFFLYSKLTVDKSRWQQGNVFSCSVMHEA | Positions 341-447 EU |
| | LHNHYTQKSLSLSPGK | index numbering |
| 4 | APELLGGPSVFLFPPKPKDTLMISRTPEVTCVVVDVSHEDPEVKFN | Clone CH3C.1 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGLVWVGYKTTPPVLDSDGSFFLYSKLTV | |
| | AKSTWQQGWVFSCSVMHEALHNHYTQKSLSLSPGK | |
| 5 | APELLGGPSVFLFPPKPKDTLMISRTPEVTCVVVDVSHEDPEVKFN | Clone CH3C.2 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTVWSHYKTTPPVLDSDGSFFLYSKLTV | |
| | SKSEWQQGYVFSCSVMHEALHNHYTQKSLSLSPGK | |
| 6 | APELLGGPSVFLFPPKPKDTLMISRTPEVTCVVVDVSHEDPEVKFN | Clone CH3C.3 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSQYKTTPPVLDSDGSFFLYSKLTV | |
| | EKSDWQQGHVFSCSVMHEALHNHYTQKSLSLSPGK | |
| 7 | APELLGGPSVFLFPPKPKDTLMISRTPEVTCVVVDVSHEDPEVKFN | Clone CH3C.4 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESVGTPWALYKTTPPVLDSDGSFFLYSKLTV | |
| | LKSEWQQGWVFSCSVMHEALHNHYTQKSLSLSPGK | |
| 8 | APELLGGPSVFLFPPKPKDTLMISRTPEVTCVVVDVSHEDPEVKFN | Clone CH3C.17 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTVWSKYKTTPPVLDSDGSFFLYSKLTV | |
| | SKSEWQQGFVFSCSVMHEALHNHYTQKSLSLSPGK | |
| 9 | APELLGGPSVFLFPPKPKDTLMISRTPEVTCVVVDVSHEDPEVKFN | Clone CH3C.18 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWAVYKTTPPVLDSDGSFFLYSKLTV | |
| | PKSTWQQGWVFSCSVMHEALHNHYTQKSLSLSPGK | |
| 10 | APELLGGPSVFLFPPKPKDTLMISRTPEVTCVVVDVSHEDPEVKFN | Clone CH3C.21 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGLVWVGYKTTPPVLDSDGSFFLYSKLTV | |
| | PKSTWQQGWVFSCSVMHEALHNHYTQKSLSLSPGK | |
| 11 | APELLGGPSVFLFPPKPKDTLMISRTPEVTCVVVDVSHEDPEVKFN | Clone CH3C.25 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESMGHVWVGYKTTPPVLDSDGSFFLYSKLT | |
| | VDKSTWQQGWVFSCSVMHEALHNHYTQKSLSLSPGK | |
| 12 | APELLGGPSVFLFPPKPKDTLMISRTPEVTCVVVDVSHEDPEVKFN | Clone CH3C.34 |
| | WYVDGVEVHNAKTKPREEQYNSTYRVVSVLTVLHQDWLNGKEY | |
| | KCKVSNKALPAPIELTISKAKGQPREPQVYTLPPSRDELTKNQVSLT | |
| | CLVKGFYPSDIAVEWESLGLVWVFSKTTPPVLDSDGFFLYSKLTV | |
| | PKSTWQQGWVFSCSVMHEALHNHYTQKSLSLSPGK | |
| 13 | APELLGGPSVFLFPPKPKDTLMISRTPEVTCVVVDVSHEDPEVKFN | Clone CH3C.35 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSSYKTTPPVLDSDGSFFLYSKLTV | |
| | TKSEWQQGFVFSCSVMHEALHNHYTQKSLSLSPGK | |
| 14 | APELLGGPSVFLFPPKPKDTLMISRTPEVTCVVVDVSHEDPEVKFN | Clone CH3C.44 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSNYKTTPPVLDSDGSFFLYSKLTV | |
| | SKSEWQQGFVFSCSVMHEALHNHYTQKSLSLSPGK | |
| 15 | APELLGGPSVFLFPPKPKDTLMISRTPEVTCVVVDVSHEDPEVKFN | Clone CH3C.51 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWVGYKTTPPVLDSDGSFFLYSKLTV | |
| | SKSEWQQGWVFSCSVMHEALHNHYTQKSLSLSPGK | |
| 16 | APELLGGPSVFLFPPKPKDTLMISRTPEVTCVVVDVSHEDPEVKFN | Clone CH3C.3.1-3 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWVATKTTPPVLDSDGSFFLYSKLTV | |
| | PKSTWQQGWVFSCSVMHEALHNHYTQKSLSLSPGK | |
| 17 | APELLGGPSVFLFPPKPKDTLMISRTPEVTCVVVDVSHEDPEVKFN | Clone CH3C.3.1-9 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGPVWVHTKTTPPVLDSDGSFFLYSKLTV | |
| | PKSTWQQGWVFSCSVMHEALHNHYTQKSLSLSPGK | |
| 18 | APELLGGPSVFLFPPKPKDTLMISRTPEVTCVVVDVSHEDPEVKFN | Clone CH3C.3.2-5 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWVDQKTTPPVLDSDGSFFLYSKLTV | |
| | PKSTWQQGWVFSCSVMHEALHNHYTQKSLSLSPGK | |
| 19 | APELLGGPSVFLFPPKPKDTLMISRTPEVTCVVVDVSHEDPEVKFN | Clone CH3C.3.2-19 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWVNQKTTPPVLDSDGSFFLYSKLTV | |
| | PKSTWQQGWVFSCSVMHEALHNHYTQKSLSLSPGK | |
| 20 | APELLGGPSVFLFPPKPKDTLMISRTPEVTCVVVDVSHEDPEVKFN | Clone CH3C.3.2-1 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWVNFKTTPPVLDSDGSFFLYSKLTV | |
| | PKSTWQQGWVFSCSVMHEALHNHYTQKSLSLSPGK | |
| 21 | APELLGGPSVFLFPPKPKDTLMISRTPEVTCVVVDVSHEDPEVKFN | Clone CH3C.18 variant |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESLGHVWAVYKTTPPVLDSDGSFFLYSKLT | |
| | VPKSTWQQGWVFSCSVMHEALHNHYTQKSLSLSPGK | |
| 22 | APELLGGPSVFLFPPKPKDTLMISRTPEVTCVVVDVSHEDPEVKFN | Clone CH3C.18 variant |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESLGHVWAVYKTTPPVLDSDGSFFLYSKLTV | |
| | PKSTWQQGWVFSCSVMHEALHNHYTQKSLSLSPGK | |
| 23 | APELLGGPSVFLFPPKPKDTLMISRTPEVTCVVVDVSHEDPEVKFN | Clone CH3C.18 variant |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVYWESLGHVWAVYKTTPPVLDSDGSFFLYSKLT | |
| | VPKSTWQQGWVFSCSVMHEALHNHYTQKSLSLSPGK | |
| 24 | APELLGGPSVFLFPPKPKDTLMISRTPEVTCVVVDVSHEDPEVKFN | Clone CH3C.18 variant |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWAVYQTTPPVLDSDGSFFLYSKLTV | |
| | PKSTWQQGWVFSCSVMHEALHNHYTQKSLSLSPGK | |
| 25 | APELLGGPSVFLFPPKPKDTLMISRTPEVTCVVVDVSHEDPEVKFN | Clone CH3C.18 variant |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWAVYFTTPPVLDSDGSFFLYSKLTV | |
| | PKSTWQQGWVFSCSVMHEALHNHYTQKSLSLSPGK | |
| 26 | APELLGGPSVFLFPPKPKDTLMISRTPEVTCVVVDVSHEDPEVKFN | Clone CH3C.18 variant |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWAVHTTPPVLDSDGSFFLYSKLTV | |
| | PKSTWQQGWVFSCSVMHEALHNHYTQKSLSLSPGK | |
| 27 | APELLGGPSVFLFPPKPKDTLMISRTPEVTCVVVDVSHEDPEVKFN | Clone CH3C.35.13 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESLGHVWAVYKTTPPVLDSDGSFFLYSKLT | |
| | VPKSTWQQGWVFSCSVMHEALHNHYTQKSLSLSPGK | |
| 28 | APELLGGPSVFLFPPKPKDTLMISRTPEVTCVVVDVSHEDPEVKFN | Clone CH3C.35.14 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVWAVYQTTPPVLDSDGSFFLYSKLTV | |
| | PKSTWQQGWVFSCSVMHEALHNHYTQKSLSLSPGK | |
| 29 | APELLGGPSVFLFPPKPKDTLMISRTPEVTCVVVDVSHEDPEVKFN | Clone CH3C.35.15 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESLGHVWAVYQTTPPVLDSDGSFFLYSKLT | |
| | VPKSTWQQGWVFSCSVMHEALHNHYTQKSLSLSPGK | |
| 30 | APELLGGPSVFLFPPKPKDTLMISRTPEVTCVVVDVSHEDPEVKFN | Clone CH3C.35.16 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEEWSLGHVWVNQKTTPPVLDSDGSFFLYSKLT | |
| | VPKSTWQQGWVFSCSVMHEALHNHYTQKSLSLSPGK | |
| 31 | APELLGGPSVFLFPPKPKDTLMISRTPEVTCVVVDVSHEDPEVKFN | Clone CH3C.35.17 |
| | WYVDGVEVHANKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESLGHVEVNQQTTPPVLDSDGSFFLYSKLTV | |
| | PKSTWQQGWVFSCSVMHEALHNHYTQKSLSLSPGK | |
| 32 | APELLGGPSVFLFPPKPKDTLMISRTPEVTCVVVDVSHEDPEVKFN | Clone CH3C.35.18 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESLGHVWVNQQTTPPVLDSDGSFFLYSKLT | |
| | VPKSTWQQGWVFSCSVMHEALHNHYTQKSLSLSPGK | |
| 33 | APELLGGPSVFLFPPKPKDTLMISRTPEVTCVVVDVSHEDPEVKFN | Clone CH3C.35.19 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSSYKTTPPVLDSDGSFFLYSKLTV | |
| | TKSEWQQGFVFSCSVMHEALHNHYTQKSLSLSPGK | |
| 34 | APELLGGPSVFLFPPKPKDTLMISRTPEVTCVVVDVSHEDPEVKFN | Clone CH3C.35.20 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSSYKTTPPVLDSDGSFFLYSKLTV | |
| | TKEEWQQGFVFSCSVMHEALHNHYTQKSLSLSPGK | |
| 35 | APELLGGPSVFLFPPKPDKTLMISRTPEVTCVVVDVSHEDPEVKFN | Clone CH3C.35.21 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSSYKTTPPVLDSDGSFFLYSKLTV | |
| | TKEEWQQGFVFSCSVMHEALHNHYTQKSLSLSPGK | |
| 36 | APELLGGPSVFLFPPKPKDTLMISRTPEVTCVVVDVSHEDPEVKFN | Clone CH3C.35.22 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSNYKTTPPVLDSDGSFFLYSKLTV | |
| | TKSEWQQGFVFSCSVMHEALHNHYTQKSLSLSPGK | |
| 37 | APELLGGPSVFLFPPKPKDTLMISRTPEVTCVVVDVSHEDPEVKFN | Clone CH3C.35.23 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSNYKTTPPVLDSDGSFFLYSKLTV | |
| | TKEEWQQGFVFSCSVMHEALHNHYTQKSLSLSPGK | |
| 38 | APELLGGPSVFLPPKPKDTLMISTPEVTCVVVDVSHEDPEVKFN | Clone CH3C.35.24 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSNYKTTPPVLDSDGSFFLYSKLTV | |
| | TKEEWQQGFVFSCSVMHEALHNHYTQKSLSLSPGK | |
| 39 | APELLGGPSVFLFPPKPKDTLMISRTPEVTCVVVDVSHEDPEVKFN | Clone CH3C.35.N163 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSNYKTTPPVLDSDGSFFLYSKLTV | |
| | TKSEWQQGFVFSCSVMHEALHNHYTQKSLSLSPGK | |
| 40 | APELLGGPSVFLFPPKPKDTLMISRTPEVTCVVVDVSHEDPEVKFN | Clone CH3C.35.K165Q |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSSYQTTPPVLDSDGSFFLYSKLTV | |
| | TKSEWQQGFVFSCSVMHEALHNHYTQKSLSLSPGK | |
| 41 | APELLGGPSVFLFPPKPKDTLMISRTPEVTCVVVDVSHEDPEVKFN | Clone CHEC.35.N163.K165Q |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSNYQTTPPVLDSDGSFFLYSKLTV | |
| | TKSEWQQGFVFSCSVMHEALHNHYTQKSLSLSPGK | |
| 42 | APELLGGPSVFLFPPKPKDTLMISRTPEVTCVVVDVSHEDPEVKFN | Clone CH3C.35.21.1 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYKTTPPVLDSDGFFLYSKLTV | |
| | TKSEWQQGFVFSCSVMHEALHNHYTQKSLSLSPGK | |
| 43 | APELLGGPSVFLFPPKPKDTLMISRTPEVTCVVVDVSHEDPEVKFN | CH3C.35.21.2 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TKSEWQQGFVFSCSVMHEALHNHYTQKSLSLSPGK | |
| 44 | APELLGGPSVFLFPPKPKDTLMISRTPEVTCVVVDVSHEDPEVKFN | Clone CH3C.35.21.3 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TREEWQQGFVFSCSVMHEALHNHYTQKSLSLSPGK | |
| 45 | APELLGGPSVFLFPPKPKDTLMISRTPEVTCVVVDVSHEDPEVKFN | Clone CH3C.35.21.4 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TGEEWQQGFVFSCSVMHEALHNHYTQKSLSLSPGK | |
| 46 | APELLGGPSVFLFPPKPKDTLMISRTPEVTCVVVDVSHEDPEVKFN | Clone CH3C.35.21.5 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TREEWQQGFVFSCWVMHEALHNHYTQKSLSLSPGK | |
| 47 | APELLGGPSVFLFPPKPKDTLMISRTPEVTCVVVDVSHEDPEVKFN | Clone CH3C.35.21.6 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TKEEWQQGFVFSCWVMHEALHNHYTQKSLSLSPGK | |
| 48 | APELLGGPSVFLFPPKPKDTLMISRTPEVTCVVVDVSHEDPEVKFN | Clone CH3C.35.21.7 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TREEWQQGFVFTCWVMHEALHNHYTQKSLSLSPGK | |
| 49 | APELLGGPSVFLFPPKPKDTLMISRTPEVTCVVVDVSHEDPEVKFN | Clone CH3C.35.21.8 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TREEWQQGFVFTCGVMHEALHNHYTQKSLSLSPGK | |
| 50 | APELLGGPSVFLFPPKPKDTLMISRTPEVTCVVVDVSHEDPEVKFN | Clone CH3C.35.21.9 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TREEWQQGFVFECWVMHEALHNHYTQKSLSLSPGK | |
| 51 | APELLGGPSVFLFPPKPKDTLMISRTPEVTCVVVDVSHEDPEVKFN | Clone CH3C.35.21.10 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TREEWQQGFVFKCWVMHEALHNHYTQKSLSLSPGK | |
| 52 | APELLGGPSVFLFPPKPKDTLMISRTPEVTCVVVDVSHEDPEVKFN | Clone CH3C.35.21.11 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TPEEWQQGFVFKCWVMHEALHNHYTQKSLSLSPGK | |
| 53 | APELLGGPSVFLFPPKDTLMISRTPEVTCVVVDVSHEDPEVKFN | Clone CH3C.35.21.12 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSSYRTTPPVLDSDGSFFLYSKLTV | |
| | TREEWQQGFVFSCSVMHEALHNHYTQKSLSLSPGK | |
| 54 | APELLGGPSVFLFPPKPKDTLMISRTPEVTCVVVDVSHEDPEVKFN | Clone CH3C.35.21.13 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSSYRTTPPVLDSDGSFFLYSKLTV | |
| | TGEEWQQGFVFSCSVMHEALHNHYTQKSLSLSPGK | |
| 55 | APELLGGPSVFLFPPKPKDTLMISRTPEVTCVVVDVSHEDPEVKFN | Clone CH3C.35.21.14 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSSYRTTPPVLDSDGSFFLYSKLTV | |
| | TREEWQQGFVFTCWVMHEALHNHYTQKSLSLSPGK | |
| 56 | APELLGGPSVFLFPPKPKDTLMISRTPEVTCVVVDVSHEDPEVKFN | Clone CH3C.35.21.15 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSSYRTTPPVLDSDGSFFLYSKLTV | |
| | TGEEWQQGFVFTCWVMHEALHNHYTQKSLSLSPGK | |
| 57 | APELLGGPSVFLFPPKPKDTLMISRTPEVTCVVVDVSHEDPEVKFN | Clone CH3C.35.21.16 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSSYRTTPPVLDSDGSFFLYSKLTV | |
| | TREEWQQGFVFTCGVMHEALHNHYTQKSLSLSPGK | |
| 58 | APELLGGPSVFLFPPKPKDTLMISRTPEVTCVVVDVSHEDPEVKFN | Clone CH3C.35.21.17 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYKTTPPVLDSDGSFFLYSKLTV | |
| | TKEEWQQGFVFSCSVMHEALHNHYTQKSLSLSPGK | |
| 59 | APELLGGPSVFLFPPKPKDTLMISRTPEVTCVVVDVSHEDPEVKFN | Clone CH3C.35.21.18 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYRTTPPVLDSDGSFFLYSKLTV | |
| | TKEEWQQGFVFSCSVMHEALHNHYTQKSLSLSPGK | |
| 60 | APELLGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESFGTEWSSYKTTPPVLDSDGSFFLYSKLTVT | |
| | KEEWQQGFVFSCSVMHEALHNHYTQKSLSLSPGK | |
| 61 | APELLGGPSVFLFPPKPKDTLMISRTPEVTCVVVDVSHEDPEVKFN | Clone CH3C.35.20.2 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWASYKTTPPVLDSDGSFFLYSKLTV | |
| | TKEEWQQGFVFSCSVMHEALHNHYTQKSLSLSPGK | |
| 62 | APELLGGPSVFLFPPKPKDTLMISRTPEVTCVVVDVSHEDPEVKFN | Clone CH3C.35.20.3 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWVSYKTTPPVLDSDGSFFLYSKLTV | |
| | TKEEWQQGFVFSCSVMHEALHNHYTQKSLSLSPGK | |
| 63 | APELLGGPSVFLFPPKPKDTLMISRTPEVTCVVVDVSHEDPEVKFN | Clone CH3C.35.20.4 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSSYKTTPPVLDSDGSFFLYSKLTV | |
| | SKEEWQQGFVFSCSVMHEALHNHYTQKSLSLSPGK | |
| 64 | APELLGGPSVFLFPPKPKDTLMISRTPEVTCVVVDVSHEDPEVKFN | Clone CH3C.35.20.5 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESFGTEWASYKTTPPVLDSDGSFFLYSKLTV | |
| | TKEEWQQGFVFSCSVMHEALHNHYTQKSLSLSPGK | |
| 65 | APELLGGPSVFLFPPKPKDTLMISRTPEVTCVVVDVSHEDPEVKFN | Clone CH3C.35.20.6 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESFGTEWVSYKTTPPVLDSDGSFFLYSKLTV | |
| | TKEEWQQGFVFSCSVMHEALHNHYTQKSLSLSPGK | |
| 66 | APELLGGPSVFLFPPKPKDTLMISRTPEVTCVVVDVSHEDPEVKFN | Clone CH3C.35.21.a.1 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESFGTEWSSYKTTPPVLDSDGSFFLYSKLTV | |
| | TKEEWQQGFVFSCSVMHEALHNHYTQKSLSLSPGK | |
| 67 | APELLGGPSVFLFPPKPKDTLMISRTPEVTCVVVDVSHEDPEVKFN | Clone CH3C.35.21.a.2 |
| | WYVDGVEVHNAKTKPREEQYNSTYRVVSVLTVLHQDWLNGKEY | |
| | KCKNSNKALPAPIEKTISKAKGQPREPQVYTLPPSRDELTKNQVSLT | |
| | CLVKGFYPSDIAVWWESYGTEWASYKTTPPVLDSDGSFFLYSKLTV | |
| | TKEEQQGFVFSCSVMHEALHNHYTQKSLSLSPGK | |
| 68 | APELLGGPSVFLFPPKPKDTLMISRTPEVTCVVVDVSHEDPEVKFN | Clone CH3C.35.21.a.3 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWVSYKTTPPVLDSDGSFFLYSKLTV | |
| | TKEEWQQGFVFSCSVMHEALHNHYTQKSLSLSPGK | |
| 69 | APELLGGPSVFLFPPKPKDTLMISRTPEVTCVVVDVSHEDPEVKFN | Clone CH3C.35.21.a.4 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSSYKTTPPVLDSDGSFFLYSKLTV | |
| | SKEEWQQGFVFSCSVMHEALHNHYTQKSLSLSPGK | |
| 70 | APELLGGPSVFLFPPKPKDTLMISRTPEVTCVVVDVSHEDPEVKFN | Clone CH3C.35.21.a.5 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESFGTEWASYKTTPPVLDSDGSFFLYSKLTV | |
| | TKEEWQQGFVFSCSVMHEALHNHYTQKSLSLSPGK | |
| 71 | APELLGGPSVFLFPPKPKDTLMISRTPEVTCVVVDVSHEDPEVKFN | Clone CH3C.35.21.a.6 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESFGTEWVSYKTTPPVLDSDGSFFLYSKLTV | |
| | TKEEWQQGFVFSCSVMHEALHNHYTQKSLSLSPGK | |
| 72 | APELLGGPSVFLFPPKPKDTLMISRTPEVTCVVVDVSHEDPEVKFN | Clone CH3C.35.23.1 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESFGTEWSNYKTTPPVLDSDGSFFLYSKLTV | |
| | TKEEWQQGFVFSCSVMHEALHNHYTQKSLSLSPGK | |
| 73 | APELLGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWANYKTTPPVLDSDGSFFLYSKLTV | |
| | TKEEWQQGFVFSCSVMHEALHNHYTQKSLSLSPGK | |
| 74 | APELLGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWVNYKTTPPVLDSDGSFFLYSKLTV | |
| | TKEEWQQGFVFSCSVMHEALHNHYTQKSLSLSPGK | |
| 75 | APELLGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSNYKTTPPVLDSDGSFFLYSKLTV | |
| | SKEEWQQGFVFSCSVMHEALHNHYTQKSLSLSPGK | |
| 76 | APELLGGPSVFLFPPKPKDTLMISRTPEVTCVVVDVSHEDPEVKFN | Clone CH3C.35.23.5 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESFGTEWANYKTTPPVLDSDGSFFLYSKLTV | |
| | TKEEWQQGFVFSCSVMHEALHNHYTQKSLSLSPGK | |
| 77 | APELLGGPSVFLFPPKPKDTLMISRTPEVTCVVVDVSHEDPEVKFN | Clone CH3C.35.23.6 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESFGTEWVNYKTTPPVLDSDGSFFLYSKLTV | |
| | TKEEWQQGFVFSCSVMHEALHNHYTQKSLSLSPGK | |
| 78 | APELLGGPSVFLFPPKPKDTLMISRTPEVTCVVVDVSHEDPEVKFN | Clone CH3C.35.24.1 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESFGTEWSNYKTTPPVLDSDGSFFLYSKLTV | |
| | TKEEWQQGFVFSCSVMHEALHNHYTQKSLSLSPGK | |
| 79 | APELLGGPSVFLFPPKPKDTLMISRTPEVTCVVVDVSHEDPEVKFN | Clone CH3C.35.24.2 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWANYKTTPPVLDSDGSFFLYSKLT | |
| | VTKEEWQQGFVFSCSVMHEALHNHYTQKSLSLSPGK | |
| 80 | APELLGGPSVFLFPPKPKDTLMISRTPEVTCVVVDVSHEDPEVKFN | Clone CH3C.35.24.3 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWVNYKTTPPVLDSDGSFFLYSKLT | |
| | VTKEEWQQGFVFSCSVMHEALHNHYTQKSLSLSPGK | |
| 81 | APELLGGPSVFLFPPKPKDTLMISRTPEVTCVVVDVSHEDPEVKFN | Clone CH3C.35.24.4 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESYGTEWSNYKTTPPVLDSDGSFFLYSKLTV | |
| | SKEEWQQGFVFSCSVMHEALHNHYTQKSLSLSPGK | |
| 82 | APELLGGPSVFLFPPKPKDTLMISRTPEVTCVVVDVSHEDPEVKFN | Clone CH3C.35.24.5 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESFGTEWANYKTTPPVLDSDGSFFLYSKLTV | |
| | TKEEWQQGFVFSCSVMHEALHNHYTQKSLSLSPGK | |
| 83 | APELLGGPSVFLFPPKPKDTLMISRTPEVTCVVVDVSHEDPEVKFN | Clone CH3C.35.24.6 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVWWESFGTEWVNYKTTPPVLDSDGSFFLYSKLTV | |
| | TKEEWQQGFVFSCSVMHEALHNHYTQKSLSLSPGK | |
| 84 | APELLGGPSVFLFPPKPKDTLMISRTPEVTCVVVDVSHEDPEVKFN | Clone CH3C.35.21.17.1 |
| | WYVDGVEVHANKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESFGTEWSSYKTTPPVLDSDGSFFLYSKLTVT | |
| | KEEWQQGFVFSCSVMHEALHNHYTQKSLSLSPGK | |
| 85 | APELLGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWASYKTTPPVLDSDGSFFLYSKLTV | |
| | TKEEWQQGFVFSCSVMHEALHNHYTQKSLSLSPGK | |
| 86 | APELLGGPSVFLFPPKPKDTLMISRTPEVTCVVVDVSHEDPEVKFN | Clone CH3C.35.21.17.3 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWVSYKTTPPVLDSDGSFFLYSKLTV | |
| | TKEEWQQGFVFSCSVMHEALHNHYTQKSLSLSPGK | |
| 87 | APELLGGPSVFLFPPKPKDTLMISRTPEVTCVVVDVSHEDPEVKFN | Clone CH3C.35.21.17.4 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESYGTEWSSYKTTPPVLDSDGSFFLYSKLTV | |
| | SKEEWQQGFVFSCSVMHEALHNHYTQKSLSLSPGK | |
| 88 | APELLGGPSVFLFPPKPKDTLMISRTPEVTCVVVDVSHEDPEVKFN | Clone CH3C.35.21.17.5 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESFGTEWASYKTTPPVLDSDGSFFLYSKLTV | |
| | TKEEWQQGFVFSCSVMHEALHNHYTQKSLSLSPGK | |
| 89 | APELLGGPSVFLFPPKPKDTLMISRTPEVTCVVVDVSHEDPEVKFN | Clone CH3C.35.21.17.6 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVLWESFGTEWVSYKTTPPVLDSDGSFFLYSKLTV | |
| | TKEEWQQGFVFSCSVMHEALHNHYTQKSLSLSPGK | |
| 90 | APELLGGPSVFLFPPKPKDTLMISRTPEVTCVVVDVSHEDPEVKFN | Clone CH3C.35.N390 |
| | WYVDGVEVHNAKTKPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESYGTEWSNYKTTPPVLDSDGSFFLYSKLTV | |
| | TKSEWQQGFVFSCSVMHEALHNHYTQKSLSLSPGK | |
| 91 | EPKSCDKTHTCPPCP | Human IgG1 hinge |
| | | amino acid sequence |
| 92 | MMDQARSAFSNLFGGEPLSYTRFSLARQVDGDNSHVEMKLAVDEE | Human transerrin |
| | ENADNNTKANVTKPKRCSGSICYGTIAVIVFFLIGFMIGYLGYCKGV | receptor protein 1 |
| | EPKTECERLAGTESPVREEPGEDFPAARRLYWDDLKRKLSEKLDST | (TFR1) |
| | DFTGTIKLLNENSYVPREAGSQKDENLALYVENQFREFKLSKVWR | |
| | DQHFVKIQVKDSAQNSVIIVDKNGRLVYLVENPGGYVAYSKAATV | |
| | TGKLVHANFGTKKDFEDLYTPVNGSIVIVRAGKITFAEKVANAESL | |
| | NAIGVLIYMDQTKFPIVNAELSFFGHAHLGTGDPYTPGFPSFNHTQF | |
| | PPSRSSGLPNIPVQTISRAAAEKLFGNMEGDCPSDWKTDSTCRMVT | |
| | SESKNVKLTVSNVLKEIKILNIFGVIKGFVEPDHYVVVGAQRDAWG | |
| | PGAAKSGVGTALLLKLAQMFSDMVLKDGFQPSRSIIFASWSAGDFG | |
| | SVGATEWLEGYLSSLHLKAFTYINLDKAVLGTSNFKVSASPLLTYLI | |
| | EKTMQNVKHPVTGQFLYQDSNWASKVELKTLDNAAFPFLAYSGIP | |
| | AVSFCFCEDTDYPYLGTTMDTYKELIERIPELNKVARAAAEVAGQF | |
| | VIKLTHDVELNLDYERYNSQLLSFVRDLNQYRADIKEMGLSLQWL | |
| | YSARGDFFRATSRLTTDFGNAEKTDRFVMKKLNDRVMRVEYHFLS | |
| | PYVSPKESPFRHVFWGSGSHTLPALLENLKLRKQNNGAFNETLFRN | |
| | QLALATWTIQGAANALSGDVWDIDNEF | |
| 93 | APELLGGPSVFLFPPKPKDTLMISRTPEVTCVVVDVSHEDPEVKFN | Clone CH3C.35.21 with |
| | WYVDGVEVHNAKTKPREEQYNSTYRVVSVLTVLHQDWLNGKEY | knob mutation |
| | KCKVSNKALPAPIEKTISKAKGQPREPQVYTLPPSRDELTKNQVSL | |
| | WCLVKGFYPSDIAVWWESYGTEWSSYKTTPPVLDSDGSFFLYSKL | |
| | TVTKEEWQQGFVFSCSVMHEALHNHYTQKSLSLSPGK | |
| 94 | APEAAGGPSVFLFPPKPKDTLMISRTPEVTCVVVSHEDPEVKFN | Clone CH3C.35.21 with |
| | WYVDGVEVHNAKTKPREEQYNSTYRVVSVLTVLHQDWLNGKEY | knob and LALA |
| | KCKVSNKALPAPIEKTISKAKGQPREPQVYTLPPSRDELTKNQVSL | mutations |
| | WCLVKGFYPSDIAVWWESYGTEWSSYKTTPPVLDSDGSFFLYSKL | |
| | TVTKEEWQQGFVFSCSVMHEALHNHYTQKSLSLSPGK | |
| 95 | APELLGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 96 | APEAAGGPSVFLFPPKPKDTLYITREPEVTCVVVDVSHEDPEVKFN | Clone CH3C.35.21 with |
| | WYVDGVEVHNAKTKPREEQYNSTYRVVSVLTVLHQDWLNKEY | knob, LALA, and YTE |
| | KCKVSNKALPAPIEKTISKAKGQPREPQVYTLPPSRDELTKNQVSL | mutations |
| | WCLVKGFYPSDIAVWWESYGTEWSSYKTTPPVLDSDGSFFLYSKL | |
| | TVTKEEWQQGFVFSCSVMHEALHNHYTQKSLSLSPGK | |
| 97 | APELLGGPSVFLFPPKPKDTLMISRTPEVTCVVVDVSHEDPEVKFN | Fc sequence with hole |
| | WYVDGVEVHNAKTKPREEQYNSTYRVVSVLTVLHQDWLNGKEY | mutations |
| | KCKVSNKALPAPIEKTISKAKGQPREPQVYTLPPSRDELTKNQVSLS | |
| | CAVKGFYPSDIAVEWESNGQPENNYKTTPPVLDSDGSFFLVSKLTV | |
| | DKSRWQQGNVFSCSVMHEALHNHYTQKSLSLSPGK | |
| 98 | APEAAGGPSVFLFPPKPKDTLMISRTPEVTCVVVDVSHEDPEVKFN | Fc sequence with hole |
| | WYVDGVEVHNAKTKPREEQYNSTYRVVSVLTVLHQDWLNGKEY | and LALA mutations |
| | KCKVSNKALPAPIEKTISKAKGQPREPQVYTLPPSRDELTKNQVSLS | |
| | CAVKGFYPSDIAVEWESNGQPENNYKTTPPVLDSDGSFFLVSKLTV | |
| | DKSRWQQGNVFSCSVMHEALHNHYTQKSLSLSPGK | |
| 99 | APELLGGPSVFLFPPKPKDTLYITREPEVTCVVVDVSHEDPEVKFNW | Fc sequence with hole |
| | YVDGVEVHNAKTKPREEQYNSTYRVVSVLTVLHQDWLNGKEYKC | and YTE mutations |
| | KVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDK | |
| | SRWQQGNVFSCSVMHEALHNHYTQKSLSLSPGK | |
| 100 | APEAAGGPSVFLFPPKPKDTLYITREPEVTCVVVDVSHEDPEVKFN | Fc sequence with hole, |
| | WYVDGVEVHNAKTKPREEQYNSTYRVVSVLTVLHQDWLNGKEY | LALA, and YTE |
| | KCKVSNKALPAPIEKTISKAKGQPREPQVYTLPPSRDELTKNQVSLS | mutations |
| | CAVKGFYPSDIAVEWESNGQPENNYKTTPPVLDSDGSFFLVSKLTV | |
| | DKSRWQQGNVFSCSVMHEALHNHYTQKSLSLSPGK | |
| 101 | APELLGGPSVFLFPPKPKDTLMISRTPEVTCVVVDVSHEDPEVKFN | Clone CH3C.35.21 with |
| | WYVDGVEVHNAKTKPREEQYNSTYRVVSVLTVLHQDWLNGKEY | hole mutations |
| | KCKVSNKALPAPIEKTISKAKGQPREPQVYTLPPSRDELTKNQVSLS | |
| | CAVKGFYPSDIAVWWESYGTEWSSYKTTPPVLDSDGSFFLVSKLTV | |
| | TKEEWQQGFVFSCSVMHEALHNHYTQKSLSLSPGK | |
| 102 | APEAAGGPSVFLFPPKPKDTLMISRTPEVTCVVVDVSHEDPEVKFN | Clone CH3C.35.21 with |
| | WYVDGVEVHNAKTKPREEQYNSTYRVVSVLTVLHQDWLNGKEY | hole and LALA |
| | KCKVSNKALPAPIEKTISKAKGQPREPQVYTLPPSRDELTKNQVSLS | mutations |
| | CAVKGFYPSDIAVWWESYGTEWSSYKTTPPVLDSDGSFFLVSKLTV | |
| | TKEEWQQGFVFSCSVMHEALHNHYTQKSLSLSPGK | |
| 103 | APELLGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | hole and YTE mutations |
| | KVSNKALPAPIEKTISKAKGQPREPVYTLPPSRDELTKNQVSLSCA | |
| | VKGFYPSDIAVWWESYGTEWSSYKTTPPVLDSDGSFFLVSKLTVTK | |
| | EEWQQGFVFSCSVMHEALHNHYTQKSLSLSPGK | |
| 104 | APEAAGGPSVFLFPPKPKDTLYITREPEVTCVVVDVSHEDPEVKFN | Clone CH3C.35.21 with |
| | WYVDGVEVHNAKTKPREEQYNSTYRVVSVLTVLHQDWLNGKEY | hole, LALA, and YTE |
| | KCKVSNKALPAPIEKTISKAKGQPREPQVYTLPPSRDELTKNQVSLS | mutations |
| | CAVKGFYPSDIAVWWESYGTEWSSYKTTPPVLDSDGSFFLVSKLTV | |
| | TKEEWQQGFVFSCSVMHEALHNHYTQKSLSLSPGK | |
| 105 | APELLGGPSVFLFPPKPKDTLMISRTPEVTCVVVDVSHEDPEVKFN | Fc sequence with knob |
| | WYVDGVEVHNAKTKPREEQYNSTYRVVSVLTVLHQDWLNGKEY | mutation |
| | KCKVSNKALPAPIEKTISKAKGQPREPQVYTLPPSRDELTKNQVSL | |
| | WCLVKGFYPSDIAVEWESNGQPENNYKTTPPVLDSDGSFFLYSKLT | |
| | VDKSRWQQGNVFSCSVMHEALHNHYTQKSLSLSPGK | |
| 106 | APEAAGGPSVFLFPPKPKDTLMISRTPEVTCVVVDVSHEDPEVKFN | Fc sequence with knob |
| | WYVDGVEVHNAKTKPREEQYNSTYRVVSVLTVLHQDWLNGKEY | and LALA mutations |
| | KCKVSNKALPAPIEKTISKAKGQPREPQVYTLPPSRDELTKNQVSL | |
| | WCLVKGFYPSDIAVEWESNGQPENNYKTTPPVLDSDGSFFLYSKLT | |
| | VDKSRWQQGNVFSCSVMHEALHNHYTQKSLSLSPGK | |
| 107 | APELLGGPSVFLFPPKPKDTLYITREPEVTCVVVDVSHEDPEVKFNW | Fc sequence with knob |
| | YVDGVEVHNAKTKPREEQYNSTYRVVSVLTVLHQDWLNGKEYKC | and YTE mutations |
| | KVSNKALPAPIEKTISKAKGQPREPQVYTLPPSRDELTKNQVSLWCL | |
| | VKGFYPSDIAVEWESNGPENNYKTTPPVLDSDGSFFLYSKLTVDK | |
| | SRWQQGNVFSCSVMHEALHNHYTQKSLSLSPGK | |
| 108 | APEAAGGPSVFLFPPKPKDTLYITREPEVTCVVVDVSHEDPEVKFN | Fc sequence with knob, |
| | WYVDGVEVHNAKTKPREEQYNSTYRVVSVLTVLHQDSLNGKEY | LALA, and YTE |
| | KCKVSNKALPAPIEKTISKAKGQPREPQVYTLPPSRDELTKNQVSL | mutations |
| | WCLVKGFYPSDIAVEWESNGQPENNYKTTPPVLDSDGSFFLYSKLT | |
| | VDKSRWQQGNVFSCSVMHEALHNHYTQKSLSLSPGK | |
| 109 | DKTHTCPPCP | Portion of human IgG1 |
| | | hinge sequence (Partial |
| | | hinge) |
| 110 | DKTHTCPPCPAPEAAGGPSVFLFPPKPKDTLMISRTPEVTCVVVDVS | Clone CH3C.35.21 with |
| | HEDPEVKFNWYVDGVEVHNAKTKPREEQYNSTYRVVSVLTVLHQ | knob and LALA |
| | DWLNGKEYKCKVSNKALPAPIEKTISKAKGQPREPQVYTLPPSRDE | mutations and portion of |
| | LTKNQVSLWCLVKGFYPSDIAVWWESYGTEWSSYKTTPPVLDSDG | human IgG1 hinge |
| | SFFLYSKLTVTKEEWQQGFVFSCSVMHEALHNHYTQKSLSLSPGK | sequence |
| 111 | APELLGGPSVFLFPPKPKDTLMISRTPEVTCVVVDVSHEDPEVKFN | Clone CH3B.1 |
| | WYVDGVEVHNAKTKPREEQYNSTYRVVSVLTVLHQDWLNGKEY | |
| | KCKVSNKALPAPIEKTISKAKGQPRFDYVTTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYGFHDLSLSPGK | |
| 112 | APELLGGPSVFLFPPKPKDTLMISRTPEVTCVVVDVSHEDPEVKFN | Clone CH3B.2 |
| | WYVDGVEVHNAKTKPREEQYNSTYRVVSVLTVLHQDWLNGKEY | |
| | KCKVSNKALPAPIEKTISKAKGQPRFDMVTTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYGFHDSLSLPGK | |
| 113 | APELLGGPSVFLFPPKPKDTLMISRTPEVTCVVVDVSHEDPEVKFN | Clone CH3B.3 |
| | WYVDGVEVHNAKTKPREEQYNSTYRVVSVLTVLHQDWLNGKEY | |
| | KCKVSNKALPAPIEKTISKAKGQPRFEYVTTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYGFHDLSLSPGK | |
| 114 | APELLGGPSVFLFPPKPKDTLMISRTPEVTCVVVDVSHEDPEVKFN | Clone CH3B.4 |
| | WYVDGVEVHNAKTKPREEQYNSTYRVVSVLTVLHQDWLNGKEY | |
| | KCKVSNKALPAPIEKTISKAKGQPRFEMVTTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYGFHDLSLSPGK | |
| 115 | APELLGGPSVFLFPPKPKDTLMISRTPEVTCVVVDVSHEDPEVKFN | Clone CH3B.5 |
| | WYVDGVEVHNAKTKPREEQYNSTYRVVSVLTVLHQDWLNGKEY | |
| | KCKVSNKALPAPIEKTISKAKGQPRFELVTTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYGFHDLSLSPGK | |
| 116 | APELLGGPSVFLFPPKPKDTLMISRTPEVTCVVVDVSHEDPEVEFIW | Clone CH2A2.1 |
| | YVDGVDVRYEWQLPREEQYNSTYRVVSVLTVLHQDWLNGKEYK | |
| | CKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVD | |
| | KSRWQQGNVFSCSVMHEALHNHYTQKSLSLSPGK | |
| 117 | APELLGGPSVFLFPPKPKDTLMISRTPEVTCVVVDVSHEDPEVGFV | Clone CH2A2.2 |
| | WYVDGVPVSWEWYWPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHTYQKSLSLSPGK | |
| 118 | APELLGGPSVFLFPPKPKDTLMISRTPEVTCVVVDVSHEDPEVQFD | Clone CH2A2.3 |
| | WYVDGVMVRREWHRPREEQYNSTYRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 119 | APELLGGPSVFLFPPKPKDTLMISRTPEVTCVVVDVSHEDPEVSFEW | Clone CH2A2.4 |
| | YVDGVPVRWEWQWPREEQYNSTYRVVSVLTVLHQDWLNGKEYK | |
| | CKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVD | |
| | KSRWQQGNVFSCSVMHEALHNHYTQKSLSLSPGK | |
| 120 | APELLGGPSVFLFPPKPKDTLMISRTPEVTCVVVDVSHEDPEVAFTW | Clone CH2A2.5 |
| | YVDGVPVRWEWQNPREEQYNSTYRVVSVLTVLHQDWLNGKEYK | |
| | CKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVD | |
| | KSRWQQGNVFSCSVMHEALHNHYTQKSLSLSPGK | |
| 121 | APELLGGPSVFLFPPKPKDTLMISRTPEVTCVVVDPQTPPWEVKFN | Clone CH2C.1 |
| | WYVDGVEVHNAKTKPREEEYYTYYRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 122 | APELLGGPSVFLFPPKPKDTLMISRTPEVTCVVVDPPSPPWEVKFNW | Clone CH2C.2 |
| | YVDGVEVHNAKTKPREEEYYSNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDK | |
| | SRWQQGNVFSCSVMHEALHNHYTQKSLSLSPGK | |
| 123 | APELLGGPSVFLFPPKPKDTLMISRTPEVTCVVVDPQTPPWEVKFN | Clone CH2C.3 |
| | WYVDGVEVHNAKTKPREEEYYSNYRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 124 | APELLGGPSVFLFPPKPKDTLMISRTPEVTCVVVDFRGPPWEVKFN | Clone CH2C.4 |
| | WYVDGVEVHNAKTKPREEEYYHDYRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 125 | APELLGGPSVFLFPPKPKDTLMISRTPEVTCVVVDPQTVPWEVKFN | Clone CH2C.5 |
| | WYVDGVEVHNAKTKPREEEYYSNYRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 126 | APELLGGPSVFLFPPKPKDTLMISRTPEVTCVVVDVSVPPRMVKFN | Clone CH2D.1 |
| | WYVDGVEVHNAKTKSLTSQHNSTVRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 127 | APELLGGPSVFLFPPKPKDTLMISRTPEVTCVVVDVSVPPWMVKFN | Clone CH2D.2 |
| | WYVDGVEVHNAKTKSLTSQHNSTVRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 128 | APELLGGPSVFLFPPKPKDTLMISRTPEVTCVVVDVSDMWEYVKFN | Clone CH2D.3 |
| | WYVDGVEVHNAKTKPWVKQLNSTWRVVSVLTVLHQDWLNGKE | |
| | YKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLT | |
| | VDKSRWQQGNVFSCSVMHEALHNHYTQKSLSLSPGK | |
| 129 | APELLGGPSVFLFPPKPKDTLMISRTPEVTCVVVDVSDDWTWVKFN | Clone CH2D.4 |
| | WYVDGVEVHNAKTKPWIAQPNSTWRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 130 | APELLGGPSVFLFPPKPKDTLMISRTPEVTCVVVDVSDDWEWVKFN | Clone CH2D.5 |
| | WYVDGVEVHNAKTKPWKLQLNSTWRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 131 | APELLGGPSVFLFPPKPKDTLMISRTPEVTCVVVDVSHEDPWVWFY | Clone CH2E3.1 |
| | WYVDGVEVHNAKTKPREEQYNSTYRVVSVLTVLHQDWLNGKEY | |
| | KCSVVNIALWWSIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 132 | APELLGGPSVFLFPPKPKDTLMISRTPEVTCVVVDVSHEDPVVGFR | Clone CH2E3.2 |
| | WYVDGVEVHNAKTKPREEQYNSTYRVVSVLTVLHQDWLNGKEY | |
| | KCRVSNSALTWKIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 133 | APELLGGPSVFLFPPKPKDTLMISRTPEVTCVVVDVSHEDPVVGFR | Clone CH2E3.3 |
| | WYVDGVEVHNAKTKPREEQYNSTYRVVSVLTVLHQDWLNGKEY | |
| | KCRVSNSALSWRIEKTISKAKGQPREPQVYTLPPSRDELTKNQVSLT | |
| | CLVKGFYPSDIAVEWESNGQPENNYKTTPPVLDSDGSFFLYSKLTV | |
| | DKSRWQQGNVFSCSVMHEALHNHYTQKSLSLSPGK | |
| 134 | APELLGGPSVFLFPPKPKDTLMISRTPEVTCVVVDVSHEDPIVGFRW | Clone CH2E3.4 |
| | YVDGVEVHNAKTKPREEQYNSTYRVVSVLTVLHQDWLNGKEYKC | |
| | RVSNSALRWRIEKTISKAKGQPREPQVYTLPPSRDELTKNQVSLTCL | |
| | VKGFYPSDIAVEWESNGQPENNYKTTPPVLDSDGSFFLYSKLTVDK | |
| | SRWQQGNVFSCSVMHEALHNHYTQKSLSLSPGK | |
| 135 | APELLGGPSVFLFPPKPKDTLMISRTPEVTCVVVDVSHEDPAVGFE | Clone CH2E3.5 |
| | WYVDGVEVHNAKTKPREEQYNSTYRVVSVLTVLHQDWLNGKEY | |
| | KCQVFNWALDWVIEKTISKAKGQPREPQVYTLPPSRDELTKNQVSL | |
| | TCLVKGFYPSDIAVEWESNGQPENNYKTTPPVLDSDGSFFLYSKLT | |
| | VDKSRWQQGNVFSCSVMHEALHNHYTQKSLSLSPGK | |
| 136 | APEAAGGPSVFLFPPKPKDTLMISRTPEVTCVVVDVSHEDPEVKFN | Clone CH3C.35.21.17 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSL | mutations |
| | WCLVKGFYPSDIAVLWESYGTEWSSYKTTPPVLDSDGSFFLYSKLT | |
| | VTKEEWQQGFVFSCSVMHEALHNHYTQKSLSLSPGK | |
| 137 | APELLGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with knob mutation |
| | KCKVSNKALPAPIEKTISKAKGQPREPQVYTLPPSRDELTKNQVSL | |
| | WCLVKGFYPSDIAVEWESFGTEWSSYKTTPPVLDSDGSFFLYSKLT | |
| | VTKEEWQQGFVFSCSVMHEALHNHYTQKSLSLSPGK | |
| 138 | APEAAGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSL | mutations |
| | WCLVKGFYPSDIAVEWESFGTEWSSYKTTPPVLDSDGSFFLYSKLT | |
| | VTKEEWQQGFVFSCSVMHEALHHYTQKSLSLSPGK | |
| 139 | APEAAGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVEWESFGTEWSSYKTTPPVLDSDGSFFLYSKLT | |
| | VTKEEWQQGFVFSCSVMHEALHNHYTQKSLSLSPGK | |
| 140 | APELLGGPSVFLFPPKPKDTLYITREPEVTCVVVDVSHEDPEVKFNW | Clone CH3C.35.20.1 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 141 | APEAAGGPSVFLFPPKPKDTLYITREPEVTCVVVDVSHEDPEVKFN | Clone CH3C.35.20.1 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSL | YTE mutations |
| | WCLVKGFYPSDIAVEWESFGTEWSSYKTTPPVLDSDGSFFLYSKLT | |
| | VTKEEWQQGFVFSCSVMHEALHNHYTQKSLSLSPGK | |
| 142 | APEAAGGPSVFLFPPKPKDTLYITREPEVTCVVVDVSHEDPEVKFN | Clone CH3C.35.20.1 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTE mutations |
| | WCLVKGFYPSDIAVEWESFGTEWSSYKTTPPVLDSDGSFFLYSKLT | |
| | VTKEEWQQGFVFSCSVMHEALHNHYTQKSLSLSPGK | |
| 143 | APELLGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with hole mutations |
| | KCKVSNKALPAPIEKTISKAKGQPREPQVYTLPPSRDELTKNQVSLS | |
| | CAVKGFYPSDIAVEWESFGTEWSSYKTTPPVLDSDGSFFLVSKLTV | |
| | TKEEWQQGFVFSCSVMHEALHNHYTQKSLSLSPGK | |
| 144 | APEAAGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with hole and LALA |
| | KCKVSNKALPAPIEKTISKAKGQPREPQVYTLPPSRDELTKNQVSLS | mutations |
| | CAVKGFYPSDIAVEWESFGTEWSSYKTTPPVLDSDGSFFLVSKLTV | |
| | TKEEWQQGFVFSCSVMHEALHNHYTQKSLSLSPGK | |
| 145 | APEAAGGPSVFLFPPKPKDTLMISRTPEVTCVVVDVSHEDPEVKFN | Clone CH3C.35.20.1 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVEWESFGTEWSSYKTTPPVLDSDGSFFLVSKLTV | |
| | TKEEWQQGFVFSCSVMHEALHNHYTQKSLSLSPGK | |
| 146 | APELLGGPSVFLFPPKPKDTLYITREPEVTCVVVDVSHEDPEVKFNW | Clone CH3C.35.20.1 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESFGTEWSSYKTTPPVLDSDGSFFLVSKLTVTKE | |
| | EWQQGFVFSCSVMHEALHNHYTQKSLSLSPGK | |
| 147 | APEAAGGPSVFLFPPKPKDTLYITREPEVTCVVVDVSHEDPEVKFN | Clone CH3C.35.20.1 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTE mutations |
| | CAVKGFYPSDIAVEWESFGTEWSSYKTTPPVLDSDGSFFLYVSKLTV | |
| | TKEEWQQGFVFSCSVMHEALHNHYTQKSLSLSPGK | |
| 148 | APEAAGGPSVFLFPPKPKDTLYITREPEVTCVVVDVSHEDPEVKFN | Clone CH3C.35.20.1 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTE mutations |
| | CAVKGFYPSDIAVEWESFGTEWSSYKTTPPVLDSDGSFFLVSKLTV | |
| | TKEEWQQGFVFSCSVMHEALHNHYTQKSLSLSPGK | |
| 149 | APELLGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with knob mutation |
| | KCKVSNKALPAPIEKTISKAKGQPREPQVYTLPPSRDELTKNQVSL | |
| | WCLVKGFYPSDIAVEWESYGTEWANYKTTPPVLDSDGSFFLYSKL | |
| | TVTKEEWQQGFVFSCSVMHEALHNHYTQKSLSLSPGK | |
| 150 | APEAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSL | mutations |
| | WCLVKGFYPSDIAVEWESYGTEWANYKTTPPVLDSDGSFFLYSKL | |
| | TVTKEEWQQGFVFSCSVMHEALHNHYTQKSLSLSPGK | |
| 151 | APEAAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVEWESYGTEWANYKTTPPVLDSDGFFLYSKL | |
| | TVTKEEWQQGFVFSCSVMHEALHNHYTQKSLSLSPGK | |
| 152 | APELLGGPSVFLFPPKPKDTLYITREPEVTCVVVDVSHEDPEVKFNW | Clone CH3C.35.23.2 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 153 | APEAAGGPSVFLFPPKPKDTLYITREPEVTCVVVDVSHEDPEVKFN | Clone CH3C.35.23.2 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSL | YTE mutations |
| | WCLVKGFYPSDIAVEWESYGTEWANYKTTPPVLDSDGSFFLYSKL | |
| | TVTKEEWQQGFVFSCSVMHEALHNHYTQKSLSLSPGK | |
| 154 | APEAAGGPSVFLFPPKPKDTLYITREPEVTCVVVDVSHEDPEVKFN | Clone CH3C.35.23.2 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTE mutations |
| | WCLVKGFYPSDIAVEWESYGTEWANYKTTPPVLDSDGSFFLYSKL | |
| | TVTKEEWQQGFVFSCSVMHEALHNHYTQKSLSLSPGK | |
| 155 | APELLGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with hole mutations |
| | KCKVSNKALPAPIEKTISKAKGQPREPQVYTLPPSRDELTKNQVSLS | |
| | CAVKGFYPSDIAVEWESYGTEWANYKTTPPVLDSDGSFFLVSKLTV | |
| | TKEEWQQGFVFSCSVMHEALHNHYTAKSLSLSPGK | |
| 156 | APEAAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with hole and LALA |
| | KCKVSNKALPAPIEKTISKAKGQPREPQVYTLPPSRDELTKNQVSLS | mutations |
| | CAVKGFYPSDIAVEWESYGTEWANYKTTPPVLDSDGSFFLVSKLTV | |
| | TKEEWQQGFVFSCSVMHEALHNHYTQKSLSLSPGK | |
| 157 | APEAAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVEWESYGTEWANYKTTPPVLDSDGSFFLVSKLTV | |
| | TKEEWQQGFVFSCSVMHEALHNHYTQKSLSLSPGK | |
| 158 | APELLGGPSVFLFPPKPKDTLYITREPEVTCVVVDVSHEDPEVKFNW | Clone CH3C.35.23.2 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESYGTEWANYKTTPPVLDSDGSFFLVSKLTVTK | |
| | EEWQQGFVFSCSVMHEALHNHYTQKSLSLSPGK | |
| 159 | APEAAGGPSVFLFPPKPKDTLYITREPEVTCVVVDVSHEDPEVKFN | Clone CH3C.35.23.2 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTE mutations |
| | CAVKGFYPSDIAVEWESYGTEWANYKTTPPVLDSDGSFFLVSKLTV | |
| | TKEEWQQGFVFSCSVMHEALHNHYTQKSLSLSPGK | |
| 160 | APEAAGGPSVFLFPPKPKDTLYITREPEVTCVVVDVSHEDPEVKFN | Clone CH3C.35.23.2 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTE mutations |
| | CAVKGFYPSDIAVEWESYGTEWANYKTTPPVLDSDGSFFLVSKLTV | |
| | TKEEWQQGFVFSCSVMHEALHNHYTQKSLSLSPGK | |
| 161 | APELLGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with knob mutation |
| | KCKVSNKALPAPIEKTISKAKGQPREPQVYTLPPSRDELTKNQVSL | |
| | WCLVKGFYPSDIAVEWESYGTEWVNYKTTPPVLDSDGSFFLYSKL | |
| | TVTKEEWQQGFVFSCSVMHEALHNHYTQKSLSLSPGK | |
| 162 | APEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSL | mutations |
| | WCLVKGFYPSDIAVEWESYGTEWVNYKTTPPVLDSDGSFFLYSKL | |
| | TVTKEEWQQGFVFSCSVMHEALHNHYTQKSLSLSPGK | |
| 163 | APEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVEWESYGTEWVNYKTTPPVLDSDGSFFLYSKL | |
| | TVTKEEWQQGFVFSCSVMHEALHNHYTQKSLSLSPGK | |
| 164 | APELLGGPSVFLFPPKPKDTLYITREPEVTCVVVDVSHEDPEVKFNW | Clone CH3C.35.23.3 |
| | YVDGVEVHNAKTKPREEQYNSTYRVVSVLTVLHQDWLNGKEYC | with knob and YTE |
| | KVSNKALPAPIEKTISKAKGQPREPQVYTLPPSRDELTKNQVSLWCL | mutations |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 165 | APEAAGGPSVFLFPPKPKDTLYITREPEVTCVVVDVSHEDPEVKFN | Clone CH3C.35.23.3 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSL | YTE mutations |
| | WCLVKGFYPSDIAVEWESYGTEWVNYKTTPPVLDSDGSFFLYSKL | |
| | TVTKEEWQQGFVFSCSVMHEALHNHYTQKSLSLSPGK | |
| 166 | APEAAGGPSVFLFPPKPKDTLYITREPEVTCVVVDVSHEDPEVKFN | Clone CH3C.35.23.3 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTE mutations |
| | WCLVKGFYPSDIAVEWESYGTEWVNYKTTPPVLDSDGSFFLYSKL | |
| | TVTKEEWQQGFVFSCSVMHEALHNHYTQKSLSLSPGK | |
| 167 | APELLGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with hole mutations |
| | KCKVSNKALPAPIEKTISKAKGQPREPQVYTLPPSRDELTKNQVSLS | |
| | CAVKGFYPSDIAVEWESYGTEWVNYKTTPPVLDSDGSFFLVSKLTV | |
| | TKEEWQQGFVFSCSVMHEALHNHYTQKSLSLSPGK | |
| 168 | APEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with hole and LALA |
| | KCKVSNKALPAPIEKTISKAKGQPREPQVYTLPPSRDELTKNQVSLS | mutations |
| | CAVKGFYPSDIAVEWESYGTEWVNYKTTPPVLDSDGSFFLVSKLTV | |
| | TKEEWQQGFVFSCSVMHEALHNHYTQKSLSLSPGK | |
| 169 | APEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVEWESYGTEWVNYKTTPPVLDSDGSFFLVSKLTV | |
| | TKEEWQQGFVFSCSVMHEALHNHYTQKSLSLSPGK | |
| 170 | APELLGGPSVFLFPKKPKDTLYITREPEVTCVVVDVSHEDPEVKFNW | Clone CH3C.35.23.3 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESYGTEWVNYKTTPPVLDSDGSFFLVSKLTVTK | |
| | EEWQQGFVFSCSVMHEALHNHYTQKSLSLSPGK | |
| 171 | APEAAGGPSVFLFPPKPKDTLYITREPEVTCVVVDVSHEDPEVKFN | Clone CH3C.35.23.3 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTE mutations |
| | CAVKGFYPSDIAVEWESYGTEWVNYKTTPPVLDSDGSFFLVSKLTV | |
| | TKEEWQQGFVFSCSVMHEALHNHYTQKSLSLSPGK | |
| 172 | APEAAGGPSVFLFPPKPKDTLYITREPEVTCVVVDVSHEDPEVKFN | Clone CH3C.35.23.3 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTE mutations |
| | CAVKGFYPSDIAVEWESYGTEWVNYKTTPPVLDSDGSFFLVSKLTV | |
| | TKEEWQQGFVFSCSVMHEALHNHYTQKSLSLSPGK | |
| 173 | APELLGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with knob mutation |
| | KCKVSNKALPAPIEKTISKAKGQPREPQVYTLPPSRDELTKNQVSL | |
| | WCLVKGFYPSDIAVEWESYGTEWSNYKTTPPVLDSDGSFFLYSKLT | |
| | VSKEEWQQGFVFSCSVMHEALHNHYTQKSLSLSPGK | |
| 174 | APEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSL | mutations |
| | WCLVKGFYPSDIAVEWESYGTEWSNYKTTPPVLDSDGSFFLYSKLT | |
| | VSKEEWQQGFVFSCSVMHEALHNHYTQKSLSLSPGK | |
| 175 | APEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVEWESYGTEWSNYKTTPPVLDSDGSFFLYSKLT | |
| | VSKEEWQQGFVFSCSVMHEALHNHYTQKSLSLSPGK | |
| 176 | APELLGGPSVFLFPPKPKDTLYITREPEVTCVVVDVSHEDPEVKFNW | Clone CH3C.35.23.4 |
| | YVDGVEVHNAKTKPREEQYNSTYRVVSVLTVLHQDWLNGKEYKC | with knob and YTE |
| | KVSNKALPAPIEKISKAKGQPREPQVYTLPPSRDELTKNQVSLWCL | mutations |
| | VKGFYPSDIAVEWESYGTEWSNKTTPPVLDSDGSFFLYSKLTVSK | |
| | EEWQQGFVFSCSVMHEALHNHYTQKSLSLSPGK | |
| 177 | APEAAGGPSVFLFPPKPKDTLYITREPEVTCVVVDVSHEDPEVKFN | Clone CH3C.35.23.4 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSL | YTE mutations |
| | WCLVKGFYPSDIAVEWESYGTEWSNYKTTPPVLDSDGSFFLYSKLT | |
| | VSKEEWQQGFVFSCSVMHEALHNHYTQKSLSLSPGK | |
| 178 | APEAAGGPSVFLFPPKPKDTLYITREPEVTCVVVDVSHEDPEVKFN | Clone CH3C.35.23.4 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTE mutations |
| | WCLVKGFYPSDIAVEWESYGTEWSNYKTTPPVLDSDGSFFLYSKLT | |
| | VSKEEWQQGFVFSCSVMHEALHNHYTQKSLSLSPGK | |
| 179 | APELLGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with hole mutations |
| | KCKVSNKALPAPIEKTISKAKGQPREPQVYTLPPSRDELTKNQVSLS | |
| | CAVKGFYPSDIAVEWESYGTEWSNYKTTPPVLDSDGSFFLVSKLTV | |
| | SKEEWQQGFVFSCSVMHEALHNHYTQKSLSLSPGK | |
| 180 | APEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with hole and LALA |
| | KCKVSNKALPAPIEKTISKAKGQPREPQVYTLPPSRDELTKNQVSLS | mutations |
| | CAVKGFYPSDIAVEWESYGTEWSNYKTTPPVLDSDGSFFLYVSKLTV | |
| | SKEEWQQGFVFSCSVMHEALHNHYTQKSLSLSPGK | |
| 181 | APEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVEWESYGTEWSNYKTTPPVLDSDGSFFLVSKLTV | |
| | SKEEWQQGFVFSCSVMHEALHNHYTQKSLSLSPGK | |
| 182 | APELLGGPSVFLFPPKPKDTLYITREPEVTCVVVDVSHEDPEVKFNW | Clone CH3C.35.23.4 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESYGTEWSNYKTTPPVLDSDGSFFLVSKLTVSK | |
| | EEWQQGFVFSCSVMHEALHNHYTQKSLSLSPGK | |
| 183 | APEAAGGPSVFLFPPKPKDTLYITREPEVTCVVVDVSHEDPEVKFN | Clone CH3C.35.23.4 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTE mutations |
| | CAVKGFYPSDIAVEWESYGTEWSNYKTTPPVLDSDGSFFLVSKLTV | |
| | SKEEWQQGFVFSCSVMHEALHNHYTQKSLSLSPGK | |
| 184 | APEAAGGPSVFLFPPKPKDTLYITREPEVTCVVVDVSHEDPEVKFN | Clone CH3C.35.23.4 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTE mutations |
| | CAVKGFYPSDIAVEWESYGTEWSNYKTTPPVLDSDGSFFLVSKLTV | |
| | SKEEWQQGFVFSCSVMHEALHNHYTQKSLSLSPGK | |
| 185 | APELLGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with knob mutation |
| | KCKVSNKALPAPIEKTISKAKGQPREPQVYTLPPSRDELTKNQVSL | |
| | WCLVKGFYPSDIAVLWESYGTEWASYKTTPPVLDSDGSFFLYSKLT | |
| | VTKEEWQQGFVFSCSVMHEALHNHYTQKSLSLSPGK | |
| 186 | APEAAGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with knob and LALA |
| | KCKVSNKALPAPIEKTISKAKGQPREPQVYTLPPSRDELTKNQVSL | mutations |
| | WCLVKGFYPSDIAVLWESYGTEWASYKTTPPVLDSDGSFFLYSKLT | |
| | VTKEEWQQGFVFSCSVMHEALHNHYTQKSLSLSPGK | |
| 187 | APEAAGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with knob and LALAPG |
| | KCKVSNKALGAPIEKTISKAKGQPREPQVYTLPPSRDELTKNQVSL | mutations |
| | WCLVKGFYPSDIAVLWESYGTEWASYKTTPPVLDSDGSFFLYSKLT | |
| | VTKEEWQQGFVFSCSVMHEALHNHYTQKSLSLSPGK | |
| 188 | APELLGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVLWESYGTEWASYKTTPPVLDSDGSFFLYSKLTVTK | |
| | EEWQQGFVFSCSVMHEALHNHYTQKSLSLSPGK | |
| 189 | APEAAGGPSVFLFPPKPKDTLYITREPEVTCVVVDVSHEDPEVKFN | Clone CH3C.35.21.17.2 |
| | WYVDGVEVHNAKTKPREEQYNSTYRVVSVLTVLHQDWLNGKEY | with knob, LALA, and |
| | KCKVSNKALPAPIEKTISKAKGQPREPQVYTLPPSRDELTKNQVSL | YTE mutations |
| | WCLVKGFYPSDIAVLWESYGTEWASYKTTPPVLDSDGSFFLYSKLT | |
| | VTKEEWQQGFVFSCSVMHEALHNHYTQKSLSLSPGK | |
| 190 | APEAAGGPSVFLFPPKPKDTLYITREPEVTCVVVDVSHEDPEVKFN | Clone CH3C.35.21.17.2 |
| | WYVDGVEVHNAKTKPREEQYNSTYRVVSVLTVLHQDWLNGKEY | with knob, LALAPG, |
| | KCKVSNKALGAPIEKTISKAKGQPREPQVYTLPPSRDELTKNQVSL | and YTE mutations |
| | WCLVKGFYPSDIAVLWESYGTEWASYKTTPPVLDSDGSFFLYSKLT | |
| | VTKEEWQQGFVFSCSVMHEALHNHYTQKSLSLSPGK | |
| 191 | APELLGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with hole mutations |
| | KCKVSNKALPAPIEKTISKAKGQPREPQVYTLPPSRDELTKNQVSLS | |
| | CAVKGFYPSDIAVLWESYGTEWASYKTTPPVLDSDGSFFLVSKLTV | |
| | TKEEWQQGFVFSCSVMHEALHNHYTQKSLSLSPGK | |
| 192 | APEAAGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with hold and LALA |
| | KCKVSNKALPAPIEKTISKAKGQPREPQVYTLPPSRDELTKNQVSLS | mutations |
| | CAVKGFYPSDIAVLWESYGTEWASYKTTPPVLDSDGSFFLVSKLTV | |
| | TKEEWQQGFVFSCSVMHEALHNHYTQKSLSLSPGK | |
| 193 | APEAAGGPSVFLFPPKPKDTLMISRTPEVTCVVVDVSHEDPEVKFN | Clone CH3C.35.21.17.2 |
| | WYVDGVEVHNAKTKPREEQYNSTYRVVSVLTVLHQDWLNGKEY | with hole and LALAPG |
| | KCKVSNKALGAPIEKTISKAKGQPREPQVYTLPPSRDELTKNQVSLS | mutations |
| | CAVKGFYPSDIAVLWESYGTEWASYKTTPPVLDSDGSFFLVSKLTV | |
| | TKEEWQQGFVFSCSVMHEALHNHYTQKSLSLSPGK | |
| 194 | APELLGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVLWESYGTEWASYKTTPPVLDSDGSFFLVSKLTVTK | |
| | EEWQQGFVFSCSVMHEALHNHYTQKSLSLSPGK | |
| 195 | APEAAGGPSVFLFPPKPKDTLYITREPEVTCVVVDVSHEDPEVKFN | Clone CH3C.35.21.17.2 |
| | WYVDGVEVHNAKTKPREEQYNSTYRVVSVLTVLHQDWLNGKEY | with hole, LALA, and |
| | KCKVSNKALPAPIEKTISKAKGQPREPQVYTLPPSRDELTKNQVSLS | YTE mutations |
| | CAVKGFYPSDIAVLWESYGTEWASYKTTPPVLDSDGSFFLVSKLTV | |
| | TKEEWQQGFVFSCSVMHEALHNHYTQKSLSLSPGK | |
| 196 | APEAAGGPSVFLFPPKPKDTLYITREPEVTCVVVDVSHEDPEVKFN | Clone CH3C.35.21.17.2 |
| | WYVDGVEVHNAKTKPREEQYNSTYRVVSVLTVLHQDWLNGKEY | with hole, LALAPG, and |
| | KCKVSNKALGAPIEKTISKAKGQPREPQVYTLPPSRDELTKNQVSLS | YTE mutations |
| | CAVKGFYPSDIAVLWESYGTEWASYKTTPPVLDSDGSFFLVSKLTV | |
| | TKEEWQQGFVFSCSVMHEALHNHYTQKSLSLSPGK | |
| 197 | APELLGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | knob mutation |
| | KCKVSNKALPAPIEKTISKAKGQPREPQVYTLPPSRDELTKNQVSL | |
| | WCLVKGFYPSDIAVEWESYGTEWSNYKTTPPVLDSDGSFFLYSKLT | |
| | VTKEEWQQGFVFSCSVMHEALHNHYTQKSLSLSPGK | |
| 198 | APEAAGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | knob and LALA |
| | KCKVSNKALPAPIEKTISKAKGQPREPQVYTLPPSRDELTKNQVSL | mutations |
| | WCLVKGFYPSDIAVEWESYGTEWSNYKTTPPVLDSDGSFFLYSKLT | |
| | VTKEEWQQGFVFSCSVMHEALHNHYTQKSLSLSPGK | |
| 199 | APEAAGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | knob and LALAPG |
| | KCKVSNKALGAPIEKTISKAKGQPREPQVYTLPPSRDELTKNQVSL | mutations |
| | WCLVKGFYPSDIAVEWESYGTEWSNYKTTPPVLDSDGSFFLYSKLT | |
| | VTKEEWQQGFVFSCSVMHEALHNHYTQKSLSLSPGK | |
| 200 | APELLGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVTK | |
| | EEWQQGFVFSCSVMHEALHNHYTQKSLSLSPGK | |
| 201 | APEAAGGPSVFLFPPKPKDTLYITREPEVTCVVVDVSHEDPEVKFN | Clone CH3C.35.23 with |
| | WYVDGVEVHNAKTKPREEQYNSTYRVVSVLTVLHQDWLNGKEY | knob, LALA, and YTE |
| | KCKVSNKALPAPIEKTISKAKGQPREPQVYTLPPSRDELTKNQVSL | mutations |
| | WCLVKGFYPSDIAVEWESYGTEWSNYKTTPPVLDSDGSFFLYSKLT | |
| | VTKEEWQQGFVFSCSVMHEALHNHYTQKSLSLSPGK | |
| 202 | APEAAGGPSVFLFPPKPKDTLYITREPEVTCVVVDVSHEDPEVKFN | Clone CH3C.35.23 with |
| | WYVDGVEVHNAKTKPREEQYNSTYRVVSVLTVLHQDWLNGKEY | knob, LALAPG, and |
| | KCKVSNKALGAPIEKTISKAKGQPREPQVYTLPPSRDELTKNQVSL | YTE mutations |
| | WCLVKGFYPSDIAVEWESYGTEWSNYKTTPPVLDSDGSFFLYSKLT | |
| | VTKEEWQQGFVFSCSVMHEALHNHYTQKSLSLSPGK | |
| 203 | APELLGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | hole mutations |
| | KCKVSNKALPAPIEKTISKAKGQPREPQVYTLPPSRDELTKNQVSLS | |
| | CAVKGFYPSDIAVEWESYGTEWSNYKTTPPVLDSDGSFFLVSKLTV | |
| | TKEEWQQGFVFSCSVMHEALHNHYTQKSLSLSPGK | |
| 204 | APEAAGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | hole and LALA |
| | KCKVSNKALPAPIEKTISKAKGQPREPQVYTLPPSRDELTKNQVSLS | mutations |
| | CAVKGFYPSDIAVEWESYGTEWSNYKTTPPVLDSDGSFFLVSKLTV | |
| | TKEEWQQGFVFSCSVMHEALHNHYTQKSLSLSPGK | |
| 205 | APEAAGGPSVFLFPPKPKDTLMISRTPEVTCVVVDVSHEDPEVKFN | Clone CH3C.35.23 with |
| | WYVDGVEVHNAKTKPREEQYNSTYRVVSVLTVLHQDWLNGKEY | hole and LALAPG |
| | KCKVSNKALGAPIEKTISKAKGQPREPQVYTLPPSRDELTKNQVSLS | mutations |
| | CAVKGFYPSDIAVEWESYGTEWSNYKTTPPVLDSDGSFFLVSKLTV | |
| | TKEEWQQGFVFSCSVMHEALHNHYTQKSLSLSPGK | |
| 206 | APELLGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | hole and YTE mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVTK | |
| | EEWQQGFVFSCSVMHEALHNHYTQKSLSLSPGK | |
| 207 | APEAAGGPSVFLFPPKPKDTLYITREPEVTCVVVDVSHEDPEVKFN | Clone CH3C.35.23 with |
| | WYVDGVEVHNAKTKPREEQYNSTYRVVSVLTVLHQDWLNGKEY | hole, LALA, and YTE |
| | KCKVSNKALPAPIEKTISKAKGQPREPQVYTLPPSRDELTKNQVSLS | mutations |
| | CAVKGFYPSDIAVEWESYGTEWSNYKTTPPVLDSDGSFFLVSKLTV | |
| | TKEEWQQGFVFSCSVMHEALHNHYTQKSLSLSPGK | |
| 208 | APEAAGGPSVFLFPPKPKDTLYITREPEVTCVVVDVSHEDPEVKFN | Clone CH3C.35.23 with |
| | WYVDGVEVHNAKTKPREEQYNSTYRVVSVLTVLHQDWLNGKEY | hole, LALAPG, and |
| | KCKVSNKALGAPIEKTISKAKGQPREPQVYTLPPSRDELTKNQVSLS | YTE mutations |
| | CAVKGFYPSDIAVEWESYGTEWSNYKTTPPVLDSDGSFFLVSKLTV | |
| | TKEEWQQGFVFSCSVMHEALHNHYTQKSLSLSPGK | |
| 209 | DKTHTCPPCPAPEAAGGPSVFLFPPKPKDTLMISRTPEVTCVVVDVS | Clone CH3C.35.21.17.2 |
| | HEDPEVKFNWYVDGVEVHNAKTKPREEQYNSTYRVVSVLTVLHQ | with knob and LALA |
| | DWLNGKEYKCKVSNKALPAPIEKTISKAKGQPREPQVYTLPPSRDE | mutations and portion of |
| | LTKNQVSLWCLVKGFYPSDIAVLWESYGTEWASYKTTPPVLDSDG | human IgG1 hinge |
| | SFFLYSKLTVTKEEWQQGFVFSCSVMHEALHNHYTQKSLSLSPGK | sequence |
| 210 | DKTHTCPPCPAPEAAGGPSVFLFPPKPKDTLMISRTPEVTCVVVDVS | Clone CH3C.35.23.2 |
| | HEDPEVKFNWYVDGVEVHNAKTKPREEQYNSTYRVVSVLTVLHQ | with knob and LALA |
| | DWLNGKEYKCKVSNKALPAPIEKTISKAKGQPREPQVYTLPPSRDE | mutations and portion of |
| | LTKNQVSLWCLVKGFYPSDIAVEWESYGTEWANYKTTPPVLDSDG | human IgG1 hinge |
| | SFFLYSKLTVTKEEWQQGFVFSCSVMHEALHNHYTQKSLSLSPGK | sequence |
| 211 | MWTLVSWVALTAGLVAGTRCPDGQFCPVACCLDPGGASYSCCRP | pre-mature progranulin |
| | LLDKWPTTLSRHLGGPCQVDAHCSAGHSCIFTVSGTSSCCPFPEAV | (PGRN) polypeptide |
| | ACGDGHHCCPRGFHCSADGRSCFQRSGNNSVGAIQCPDSQFECPDF | |
| | STCCVMVDGSWGCCPMPQASCCEDRVHCCPHGAFCDLVHTRCITP | |
| | TGTHPLAKKLPAQRTNRAVALSSSVMCPDARSRCPDGSTCCELPSG | |
| | KYGCCPMPNATCCSDHLHCCPQDTVCDLIQSKCLSKENATTDLLTK | |
| | LPAHTVGDVKCDMEVSCPDGYTCCRLQSGAWGCCPFTQAVCCED | |
| | HIHCCPAGFTCDTQKGTCEQGPHQVPWMEKAPAHLSLPDPQALKR | |
| | DVPCDNVSSCPSSDTCCQLTSGEWGCCPIPEAVCCSDHQHCCPQGY | |
| | TCVAEGQCQRGSEIVAGLEKMPARRASLSHPRDIGCDQHTSCPVGQ | |
| | TCCPSLGGSWACCQLPHAVCCEDRQHCCPAGYTCNVKARSCEKEV | |
| | VSAQPATFLARSPHVGVKDVECGEGHFCHDNQTCCRDNRQGWAC | |
| | CPYRQGVCCADRRHCCPAGFRCAARGTKCLRREAPRWDAPLRDP | |
| | ALRQLL | |
| 212 | TRCPDGQFCPVACCLDPGGASYSCCRPLLDKWPTTLSRHLGGPCQV | mature PGRN |
| | DAHCSAGHSCIFTVSGTSSCCPFPEAVACGDGHHCCPRGFHCSADG | polypeptide |
| | RSCFQRSGNNSVGAIQCPDSQFECPDFSTCCVMVDGSWGCCPMPQ | |
| | ASCCEDRVHCCPHGAFCDLVHTRCITPTGTHPLAKKLPAQRTNRAV | |
| | ALSSSVMCPDARSRCPDGSTCCELPSGKYGCCPMPNATCCSDHLHC | |
| | CPQDTVCDLIQSKCLSKENATTDLLTKPAHTVGDVKCDMEVSCP | |
| | DGYTCCRLQSGAWGCCPFTQAVCCEDHIHCCPAGFTCDTQKGTC | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLL | |
| 213 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole |
| | DWLNGKEYKCKVSNKALPAPIEKTISKAKGQPREPQVYTLPPSRDE | mutations-(G₄S)₂-PGRN |
| | LTKNQVSLSCAVKGFYPSDIAVEWESNGQPENNYKTTPPVLDSDGS | |
| | FFLVSKLTVDKSRWQQGNVFSCSVMHEALHNHYTQKSLSLSPGKG | |
| | GGGSGGGGSTRCPDGQFCPVACCLDPGGASYSCCRPLLDKWPTTL | |
| | SRHLGGPCQVDAHCSAGHSCIFTVSGTSSCCPFPEAVACGDGHHCC | |
| | PRGFHCSADGRSCFQRSGNNSVGAIQCPDSQFECPDFSTCCVMVDG | |
| | SWGCCPMPQASCCEDRVHCCPHGAFCDLVHTRCITPTGTHPLAKK | |
| | LPAQRTNRAVALSSSVMCPDARSRCPDGSTCCELPSGKYGCCPMPN | |
| | ATCCSDHLHCCPQDTVCDLIQSKCLSKENATTDLLTKLPAHTVGDV | |
| | KCDMEVSCPDGYTCCRLQSGAWGCCPFTQAVCCEDHIHCCPAGFT | |
| | CDTQKGTCEQGPHQVPWMEKAPAHLSLPDPQALKRDVPCDNVSSC | |
| | PSSDTCCQLTSGEWGCCPIPEAVCCSDHQHCCPQGYTCVAEGQCQR | |
| | GSEIVAGLEKMPARRASLSHPRDIGCDQHTSCPVGQTCCPSLGGSW | |
| | ACCQLPHAVCCEDRQHCCPAGYTCNVKARSCEKEVVSAQPATFLA | |
| | RSPHVGVKDVECGEGHFCHDNQTCCRDNRQGWACCPYRQGVCCA | |
| | DRRHCCPAGFRCAARGTKCLRREAPRWDAPLRDPALRQLL | |
| 214 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole |
| | DWLNGKEYKCKVSNKALPAPIEKTISKAKGQPREPQVYTLPPSRDE | mutations-G₄S-PGRN |
| | LTKNQVSLSCAVKGFYPSDIAVEWESNGQPENNYKTTPPVLDSDGS | |
| | FFLVSKLTVDKSRWQQGNVFSCSVMHEALHNHYTQKSLSLSPGKG | |
| | GGGSTRCPDGQFCPVACCLDPGGASYSCCRPLLDKWPTTLSRHLG | |
| | GPCQVDAHCSAGHSCIFTVSGTSSCCPFPEAVACGDGHHCCPRGFH | |
| | CSADGRSCFQRSGNNSVGAIQCPDSQFECPDFSTCCVMVDGSWGC | |
| | CPMPQASCCEDRVHCCPHGAFCDLVHTRCITPTGTHPLAKKLPAQR | |
| | TNRAVALSSSVMCPDARSRCPDGSTCCELPSGKYGCCPMPNATCCS | |
| | DHLHCCPQDTVCDLIQSKCLSKENATTDLLTKPAHTVGDVKCDM | |
| | EVSCPDGYTCCRLQSGAWGCCPFTQAVCCEDHIHCCPAGFTCDTQ | |
| | KGTCEQGPHQVPWMEKAPAHLSLPDPQALKRDVPCDNVSSCPSSD | |
| | TCCQLTSGEWGCCPIPEAVCCSDHQHCCPQGYTCVAEGQCQRGSEI | |
| | VAGLEKMPARRASLSHPRDIGCDQHTSCPVGQTCCPSLGGSWACC | |
| | QLPHAVCCEDRQHCCPAGYTCNVKARSCEKEVVSAQPATFLARSP | |
| | HVGVKDVECGEGHFCHDNQTCCRDNRQGWACCPYRQGVCCADR | |
| | RHCCPAGFRCAARGTKCLRREAPRWDAPLRDPALRQLL | |
| 215 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole |
| | DWLNGKEYKCKVSNKALPAPIEKTISKAKGQPREPQVYTLPPSRDE | and LALA mutations- |
| | LTKNQVSLSCAVKGFYPSDIAVEWESNGQPENNYKTTPPVLDSDGS | (G₄S)₂-PGRN |
| | FFLVSKLTVDKSRWQQGNVFSCSVMHEALHNHYTQKSLSLSPGKG | |
| | GGGSGGGGSTRCPDGQFCPVACCLDPGGASYSCCRPLLDKWPTTL | |
| | SRHLGGPCQVDAHCSAGHSCIFTVSGTSSCCPFPEAVACGDGHHCC | |
| | PRGFHCSADGRSCFQRSGNNSVGAIQCPDSQFECPDFSTCCVMVDG | |
| | SWGCCPMPQASCCEDRVHCCPHGAFCDLVHTRCITPTGTHPLAKK | |
| | LPAQRTNRAVALSSSVMCPDARSRCPDGSTCCELPSGKYGCCPMPN | |
| | ATCCSDHLHCCPQDTVCDLIQSKCLSKENATTDLLTKPAHTVGDV | |
| | KCDMEVSCPDGYTCCRLQSGAWGCCPFTQAVCCEDHIHCCPAGFT | |
| | CDTQKGTCEQGPHQVPWMEKAPAHLSLPDPQALKRDVPCDNVSSC | |
| | PSSDTCCQLTSGEWGCCPIPEAVCCSDHQHCCPQGYTCVAEGQCQR | |
| | GSEIVAGLEKMPARRASLSHPRDIGCDQHTSCPVGQTCCPSLGGSW | |
| | ACCQLPHAVCCEDRQHCCPAGYTCNVKARSCEKEVVSAQPATFLA | |
| | RSPHVGVKDVECGEGHFCHDNQTCCRDNRQGWACCPYRQGVCCA | |
| | DRRHCCPAGFRCAARGTKCLRREAPRWDAPLRDPALRQLL | |
| 216 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole |
| | DWLNGKEYKCKVSNKALPAPIEKTISKAKGQPREPQVYTLPPSRDE | and LALA mutations- |
| | LTKNQVSLSCAVKGFYPSDIAVEWESNGQPENNYKTTPPVLDSDGS | G₄S-PGRN |
| | FFLVSKLTVDKSRWQQGNVFSCSVMHEALHNHYTQKSLSLSPGKG | |
| | GGGSTRCPDGQFCPVACCLDPGGASYSCCRPLLDKWPTTLSRHLG | |
| | GPCQVDAHCSAGHSCIFTVSGTSSCCPFPEAVACGDGHHCCPRGFH | |
| | CSADGRSCFQRSGNNSVGAIQCPDSQFECPDFSTCCVMVDGSWGC | |
| | CPMPQASCCEDRVHCCPHGAFCDLVHTRCITPTGTHPLAKKLPAQR | |
| | TNRAVALSSSVMCPDARSRCPDGSTCCELPSGKYGCCPMPNATCCS | |
| | DHLHCCPQDTVCDLIQSKCLSKENATTDLLTKLPAHTVGDVKCDM | |
| | EVSCPDGYTCCRLQSGAWGCCPFTQAVCCEDHIHCCPAGFTCDTQ | |
| | KGTCEQGPHQVPWMEKAPAHLSLPDPQALKRDVPCDNVSSCPSSD | |
| | TCCQLTSGEWGCCPIPEAVCCSDHQHCCPQGYTCVAEGQCQRGSEI | |
| | VAGLEKMPARRASLSHPRDIGCDQHTSCPVGQTCCPSLGGSWACC | |
| | QLPHAVCCEDRQHCCPAGYTCNVKARSCEKEVVSAQPATFLARSP | |
| | HVGVKDVECGEGHFCHDNQTCCRDNRQGWACCPYRQGVCCADR | |
| | RHCCPAGFRCAARGTKCLRREAPRWDAPLRDPALRQLL | |
| 217 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole |
| | DWLNGKEYKCKVSNKALGAPIEKTISKAKGQPREPQVYTLPPSRDE | and LALAPG mutations- |
| | LTKNQVSLSCAVKGFYPSDIAVEWESNGQPENNYKTTPPVLDSDGS | (G₄S)₂-PGRN |
| | FFLVSKLTVDKSRWQQGNVFSCSVMHEALHNHYTQKSLSLSPGKG | |
| | GGGSGGGGSTRCPDGQFCPVACCLDPGGASYSCCRPLLDKWPTTL | |
| | SRHLGGPCQVDAHCSAGHSCIFTVSGTSSCCPFPEAVACGDGHHCC | |
| | PRGFHCSADGRSCFQRSGNNSVGAIQCPDSQFECPDFSTCCVMVDG | |
| | SWGCCPMPQASCCEDRVHCCPHGAFCDLVHTRCITPTGTHPLAKK | |
| | LPAQRTNRAVALSSSVMCPDARSRCPDGSTCCELPSGKYGCCPMPN | |
| | ATCCSDHLHCCPQDTVCDLIQSKCLSKENATTDLLTKLPAHTVGDV | |
| | KCDMEVSCPDGYTCCRLQSGAWGCCPFTQAVCCEDHIHCCPAGFT | |
| | CDTQKGTCEQGPHQVPWMEKAPAHLSLPDPQALKRDVPCDNVSSC | |
| | PSSDTCCQLTSGEWGCCPIPEAVCCSDHQHCCPQGYTCVAEGQCQR | |
| | GSEIVAGLEKMPARRASLSHPRDIGCDQHTSCPVGQTCCPSLGGSW | |
| | ACCQLPHAVCCEDRQHCCPAGYTCNVKARSCEKEVVSAQPATFLA | |
| | RSPHVGVKDVECGEGHFCHDNQTCCRDNRQGWACCPYRQGVCCA | |
| | DRRHCCPAGFRCAARGTKCLRREAPRWDAPLRDPALRQLL | |
| 218 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole |
| | DWLNGKEYKCKVSNKALGAPIEKTISKAKGQPREPQVYTLPPSRDE | and LALAPG mutations- |
| | LTKNQVSLSCAVKGFYPSDIAVEWESNGQPENNYKTTPPVLDSDGS | G₄S-PGRN |
| | FFLVSKLTVDKSRWQQGNVFSCSVMHEALHNHYTQKSLSLSPGKG | |
| | GGGSTRCPDGQFCPVACCLDPGGASYSCCRPLLDKWPTTLSRHLG | |
| | GPCQVDAHCSAGHSCIFTVSGTSSCCPFPEAVACGDGHHCCPRGFH | |
| | CSADGRSCFQRSGNNSVGAIQCPDSQFECPDFSTCCVMVDGSWGC | |
| | CPMPQASCCEDRVHCCPHGAFCDLVHTRCITPTGTHPLAKKLPAQR | |
| | TNRAVALSSSVMCPDARSRCPDGSTCCELPSGKYGCCPMPNATCCS | |
| | DHLHCCPQDTVCDLIQSKCLSKENATTDLLTKLPAHTVGDVKCDM | |
| | EVSCPDGYTCCRLQSGAWGCCPFTQAVCCEDHIHCCPAGFTCDTQ | |
| | KGTCEQGPHQVPWMEKAPAHLSLPDPQALKRDVPCDNVSSCPSSD | |
| | TCCQLTSGEWGCCPIPEAVCCSDHQHCCPQGYTCVAEGQCQRGSEI | |
| | VAGLEKMPARRASLSHPRDIGCDQHTSCPVGQTCCPSLGGSWACC | |
| | QLPHAVCCEDRQHCCPAGYTCNVKARSCEKEVVSAQPATFLARSP | |
| | HVGVKDVECGEGHFCHDNQTCCRDNRQGWACCPYRQGVCCADR | |
| | RHCCPAGFRCAARGTKCLRREAPRWDAPLRDPALRQLL | |
| 219 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole |
| | DWLNGKEYKCKVSNKALPAPIEKTISKAKGQPREPQVYTLPPSRDE | and LS mutations- |
| | LTKNQVSLSCAVKGFYPSDIAVEWESNGQPENNYKTTPPVLDSDGS | (G₄S)₂-PGRN |
| | FFLVSKLTVDKSRWQQGNVFSCSVLHEALHSHYTQKSLSLSPGKGG | |
| | GGSGGGGSTRCPDGQFCPVACCLDPGGASYSCCRPLLDKWPTTLSR | |
| | HLGGPCQVDAHCSAGHSCIFTVSGTSSCCPFPEAVACGDGHHCCPR | |
| | GFHCSADGRSCFQRSGNNSVGAIQCPDSQFECPDFSTCCVMVDGS | |
| | WGCCPMPQASCCEDRVHCCPHGAFCDLVHTRCITPTGTHPLAKKL | |
| | PAQRTNRAVALSSSVMCPDARSRCPDGSTCCELPSGKYGCCPMPN | |
| | ATCCSDHLHCCPQDTVCDLIQSKCLSKENATTDLLTKLPAHTVGDV | |
| | KCDMEVSCPDGYTCCRLQSGAWGCCPFTQAVCCEDHIHCCPAGFT | |
| | CDTQKGTCEQGPHQVPWMEKAPAHLSLPDPQALKRDVPCDNVSSC | |
| | PSSDTCCQLTSGEWGCCPIPEAVCCSDHQHCCPQGYTCVAEGQCQR | |
| | GSEIVAGLEKMPARRASLSHPRDIGCDQHTSCPVGQTCCPSLGGSW | |
| | ACCQLPHAVCCEDRQHCCPAGYTCNVKARSCEKEVVSAQPATFLA | |
| | RSPHVGVKDVECGEGHFCHDNQTCCRDNRQGWACCPYRQGVCCA | |
| | DRRHCCPAGFRCAARGTKCLRREAPRWDAPLRDPALRQLL | |
| 220 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole |
| | DWLNGKEYKCKVSNKALPAPIEKTISKAKGQPREPQVYTLPPSRDE | and LS mutations-G₄S- |
| | LTKNQVSLSCAVKGFYPSDIAVEWESNGQPENNYKTTPPVLDSDGS | PGRN |
| | FFLVSKLTVDKSRWQQGNVFSCSVLHEALHSHYTQKSLSLSPGKGG | |
| | GGSTRCPDGQFCPVACCLDPGGASYSCCRPLLDKWPTTLSRHLGGP | |
| | CQVDAHCSAGHSCIFTVSGTSSCCPFPEAVACGDGHHCCPRGFHCS | |
| | ADGRSCFQRSGNNSVGAIQCPDSQFECPDFSTCCVMVDGSWGCCP | |
| | MPQASCCEDRHCCPHGAFCDLVHTRCITPTGTHPLAKKLPAQRTN | |
| | RAVALSSSVMCPDARSRCPDGSTCCELPSGKYGCCPMPNATCCSD | |
| | HLHCCPQDTVCDLIQSKCLSKENATTDLLTKLPAHTVGDVKCDME | |
| | VSCPDGYTCCRLQSGAWGCCPFTQAVCCEDHIHCCPAGFTCDTQK | |
| | GTCEQGPHQVPWMEKAPAHLSLPDPQALKRDVPCDNVSSCPSSDT | |
| | CCQLTSGEWGCCPIPEAVCCSDHQHCCPQGYTCVAEGQCQRGSEIV | |
| | AGLEKMPARRASLSHPRDIGCDQHTSCPVGQTCCPSLGGSWACCQ | |
| | LPHAVCCEDRQHCCPAGYTCNVKARSCEKEVVSAQPATFLARSPH | |
| | VGVKDVECGEGHFCHDNQTCCRDNRQGWACCPYRQGVCCADRR | |
| | HCCPAGFRCAARGTKCLRREAPRWDAPLRDPALRQLL | |
| 221 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTRVVSVLTVLHQ | polypeptide with hole |
| | DWLNGKEYKCKVSNKALPAPIEKTISKAKGQPREPQVYTLPPSRDE | LALA, and LS |
| | LTKNQVSLSLCAVKGFYPSDIAVEWESNGQPENNYKTTPPVLDSDGS | mutations-(G₄S)₂-PGRN |
| | FFLVSKLTVDKSRWQQGNVFSCSVLHEALHSHYTQKSLSLSPGKGG | |
| | GGSGGGGSTRCPDGQFCPVACCLDPGGASYSCCRPLLDKWPTTLSR | |
| | HLGGPCQVDAHCSAGHSCIFTVSGTSSCCPFPEAVACGDGHHCCPR | |
| | GFHCSADGRSCFQRSGNNSVGAIQCPDSQFECPDFSTCCVMVDGS | |
| | WGCCPMPQASCCEDRVHCCPHGAFCDLVHTRCITPTGTHPLAKKL | |
| | PAQRTNRAVALSSSVMCPDARSRCPDGSTCCELPSGKYGCCPMPN | |
| | ATCCSDHLHCCPQDTVCDLIQSKCLSKENATTDLLTKLPAHTVGDV | |
| | KCDMEVSCPDGYTCCRLQSGAWGCCPFTQAVCCEDHIHCCPAGFT | |
| | CDTQKGTCEQGPHQVPWMEKAPAHLSLPDPQALKRDVPCDNVSSC | |
| | PSSDTCCQLTSGEWGCCPIPEAVCCSDHQHCCPGQGYTCVAEGQCQR | |
| | GSEIVAGLEKMPARRASLSHPRDIGCDQHTSCPVGQTCCPSLGGSW | |
| | ACCQLPHAVCCEDRQHCCPAGYTCNVKARSCEKEVVSAQPATFLA | |
| | RSPHVGVKDVECGEGHFCHDNQTCCRDNRQGWACCPYRQGVCCA | |
| | DRRHCCPAGFRCAARGTKCLRREAPRWDAPLRDPALRQLL | |
| 222 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole, |
| | DWLNGKEYKCKVSNKALPAPIEKTISKAKGQPREPQVYTLPPSRDE | LALA, and LS |
| | LTKNQVSLSCAVKGFYPSDIAVEWESNGQPENNYKTTPPVLDSDGS | mutations-G₄S-PGRN |
| | FFLVSKLTVDKSRWQQGNVFSCSVLHEALHSHYTQKSLSLSPGKGG | |
| | GGSTRCPDGQFCPVACCLDPGGASYSCCRPLLDKWPTTLSRHLGGP | |
| | CQVDAHCSAGHSCIFTVSGTSSCCPFPEAVACGDGHHCCPRGFHCS | |
| | ADGRSCFQRSGNNSVGAIQCPDSQFECPDFSTCCVMVDGSWGCCP | |
| | MPQASCCEDRVHCCPHGAFCDLVHTRCITPTGTHPLAKKLPAQRTN | |
| | RAVALSSSVMCPDARSRCPDGSTCCELPSGKYGCCPMPNATCCSD | |
| | HLHCCPQDTVCDLIQSKCLSKENATTDLLTKLPAHTVGDVKCDME | |
| | VSCPDGYTCCRLQSGAWGCCPFTQAVCCEDHIHCCPAGFTCDTQK | |
| | GTCEQGPHQVPWMEKAPAHLSLPDPQALKRDVPCDNVSSCPSSDT | |
| | CCQLTSGEWGCCPIPEAVCCSDHQHCCPQGYTCVAEGQCQRGSEIV | |
| | AGLEKMPARRASLSHPRDIGCDQHTSCPVGQTCCPSLGGSWACCQ | |
| | LPHAVCCEDRQHCCPAGYTCNVKARSCEKEVVSAQPATFLARSPH | |
| | VGVKDVECGEGHFCHDNQTCCRDNRQGWACCPYRQGVCCADRR | |
| | HCCPAGFRCAARGTKCLRREAPRWDAPLRDPALRQLL | |
| 223 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole, |
| | DWLNGKEYKCKVSNKALGAPIEKTISKAKGQPREPQVYTLPPSRDE | LALAPG, and LS |
| | LTKNQVSLSCAVKGFYPSDIAVEWESNGQPENNYKTTPPVLDSDGS | mutations-(G₄S)₂-PGRN |
| | FFLVSKLTVDKSRWQQGNVFSCSVLHEALHSHYTQKSLSLSPGKGG | |
| | GGSGGGGSTRCPDGQFCPVACCLDPGGASYSCCRPLLDKWPTTLSR | |
| | HLGGPCQVDAHCSAGHSCIFTVSGTSSCCPFPEAVACGDGHHCCPR | |
| | GFHCSADGRSCFQRSGNNSVGAIQCPDSQFECPDFSTCCVMVDGS | |
| | WGCCPMPQASCCEDRVHCCPHGAFCDLVHTRCITPTGTHPLAKKL | |
| | PAQRTNRAVALSSSVMCPDARSRCPDGSTCCELPSGKYGCCPMPN | |
| | ATCCSDHLHCCPQDTVCDLIQSKCLSKENATTDLLTKLPAHTVGDV | |
| | KCDMEVSCPDGYTCCRLQSGAWGCCPFTQAVCCEDHIHCCPAGFT | |
| | CDTQKGTCEQGPHQVPWMEKAPAHLSLPDPQALKRDVPCDNVSSC | |
| | PSSDTCCQLTSGEWGCCPIPEAVCCSDHQHCCPQGYTCVAEGQCQR | |
| | GSEIVAGLEKMPARRASLSHPRDIGCDQHTSCPVGQTCCPSLGGSW | |
| | ACCQLPHAVCCEDRQHCCPAGYTCNVKARSCEKEVVSAQPATFLA | |
| | RSPHVGVKDVECGEGHFCHDNQTCCRDNRQGWACCPYRQGVCCA | |
| | DRRHCCPAGFRCAARGTKCLRREAPRWDAPLRDPALRQLL | |
| 224 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole, |
| | DWLNGKEYKCKVSNKALGAPIEKTISKAKGQPREPQVYTLPPSRDE | LALAPG, and LS |
| | LTKNQVSLSCAVKGFYPSDIAVEWESNGQPENNYKTTPPVLDSDGS | mutations-G₄S-PGRN |
| | FFLVSKLTVDKSRWQQGNVFSCSVLHEALHSHYTQKSLSLSPGKGG | |
| | GGSTRCPDGQFCPVACCLDPGGASYSCCRPLLDKWPTTLSRHLGGP | |
| | CQVDAHCSAGHSCIFTVSGTSSCCPFPEAVACGDGHHCCPRGFHCS | |
| | ADGRSCFQRSGNNSVGAIQCPDSQFECPDFSTCCVMVDGSWGCCP | |
| | MPQASCCEDRVHCCPHGAFCDLVHTRCITPTGTHPLAKKLPAQRTN | |
| | RAVALSSSVMCPDARSRCPDGSTCCELPSGKYGCCPMPNATCCSD | |
| | HLHCCPQDTVCDLIQSKCLSKENATTDLLTKLPAHTVGDVKCDME | |
| | VSCPDGYTCCRLQSGAWGCCPFTQAVCCEDHIHCCPAGFTCDTQK | |
| | GTCEQGPHQVPWMEKAPAHLSLPDPQALKRDVPCDNVSSCPSSDT | |
| | CCQLTSGEWGCCPIPEAVCCSDHQHCCPQGYTCVAEGQCQRGSEIV | |
| | AGLEKMPARRASLSHPRDIGCDQHTSCPVGQTCCPSLGGSWACCQ | |
| | LPHAVCCEDRQHCCPAGYTCNVKARSCEKEVVSAQPATFLARSPH | |
| | VGVKDVECGEGHFCHDNQTCCRDNRQGWACCPYRQGVCCADRR | |
| | HCCPAGFRCAARGTKCLRREAPRWDAPLRDPALRQLL | |
| 225 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole mutations |
| | ASCCEDRVHCCPHGAFCDLVHTRCITPTGTHPLAKKLPAQRTNRAV | |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALPAPIEKTISKAKGQPREPQVYTLPPSRDELTK | |
| | NQVSLSCAVKGFYPSDIAVEWESNGQPENNYKTTPPVLDSDGSFFL | |
| | VSKLTVDKSRWQQGNVFSCSVMHEALHNHYTQKSLSLSPGK | |
| 226 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole mutations |
| | ASCCEDRVHCCPHGAFCDLVHTRCITPTGTHPLAKKLPAQRTNRAV | |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALPAPIEKTISKAKGQPREPQVYTLPPSRDELTKNQVSL | |
| | SCAVKGFYPSDIAVEWESNGQPENNYKTTPPVLDSDGSFFLVSKLT | |
| | VDKSRWQQGNVFSCSVMHEALHNHYTQKSLSLSPGK | |
| 227 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole and LALA |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALPAPIEKTISKAKGQPREPQVYTLPPSRDELTK | |
| | NQVSLSCAVKGFYPSDIAVEWESNGQPENNYKTTPPVLDSDGSFFL | |
| | VSKLTVDKSRWQQGNVFSCSVMHEALHNHYTQKSLSLSPGK | |
| 228 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole and LALA |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPEAAGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALPAPIEKTISKAKGQPREPQVYTLPPSRDELTKNQVSL | |
| | SCAVKGFYPSDIAVEWESNGQPENNYKTTPPVLDSDGSFFLVSKLT | |
| | VDKSRWQQGNVFSCSVMHEALHNHYTQKSLSLSPGK | |
| 229 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole and LALAPG |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALGAPIEKTISKAKGQPREPQVYTLPPSRDELTK | |
| | NQVSLSCAVKGFYPSDIAVEWESNGQPENNYKTTPPVLDSDGSFFL | |
| | VSKLTVDKSRWQQGNVFSCSVMEALHNHYTQKSLSLSPGK | |
| 230 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole and LALAPG |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPEAAGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALGAPIEKTISKAKGQPREPQVYTLPPSRDELTKNQVS | |
| | LSCAVKGFYPSDIAVEWESNGQPENNYKTTPPVLDSDGSFFLVSKL | |
| | TVDKSRWQQGNVFSCSVMHEALHNHYTQKSLSLSPGK | |
| 231 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole and LS |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALPAPIEKTISKAKGQPREPQVYTLPPSRDELTK | |
| | NQVSLSCAVKGFYPSDIAVEWESNGQPENNYKTTPPVLDSDGSFFL | |
| | VSKLTVDKSRWQQGNVFSCSVLHEALHSHYTQKSLSLSPGK | |
| 232 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole and LS |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALPAPIEKTISKAKGQPREPQVYTLPPSRDELTKNQVSL | |
| | SCAVKGFYPSDIAVEWESNGQPENNYKTTPPVLDSDGSFFLVSKLT | |
| | VDKSRWQQGNVFSCSVLHEALHSHYTQKSLSLSPGK | |
| 233 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole, LALA, and |
| | ASCCEDRVHCCPHGAFCDLVHTRCITPTGTHPLAKKLPAQRTNRAV | LS mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALPAPIEKTISKAKGQPREPQVYTLPPSRDELTK | |
| | NQVSLSCAVKGFYPSDIAVEWESNGQPENNYKTTPPVLDSDGSFFL | |
| | VSKLTVDKSRWQQGNVFSCSVLHEALHSHYTQKSLSLSPGK | |
| 234 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole, LALA, and |
| | ASCCEDRVHCCPHGAFCDLVHTRCITPTGTHPLAKKLPAQRTNRAV | LS mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPEAAGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALPAPIEKTISKAKGQPREPQVYTLPPSRDELTKNQVSL | |
| | SCAVKGFYPSDIAVEWESNGQPENNYKTTPPVLDSDGSFFLVSKLT | |
| | VDKSRWQQGNVFSCSVLHEALHSHYTQKSLSLSPGK | |
| 235 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole, LALAPG, and |
| | ASCCEDRVHCCPHGAFCDLVHTRCITPTGTHPLAKKLPAQRTNRAV | LS mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALGAPIEKTISKAKGQPREPQVYTLPPSRDELTK | |
| | NQVSLSCAVKGFYPSDIAVEWESNGQPENNYKTTPPVLDSDGSFFL | |
| | VSKLTVDKSRWQQGNVFSCSVLHEALHSHYTQKSLSLSPGK | |
| 236 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with hole, LALAPG, and |
| | ASCCEDRVHCCPHGAFCDLVHTRCITPTGTHPLAKKLPAQRTNRAV | LS mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPEAAGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALGAPIEKTISKAKGQPREPQVYTLPPSRDELTKNQVS | |
| | LSCAVKGFYPSDIAVEWESNGQPENNYKTTPPVLDSDGSFFLVSKL | |
| | TVDKSRWQQGNVFSCSVLHEALHSHYTQKSLSLSPGK | |
| 237 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with knob |
| | DWLNGKEYKCKVSNKALPAPIEKTISKAKGQPREPQVYTLPPSRDE | mutations-(G₄S)₂-PGRN |
| | LTKNQVSLWCLVKGFYPSDIAVEWESNGQPENNYKTTPPVLDSDG | |
| | SFFLYSKLTVDKSRWQQGNVFSCSVMHEALHNHYTQKSLSLSPGK | |
| | GGGGSGGGGSTRCPDGQFCPVACCLDPGGASYSCCRPLLDKWPTT | |
| | LSRHLGGPCQVDAHCSAGHSCIFTVSGTSSCCPFPEAVACGDGHHC | |
| | CPRGFHCSADGRSCFQRSGNNSVGAIQCPDSQFECPDFSTCCVMVD | |
| | GSWGCCPMPQASCCEDRVHCCPHGAFCDLVHTRCITPTGTHPLAK | |
| | KLPAQRTNRAVALSSSVMCPDARSRCPDGSTCCELPSGKYGCCPMP | |
| | NATCCSDHLHCCPQDTVCDLIQSKCLSKENATTDLLTKLPAHTVGD | |
| | VKCDMEVSCPDGYTCCRLQSGAWGCCPFTQAVCCEDHIHCCPAGF | |
| | TCDTQKGTCEQGPHQVPWMEKAPAHLSLPDPQALKRDVPCDNVSS | |
| | CPSSDTCCQLTSGEWGCCPIPEAVCCSDHQHCCPQGYTCVAEGQCQ | |
| | RGSEIVAGLEKMPARRASLSHPRDIGCDQHTSCPVGQTCCPSLGGS | |
| | WACCQLPHAVCCEDRQHCCPAGYTCNVKARSCEKEVVSAQPTAF | |
| | LARSPHVGVKDVECGEGHFCHDNQTCCRDNRQGWACCPYRQGVC | |
| | CADRRHCCPAGFRCAARGTKCLRREAPRWDAPLRDPALRQLL | |
| 238 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with knob |
| | DWLNGKEYKCKVSNKALPAPIEKTISKAKGQPREPQVYTLPPSRDE | mutations-G₄S-PGRN |
| | LTKNQVSLWCLVKGFYPSDIAVEWESNGQPENNYKTTPPVLDSDG | |
| | SFFLYSKLTVDKSRWQQGNVFSCSVMHEALHNHYTQKSLSLSPGK | |
| | GGGGSTRCPDGQFCPVACCLDPGGASYSCCRPLLDKWPTTLSRHL | |
| | GGPCQVDAHCSAGHSCIFTVSGTSSCCPFPEAVACGDGHHCCPRGF | |
| | HCSADGRSCFQRSGNNSVGAIQCPDSQFECPDFSTCCVMVDGSWG | |
| | CCPMPQASCCEDRVHCCPHGAFCDLVHTRCITPTGTHPLAKKLPAQ | |
| | RTNRAVALSSSVMCPDARSRCPDGSTCCELPSGKYGCCPMPNATC | |
| | CSDHLHCCPQDTVCDLIQSKCLSKENATTDLLTKLPAHTVGDVKCD | |
| | MEVSCPDGYTCCRLQSGAWGCCPFTQAVCCEDHIHCCPAGFTCDT | |
| | QKGTCEQGPHQVPWMEKAPAHLSLPDPQALKRDVPCDNVSSCPSS | |
| | DTCCQLTSGEWGCCPIPEAVCCSDHQHCCPQGYTCVAEGQCQRGS | |
| | EIVAGLEKMPARRASLSHPRDIGCDQHTSCPVGQTCCPSLGGSWAC | |
| | CQLPHAVCCEDRQHCCPAGYTCNVKARSCEKEVVSAQPATFLARS | |
| | PHVGVKDVECGEGHFCHDNQTCCRDNRQGWACCPYRQGVCCAD | |
| | RRHCCPAGFRCAARGTKCLRREAPRWDAPLRDPALQLL | |
| 239 | DKTHTCPPCPAPEAAGGPSVFLFPPKPKDTLMISRTPEVTCVVVDVS | Partial hinge-Fc |
| | HEDPEVKFNWYVDGVEVHANKTKPREEQYNSTYRVVSVLTVLHQ | polypeptide with knob |
| | DWLNGKEYKCKVSNKALPAPIEKTISKAKGQPREPQVYTLPPSRDE | and LALA mutations- |
| | LTKNQVSLWCLVKGFYPSDIAVEWESNGQPENNYKTTPPVLDSDG | (G₄S)₂-PGRN |
| | SFFLYSKLTVDKSRWQQGNVFSCSVMHEALHNHYTQKSLSLSPGK | |
| | GGGGSGGGGSTRCPDGQFCPVACCLDPGGASYSCCRPLLDKWPTT | |
| | LSRHLGGPCQVDAHCSAGHSCIFTVSGTSSCCPFPEAVACGDGHHC | |
| | CPRGFHCSADGRSCFQRSGNNSVGAIQCPDSQFECPDFSTCCVMVD | |
| | GSWGCCPMPQASCCEDRVHCCPHGAFCDLVHTRCITPTGTHPLAK | |
| | KLPAQRTNRAVALSSSVMCPDARSRCPDGSTCCELPSGKYGCCPMP | |
| | NATCCSDHLHCCPQDTVCDLIQSKCLSKENATTDLLTKLPAHTVGD | |
| | VKCDMEVSCPDGYTCCRLQSGAWGCCPFTQAVCCEDHIHCCPAGF | |
| | TCDTQKGTCEQGPHQVPWMEKAPAHLSLPDPQALKRDVPCDNVSS | |
| | CPSSDTCCQLTSGEWGCCPIPEAVCCSDHQHCCPQGYTCVAEGQCQ | |
| | RGSEIVAGLEKMPARRASLSHPRDIGCDQHTSCPVGQTCCPSLGGS | |
| | WACCQLPHAVCCEDRQHCCPAGYTCNVKARSCEKEVVSAQPATF | |
| | LARSPHVGVKDVECGEGHFCHDNQTCCRDNRQGWACCPYRQGVC | |
| | CADRRHCCPAGFRCAARGTKCLRREAPRWDAPLRDPALRQLL | |
| 240 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with knob |
| | DWLNGKEYKCKVSNKALPAPIEKTISKAKGQPREPQVYTLPPSRDE | and LALA mutations- |
| | LTKNQVSLWCLVKGFYPSDIAVEWESNGQPENNYKTTPPVLDSDG | G₄S-PGRN |
| | SFFLYSKLTVDKSRWQQGNVFSCSVMHEALHNHYTQKSLSLSPGK | |
| | GGGGSTRCPDGQFCPVACCLDPGGASYSCCRPLLDKWPTTLSRHL | |
| | GGPCQVDAHCSAGHSCIFTVSGTSSCCPFPEAVACGDGHHCCPRGF | |
| | HCSADGRSCFQRSGNNSVGAIQCPDSQFECPDFSTCCVMVDGSWG | |
| | CCPMPQASCCEDRVHCCPHGAFCDLVHTRCITPTGTHPLAKKLPAQ | |
| | RTNRAVALSSSVMCPDARSRCPDGSTCCELPSGKYGCCPMPNATC | |
| | CSDHLHCCPQDTVCDLIQSKCLSKENATTDLLTKLPAHTVGDVKCD | |
| | MEVSCPDGYTCCRLQSGAWGCCPFTQAVCCEDHIHCCPAGFTCDT | |
| | QKGTCEQGPHQVPWMEKAPAHLSLPDPQALKRDVPCDNVSSCPSS | |
| | DTCCQLTSGEWGCCPIPEAVCCSDHQHCCPQGYTCVAEGQCQRGS | |
| | EIVAGLEKMPARRASLSHPRDIGCDQHTSCPVGQTCCPSLGGSWAC | |
| | CQLPHAVCCEDRQHCCPAGYTCNVKARSCEKEVVSAQPATFLARS | |
| | PHVGVKDVECGEGHFCHDNQTCCRDNRQGWACCPYRQGVCCAD | |
| | RRHCCPAGFRCAARGTKCLRREAPRWDAPLRDPALRQLL | |
| 241 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with knob |
| | DWLNGKEYKCKVSNKALGAPIEKTISKAKGQPREPQVYTLPPSRDE | and LALAPG mutations- |
| | LTKNQVSLWCLVKGFYPSDIAVEWESNGQPENNYKTTPPVLDSDG | (G₄S)₂-PGRN |
| | SFFLYSKLTVDKSRWQQGNVFSCSVMHEALHNHYTQKSLSLSPGK | |
| | GGGGSGGGGSTRCPDGQFCPVACCLDPGGASYSCCRPLLDKWPTT | |
| | LSRHLGGPCQVDAHCSAGHSCIFTVSGTSSCCPFPEAVACGDGHHC | |
| | CPRGFHCSADGRSCFQRSGNNSVGAIQCPDSQFECPDFSTCCVMVD | |
| | GSWGCCPMPQASCCEDRVHCCPHGAFCDLVHTRCITPTGTHPLAK | |
| | KLPAQRTNRAVALSSSVMCPDARSRCPDGSTCCELPSGKYGCCPMP | |
| | NATCCSDHLHCCPQDTVCDLIQSKCLSKENATTDLLTKLPAHTVGD | |
| | VKCDMEVSCPDGYTCCRLQSGAWGCCPFTQAVCCEDHIHCCPAGF | |
| | TCDTQKGTCEQGPHQVPWMEKAPAHLSLPDPQALKRDVPCDNVSS | |
| | CPSSDTCCQLTSGEWGCCPIPEAVCCSDHQHCCPQGYTCVAEGQCQ | |
| | RGSEIVAGLEKMPARRASLSHPRDIGCDQHTSCPVGQTCCPSLGGS | |
| | WACCQLPHAVCCEDRQHCCPAGYTCNVKARSCEKEVVSAQPATF | |
| | LARSPHVGVKDVECGEGHFCHDNQTCCRDNRQGWACCPYRQGVC | |
| | CADRRHCCPAGFRCAARGTKCLRREAPRWDAPLRDPALRQLL | |
| 242 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with knob |
| | DWLNGKEYKCKVSNKALGAPIEKTISKAKQPREPQVYTLPPSRDE | and LALAPG mutations- |
| | LTKNQVSLWCLVKGFYPSDIAVEWESNGQPENNYKTTPPVLDSDG | G₄S-PGRN |
| | SFFLYSKLTVDKSRWQQGNVFSCSVMHEALHNHYTQKSLSLSPGK | |
| | GGGGSTRCPDGQFCPVACCLDPGGASYSCCRPLLDKWPTTLSRHL | |
| | GGPCQVDAHCSAGHSCIFTVSGTSSCCPFPEAVACGDGHHCCPRGF | |
| | HCSADGRSCFQRSGNNSVGAIQCPDSQFECPDFSTCCVMVDGSWG | |
| | CCPMPQASCCEDRVHCCPHGAFCDLVHTRCITPTGTHPLAKKLPAQ | |
| | RTNRAVALSSSVMCPDARSRCPDGSTCCELPSGKYGCCPMPNATC | |
| | CSDHLHCCPQDTVCDLIQSKCLSKENATTDLLTKPLAHTVGDVKCD | |
| | MEVSCPDGYTCCRLQSGAWGCCPFTQAVCCEDHIHCCPAGFTCDT | |
| | QKGTCEQGPHQVPWMEKAPAHLSLPDPQALKRDVPCDNVSSCPSS | |
| | DTCCQLTSGEWGCCPIPEAVCCSDHQHCCPQGYTCVAEGQCQRGS | |
| | EIVAGLEKMPARRASLSHPRDIGCDQHTSCPVGQTCCPSLGGSWAC | |
| | CQLPAHVCCEDRQHCCPAGYTCNVKARSCEKEVVSAQPATFLARS | |
| | PHVGVKDVECGEGHFCHDNQTCCRDNRQGWACCPYRQGVCCAD | |
| | RRHCCPAGFRCAARGTKCLRREAPRWDAPLRDPALQLL | |
| 246 | DKTHTCPPCPAPEAAGGPSVFLFPPKPKDTLMISRTPEVTCVVVDVS | Partial hinge-Fc |
| | HEDPEVKFNWYVDGVEVHNAKTKTKPREEQYNSTYRVVSVLTVLHQ | polypeptide with knob, |
| | DWLNGKEYKCKVSNKALPAPIEKTISKAKGQPREPQVYTLPPSRDE | LALA, and LS |
| | LTKNQVSLWCLVKGFYPSDIAVEWESNGQPENNYKTTPPVLDSDG | mutations-G₄S-PRGN |
| | SFFLYSKLTVDKSRWQQGNVFSCSVLHEALHSHYTQKSLSLSPGKG | |
| | GGGSTRCPDGQFCPVACCLDPGGASYSCCRPLLDKWPTTLSRHLG | |
| | GPCQVDAHCSAGHSCIFTVSGTSSCCPFPEAVACGDGHHCCPRGFH | |
| | CSADGRSCFQRSGNNSVGAIQCPDSQFECPDFSTCCVMVDGSWGC | |
| | CPMPQASCCEDRVHCCPHGAFCDLVHTRCITPTGTHPLAKKLPAQR | |
| | TNRAVALSSSVMCPDARSRCPDGSTCCELPSGKYGCCPMPNATCCS | |
| | DHLHCCPQDTVCDLIQSKCLSKENATTDLLTKLPAHTVGDVKCDM | |
| | EVSCPDGYTCCRLQSGAWGCCPFTQAVCCEDHIHCCPAGFTCDTQ | |
| | KGTCEQGPHQVPWMEKAPAHLSLPDPQALKRDVPCDNVSSCPSSD | |
| | TCCQLTSGEWGCCPIPEAVCCSDHQHCCPQGYTCVAEGQCQRGSEI | |
| | VAGLEKMPARRASLSHPRDIGCDQHTSCPVGQTCCPSLGGSWACC | |
| | QLPHAVCCEDRQHCCPAGYTCNVKARSCEKEVVSAQPATFLARSP | |
| | HVGVKDVECGEGHFCHDNQTCCRDNRQGWACCPYRQGVCCADR | |
| | RHCCPAGFRCAARGTKCLRREAPRWDAPLRDPALRQLL | |
| 247 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with knob, |
| | DWLNGKEYKCKVSNKALGAPIEKTISKAKGQPREPQVYTLPPSRDE | LALAPG, and LS |
| | LTKNQVSLWCLVKGFYPSDIAVEWESNGQPENNYKTTPPVLDSDG | mutations-(G₄S)₂-PGRN |
| | SFFLYSKLTVDKSRWQQGNVFSCSVLHEALHSHYTQKSLSLSPGKG | |
| | GGGSGGGGSTRCPDGQFCPVACCLDPGGASYSCCRPLLDKWPTTL | |
| | SRHLGGPCQVDAHCSAGHSCIFTVSGTSSCCPFPEAVACGDGHHCC | |
| | PRGFHCSADGRSCFQRSGNNSVGAIQCPDSQFECPDFSTCCVMVDG | |
| | SWGCCPMPQASCCEDRVHCCPHGAFCDLVHTRCITPTGTHPLAKK | |
| | LPAQRTNRAVALSSSVMCPDARSRCPDGSTCCELPSGKYGCCPMPN | |
| | ATCCSDHLHCCPQDTVCDLIQSKCLSKENATTDLLTKLPAHTVGDV | |
| | KCDMEVSCPDGYTCCRLQSGAWGCCPFTQAVCCEDHIHCCPAGFT | |
| | CDTQKGTCEQGPHQVPWMEKAPAHLSLPDPQALKRDVPCDNVSSC | |
| | PSSDTCCQLTSGEWGCCPIPEAVCCSDHQHCCPQGYTCVAEGQCQR | |
| | GSEIVAGLEKMPARRASLSHPRDIGCDQHTSCPVGQTCCPSLGGSW | |
| | ACCQLPHAVCCEDRQHCCPAGYTCNVKARSCEKEVVSAQPATFLA | |
| | RSPHVGVKDVECGEGHFCHDNQTCCRDNRQGWACCPYRQGVCCA | |
| | DRRHCCPAGFRCAARGTKCLRREAPRWDAPLRDPALRQLL | |
| 248 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with knob, |
| | DWLNGKEYKCKVSNKALGAPIEKTISKAKGQPREPQVYTLPPSRDE | LALAPG, and LS |
| | LTKNQVSLWCLVKGFYPSDIAVEWESNGQPENNYKTTPPVLDSDG | mutations-G₄S-PGRN |
| | SFFLYSKLTVDKSRWQQGNVFSCSVLHEALHSHYTQKSLSLSPGKG | |
| | GGGSTRCPDGQFCPVACCLDPGGASYSCCRPLLDKWPTTLSRHLG | |
| | GPCQVDAHCSAGHSCIFTVSGTSSCCPFPEAVACGDGHHCCPRGFH | |
| | CSADGRSCFQRSGNNSVGAIQCPDSQFECPDFSTCCVMVDGSWGC | |
| | CPMPQASCCEDRVHCCPHGAFCDLVHTRCITPTGTHPLAKKLPAQR | |
| | TNRAVALSSSVMCPDARSRCPDGSTCCELPSGKYGCCPMPNATCCS | |
| | DHLHCCPQDTVCDLIQSKCLSKENATTDLLTKLPAHTVGDVKCDM | |
| | EVSCPDGYTCCRLQSGAWGCCPFTQAVCCEDHIHCCPAGFTCDTQ | |
| | KGTCEQGPHQVPWMEKAPAHLSLPDPQALKRDVPCDNVSSCPSSD | |
| | TCCQLTSGEWGCCPIPEAVCCSDHQHCCPQGYTCVAEGQCQRGSEI | |
| | VAGLEKMPARRASLSHPRDIGCDQHTSCPVGQTCCPSLGGSWACC | |
| | QLPHAVCCEDRQHCCPAGYTCNVKARSCEKEVVSAQPATFLARSP | |
| | HVGVKDVECGEGHFCHDNQTCCRDNRQGWACCPYRQGVCCADR | |
| | RHCCPAGFRCAARGTKCLRREAPRWDAPLRDPALRQLL | |
| 249 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob mutations |
| | ASCCEDRVHCCPHGAFCDLVHTRCITPTGTHPLAKKLPAQRTNRAV | |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALPAPIEKTISKAKGQPREPQVYTLPPSRDELTK | |
| | NQVSLWCLVKGFYPSDIAVEWESNGQPENNYKTTPPVLDSDGSFFL | |
| | YSKLTVDKSRWQQGNVFSCSVMHEALHNHYTQKSLSLSPGK | |
| 250 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob mutations |
| | ASCCEDRVHCCPHGAFCDLVHTRCITPTGTHPLAKKLPAQRTNRAV | |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALPAPIEKTISKAKGQPREPQVYTLPPSRDELTKNQVSL | |
| | WCLVKGFYPSDIAVEWESNGQPENNYKTTPPVLDSDGSFFLYSKLT | |
| | VDKSRWQQGNVFSCSVMHEALHNHYTQKSLSLSPGK | |
| 251 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob and LALA |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALPAPIEKTISKAKGQPREPQVYTLPPSRDELTK | |
| | NQVSLWCLVKGFYPSDIAVEWESNGQPENNYKTTPPVLDSDGSFFL | |
| | YSKLTVDKSRWQQGNVFSCSVMHEALHNHYTQKSLSLSPGK | |
| 252 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob and LALA |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPEAAGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALPAPIEKTISKAKGQPREPQVYTLPPSRDELTKNQVSL | |
| | WCLVKGFYPSDIAVEWESNGQPENNYKTTPPVLDSDGSFFLYSKLT | |
| | VDKSRWQQGNVFSCSVMHEALHNHYTQKSLSLSPGK | |
| 253 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob and LALAPG |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALGAPIEKTISKAKGQPREPQVYTLPPSRDELTK | |
| | NQVSLWCLVKGFYPSDIAVEWESNGQPENNYKTTPPVLDSDGSFFL | |
| | YSKLTVDKSRWQQGNVFSCSVMHEALHNHYTQKSLSLSPGK | |
| 254 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob and LALAPG |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPEAAGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALGAPIEKTISKAKGQPREPQVYTLPPSRDELTKNQVS | |
| | LWCLVKGFYPSDIAVEWESNGQPENNYKTTPPVLDSDGSFFLYSKL | |
| | TVDKSRWQQGNVFSCSVMHEALHNHYTQKSLSLSPGK | |
| 255 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob and LS |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALPAPIEKTISKAKGQPREPQVYTLPPSRDELTK | |
| | NQVSLWCLVKGFYPSDIAVEWESNGQPENNYKTTPPVLDSDGSFFL | |
| | YSKLTVDKSRWQQGNVFSCSVLHEALHSHYTQKSLSLSPGK | |
| 256 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob and LS |
| | ASCCEDRVHCCPHGAFCDLVHTRCITPTGTHPLAKKLPAQRTNRAV | mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALPAPIEKTISKAKGQPREPQVYTLPPSRDELTKNQVSL | |
| | WCLVKGFYPSDIAVEWESNGQPENNYKTTPPVLDSDGSFFLYSKLT | |
| | VDKSRWQQGNVFSCSVLHEALHSHYTQKSLSLSPGK | |
| 257 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob, LALA, and |
| | ASCCEDRVHCCPHGAFCDLVHTRCITPTGTHPLAKKLPAQRTNRAV | LS mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPGGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALPAPIEKTISKAKGQPREPQVYTLPPSRDELTK | |
| | NQVSLWCLVKGFYPSDIAVEWESNGQPENNYKTTPPVLDSDGSFFL | |
| | YSKLTVDKSRWQQGNVFSCSVLHEALHYTQKSLSLSPGK | |
| 258 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob, LALA, and |
| | ASCCEDRVHCCPHGAFCDLVHTRCITPTGTHPLAKKLPAQRTNRAV | LS mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVEDGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPEAAGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALPAPIEKTISKAKGQPREPQVYTLPPSRDELTKNQVSL | |
| | WCLVKGFYPSDIAVEWESNGQPENNYKTTPPVLDSDGSFFLYSKLT | |
| | VDKSRWQQGNVFSCSVLHEALHSHYTQKSLSLSPGK | |
| 259 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob, LALAPG, |
| | ASCCEDRVHCCPHGAFCDLVHTRCITPTGTHPLAKKLPAQRTNRAV | and LS mutations |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPEAAGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALGAPIEKTISKAKGQPREPQVYTLPPSRDELTK | |
| | NQVSLWCLVKGFYPSDIAVEWESNGQPENNYKTTPPVLDSDGSFFL | |
| | YSKLTVDKSRWQQGNVFSCSVLHEALHSHYTQKSLSLSPGK | |
| 260 | TRCPDGQFCPVACCLDPGGASYSCCRPLLDKWPTTLSRHLGGPCQV | PGRN-G₄S-Partial |
| | DAHCSAGHSCIFTVSGTSSCCPFPEAVACGDGHHCCPRGFHCSADG | hinge-Fc polypeptide |
| | RSCFQRSGNNSVGAIQCPDSQFECPDFSTCCVMVDGSWGCCPMPQ | with knob, LALAPG, |
| | ASCCEDRVHCCPHGAFCDLVHTRCITPTGTHPLAKKLPAQRTNRAV | and LS mutations |
| | ALSSSVMCPDARSRCPDGSTCCELPSGKYGCCPMPNATCCSDHLHC | |
| | CPQDTVCDLIQSKCLSKENATTDLLTKPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSDKTHTCPP | |
| | CPAPEAAGGPSVFLFPPKPKDTLMISRTPEVTCVVVDVSHEDPEVKF | |
| | NWYVDGVEVHNAKTKPREEQYNSTYRVVSVLTVLHQDWLNGKE | |
| | YKCKVSNKALGAPIEKTISKAKGQPREPQVYTLPPSRDELTKNQVS | |
| | LWCLVKGFYPSDIAVEWESNGQPENNYKTTPPVLDSDGSFFLYSKL | |
| | TVDKSRWQQGNVFSCSVHEALHSHYTQKSLSLSPGK | |
| 261 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with knob |
| | DWLNGKEYKCKVSNKALPAPIEKTISKAKGQPREPQVYTLPPSRDE | mutation |
| | LTKNQVSLWCLVKGFYPSDIAVEWESNGQPENNYKTTPPVLDSDG | |
| | SFFLYSKLTVDKSRWQQGNVFSCSVMHEALHNHYTQKSLSLSPGK | |
| 262 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with knob and |
| | DWLNGKEYKCKVSNKALPAPIEKTISKAKGQPREPQVYTLPPSRDE | LALA mutations |
| | LTKNQVSLWCLVKGFYPSDIAVEWESNGQPENNYKTTPPVLDSDG | |
| | SFFLYSKLTVDKSRWQQGNVFSCSVMHEALHNHYTQKSLSLSPGK | |
| 263 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with knob and |
| | DWLNGKEYKCKVSNKALGAPIEKTISKAKGQPREPQVYTLPPSRDE | LALAPG mutations |
| | LTKNQVSLWCLVKGFYPSDIAVEWESNGQPENNYKTTPPVLDSDG | |
| | SFFLYSKLTVDKSRWQQGNVFSCSVMHEALHNHYTQKSLSLSPGK | |
| 264 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with knob and |
| | DWLNGKEYKCKVSNKALPAPIEKTISKAKGQPREPQVYTLPPSRDE | LS mutations |
| | LTKNQVSLWCLVKGFYPSDIAVEWESNGQPENNYKTTPPVLDSDG | |
| | SFFLYSKLTVDKSRWQQGNVFSCSVLHEALHSHYTQKSLSLSPGK | |
| 265 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with knob, |
| | DWLNGKEYKCKVSNKALPAPIEKTISKAKGQPREPQVYTLPPSRDE | LALA, and LS |
| | LTKNQVSLWCLVKGFYPSDIAVEWESNGQPENNYKTTPPVLDSDG | mutations |
| | SFFLYSKLTVDKSRWQQGNVFSCSVLHEALHSHYTQKSLSLSPGK | |
| 266 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with knob, |
| | DWLNGKEYKCKVSNKALGAPIEKTISKAKGQPREPQVYTLPPSRDE | LALAPG, and LS |
| | LTKNQVSLWCLVKGFYPSDIAVEWESNGQPENNYKTTPPVLDSDG | mutations |
| | SFFLYSKLTVDKSRWQQGNVFSCSVLHEALHNHYTQKSLSLSPGK | |
| 267 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with hole |
| | DWLNGKEYKCKVSNKALPAPIEKTISKAKGQPREPQVYTLPPSRDE | mutations |
| | LTKNQVSLSCAVKGFYPSDIAVEWESNGQPENNYKTTPPVLDSDGS | |
| | FFLVSKLTVDKSRWQQGNVFSCSVMHEALHNHYTQKSLSLSPGK | |
| 268 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with hole and |
| | DWLNGKEYKCKVSNKALPAPIEKTISKAKGQPREPQVYTLPPSRDE | LALA mutations |
| | LTKNQVSLSCAVKGFYPSDIAVEWESNGQPENNYKTTPPVLDSDGS | |
| | FFLVSKLTVDKSRWQQGNVFSCSVMHEALHNHYTQKSLSLSPGK | |
| 269 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with hole and |
| | DWLNGKEYKCKVSNKALGAPIEKTISKAKGQPREPQVYTLPPSRDE | LALAPG mutations |
| | LTKNQVSLSCAVKGFYPSDIAVEWESNGQPENNYKTTPPVLDSDGS | |
| | FFLVSKLTVDKSRWQQGNVFSCSVMHEALHNHYTQKSLSLSPGK | |
| 270 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with hole and |
| | DWLNGKEYCKCSVSNKALPAPIEKTISKAKGQPREPQVYTLPPSRDE | LS mutations |
| | LTKNQVSLSCAVKGFYPSDIAVEWESNGQPENNYKTTPPVLDSDGS | |
| | FFLVSKLTVDKSRWQQGNVFSCSVLHEALHSHYTQKSLSLSPGK | |
| 271 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with hole, |
| | DWLNGKEYKCKVSNKALPAPIEKTISKAKGQPREPQVYTLPPSRDE | LALA, and LS |
| | LTKNQVSLSCAVKGFYPSDIAVEWESNGQPENNYKTTPPVLDSDGS | mutations |
| | FFLVSKLTVDKSRWQQGNVFSCSVLHEALHSHYTQKSLSLSPGK | |
| 272 | DKTHTCPPCPAPEAAGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | sequence with hole, |
| | DWLNGKEYKCKVSNKALGAPIEKTISKAKGQPREPQVYTLPPSRDE | LALAPG, and LS |
| | LTKNQVSLSCAVKGFYPSDIAVEWESNGQPENNYKTTPPVLDSDGS | mutations |
| | FFLVSKLTVDKSRWQQGNVFSCSVLHEALHSHYTQKSLSLSPGK | |
| 273 | DKTHTCPPCPAPELLGGPSVFLFPPKPKDTLMISRTPEVTCVVVDVS | Partial hinge- |
| | HEDPEVKFNWYVDGVEVHNAKTKPREEQYNSTYRVVSVLTVLHQ | CH3C.35.21.17 Fc knob |
| | DWLNGKEYKCKVSNKALPAPIEKTISKAKGQPREPQVYTLPPSRDE | |
| | LTKNQVSLWCLVKGFYPSDIAVLWESYGTEWSSYKTTPPVLDSDG | |
| | SFFLYSKLTVTKEEWQQGFVFSCSVMHEALHNHYTQKSLSLSPGK | |
| 274 | DKTHTCPPCPAPELLGGPSVFLFPPKPKDTLMISRTPEVTCVVVDVS | Partial hinge- |
| | HEDPEVKFNWYVDGVEVHNAKTKPREEQYNSTYRVVSVLTVLHQ | CH3C.35.21.17 Fc knob- |
| | DWLNGKEYKCKVSNKALPAPIEKTISKAKGQPREPQVYTLPPSRDE | (G₄S)₂-PGRN |
| | LTKNQVSLWCLVKGFYPSDIAVLWESYGTEWSSYKTTPPVLDSDG | |
| | SFFLYSKLTVTKEEWQQGFVFSCSVMHEALHNHYTQKSLSLSPGKG | |
| | GGGSGGGGSTRCPDGQFCPVACCLDPGGASYSCCRPLLDKWPTTL | |
| | SRHLGGPCQVDAHCSAGHSCIFTVSGTSSCCPFPEAVACGDGHHCC | |
| | PRGFHCSADGRSCFQRSGNNSVGAIQCPDSQFECPDFSTCCVMVDG | |
| | SWGCCPMPQASCCEDRVHCCPHGAFCDLVHTRCITPTGTHPLAKK | |
| | LPAQRTNRAVALSSSVMCPDARSRCPDGSTCCELPSGKYGCCPMPN | |
| | ATCCSDHLHCCPQDTVCDLIQSKCLSKENATTDLLTKLPAHTVGDV | |
| | KCDMEVSCPDGYTCCRLQSGAWGCCPFTQAVCCEDHIHCCPAGFT | |
| | CDTQKGTCEQGPHQVPWMEKAPAHLSLPDPQALKRDVPCDNVSSC | |
| | PSSDTCCQLTSGEWGCCPIPEAVCCSDHQHCCPQGYTCVAEGQCQR | |
| | GSEIVAGLEKMPARRASLSHPRDIGCDOHTSCPVGOTCCPSLGGSW | |
| | ACCQLPHAVCCEDRQHCCPAGYTCNVKARSCEKEVVSAQPATFLA | |
| | RSPHVGVKDVECGEGHFCHDNQTCCRDNRQGWACCPYRQGVCCA | |
| | DRRHCCPAGFRCAARGTKCLRREAPRWDAPLRDPALRQLL | |
| 275 | TRCPDGQFCPVACCLDPGGASYSCCRPLLDKWPTTLSRHLGGPCQV | PGRN-(G₄S)₂-Partial |
| | DAHCSAGHSCIFTVSGTSSCCPFPEAVACGDGHHCCPRGFHCSADG | hinge-CH3C.35.21.17 Fc |
| | RSCFQRSGNNSVGAIQCPDSQFECPDFSTCCVMVDGSWGCCPMPQ | knob |
| | ASCCEDRVHCCPHGAFCDLVHTRCITPTGTHPLAKKLPAQRTNRAV | |
| | ALSSSVMCPDARSRCPDGSTCCELPSGKYGCCPMPNATCCSDHLHC | |
| | CPQDTVCDLIQSKCLSKENATTDLLTKLPAHTVGDVKCDMEVSCP | |
| | DGYTCCRLQSGAWGCCPFTQAVCCEDHIHCCPAGFTCDTQKGTCE | |
| | QGPHQVPWMEKAPAHLSLPDPQALKRDVPCDNVSSCPSSDTCCQL | |
| | TSGEWGCCPIPEAVCCSDHQHCCPQGYTCVAEGQCQRGSEIVAGLE | |
| | KMPARRASLSHPRDIGCDQHTSCPVGQTCCPSLGGSWACCQLPHA | |
| | VCCEDRQHCCPAGYTCNVKARSCEKEVVSAQPATFLARSPHVGVK | |
| | DVECGEGHFCHDNQTCCRDNRQGWACCPYRQGVCCADRRHCCPA | |
| | GFRCAARGTKCLRREAPRWDAPLRDPALRQLLGGGGSGGGGSDKT | |
| | HTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWL | |
| | NGKEYKCKVSNKALPAPIEKTISKAKGQPREPQVYTLPPSRDELTK | |
| | NQVSLWCLVKGFYPSDIAVLWESYGTEWSSYKTTPPVLDSDGSFFL | |
| | YSKLTVTKEEWQQGFVFSCSVMHEALHNHYTQKSLSLSPGK | |
| 276 | GGGGSGGGGS | Polypeptide linker |
| 277 | GGGGS | Polypeptide linker |
| 278 | MWTLVSWVALTAGLVAG | Progranulin signal |
| | | sequence |
| 279 | DVECGEGHFCHDNQTCCRDNRQGWACCPYRQGVCCADRRHCCPA | Granulin E (amine acids |
| | GFRCAARGTKCL | 518-573 of SEQ ID |
| | | NO: 211) |
| 280 | DKTHTCPPCPAPELLGGPSVFLFPPKPKDTLMISRTPEVTCVVVDVS | Partial hinge-Fc |
| | HEDPEVKFNWYVDGVEVHNAKTKPREEQYNSTYRVVSVLTVLHQ | polypeptide with hole |
| | DWLNGKEYKCKVSNKALPAPIEKTISKAKGQPREPQVYTLPPSRDE | mutations-(G₄S)₂- |
| | LTKNQVSLSCAVKGFYPSDIAVEWESNGQPENNYKTTPPVLDSDGS | Granulin E fusion |
| | FFLVSKLTVDKSRWQQGNVFSCSVMHEALHNHYTQKSLSLSPGKG | (amino acids 497-593 of |
| | GGGSGGGGSKEVVSAQPATFLARSPHVGVKDVECGEGHFCHDNQT | SEQ ID NO: 211) |
| | CCRDNRQGWACCPYRQGVCCADRRHCCPAGFRCAARGTKCLRRE | |
| | APRWDAPLRDPALRQLL | |
| 281 | DKTHTCPPCPAPELLGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.23.2 with knob |
| | DWLNGKEYKCKVSNKALPAPIEKTISKAKGQPREPQVYTLPPSRDE | mutation |
| | LTKNQVSLWCLVKGFYPSDIAVEWESYGTEWANYKTTPPVLDSDG | |
| | SFFLYSKLTVTKEEWQQGFVFSCSVMHEALHNHYTQKSLSLSPGK | |
| 282 | DKTHTCPPCPAPEAAGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.23.2 with knob |
| | DWLNGKEYKCKVSNKALGAPIEKTISKAKGQPREPQVYTLPPSRDE | and LALAPG mutations |
| | LTKNQVSLWCLVKGFYPSDIAVEWESYGTEWANYKTTPPVLDSDG | |
| | SFFLYSKLTVTKEEWQQGFVFSCSVMHEALHNHYTQKSLSLSPGK | |
| 283 | DKTHTCPPCPAPELLGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.23.2 with knob |
| | DWLNGKEYKCKVSNKALPAPIEKTISKAKGQPREPQVYTLPPSRDE | and LS mutations |
| | LTKNQVSLWCLVKGFYPSDIAVEWESYGTEWANYKTTPPVLDSDG | |
| | SFFLYSKLTVTKEEWQQGFVFSCSVLHEALHSHYTQKSLSLPGK | |
| 284 | DKTHTCPPCPAPEAAGGPSVFLFPPKPKDTLMISRTPEVTCVVVDVD | Partial hinge-Clone |
| | HEDPEVKFNWYVDGVEVHNAKTKPREEQYNSTYRVVSVLTVLHQ | CH3C.35.23.2 with |
| | DWLNGKEYKCKVSNKALPAPIEKTISKAKGQPREPQVYTLPPSRDE | knob, LALA, and LS |
| | LTKNQVSLWCLVKGFYPSDIAVEWESYGTEWANYKTTPPVLDSDG | mutations |
| | SFFLYSKLTVTKEEWQQGFVFSCSVLHEALHSHYTQKSLSLSPGK | |
| 285 | DKTHTCPPCPAPEAAGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.23.2 with |
| | DWLNGKEYKCKVSNKALGAPIEKTISKAKGQPREPQVYTLPPSRDE | knob, LALAPG, and LS |
| | LTKNQVSLWCLVKGFYPSDIAVEWESYGTEWANYKTTPPVLDSDG | mutations |
| | SFFLYSKLTVTKEEWQQGFVFSCSVLHEALHSHYTQKSLSLSPGK | |
| 286 | DKTHTCPPCPAPELLGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17.2 with |
| | DWLNGKEYKCKVSNKALPAPIEKTISKAKGQPREPQVYTLPPSRDE | knob mutation |
| | LTKNQVSLWCLVKGFYPSDIAVLWESYGTEWASYKTTPPVLDSDG | |
| | SFFLYSKLTVTKEEWQQGFVFSCSVMHEALHNHYTQKSLSLSPGK | |
| 287 | DKTHTCPPCPAPEAAGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17.2 with |
| | DWLNGKEYKCKVSNKALGAPIEKTISKAKGQPREPQVYTLPPSRDE | knob and LALAPG |
| | LTKNQVSLWCLVKGFYPSDIAVLWESYGTEWASYKTTPPVLDSDG | mutations |
| | SFFLYSKLTVTKEEWQQGFVFSCSVMHEALHNHYTQKSLSLSPGK | |
| 288 | DKTHTCPPCPAPELLGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17.2 with |
| | DWLNGKEYKCKVSNKALPAPIEKTISKAKGQPREPQVYTLPPSRDE | knob and LS mutations |
| | LTKNQVSLWCLVKGFYPSDIAVLWESYGTEWASYKTTPPVLDSDG | |
| | SFFLYSKLTVTKEEWQQGFVFSCSVLHEALHSHYTQKSLSLSPGK | |
| 289 | DKTHTCPPCPAPEAAGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17.2 with |
| | DWLNGKEYKCKVSNKALPAPIEKTISKAKGQPREPQVYTLPPSRDE | knob, LALA, and LS |
| | LTKNQVSLWCLVKGFYPSDIAVLWESYGTEWASYKTTPPVLDSDG | mutations |
| | SFFLYSKLTVTKEEWQQGFVFSCSVLHEALSHYTQKSLSLSPGL | |
| 290 | DKTHTCPPCPAPEAAGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17.2 with |
| | DWLNGKEYKCKVSNKALGAPIEKTISKAKGQPREPQVYTLPPSRDE | knob, LALAPG, and LS |
| | LTKNQVSLWCLVKGFYPSDIAVLWESYGTEWASYKTTPPVLDSDG | mutations |
| | SFFLYSKLTVTKEEWQQGFVFSCSVLHEALHSHYTQKSLSLSPGK | |
| 291 | DKTHTCPPCPAPEAAGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17 with |
| | DWLNGKEYKCKVSNKALPAPIEKTISKAKGQPREPQVYTLPPSRDE | knob and LALA |
| | LTKNQVSLWCLVKGFYPSDIAVLWESYGTEWSSYKTTPPVLDSDG | mutations |
| | SFFLYSKLTVTKEEWQQGFVFSCSVMHEALHNHYTQKSLSLSPGK | |
| 292 | DKTHTCPPCPAPEAAGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17 with |
| | DWLNGKEYKCKVSNKALGAPIEKTISKAKGQPREPQVYTLPPSRDE | knob and LALAPG |
| | LTKNQVSLWCLVKGFYPSDIAVLWESYGTEWSSYKTTPPVLDSDG | mutations |
| | SFFLYSKLTVTKEEWQQGFVFSCSVMHEALHNHYTQKSLSLSPGK | |
| 293 | DKTHTCPPCPAPELLGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17 with |
| | DWLNGKEYKCKVSNKALPAPIEKTISKAKGQPREPQVYTLPPSRDE | knob and LS mutations |
| | LTKNQVSLWCLVKGFYPSDIAVLWESYGTEWSSYKTTPPVLDSDG | |
| | SFFLYSKLTVTKEEWQQGFVFSCSVLHEALHSHYTQKSLSLSPGK | |
| 294 | DKTHTCPPCPAPEAAGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17 with |
| | DWLNGKEYKCKVSNKALPAPIEKTISKAKGQPREPQVYTLPPSRDE | knob, LALA, and LS |
| | LTKNQVSLWCLVKGFYPSDIAVLEWSYGTEWSSYKTTPPVLDSDG | mutations |
| | SFFLYSKLTVTKEEWQQGFVFSCSVLHEALHSHYTQKSLSLSPGK | |
| 295 | DKTHTCPPCPAPEAAGGPSVFLFPPKPKDTLMISRTPEVTCVVVDVS | Partial hinge-Clone |
| | HEDPEVKFNWYVDGVEVHNAKTKPREEQYNSTYRVVSVLTVLHQ | CH3C.35.21.17 with |
| | DWLNGKEYKCKVSNKALGAPIEKTISKAKGQPREPQVYTLPPSRDE | knob, LALAPG, and LS |
| | LTKNQVSLWCLVKGFYPSDIAVLWESYGTEWSSYKTTPPVLDSDG | mutations |
| | SFFLYSKLTVTKEEWQQGFVFSCSVLHEALHSHYTQKSLSLSPGK | |
| 296 | AQNSVIIVDKNGRLVYLVENPGGYVAYSKAATVTGKLVHANFGTK | Apical domain insert of |
| | KDFEDLYTPVNGSIVIVRAGKITFAEKVANAESLNAIGVLIYMDQTK | human transferrin |
| | FPIVNAELSFFGHAHLGTGDPYTPGFPSFNHTQFPPSRSSGLPNIPVQ | receptor protein 1 |
| | TISRAAAEKLFGNMEGDCPSDWKTDSTCRMVTSESKNVKLTVSN | (TFR1) |
| 297 | AQNSVIIVDKNGGLVYLVENPGGYVAYSKAATVTGKLVHANFGTK | Apical domian of |
| | KDFEDLDSPVNGSIVIVRAGKITFAEKVANAESLNAIGVLIYMDQTK | Macaca mulatta (rhesus |
| | FPIVKADLSFFGHAHLGTGDPYTPGFPSFNHTQFPPSQSSGLPNIPVQ | monkey) TfR (NCBI |
| | TISRAAAEKLFGNMEGDCPSDWKTDSTCKMVTSENKSVKLTVSN | Reference Sequence |
| | | NP_001244232.1); it has |
| | | 95% identity to the |
| | | apical domain of the |
| | | native human TfR |
| 298 | AQNSVIIVDKNGLVYLVENPGGYVAYSKAATVTGKLVHANFGTK | Apical domain of |
| | KDFEDLHTPVNGSIVIVRAGKITFAEKVANAESLNAIGVLIYMDQTK | chimpanzee TfR (NCBI |
| | FPIVNAELSFFGHAHLGTGDPYTPGFPSFNHTQFPPSRSSGLPNIPVQ | Reference Sequence |
| | TVSRAAAEKLFGNMEGDCPSDWKTDSTCRMVTSESKNVKLTVSN | XP_003310238.1); it is |
| | | 98% identical to the |
| | | apical domain of the |
| | | native human TfR |
| 299 | AQNSVIIVDKNGGLVYLVENPGGYVAYSKAATVTGKLVHANFGTK | Apical domain of |
| | KDFEDLDSPVNGSIVIVRAGKITFAEKVANAESLNAIGVLIYMDQTK | Macaca fascicularis |
| | FPIVKADLSFFGHAHLGTGDPYTPGFPSFNHTQFPPSQSSGLPNIPVQ | (cynomolgous monkey) |
| | TISRAAAEKLFGNMEGDCPSDWKTDSTCKMVTSENKSVKLTVSN | TfR (NCBI Reference |
| | | Sequence |
| | | XP_005545315); it is |
| | | 96% identical to the |
| | | apical domain of the |
| | | native human TfR |
| 300 | 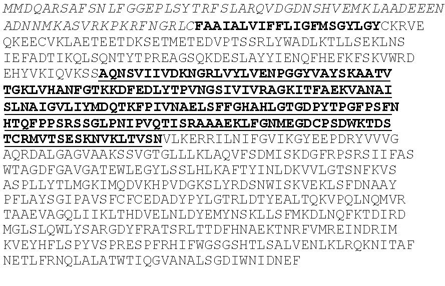 | Chimeric TfR polypeptide sequence expressed in transgenic mouse (The italicized portion represents the cytoplasmic domain, the bolded portion represents the transmembrane domain, the portion in grey represents the extracellular domain, and the bold and underlined portion represents the apical domain) |
| 301 | GCTCAGAACTCCGTGATCATCGTGGATAAGAACGGCCGGCTGGT | DNA sequence of human |
| | GTACCTGGTGGAGAACCCTGGCGGATACGTGGCTTACTCTAAGG | apical domain insert |
| | CCGCTACCGTGACAGGCAAGCTGGTGCACGCCAACTTCGGAACC | |
| | AAGAAGGACTTTGAGGATCTGTACACACCAGTGAACGGCTCTAT | |
| | CGTGATCGTGCGCGCTGGAAAGATCACCTTCGCCGAGAAGGTGG | |
| | CTAACGCCGAGAGCCTGAACGCCATCGGCGTGCTGATCTACATG | |
| | GATCAGACAAAGTTTCCCATCGTGAACGCTGAGCTGTCTTTCTTT | |
| | GGACACGCTCACCTGGGCACCGGAGACCCATACACACCCGGATT | |
| | CCCTAGCTTTAACCACACCCAGTTCCCCCCTTCCAGGTCTAGCGG | |
| | ACTGCCAAACATCCCCGTGCAGACAATCAGCAGAGCCGCTGCCG | |
| | AGAAGCTGTTTGGCAACATGGAGGGAGACTGCCCCTCCGATTGG | |
| | AAGACCGACTCTACATGTAGGATGGTGACCTCCGAGTCAAAAAA | |
| | TGTCAAACTCACCGTGTCCAAT | |
| 302 | YxTEWSS | Library motif |
| 303 | 6xHis | His tag |
| 304 | GGSG | Polypeptide linker |
| 305 | SGGG | Polypeptide linker |
| 306 | KESGSVSSEQLAQFRSLD | Polypeptide linker |
| 307 | EGKSSGSGSESKST | Polypeptide linker |
| 308 | GSAGSAAGSGEF | Polypeptide linker |
| 309 | AEAAAKA | Polypeptide linker |

1-159. (canceled)

160. A protein comprising:
(a) a first Fc polypeptide that is linked to a progranulin polypeptide or a variant thereof, and
(b) a second Fc polypeptide that forms an Fc dimer with the first Fc polypeptide, wherein the second Fc polypeptide specifically binds to a transferrin receptor.

161. The protein of claim 160, wherein the protein comprises exactly one progranulin polypeptide or variant thereof.

162. The protein of claim 160, wherein the second Fc polypeptide is linked to a second progranulin polypeptide or variant thereof.

163. The protein of claim 160, wherein the first Fc polypeptide and the second Fc polypeptide do not include an immunoglobulin heavy and/or light chain variable region sequence or an antigen-binding portion thereof.

164. The protein of claim 160, wherein the progranulin polypeptide comprises an amino acid sequence having at least 90% identity, or at least 95% identity to the amino acid sequence of SEQ ID NO:212.

165. The protein of claim 160, wherein the first Fc polypeptide is linked to the progranulin polypeptide or the variant thereof by a peptide bond or by a polypeptide linker.

166. The protein of claim 165, wherein the polypeptide linker is 1 to 50 amino acids in length.

167. The protein of claim 165, wherein the polypeptide linker is a flexible polypeptide linker.

168. The protein of claim 167, wherein the flexible polypeptide linker is a glycine-rich linker.

169. The protein of claim 160, wherein the N-terminus or the C-terminus of the first Fc polypeptide is linked to the progranulin polypeptide.

170. The protein of claim 162, wherein the N-terminus or the C-terminus of the second Fc polypeptide is linked to the second progranulin polypeptide.

171. The protein of claim 160, wherein the first Fc polypeptide and the second Fc polypeptide each contain modifications that promote heterodimerization.

172. The protein of claim 171, wherein one of the Fc polypeptides has a T366W substitution and the other Fc polypeptide has T366S, L368A, and Y407V substitutions, according to EU numbering.

173. The protein of claim 172, wherein the first Fc polypeptide contains the T366S, L368A, and Y407V substitutions and the second Fc polypeptide contains the T366W substitution.

174. The protein of claim 173, wherein the first Fc polypeptide is linked to the progranulin polypeptide and comprises the amino acid sequence of any one of SEQ ID NOS:213, 214, 225, and 226, and the second Fc polypeptide comprises the amino acid sequence of SEQ ID NO:261.

175. The protein of claim 160, wherein the first Fc polypeptide and/or the second Fc polypeptide comprises a native FcRn binding site.

176. The protein of claim 160, wherein the first Fc polypeptide and/or the second Fc polypeptide includes a modification that reduces effector function.

177. The protein of claim 176, wherein the modification that reduces effector function is the substitutions of Ala at position 234 and Ala at position 235, according to EU numbering.

178. The protein of claim 160, wherein the first Fc polypeptide and/or the second Fc polypeptide comprises amino acid changes relative to the native Fc sequence that extend serum half-life.

179. The protein of claim 178, wherein:
(a) the amino acid changes comprise substitutions of Leu at position 428 and Ser at position 434, according to EU numbering; or
(b) the amino acid changes comprise a substitution of Ser or Ala at position 434, according to EU numbering.

180. The protein of claim 160, wherein the second Fc polypeptide binds to the apical domain of the transferrin receptor.

181. The protein of claim 180, wherein the second Fc polypeptide comprises at least two substitutions at positions selected from the group consisting of 384, 386, 387, 388, 389, 390, 413, 416, and 421, according to EU numbering.

182. The protein of claim 181, wherein the second Fc polypeptide includes substitutions at at least three, four, five, six, seven, eight, or nine of the positions.

183. The protein of claim 181, wherein the second Fc polypeptide further comprises one, two, three, or four substitutions at positions comprising 380, 391, 392, and 415, according to EU numbering.

184. The protein of claim 181, wherein the second Fc polypeptide further comprises one, two, or three substitutions at positions comprising 414, 424, and 426, according to EU numbering.

185. The protein of claim 181, wherein the second Fc polypeptide comprises (i) Trp at position 388 and/or (ii) an aromatic amino acid at position 421.

186. The protein of claim 185, wherein the aromatic amino acid at position 421 is Trp or Phe.

187. The protein of claim 181, wherein the second Fc polypeptide comprises at least one position selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

188. The protein of claim 187, wherein the second Fc polypeptide comprises 2, 3, 4, 5, 6, 7, 8, 9, 10, or 11 positions selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

189. The protein of claim 188, wherein the second Fc polypeptide comprises 11 positions as follows: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

190. The protein of claim 188, wherein the second Fc polypeptide has a CH3 domain with at least 85% identity, at least 90% identity, or at least 95% identity to amino acids 111-217 of any one of SEQ ID NOS:34-38, 58, 60-90, 136, and 137-210.

191. The protein of claim 190, wherein the residues at at least 5, 6, 7, 8, 9, 10, 11, 12, 13, 14, 15, or 16 of the positions corresponding to EU index positions 380, 384, 386, 387, 388, 389, 390, 391, 392, 413, 414, 415, 416, 421, 424 and 426 of any one of SEQ ID NOS:34-38, 58, 60-90, 136, and 137-210 are not deleted or substituted.

192. The protein of claim 190, wherein the second Fc polypeptide comprises the amino acid sequence of any one of SEQ ID NOS:136-210.

193. The protein of claim 192, wherein the second Fc polypeptide comprises the amino acid sequence of any one of SEQ ID NOS:136, 138, 150, 162, 174, 186, and 198.

194. The protein of claim 160, wherein the binding of the protein to the transferrin receptor does not substantially inhibit binding of transferrin to the transferrin receptor.

195. The protein of claim 160, wherein the progranulin polypeptide or the variant thereof binds to sortilin or prosaposin.

196. The protein of claim 160, wherein (a) comprises the sequence of any one of SEQ ID NOS:213, 214, 225, and 226 and (b) comprises the sequence of SEQ ID NO:281 or 286.

197. The protein of claim 196, wherein the first Fc polypeptide and/or the second Fc polypeptide further comprises L234A and L235A mutations with or without P329G mutation, and/or M428L and N434S mutations, according to EU numbering scheme.

198. The protein of claim 197, wherein:
(i) (a) comprises the sequence of any one of SEQ ID NOS:213, 214, 225, and 226 and (b) comprises the sequence of any one of SEQ ID NOS:209, 210, 282-285, and 287-291; or
(ii) (a) comprises the sequence of any one of SEQ ID NOS:215-224 and 227-236 and (b) comprises the sequence of SEQ ID NO:281 or 286; or
(iii) (a) comprises the sequence of any one of SEQ ID NOS:215-224 and 227-236 and (b) comprises the sequence of SEQ ID NO:209, 210, 282-285, and 287-291.

199. A polypeptide comprising an Fc polypeptide that is linked to a progranulin polypeptide or a variant thereof, wherein the Fc polypeptide contains one or more modifications that promote its heterodimerization to another Fc polypeptide.

200. A method of treating a progranulin-associated disorder, the method comprising administering the protein of claim 160 to a patient in need thereof, wherein the progranulin-associated disorder is selected from the group consisting of a neurodegenerative disease, atherosclerosis, a disorder associated with TDP-43, and age-related macular degeneration (AMD).

201. A method of increasing the amount of a progranulin polypeptide or a variant thereof in a patient having a progranulin-associated disorder, the method comprising administering the protein of claim 160 to the patient, wherein the progranulin-associated disorder is selected from the group consisting of a neurodegenerative disease, atherosclerosis, a disorder associated with TDP-43, and age-related macular degeneration (AMD).

202. A method of decreasing cathepsin D activity in a patient having a progranulin-associated disorder, the method comprising administering the protein of claim 160 to the patient, wherein the progranulin-associated disorder is selected from the group consisting of a neurodegenerative disease, atherosclerosis, a disorder associated with TDP-43, and age-related macular degeneration (AMD).

203. A method of increasing lysosomal degradation in a patient having a progranulin-associated disorder, the method comprising administering the protein of claim 160 to the patient, wherein the progranulin-associated disorder is selected from the group consisting of a neurodegenerative disease, atherosclerosis, a disorder associated with TDP-43, and age-related macular degeneration (AMD).

204. A pharmaceutical composition comprising the protein of claim 160 and a pharmaceutically acceptable carrier.
